(19) United States
(12) Patent Application Publication
(10) Pub. No.: US 2022/0227796 A1
(43) Pub. Date: Jul. 21, 2022

(54) EGFR INHIBITORS, COMPOSITIONS AND METHODS THERE OF

(71) Applicant: Betta Pharmaceuticals Co., Ltd., Zhejiang (CN)

(72) Inventors: Xiangyong LIU, Beijing (CN); Changyong QIU, Beijing (CN); Qichao SHEN, Beijing (CN); Mengqiang LIU, Beijing (CN); Haitong SHENG, Beijing (CN); Guolong DU, Beijing (CN); Jiabing WANG, Beijing (CN); Lieming DING, Zhejiang (CN)

(21) Appl. No.: 17/439,324

(22) PCT Filed: Mar. 31, 2020
(86) PCT No.: PCT/CN2020/082347

§ 371 Date: Sep. 14, 2021

(30) Foreign Application Priority Data
Apr. 4, 2019 (CN) PCT/CN2019/081615
Apr. 4, 2019 (CN) PCT/CN2019/081616
Apr. 4, 2019 (CN) PCT/CN2019/081617
Apr. 4, 2019 (CN) PCT/CN2019/081618

(51) Int. Cl. C07F 9/6512; C07F 9/6561; A61P 35/00

(52) U.S. Cl. CPC C07F 9/6512; A61P 35/00; C07F 9/6561

(57) ABSTRACT

The present invention relates to compounds of Formula I, methods of using the compounds as EGFR inhibitors, and pharmaceutical compositions comprising such compounds. The compounds are useful in treating, preventing or ameliorating diseases or disorders such as cancer or infections.

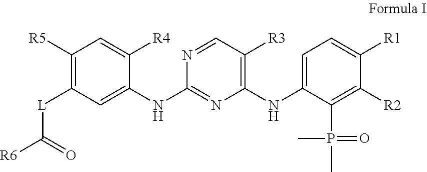

### FIELD OF THE INVENTION

The present invention relates to pharmaceutically active compounds, deuterinated compounds (hydrogen replaced with deuterium), and pharmaceutically acceptable salt thereof which may be useful in treatment or prevention of a disease or medical condition mediated through certain mutated forms of Epidermal Growth Factor Receptor (for example the L858R activating mutant, the Exon19 deletion activating mutant, the T790M resistance mutant, and the C797S resistance mutant). The invention also relates to pharmaceutical compositions comprising said compounds and to methods of treatment of diseases mediated by various different forms of EGFR mutant using said compounds, deuterinated compounds and salts thereof.

### BACKGROUND OF THE INVENTION

Epidermal Growth Factor Receptor (EGFR) is a transmembrane glycoprotein that belongs to ErbB family of tyrosine kinase receptors. Activation of EGFR leads to autophosphorylation of receptor tyrosine kinase that initiates a cascade of downstream signaling pathways involved in regulating cellular proliferation, differentiation, and survival. EGFR is abnormally activated by various mechanisms like receptor overexpression, mutation, ligand-dependent receptor dimerization, ligand-independent activation and is associated with the development of variety of human cancers.

EGFR inhibition is one of the key targets for cancer therapy. Although the previous generations of EGFR-TKIs have developed rapidly, the problem of drug resistance has also followed with the development of drugs. Most of the drug resistance is the T790M mutation in the ATP receptor. The recently developed third-generation series of irreversible inhibitors have very good inhibitory activity against T790M, but inevitably, the acquired mutation of C797S occurs, such as osimertinib. A high percentage of these treated patients developed a tertiary cystein-797 to serine-790 (C797S) mutation in the EGFR kinase domain. This C797S mutation is thought to induce resistance to all current irreversible EGFR TKIs.

In view of the importance of this mutation in resistance to existing therapies targeting EGFR, we believe that compounds which can inhibit EGFR harbouring the L858R, the Δ19del, the T790M, and the C797S may be especially useful in the treatment of cancer.

Osimertinib, also known as AZD9291, is a 3^{rd} EGFR-TKI. It can't inhibit EGFR C797S.

Earlier application, WO2018108064 disclosed a series of 4^{th} EGFR-TKIs which showed very high inhibition of Δ19del/T790M/C797S (triple mutant) cellular phosphorylation.

However, there is currently no inhibitor which possesses properties of 1^{st}, 3^{rd} and 4^{th} EGFR-TKI.

In this regard, there remains a need for compounds that show high inhibition of certain activating mutant (such as L858R) or deletion activating mutant (such as Exon19), while at the same time showing high inhibition of certain resistance mutant (such as T790M, C797S). The applicants have surprisingly found that one or more phenylacrylamide compounds have high potency against several different forms of EGFR, for example, the L858R, the Δ19del, the T790M, and the C797S.

The compounds of the invention may also exhibit advantageous physical properties (for example, higher aqueous solubility, higher permeability, and/or lower plasma protein binding) and/or favorable toxicity profiles (for example, a decreased hERG blocking liability) and/or favorable metabolic profiles in comparison with other known EGFR-TMs. Therefore, such compounds maybe especially useful in the treatment of diseases mediated by various different forms of EGFR mutant, for example in the treatment of cancer.

### SUMMARY OF INVENTION

The present invention relates to compounds which can inhibit various different forms of EGFR harbouring the L858R, the Δ19del, the T790M, and the C797S. These compounds are useful in the treatment of cancers and infectious diseases.

A compound of Formula I, or a stereoisomer, tautomer, deuterinated compound, pharmaceutically acceptable salt, prodrug, chelate, non-covalent complex, or solvate thereof,

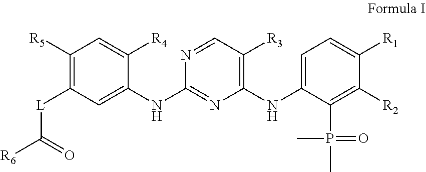

wherein,

R₁ and R₂ are each independently selected from is H, halogen, CN, —C₁₋₆ alkyl or —C₁₋₆ alkoxyl; or

R₁ and R₂ together with the atoms to which they are attached form a 5- to 6-membered heteroaryl ring optionally comprising for 2 hetero atoms independently selected from N, S, or O; or

R₁ and R₂ together with the atoms to which they are attached form an aryl ring;

R₃ is H, halogen, —C₁₋₆ alkyl;

R₄ is H, halogen, —C₁₋₆ alkyl or —C₁₋₆ alkoxyl;

R₅ is —OR₇, —O(CH₂)ₜ—NR₈R₉, —NR₈R₉,

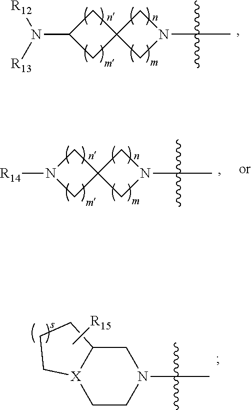

R₆ is H, —C₁₋₆ alkyl, C₂₋₆ alkenyl, C₂₋₆ alkynyl; wherein —C₁₋₆ alkyl, C₂₋₆ alkenyl, C₂₋₆ alkynyl optionally substituted with one or more substituents independently selected from halogen, —C₁₋₆ alkyl, —C₁₋₄ haloalkyl or —NR₁₆R₁₇;

R₇ is C₁₋₆ alkyl, C₃₋₁₀ heteocyclyl, or C₃₋₁₀ heteroaryl;

R₈ and R₉ are each independently selected from —C₁₋₆ alkyl, or —C₁₋₆ alkylene-NR₁₀R₁₁, wherein R₁₀ and R₁₁ are each independently selected from H or —C₁₋₆ alkyl; or R₁₀ and R₁₁ together with the atoms to which they are attached form a 5- to 6-membered heterocyclic ring; or

R₈ and R₉ together with the atoms to which they are attached form a 5- to 6-membered heterocyclic ring;

R₁₂, R₁₃, R₁₄ and R₁₅ are each independently selected from H or —C₁₋₆ alkyl;

R₁₂ and R₁₃ together with the atoms to which they are attached form a 4- to 6-membered ring;

L is a bond, NR₁₈ or (CH₂)ₜ;

R₁₆, R₁₇ and R₁₈ are each independently selected from H, or —C₁₋₆ alkyl;

X is CH or N;

m, n, m′, n′ are each independently selected from 1 or 2;

s and t are each independently selected from 1, 2 or 3.

In some embodiments of Formula I, wherein R₁ and R₂ are each independently selected from is H, CN, and —CH₃.

In some embodiments of Formula I, wherein R₁ and R₂ together with the atoms to which they are attached form

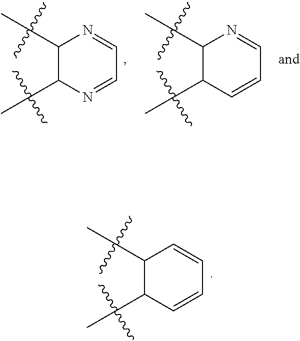 [SMILES: *C1N=CC=NC1* ; "," ; *C1C=CC=NC1* ; "and" ; *C1C=CC=CC1* ; "."]

In some embodiments of Formula I, wherein R₃ is selected from H, F, Cl, Br, CH₃.

In some embodiments of Formula I, wherein R₄ is selected from H, —CH₃, —OCH₃.

In some embodiments of Formula I, wherein R₅ is selected from

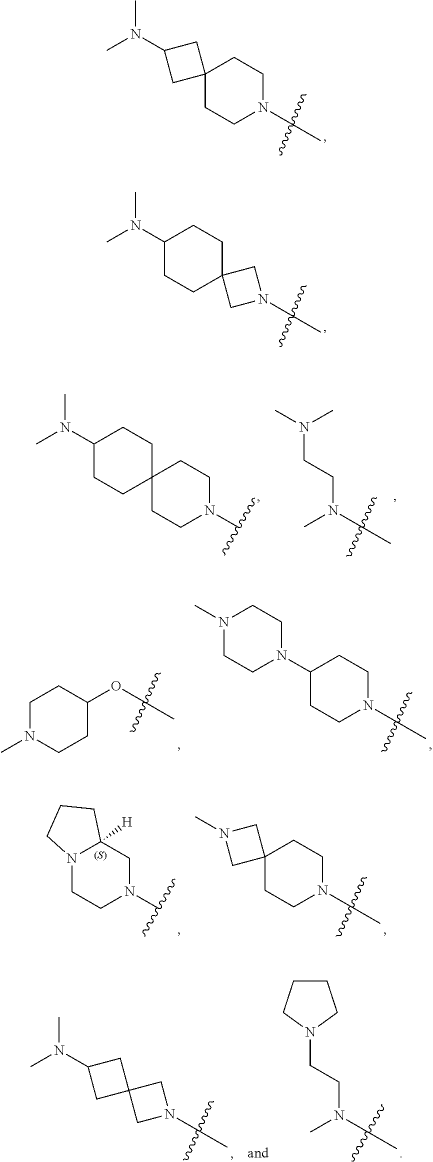

In some embodiments of Formula I, wherein L is selected from NH, and —NCH₃—.

In some embodiments of Formula I, wherein R₆ is selected from

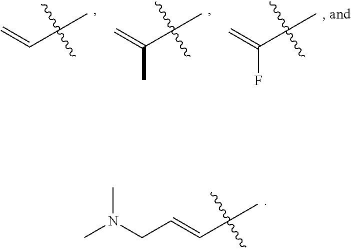

In some embodiments of Formula I, the compound is of Formula II, or a stereoisomer, tautomer, deuterinated compound, pharmaceutically acceptable salt, prodrug, chelate, non-covalent complex, or solvate thereof,

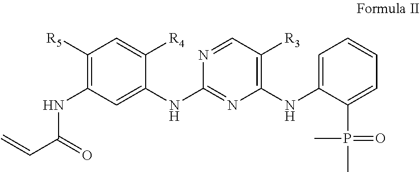

wherein,

R₃ is H, halogen, —C₁₋₆ alkyl;

R₄ is H, halogen, —C₁₋₆ alkyl or —C₁₋₆ alkoxyl;

R₅ is —OR₇, —O(CH₂)ₜ—NR₈R₉, —NR₈R₉,

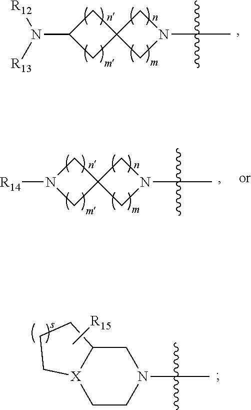

R₇ is C₁₋₆ alkyl, C₃₋₁₀ heteocyclyl, or C₃₋₁₀ heteroaryl;

R₈ and R₉ are each independently selected from —C₁₋₆ alkyl, or —C₁₋₆ alkyl-NR₁₀R₁₁, wherein R₁₀ and R₁₁ are each independently selected from H or —C₁₋₆ alkyl; or R₁₀ and R₁₁ together with the atoms to which they are attached form a 5- to 6-membered heterocyclic ring; or

R₈ and R₉ together with the atoms to which they are attached form a 5- to 6-membered heterocyclic ring;

R₁₂, R₁₃, R₁₄ and R₁₅ are each independently selected from H or —C₁₋₆ alkyl;

R₁₂ and R₁₃ together with the atoms to which they are attached form a 4- to 6-membered ring;

X is CH or N;

m, n, m′, n′ are each independently selected from 1 or 2;

s and t are each independently selected from 1, 2 or 3.

In some embodiments of Formula II, wherein R₃ is selected from H, F, Cl, CH₃.

In some embodiments of Formula II, wherein R₄ is selected from H, —OCH₃.

In some embodiments of Formula II, wherein R₅ is selected from

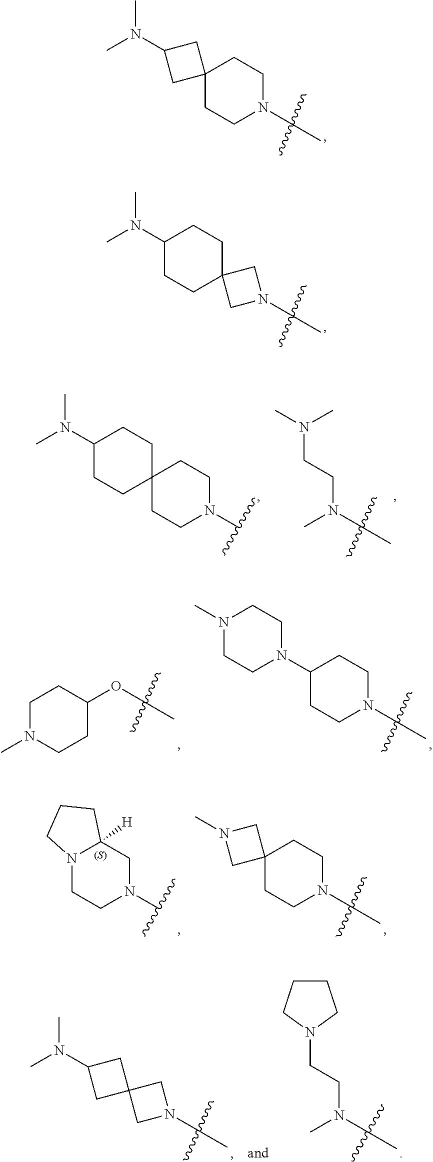

In some embodiments of Formula I, the compound is of Formula III, or a stereoisomer, tautomer, deuterinated compound, pharmaceutically acceptable salt, prodrug, chelate, non-covalent complex, or solvate thereof,

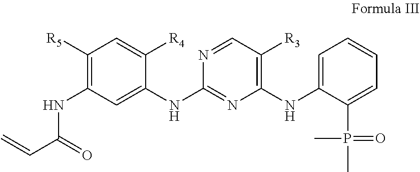

wherein,

R₃ is H, halogen, or C₁₋₆ alkyl;

R₄ is H, halogen, C₁₋₆ alkyl or C₁₋₆ alkoxyl;

R₅ is

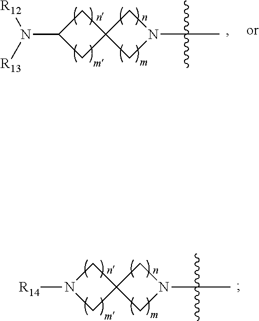

R₁₂, R₁₃, R₁₄ are each independently selected from H or C₁₋₆ alkyl;

R₁₂ and R₁₃ together with the atoms to which they are attached form a 4- to 6-membered ring;

m, n, m′, n′ are each independently selected from 1 or 2.

In some embodiments of Formula III, wherein R₃ is selected from H, Cl.

In some embodiments of Formula III, wherein R₄ is selected from H, —OCH₃.

In some embodiments of Formula III, wherein R₅ is selected from

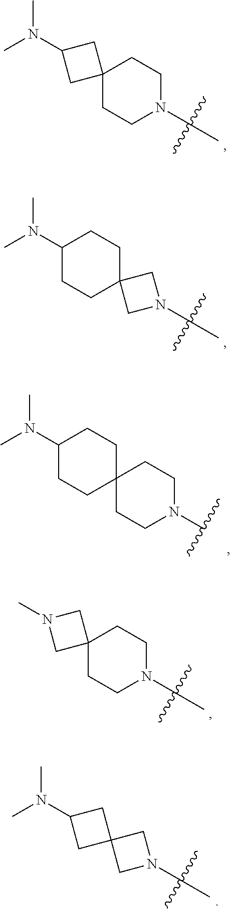 [SMILES: *N1CCC2(CC1)CC(N(C)C)C2 ; *N1CC2(CCC(N(C)C)CC2)C1 ; *N1CCC2(CCC(N(C)C)CC2)CC1 ; *N1CCC2(CC1)CN(C)C2 ; *N1CC2(CC(N(C)C)C2)C1]

In some embodiments of Formula I, the compound is of Formula IV, or a stereoisomer, tautomer, deuterinated compound, pharmaceutically acceptable salt, prodrug, chelate, non-covalent complex, or solvate thereof,

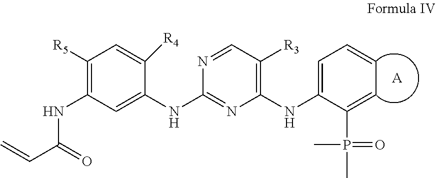

wherein,

Ring A is selected from aryl ring or 5- to 6-membered heteroaryl ring optionally comprising 1 or 2 hetero atoms independently selected from N, S, or O;

R₃ is H, halogen, —C₁₋₆ alkyl;

R₄ is H, halogen, —C₁₋₆ alkyl or —C₁₋₆ alkoxyl;

R₅ is —OR₇, —O(CH₂)ₜ—NR₈R₉, —NR₈R₉,

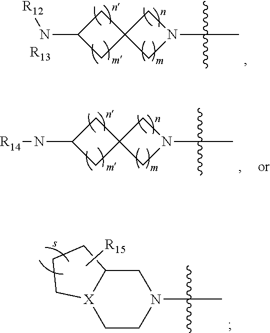

R₇ is C₁₋₆ alkyl, C₃₋₁₀ heteocyclyl, or C₃₋₁₀ heteroaryl;

R₈ and R₉ are each independently selected from —C₁₋₆ alkyl, or —C₁₋₆ alkyl-NR₁₀R₁₁, wherein R₁₀ and R₁₁ are each independently selected from H or —C₁₋₆ alkyl; or R₁₀ and R₁₁ together with the atoms to which they are attached form a 5- to 6-membered heterocyclic ring; or

R₈ and R₉ together with the atoms to which they are attached form a 5- to 6-membered heterocyclic ring;

R₁₂, R₁₃, R₁₄ and R₁₅ are each independently selected from H or —C₁₋₆ alkyl;

R₁₂ and R₁₃ together with the atoms to which they are attached form a 4- to 6-membered ring;

m, n, m′, n′ are each independently selected from 1 or 2;

s and t are each independently selected from 1, 2 or 3.

In some embodiments of Formula IV, wherein Ring A is 6 member aryl ring or 5- to 6-membered heteroaryl comprising for 2 N atoms.

In some embodiments of Formula IV, wherein Ring A is

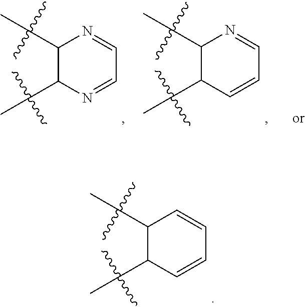 [SMILES: *C1N=CC=NC1* ; *C1C=CC=NC1* ; *C1C=CC=CC1*]

In some embodiments of Formula IV, wherein R₃ is Cl or Br.

In some embodiments of Formula IV, wherein R₄ is selected from H, —OCH₃.

In some embodiments of Formula IV, wherein R₅ is selected from

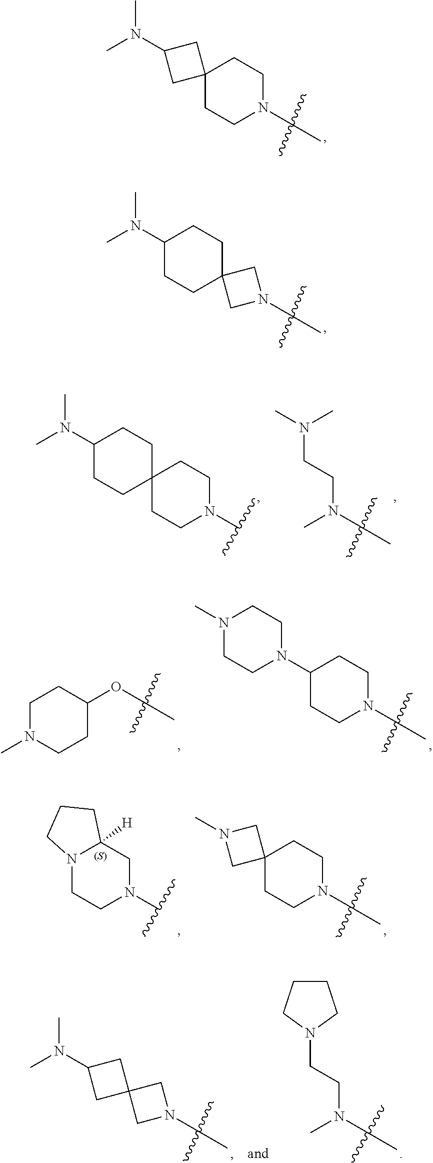

The present invention further provides some preferred technical solutions with regard to compound of Formula I, compound is:
1) N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide;
2) N-(5-((5-chloro-4-((2-(dimethylphosphoryl)phenyl)amino)pyrimidin-2-yl)amino)-2-(7-(dimethylamino)-2-azaspiro[3.5]nonan-2-yl)phenyl)acrylamide;
3) N-(5-((5-chloro-4-((2-(dimethylphosphoryl)phenyl)amino)pyrimidin-2-yl)amino)-2-(6-(dimethylamino)-2-azaspiro[3.3]heptan-2-yl)phenyl)acrylamide;
4) N-(2-(2-(dimethylamino)-7-azaspiro[3.5]nonan-7-yl)-5-((4-((2-(dimethylphosphoryl)phenyl)amino)pyrimidin-2-yl)amino)phenyl)acrylamide;
5) N-(5-((5-chloro-4-((2-(dimethylphosphoryl)phenyl)amino)pyrimidin-2-yl)amino)-2-(2-(dimethylamino)-7-azaspiro[3.5]nonan-7-yl)-4-methoxyphenyl)acrylamide;
6) N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-N-methylacrylamid;
7) N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)methacrylamide;
8) (E)-N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-4-(dimethylamino)but-2-enamide;
9) N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-2-fluoroacrylamide;
10) N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)phenyl)acrylamide;
11) N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)-4-methoxyphenyl)acrylamide;
12) N-(5-((5-bromo-4-((5-(dimethylphosphoryl)quinoxalin-6-yl)amino)pyrimidin-2-yl)amino)-2-(4-(4-methylpiperazin-1-yl)piperidin-1-yl)phenyl)acrylamide;
13) N-(5-((5-bromo-4-((5-(dimethylphosphoryl)quinoxalin-6-yl)amino)pyrimidin-2-yl)amino)-4-methoxy-2-(4-(4-methylpiperazin-1-yl)piperidin-1-yl)phenyl)acrylamide;
14) N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt;
15) N-(5-((5-chloro-4-((2-(dimethylphosphoryl)phenyl)amino)pyrimidin-2-yl)amino)-2-((2-(dimethylamino)ethyl)(methyl)amino)phenyl)acrylamide;
16) N-(5-((5-chloro-4-((2-(dimethylphosphoryl)phenyl)amino)pyrimidin-2-yl)amino)-2-(4-(4-methylpiperazin-1-yl)piperidin-1-yl)phenyl)acrylamide;
17) (S)—N-(5-((5-chloro-4-((2-(dimethylphosphoryl)phenyl)amino)pyrimidin-2-yl)amino)-2-(hexahydropyrrolo[1,2-a]pyrazin-2(1H)-yl)phenyl)acrylamide;
18) N-(5-((5-chloro-4-((2-(dimethylphosphoryl)phenyl)amino)pyrimidin-2-yl)amino)-2-(methyl(2-(pyrrolidin-1-yl)ethyl)amino)phenyl)acrylamide;
19) N-(2-((2-(dimethylamino)ethyl)(methyl)amino)-5-((4-((2-(dimethylphosphoryl)phenyl)amino)-5-methylpyrimidin-2-yl)amino)phenyl)acrylamide hydrochloric acid salt;
20) N-(5-((4-((2-(dimethylphosphoryl)phenyl)amino)-5-methylpyrimidin-2-yl)amino)-2-(4-(4-methylpiperazin-1-yl)piperidin-1-yl)phenyl)acrylamide;
21) N-(2-((2-(dimethylamino)ethyl)(methyl)amino)-5-((4-((2-(dimethylphosphoryl)phenyl)amino)-5-methylpyrimidin-2-yl)amino)-4-methoxyphenyl)acrylamide;
22) N-(5-((4-((2-(dimethylphosphoryl)phenyl)amino)-5-methylpyrimidin-2-yl)amino)-4-methoxy-2-(4-(4-methylpiperazin-1-yl)piperidin-1-yl)phenyl)acrylamide;
23) N-(5-((5-chloro-4-((1-(dimethylphosphoryl)naphthalen-2-yl)amino)pyrimidin-2-yl)amino)-2-(2-(dimethylamino)-7-azaspiro[3.5]nonan-7-yl)phenyl)acrylamide hydrochloric acid salt;
24) N-(5-((5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acrylamide;
25) N-(5-((5-chloro-4-((2-(dimethylphosphoryl)phenyl)amino)pyrimidin-2-yl)amino)-2-((2-(dimethylamino)ethyl)(methyl)amino)-4-methoxyphenyl)acrylamide;
26) N-(5-((5-chloro-4-((2-(dimethylphosphoryl)phenyl)amino)pyrimidin-2-yl)amino)-2-(7-(dimethylamino)-2-azaspiro[3.5]nonan-2-yl)-4-methoxyphenyl)acrylamide;
27) N-(2-((2-(dimethylamino)ethyl)(methyl)amino)-5-((4-((2-(dimethylphosphoryl)phenyl)amino)-5-fluoropyrimidin-2-yl)amino)-4-methoxyphenyl)acrylamide;
28) N-(5-((4-((2-(dimethylphosphoryl)phenyl)amino)-5-fluoropyrimidin-2-yl)amino)-4-methoxy-2-(4-(4-methylpiperazin-1-yl)piperidin-1-yl)phenyl)acrylamide;
29) N-(5-((4-((2-(dimethylphosphoryl)phenyl)amino)-5-fluoropyrimidin-2-yl)amino)-2-(4-(4-methylpiperazin-1-yl)piperidin-1-yl)phenyl)acrylamide;
30) N-(5-((5-chloro-4-((5-(dimethylphosphoryl)quinolin-6-yl)amino)pyrimidin-2-yl)amino)-2-(2-(dimethylamino)-7-azaspiro[3.5]nonan-7-yl)phenyl)acrylamide;
31) N-(5-((5-chloro-4-((2-(dimethylphosphoryl)phenyl)amino)pyrimidin-2-yl)amino)-2-(2-(dimethylamino)-7-azaspiro[3.5]nonan-7-yl)-4-methylphenyl)acrylamide;
32) N-(2-((2-(dimethylamino)ethyl)(methyl)amino)-5-(4-(2-(dimethylphosphoryl)phenylamino)-5-fluoropyrimidin-2-ylamino)phenyl)acrylamide;
33) N-(5-(5-chloro-4-(2-(dimethylphosphoryl)phenylamino)pyrimidin-2-ylamino)-2-(9-(dimethylamino)-3-azaspiro[5.5]undecan-3-yl)-4-methoxyphenyl)acrylamide;
34) N-(5-(5-bromo-4-(5-(dimethylphosphoryl)quinoxalin-6-ylamino)pyrimidin-2-ylamino)-2-((2-(dimethylamino)ethyl)(methyl)amino)phenyl)acrylamide;
35) N-(5-(5-bromo-4-(5-(dimethylphosphoryl)quinoxalin-6-ylamino)pyrimidin-2-ylamino)-2-(2-(dimethylamino)-7-azaspiro[3.5]nonan-7-yl)phenyl)acrylamide;
36) N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)acrylamide;
37) N-(2-((2-(dimethylamino)ethyl)(methyl)amino)-5-((4-((2-(dimethylphosphoryl)phenyl)amino)pyrimidin-2-yl)amino)phenyl)acrylamide;
38) N-(5-((4-((2-(dimethylphosphoryl)phenyl)amino)pyrimidin-2-yl)amino)-2-(4-(4-methylpiperazin-1-yl)piperidin-1-yl)phenyl)acrylamide;
39) N-(2-((2-(dimethylamino)ethyl)(methyl)amino)-5-((4-((2-(dimethylphosphoryl)phenyl)amino)pyrimidin-2-yl)amino)-4-methoxyphenyl)acrylamide;
40) N-(5-((4-((2-(dimethylphosphoryl)phenyl)amino)pyrimidin-2-yl)amino)-4-methoxy-2-(4-(4-methylpiperazin-1-yl)piperidin-1-yl)phenyl)acrylamide;
41) N-(5-((5-chloro-4-((2-(dimethylphosphoryl)phenyl)amino)pyrimidin-2-yl)amino)-2-((1-methylpiperidin-4-yl)oxy)phenyl)acrylamide.

The present invention also provides a pharmaceutical composition comprising a compound of any one of the present invention, or a pharmaceutically acceptable salt or a stereoisomer thereof, and at least one pharmaceutically acceptable carrier or excipient.

The present invention additionally provided a method of inhibiting various different forms of EGFR, including the L858R, the 19del, the T790M, and the C797S, said method comprising administering to a patient a compound of any one of the present invention or a pharmaceutically acceptable salt or a stereoisomer thereof.

The present invention further provides a method of treating an EGFR-driven cancer, said method comprising administering to a patient in need thereof a therapeutically effective amount of a compound of any one of the present invention, or a pharmaceutically acceptable salt or a stereoisomer thereof.

In some embodiments, the EGFR-driven cancer is characterized by the presence of one or more mutations selected from: (i) C797S, (ii) both L858R and C797S, (iii) both C797S and T790M, (iv) L858R, T790M, and C797S, or (v) 19del, T790M and C797S.

In some embodiments, the EGFR-driven cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

The present invention provided a method of inhibiting mutant EGFR in a patient, said method comprising administering to the patient in need thereof a therapeutically effective amount of a compound of the present invention, or a pharmaceutically acceptable salt or a stereoisomer thereof.

The present invention also provides a use of the present compound or its pharmaceutical composition for the preparation of a medicament.

In some embodiments, wherein the medicament is used for the treatment or prevention of cancer.

In some embodiments, wherein the cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

In some embodiments, wherein the medicament is used as an inhibitor of various different forms of EGFR, including the L858R, the Δ19del, the T790M, and the C797S.

The general chemical terms used in the formula above have their usual meanings. For example, the term “halogen”, as used herein, unless otherwise indicated, means fluoro, chloro, bromo or iodo. The preferred halogen groups include F, Cl and Br.

As used herein, unless otherwise indicated, alkyl includes saturated monovalent hydrocarbon radicals having straight, or branched moieties. For example, alkyl radicals include methyl, ethyl, propyl, isopropyl, n-butyl, isobutyl, sec-butyl, t-butyl, n-pentyl, 3-(2-methyl) butyl, 2-pentyl, 2-methylbutyl, neopentyl, n-hexyl, 2-hexyl, and 2-methylpentyl. Similarly, C₁₋₈, as in C₁₋₈alkyl is defined to identify the group as having 1, 2, 3, 4, 5, 6, 7 or 8 carbon atoms in a linear or branched arrangement.

Alkoxy radicals are oxygen ethers formed from the previously described straight, branched chain or cyclic alkyl groups.

The term “aryl”, as used herein, unless otherwise indicated, refers to an unsubstituted or substituted mono- or polycyclic ring system containing carbon ring atoms. The preferred aryls are mono cyclic or bicyclic 6-10 membered aromatic ring systems. Phenyl and naphthyl are preferred aryls. The most preferred aryl is phenyl.

The term “heteroaryl”, as used herein, unless otherwise indicated, represents an unsubstituted or substituted stable five or six membered monocyclic aromatic ring system or an unsubstituted or substituted nine or ten membered benzo-fused heteroaromatic ring system or bicyclic heteroaromatic ring system which consists of carbon atoms and from one to four heteroatoms selected from N, O or S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “cycloalkyl” to a cyclic saturated alkyl chain having from 3 to 12 carbon atoms, for example, cyclopropyl, cyclobutyl, cyclopentyl, cyclohexyl.

The term “substituted” refers to a group in which one or more hydrogen atoms are each independently replaced with the same or different substituent(s). Typical substituents include, but are not limited to, halogen (F, Cl, Br or I), C₁₋₈ alkyl, C₃₋₁₂ cycloalkyl, —OR¹, SR¹, ═O, ═S, —C(O)R¹, —C(S)R¹, ═NR¹, —C(O)OR¹, —C(S)OR¹, —NR¹R², —C(O)NR¹R², cyano, nitro, —S(O)₂R¹, —OS(O₂)OR¹, —OS(O)₂R¹, —OP(O)(OR¹)(OR²); wherein R¹ and R² is independently selected from —H, lower alkyl, lower haloalkyl. In some embodiments, the substituent(s) is independently selected from the group consisting of —F, —Cl, —Br, —I, —OH, trifluromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃, —OCF₃, amino, dimethylamino, methyl thio, sulfonyl and acetyl.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

Examples of substituted alkyl group include, but not limited to, 2-aminoethyl, 2-hydroxyethyl, pentachloroethyl, trifluoromethyl, methoxymethyl, pentafluoroethyl and piperazinylmethyl.

Examples of substituted alkoxy groups include, but not limited to, aminomethoxy, thrifluoromethoxy, 2-diethylaminoethoxy, 2-ethoxycarbonylethoxy, 3-hydroxypropoxy.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I-IV are shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula I-IV exists, the present invention includes any possible tautomers and pharmaceutically acceptable salts thereof, and mixtures thereof, except where specifically stated otherwise.

When the compound of Formula I-IV and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I-IV are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I-IV (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I-IV, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I-IV, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound, or a pharmaceutically acceptable salt, of Formula I-IV. The compounds of Formula I-IV, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I-IV of this invention, or a pharmaceutically acceptable salt thereof, via conventional processing methods. As an example, a cream or ointment is prepared by admixing hydrophilic material and water, together with about 5 wt % to about 1 Owt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I-IV, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, colon cancer, rectal cancer, mantle cell lymphoma, multiple myeloma, breast cancer, prostate cancer, glioblastoma, squamous cell esophageal cancer, liposarcoma, T-cell lymphoma melanoma, pancreatic cancer, glioblastoma or lung cancer, may be effectively treated by the administration of from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy, the subject disposition to the disease, and the judgment of the treating physician.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit the L858R, the Δ19del, the T790M, and the C797S according to at least one assay described herein.

### EXAMPLES

It is to be understood that the foregoing general description and the following detailed description are exemplary and explanatory only and are not restrictive of any subject matter claimed. All parts and percentages are by weight and all temperatures are degrees Celsius, unless explicitly stated otherwise. The compounds described herein can be obtained from commercial sources or synthesized by conventional methods as shown below using commercially available starting materials and reagents.

The following abbreviations have been used in the examples:
AcOH or HOAc: Acetic acid;
DCM: Dichloromethane;
DIEA: N,N-Diisopropylethylamine;
DMF: Dimethylformamide;
DMSO: Dimethyl sulfoxide;
EA or EtOAc: Ethyl acetate;
Et₂O: Diethyl ether;
HEPES: 4-(2-hydroxyethyl)-1-piperazineethanesulfonic acid;
LCMS: Liquid chromatography-mass spectrometry;
h or hrs: hour or hours;
Pd/C: Palladium on carbon;
PE: Petroleum ether;
MeOH: Methanol;
min: minute;
NMP: N-Methyl-2-pyrrolidone
rt or r.t.: room temperature;
TFA: Trifluoroacetic acid;
THF: Tetrahydrofuran;
TLC: Preparative thin layer chromatography;
NaOtBu: Sodium tert-butoxide;
Xantphos: 4,5-Bis(diphenylphosphino)-9,9-dimethylxanthene;
t-BuXPhos Pd(II): Methanesulfonato(2-di-t-butylphosphino-2′,4′,6′-tri-i-propyl-1,1′-biphenyl)(2′-amino-1,1′-biphenyl-2-yl)palladium(II).

### Example 1 Synthesis of Compound 1

### N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide

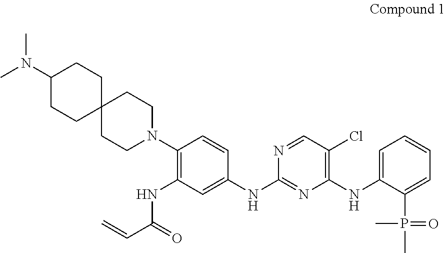 [SMILES: C=CC(=O)Nc1cc(Nc2ncc(Cl)c(Nc3ccccc3P(C)(C)=O)n2)ccc1N1CCC2(CCC(N(C)C)CC2)CC1]

### Step 1: Synthesis of (2-((2,5-dichloropyrimidin-4-yl)amino)phenyl)dimethylphosphine oxide

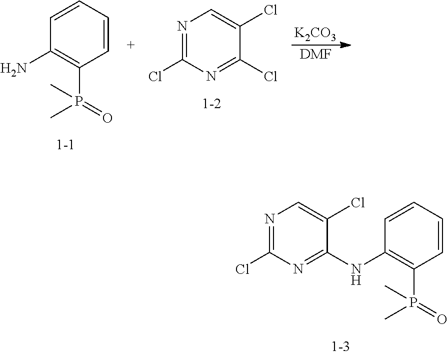

To a mixture of (2-aminophenyl)dimethylphosphine oxide (2.50 g) in DMF (30 mL), 2,4,5-trichloropyrimidine (3.52 g) and potassium carbonate (4.08 g) was added under stirring. The mixture was heated 60° C. for about 8 h. The mixture solution was poured into water and extracted with ethyl acetate (50 mL*3). The combined organic layer was washed with saturated sodium chloride aqueous solution (50 mL*2), dried over Na₂SO₄ and concentrated under reduced pressure. The crude product was recrystallized by hexane/ethyl acetate (10:1, 10 mL). After filtration, the solid was dried to obtain (2-((2,5-dichloropyrimidin-4-yl)amino)phenyl)dimethylphosphine oxide (3.00 g) as white solid. MS: 316 [M+H]⁺.

### Step 2: Synthesis of (2-((5-chloro-2-((4-fluoro-3-nitrophenyl)amino)pyrimidin-4-yl)amino)phenyl)dimethylphosphine oxide

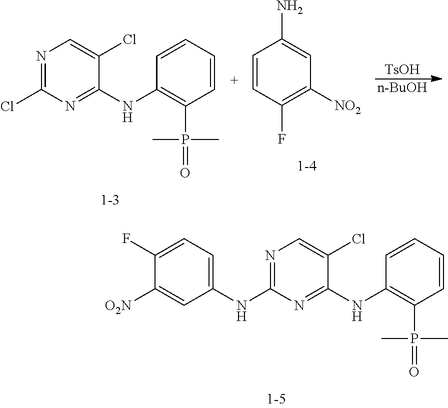

To a solution of 4-fluoro-3-nitroaniline (2.5 g) and (2-((2,5-dichloropyrimidin-4-yl)amino)phenyl)dimethylphosphine oxide (5.05 g) dissolved in n-BuOH (50 mL) was added TsOH (4.1 g). The reaction mixture was stirred at 100° C. for 2 h. The reaction mixture was cooled down to room temperature and diluted with EtOAc (150 mL). The resulting solution was washed with water and NaCl saturated aqueous solution. The mixture was dried over anhydrous sodium sulfate and concentrated under vacuum. The crude product was re-crystallized from PE to obtain 5.3 g (2-((5-chloro-2-((4-fluoro-3-nitrophenyl)amino)pyrimidin-4-yl)amino)phenyl)dimethylphosphine oxide as a yellow solid. MS: 436 [M+H]⁺.

### Step 3: Synthesis of 3-azaspiro[5.5]undecan-9-one trifluoroacetate

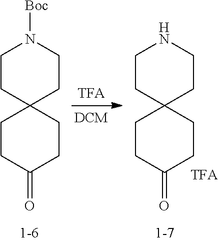 [SMILES: CC(C)(C)OC(=O)N1CCC2(CCC(=O)CC2)CC1 ; O=C1CCC2(CCNCC2)CC1]

To a stirred solution of tert-butyl 9-oxo-3-azaspiro[5.5]undecane-3-carboxylate (0.5 g) in DCM (6 mL) was added TFA (2 mL). The reaction mixture was stirred at room temperature for 2 h. After completion of the reaction (monitored by TLC), the reaction mixture was evaporated under reduced pressure to obtain 3-azaspiro[5.5]undecan-9-one trifluoroacetate (0.9 g, crude) as a yellow oil.

### Step 4: Synthesis of 3-(4-((5-chloro-4-((2-(dimethylphosphoryl)phenyl)amino)pyrimidin-2-yl)amino)-2-nitrophenyl)-3-azaspiro[5.5]undecan-9-one

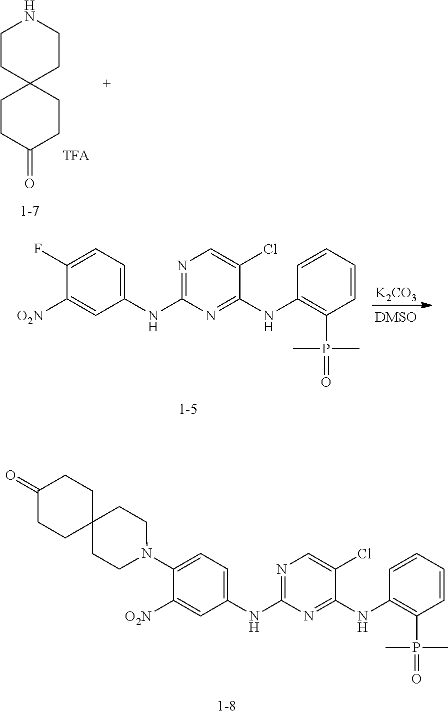

To a solution of 3-azaspiro[5.5]undecan-9-one trifluoroacetate (0.9 g, crude) and (2-((5-chloro-2-((4-fluoro-3-nitrophenyl)amino)pyrimidin-4-yl)amino)phenyl)dimethylphosphine oxide (0.5 g) dissolved in DMSO (10 mL) was added K₂CO₃ (2 g). The reaction mixture was stirred at 90° C. overnight. The reaction mixture was cooled down to room temperature and diluted with DCM (50 mL). The resulting solution was washed with water and NaCl saturated aqueous solution. The mixture was dried over anhydrous sodium sulfate and concentrated under vacuum. The crude product was re-crystallized from Et₂O to obtained 0.68 g of 3-(4-((5-chloro-4-((2-(dimethylphosphoryl)phenyl)amino)pyrimidin-2-yl)amino)-2-nitrophenyl)-3-azaspiro[5.5]undecan-9-one as a red solid. MS: 583 [M+H]⁺.

### Step 5: Synthesis of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide

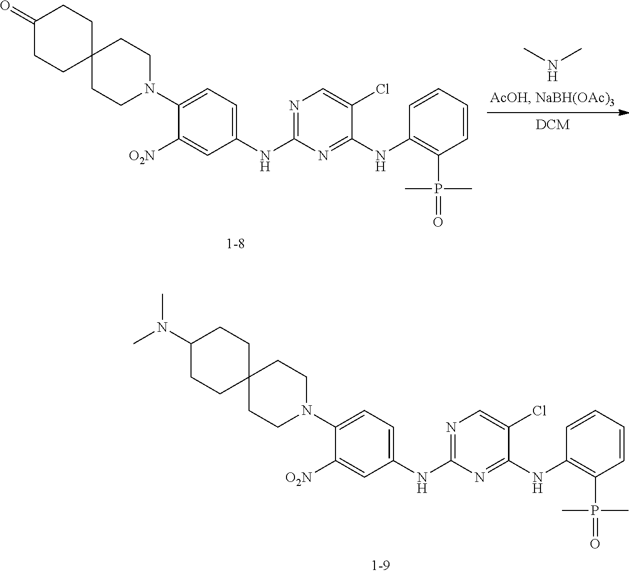

To a solution of 3-(4-((5-chloro-4-((2-(dimethylphosphoryl)phenyl)amino)pyrimidin-2-yl)amino)-2-nitrophenyl)-3-azaspiro[5.5]undecan-9-one (380 mg) in DCM (6 mL) was added dimethylamine (1.63 mL, 2N in THF) and AcOH (39 mg). The mixture was stirred at 90° C. After 1 h, sodium triacetoxyborohydride (413 mg) was added and the mixture was further stirred at room temperature for 1 h. After completion of the reaction (monitored by TLC), the reaction mixture was diluted with DCM (30 mL). The resulting solution was washed with 10% NaHCO₃ aqueous solution and NaCl saturated aqueous solution. The mixture was dried over anhydrous magnesium sulfate and concentrated under vacuum. The crude product was re-crystallized from Et₂O to obtained 370 mg of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide as a red solid. MS: 612 [M+H]⁺.

### Step 6: Synthesis of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide

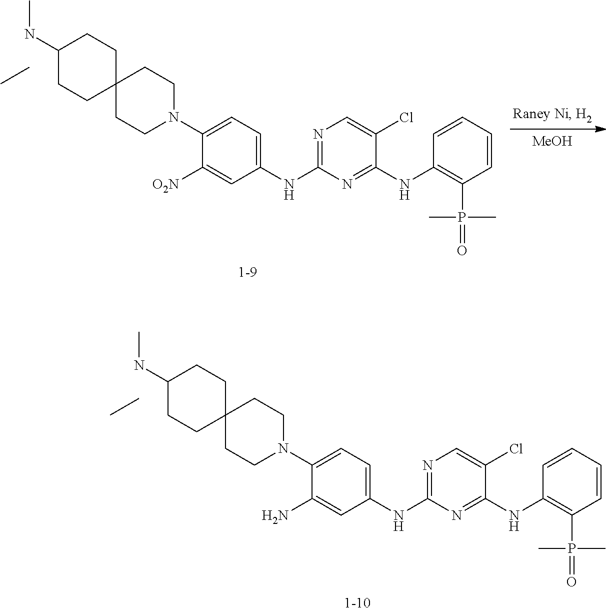

To a solution of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide (370 mg) dissolved in MeOH (10 mL) was added Raney Ni (200 mg). H₂ gas was connected via a needle to the reaction mixture which was stirred at room temperature for 3 h. The solution was filtered through diatomite to remove the Raney Ni. The solution was evaporated to give 260 mg of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide. MS: 582 [M+H]⁺

### Step 7: Synthesis of N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide (Compound 1)

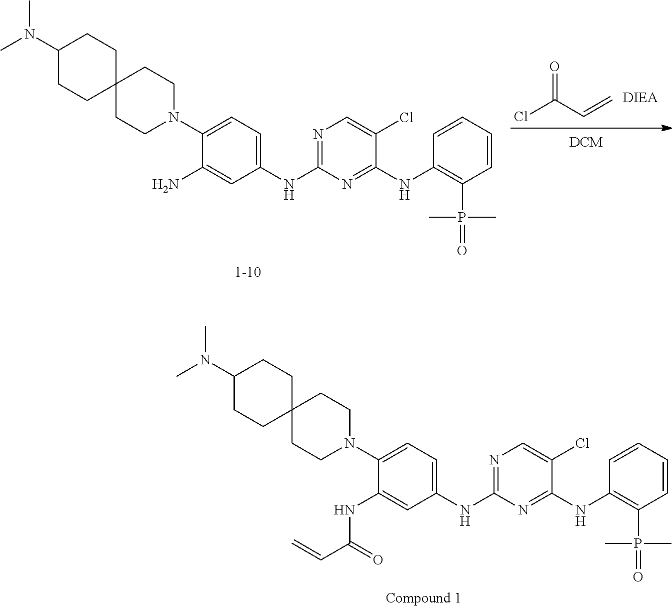

To a solution of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide (260 mg) in DCM (10 mL) was added DIEA (70 mg) at 0° C. This was followed by the addition of acryloyl chloride (44 mg, dissolved in 1 mL DCM), in portions at 0° C. The resulting solution was stirred for 2 h at 0° C. The reaction was then quenched by the addition of 10 mL of 10% NaHCO₃ aqueous solution. The resulting solution was extracted with 2*20 mL DCM and the organic layer combined. The mixture was dried over anhydrous magnesium sulfate and concentrated under vacuum. The residue was purified by column chromatography over silica gel with DCM/MeOH (8:1) to obtain 25 mg N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide (Compound 1). MS: 636 [M+H]⁺.

¹H NMR (500 MHz, DMSO-d₆) δ 11.23 (s, 1H), 9.37 (s, 1H), 9.06 (s, 1H), 8.69 (s, 1H), 8.17 (d, J=7.2 Hz, 2H), 7.57 (ddd, J=14.0, 7.7, 1.6 Hz, 1H), 7.47 (t, J=8.5 Hz, 2H), 7.14 (m, 2H), 6.67 (dd, J=16.9, 10.3 Hz, 1H), 6.22 (dd, J=17.0, 1.9 Hz, 1H), 5.76 (dd, J=10.2, 1.8 Hz, 1H), 3.08 (s, 1H), 2.72 (m, 10H), 1.88 (t, J=15.7 Hz, 4H), 1.78 (d, J=13.6 Hz, 6H), 1.71 (m, 2H), 1.67-1.55 (m, 2H), 1.52 (m, 2H), 1.21-1.12 (m, 2H).

### Example 2 Synthesis of Compound 2

### N-(5-((5-chloro-4-((2-(dimethylphosphoryl)phenyl)amino)pyrimidin-2-yl)amino)-2-(7-(dimethylamino)-2-azaspiro[3.5]nonan-2-yl)phenyl)acrylamide

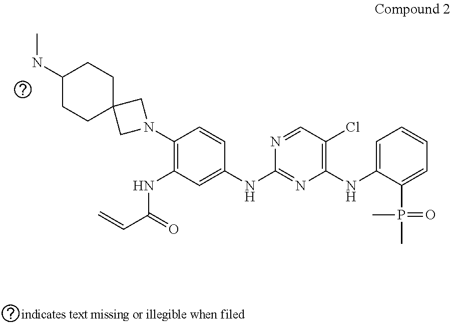 [SMILES: C=CC(=O)Nc1cc(Nc2ncc(Cl)c(Nc3ccccc3P(C)(C)=O)n2)ccc1N1CC2(CCC(NC)CC2)C1]

### Step 1: Synthesis of 2-azaspiro[3.5]nonan-7-one trifluoroacetate

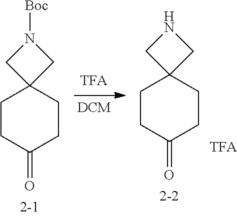 [SMILES: CC(C)(C)OC(=O)N1CC2(CCC(=O)CC2)C1 ; O=C1CCC2(CC1)CNC2 ; "TFA"]

Following the same procedure as 3-azaspiro[5.5]undecan-9-one trifluoroacetate using tert-butyl 7-oxo-2-azaspiro[3.5]nonane-2-carboxylate instead of tert-butyl 9-oxo-3-azaspiro[5.5]undecane-3-carboxylate to obtain 2-azaspiro[3.5]nonan-7-one trifluoroacetate.

### Step 2: Synthesis of 2-(4-((5-chloro-4-((2-(dimethylphosphoryl)phenyl)amino)pyrimidin-2-yl)amino)-2-nitrophenyl)-2-azaspiro[3.5]nonan-7-one

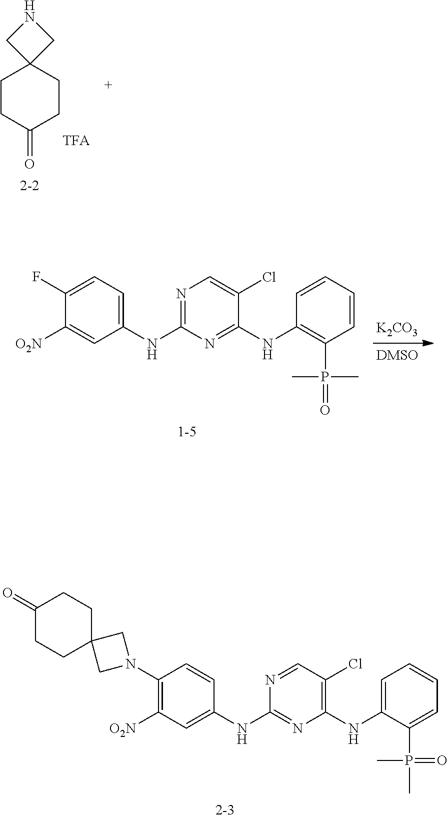

Following the same procedure as 3-(4-((5-chloro-4-((2-(dimethylphosphoryl)phenyl)amino)pyrimidin-2-yl)amino)-2-nitrophenyl)-3-azaspiro[5.5]undecan-9-one using 2-azaspiro[3.5]nonan-7-one trifluoroacetate instead of 3-azaspiro[5.5]undecan-9-one trifluoroacetate to obtain 2-(4-((5-chloro-4-((2-(dimethylphosphoryl)phenyl)amino)pyrimidin-2-yl)amino)-2-nitrophenyl)-2-azaspiro[3.5]nonan-7-one. MS: 555 [M+H]⁺.

### Step 3: Synthesis of (2-((5-chloro-2-((4-(7-(dimethylamino)-2-azaspiro[3.5]nonan-2-yl)-3-nitrophenyl)amino)pyrimidin-4-yl)amino)phenyl)dimethylphosphine oxide

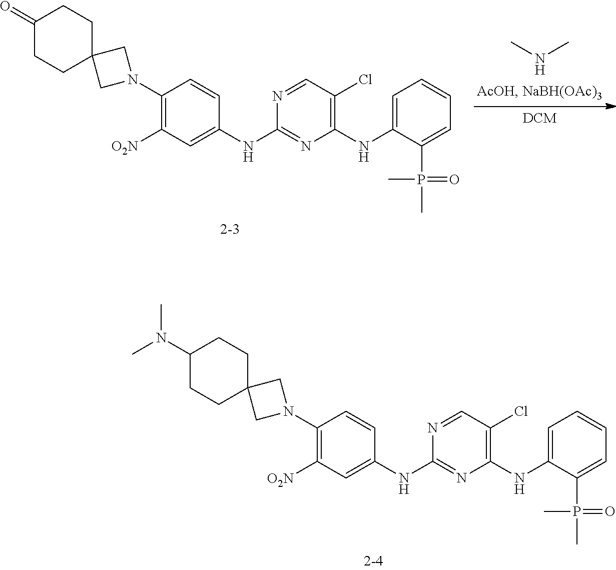

Following the same procedure as (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide using 2-(4-((5-chloro-4-((2-(dimethylphosphoryl)phenyl)amino)pyrimidin-2-yl)amino)-2-nitrophenyl)-2-azaspiro[3.5]nonan-7-one instead of 3-(4-((5-chloro-4-((2-(dimethylphosphoryl)phenyl)amino)pyrimidin-2-yl)amino)-2-nitrophenyl)-3-azaspiro[5.5]undecan-9-one to obtain (2-((5-chloro-2-((4-(7-(dimethylamino)-2-azaspiro[3.5]nonan-2-yl)-3-nitrophenyl)amino)pyrimidin-4-yl)amino)phenyl)dimethylphosphine oxide. MS: 584 [M+H]⁺.

### Step 4: Synthesis of (2-((2-((3-amino-4-(7-(dimethylamino)-2-azaspiro[3.5]nonan-2-yl)phenyl)amino)-5-chloropyrimidin-4-yl)amino)phenyl)dimethylphosphine oxide

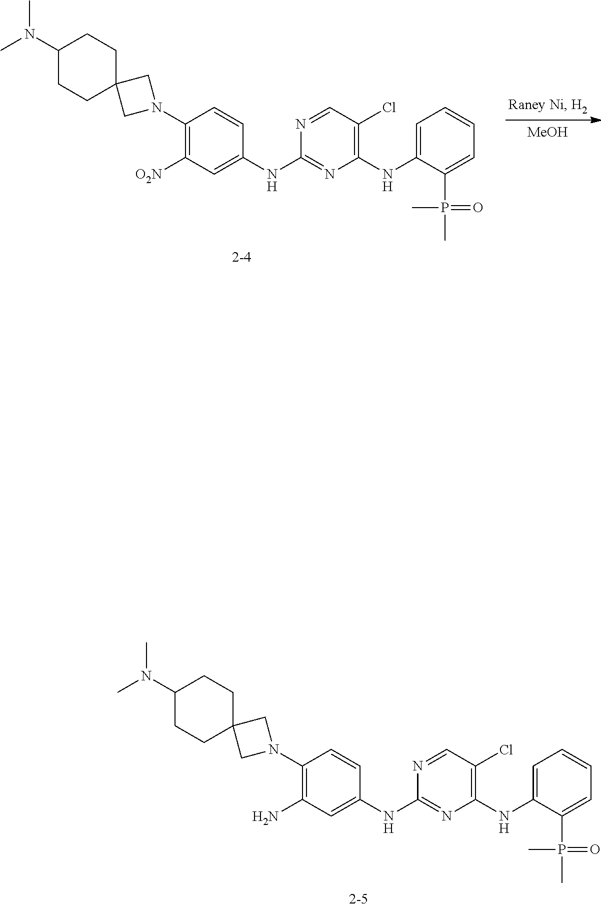 [SMILES: "MeOH" ; CN(C)C1CCC2(CC1)CN(c1ccc(Nc3ncc(Cl)c(Nc4ccccc4P(C)(C)=O)n3)cc1[N+](=O)[O-])C2 ; CN(C)C1CCC2(CC1)CN(c1ccc(Nc3ncc(Cl)c(Nc4ccccc4P(C)(C)=O)n3)cc1N)C2]

Following the same procedure as (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide using (2-((5-chloro-2-((4-(7-(dimethylamino)-2-azaspiro[3.5]nonan-2-yl)-3-nitrophenyl)amino)pyrimidin-4-yl)amino)phenyl)dimethylphosphine oxide instead of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide to obtain (2-((2-((3-amino-4-(7-(dimethylamino)-2-azaspiro[3.5]nonan-2-yl)phenyl)amino)-5-chloropyrimidin-4-yl)amino)phenyl)dimethylphosphine oxide. MS: 554 [M+H]⁺.

### Step 5: Synthesis of N-(5-((5-chloro-4-((2-(dimethylphosphoryl)phenyl)amino)pyrimidin-2-yl)amino)-2-(7-(dimethylamino)-2-azaspiro[3.5]nonan-2-yl)phenyl)acrylamide (Compound 2)

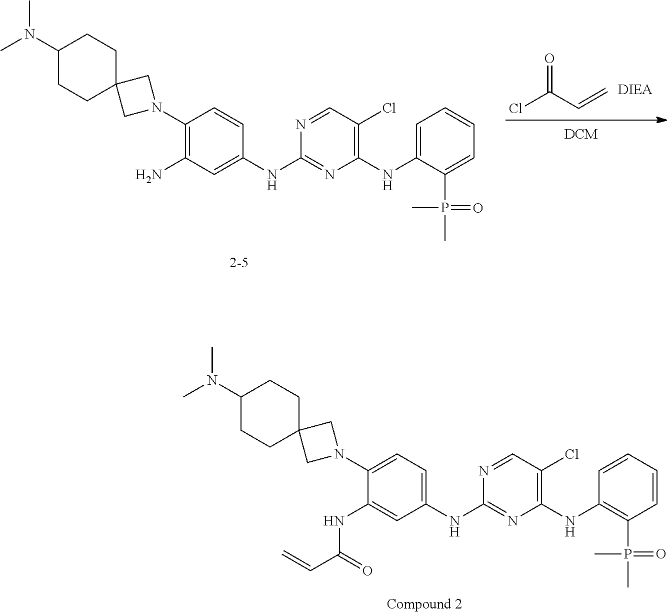

Following the same procedure as N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide using (2-((2-((3-amino-4-(7-(dimethylamino)-2-azaspiro[3.5]nonan-2-yl)phenyl)amino)-5-chloropyrimidin-4-yl)amino)phenyl)dimethylphosphine oxide instead of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide to obtain N-(5-((5-chloro-4-((2-(dimethylphosphoryl)phenyl)amino)pyrimidin-2-yl)amino)-2-(7-(dimethylamino)-2-azaspiro[3.5]nonan-2-yl)phenyl)acrylamide (Compound 2). MS: 608 [M+H]⁺.

### Example 3 Synthesis of Compound 3

### N-(5-((5-chloro-4-((2-(dimethylphosphoryl)phenyl)amino)pyrimidin-2-yl)amino)-2-(6-(dimethylamino)-2-azaspiro[3.3]heptan-2-yl)phenyl)acrylamide

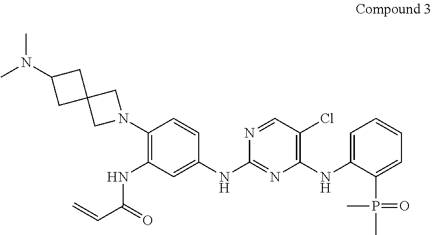 [SMILES: C=CC(=O)Nc1cc(Nc2ncc(Cl)c(Nc3ccccc3P(C)(C)=O)n2)ccc1N1CC2(CC(N(C)C)C2)C1]

### Step 1: Synthesis of 2-azaspiro[3.3]heptan-6-one trifluoroacetate

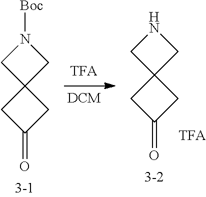 [SMILES: CC(C)(C)OC(=O)N1CC2(CC(=O)C2)C1 ; O=C1CC2(CNC2)C1 ; "TFA"]

Following the same procedure as 3-azaspiro[5.5]undecan-9-one trifluoroacetate using tert-butyl 6-oxo-2-azaspiro[3.3]heptane-2-carboxylate instead of tert-butyl 9-oxo-3-azaspiro[5.5]undecane-3-carboxylate to obtain 2-azaspiro[3.3]heptan-6-one trifluoroacetate.

### Step 2: Synthesis of 2-(4-((5-chloro-4-((2-(dimethylphosphoryl)phenyl)amino)pyrimidin-2-yl)amino)-2-nitrophenyl)-2-azaspiro[3.3]heptan-6-one

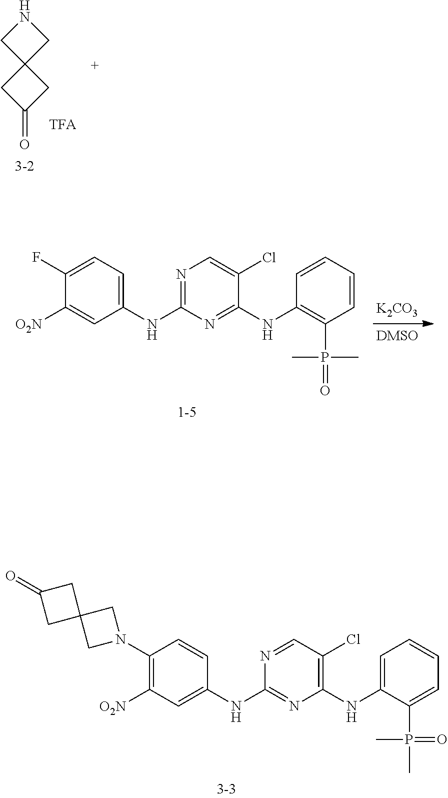

Following the same procedure as 3-(4-((5-chloro-4-((2-(dimethylphosphoryl)phenyl)amino)pyrimidin-2-yl)amino)-2-nitrophenyl)-3-azaspiro[5.5]undecan-9-one using 2-azaspiro[3.3]heptan-6-one trifluoroacetate instead of 3-azaspiro[5.5]undecan-9-one trifluoroacetate to obtain 2-(4-((5-chloro-4-((2-(dimethylphosphoryl)phenyl)amino)pyrimidin-2-yl)amino)-2-nitrophenyl)-2-azaspiro[3.3]heptan-6-one. MS: 527 [M+H]⁺.

### Step 3: Synthesis of (2-((5-chloro-2-((4-(6-(dimethylamino)-2-azaspiro[3.3]heptan-2-yl)-3-nitrophenyl)amino)pyrimidin-4-yl)amino)phenyl)dimethylphosphine oxide

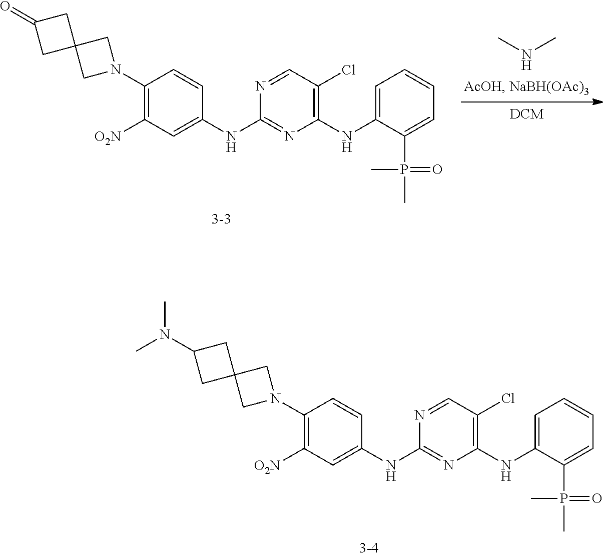

Following the same procedure as (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide using 2-(4-((5-chloro-4-((2-(dimethylphosphoryl)phenyl)amino)pyrimidin-2-yl)amino)-2-nitrophenyl)-2-azaspiro[3.3]heptan-6-one instead of 3-(4-((5-chloro-4-((2-(dimethylphosphoryl)phenyl)amino)pyrimidin-2-yl)amino)-2-nitrophenyl)-3-azaspiro[5.5]undecan-9-one to obtain (2-((5-chloro-2-((4-(6-(dimethylamino)-2-azaspiro[3.3]heptan-2-yl)-3-nitrophenyl)amino)pyrimidin-4-yl)amino)phenyl)dimethylphosphine oxide. MS: 556 [M+H]⁺.

### Step 4: Synthesis of (2-((2-((3-amino-4-(6-(dimethylamino)-2-azaspiro[3.3]heptan-2-yl)phenyl)amino)-5-chloropyrimidin-4-yl)amino)phenyl)dimethylphosphine oxide

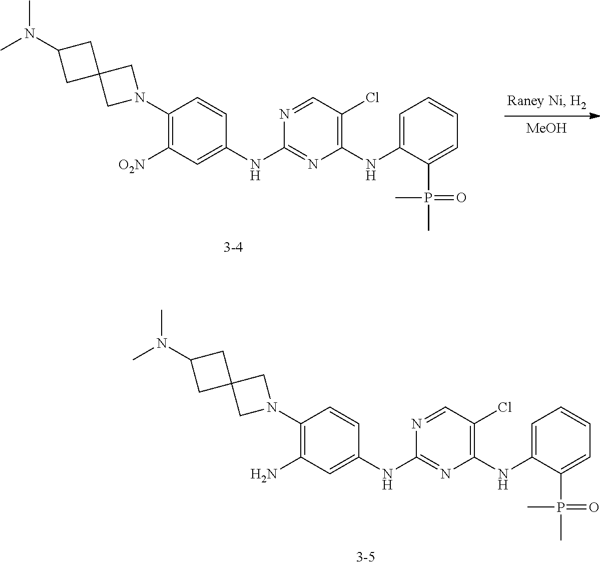 [SMILES: "MeOH" ; CN(C)C1CC2(C1)CN(c1ccc(Nc3ncc(Cl)c(Nc4ccccc4P(C)(C)=O)n3)cc1[N+](=O)[O-])C2 ; CN(C)C1CC2(C1)CN(c1ccc(Nc3ncc(Cl)c(Nc4ccccc4P(C)(C)=O)n3)cc1N)C2]

Following the same procedure as (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide using (2-((5-chloro-2-((4-(6-(dimethylamino)-2-azaspiro[3.3]heptan-2-yl)-3-nitrophenyl)amino)pyrimidin-4-yl)amino)phenyl)dimethylphosphine oxide instead of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide to obtain (2-((2-((3-amino-4-(6-(dimethylamino)-2-azaspiro[3.3]heptan-2-yl)phenyl)amino)-5-chloropyrimidin-4-yl)amino)phenyl)dimethylphosphine oxide. MS: 526 [M+H]⁺.

### Step 5: Synthesis of N-(5-((5-chloro-4-((2-(dimethylphosphoryl)phenyl)amino)pyrimidin-2-yl)amino)-2-(6-(dimethylamino)-2-azaspiro[3.3]heptan-2-yl)phenyl)acrylamide (Compound 3)

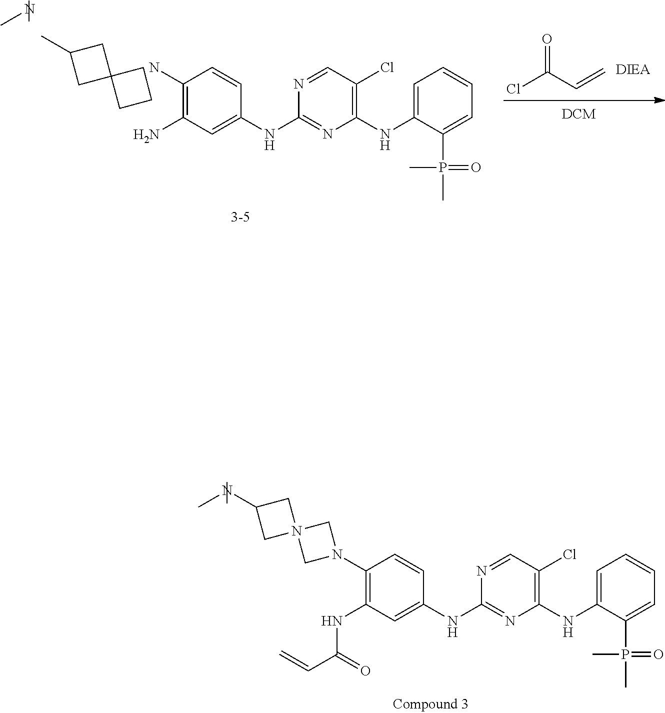

Following the same procedure as N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide using (2-((2-((3-amino-4-(6-(dimethylamino)-2-azaspiro[3.3]heptan-2-yl)phenyl)amino)-5-chloropyrimidin-4-yl)amino)phenyl)dimethylphosphine oxide instead of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide to obtain N-(5-((5-chloro-4-((2-(dimethylphosphoryl)phenyl)amino)pyrimidin-2-yl)amino)-2-(6-(dimethylamino)-2-azaspiro[3.3]heptan-2-yl)phenyl)acrylamide (Compound 3). MS: 580 [M+H]⁺.

### Example 4 Synthesis of Compound 4

### N-(2-(2-(dimethylamino)-7-azaspiro[3.5]nonan-7-yl)-5-((4-((2-(dimethylphosphoryl)phenyl)amino)pyrimidin-2-yl)amino)phenyl)acrylamide

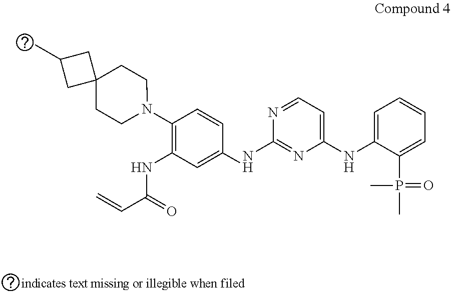 [SMILES: C=CC(=O)Nc1cc(Nc2nccc(Nc3ccccc3P(C)(C)=O)n2)ccc1N1CCC2(CC1)CC(C)C2]

### Step 1: Synthesis of (2-((2-chloropyrimidin-4-yl)amino)phenyl)dimethylphosphine oxide

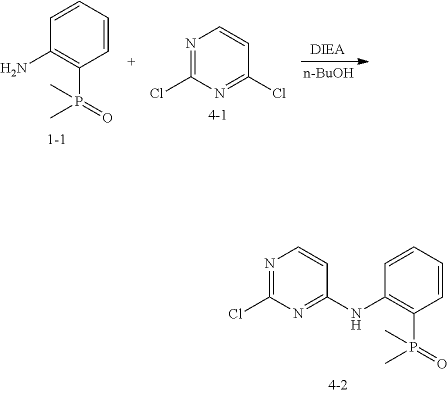

To a mixture of (2-aminophenyl)dimethylphosphine oxide (5.0 g) in n-BuOH (50 mL), 2,4-dichloropyrimidine (4.4 g) and DIEA (5.73 g) was added under stirring. The mixture was heated 120° C. overnight. The mixture solution was poured into water and extracted with ethyl acetate (50 mL*3). The combined organic layer was washed with saturated sodium chloride aqueous solution (50 mL*2), dried over Na₂SO₄ and concentrated under reduced pressure. The crude product was recrystallized by Et₂O. After filtration, the solid was dried to obtain (2-((2-chloropyrimidin-4-yl)amino)phenyl)dimethylphosphine oxide (4.9 g) as off-white solid. MS: 282 [M+H]⁺.

### Step 2: Synthesis of (2-((2-((4-fluoro-3-nitrophenyl)amino)pyrimidin-4-yl)amino)phenyl)dimethylphosphine oxide

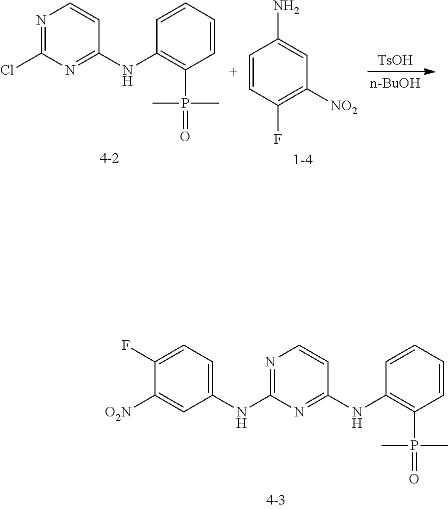

Following the same procedure as (2-((5-chloro-2-((4-fluoro-3-nitrophenyl)amino)pyrimidin-4-yl)amino)phenyl)dimethylphosphine oxide using (2-((2-chloropyrimidin-4-yl)amino)phenyl)dimethylphosphine oxide instead of (2-((2,5-dichloropyrimidin-4-yl)amino)phenyl)dimethylphosphine oxide to obtain (2-((2-((4-fluoro-3-nitrophenyl)amino)pyrimidin-4-yl)amino)phenyl)dimethylphosphine oxide. MS: 402 [M+H]⁺.

### Step 3: Synthesis of 7-(4-((5-chloro-4-((2-(dimethylphosphoryl)phenyl)amino)pyrimidin-2-yl)amino)-2-nitrophenyl)-7-azaspiro[3.5]nonan-2-one

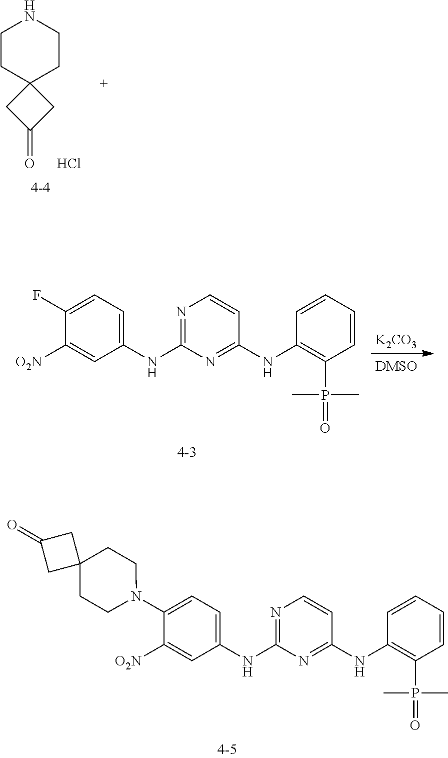

To a solution of 7-azaspiro[3.5]nonan-2-one hydrogen chloride salt (262 mg) and (2-((2-((4-fluoro-3-nitrophenyl)amino)pyrimidin-4-yl)amino)phenyl)dimethylphosphine oxide (500 mg) dissolved in DMSO (10 mL) was added K₂CO₃ (518 mg). The reaction mixture was stirred at 90° C. overnight. The reaction mixture was cooled down to room temperature and diluted with DCM (50 mL). The resulting solution was washed with water and NaCl saturated aqueous solution. The mixture was dried over anhydrous sodium sulfate and concentrated under vacuum. The crude product was re-crystallized from Et₂O to obtained 0.66 g of 7-(4-((5-chloro-4-((2-(dimethylphosphoryl)phenyl)amino)pyrimidin-2-yl)amino)-2-nitrophenyl)-7-azaspiro[3.5]nonan-2-one as a red solid. MS: 555 [M+H]⁺.

### Step 4: Synthesis of (2-((2-((4-(2-(dimethylamino)-7-azaspiro[3.5]nonan-7-yl)-3-nitrophenyl)amino)pyrimidin-4-yl)amino)phenyl)dimethylphosphine oxide

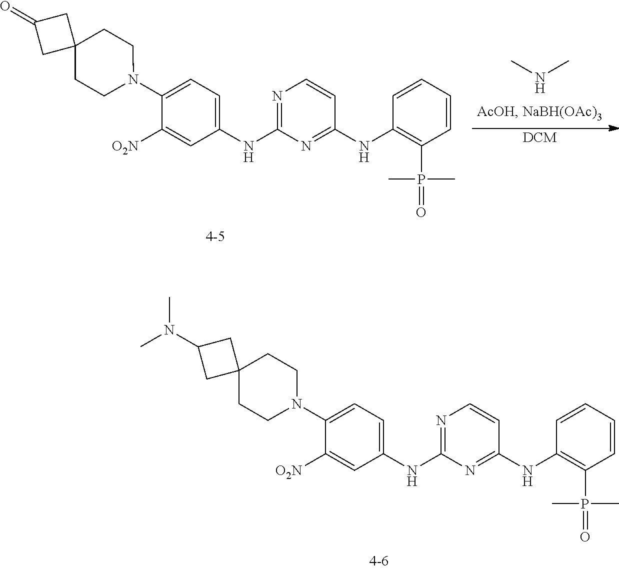

Following the same procedure as (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide using 7-(4-((5-chloro-4-((2-(dimethylphosphoryl)phenyl)amino)pyrimidin-2-yl)amino)-2-nitrophenyl)-7-azaspiro[3.5]nonan-2-one instead of 3-(4-((5-chloro-4-((2-(dimethylphosphoryl)phenyl)amino)pyrimidin-2-yl)amino)-2-nitrophenyl)-3-azaspiro[5.5]undecan-9-one to obtain (2-((2-((4-(2-(dimethylamino)-7-azaspiro[3.5]nonan-7-yl)-3-nitrophenyl)amino)pyrimidin-4-yl)amino)phenyl)dimethylphosphine oxide. MS: 584 [M+H]⁺.

### Step 5: Synthesis of (2-((2-((3-amino-4-(2-(dimethylamino)-7-azaspiro[3.5]nonan-7-yl)phenyl)amino)pyrimidin-4-yl)amino)phenyl)dimethylphosphine oxide

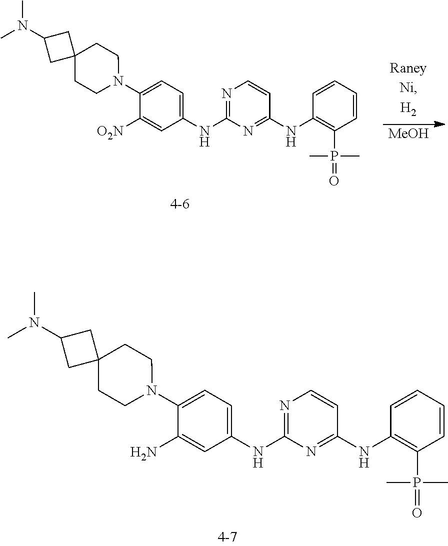

Following the same procedure as (2-((2-((4-(2-(dimethylamino)-7-azaspiro[3.5]nonan-7-yl)-3-nitrophenyl)amino)pyrimidin-4-yl)amino)phenyl)dimethylphosphine oxide using (2-((2-((4-(2-(dimethylamino)-7-azaspiro[3.5]nonan-7-yl)-3-nitrophenyl)amino)pyrimidin-4-yl)amino)phenyl)dimethylphosphine oxide instead of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide to obtain (2-((2-((3-amino-4-(2-(dimethylamino)-7-azaspiro[3.5]nonan-7-yl)phenyl)amino)pyrimidin-4-yl)amino)phenyl)dimethylphosphine oxide. MS: 520 [M+H]⁺.

### Step 6: Synthesis of N-(2-(2-(dimethylamino)-7-azaspiro[3.5]nonan-7-yl)-5-((4-((2-(dimethylphosphoryl)phenyl)amino)pyrimidin-2-yl)amino)phenyl)acrylamide (Compound 4)

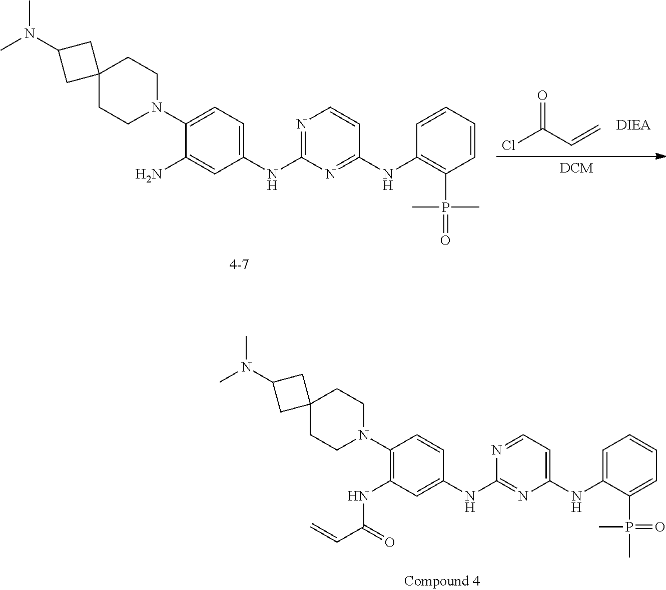

Following the same procedure as N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide using (2-((2-((3-amino-4-(2-(dimethylamino)-7-azaspiro[3.5]nonan-7-yl)phenyl)amino)pyrimidin-4-yl)amino)phenyl)dimethylphosphine oxide instead of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide to obtain N-(2-(2-(dimethylamino)-7-azaspiro[3.5]nonan-7-yl)-5-((4-((2-(dimethylphosphoryl)phenyl)amino)pyrimidin-2-yl)amino)phenyl)acrylamide (Compound 4). MS: 574 [M+H]⁺.

### Example 5 Synthesis of Compound 5

### N-(5-((5-chloro-4-((2-(dimethylphosphoryl)phenyl)amino)pyrimidin-2-yl)amino)-2-(2-(dimethylamino)-7-azaspiro[3.5]nonan-7-yl)-4-methoxyphenyl)acrylamide

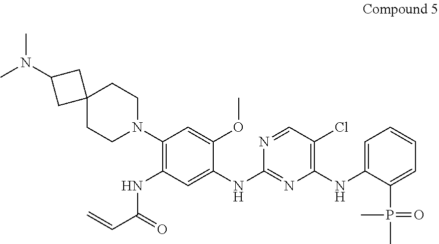 [SMILES: C=CC(=O)Nc1cc(Nc2ncc(Cl)c(Nc3ccccc3P(C)(C)=O)n2)c(OC)cc1N1CCC2(CC1)CC(N(C)C)C2]

### Step 1: Synthesis of N-(5-((5-chloro-4-((2-(dimethylphosphoryl)phenyl)amino)pyrimidin-2-yl)amino)-2-(2-(dimethylamino)-7-azaspiro[3.5]nonan-7-yl)-4-methoxyphenyl)acrylamide

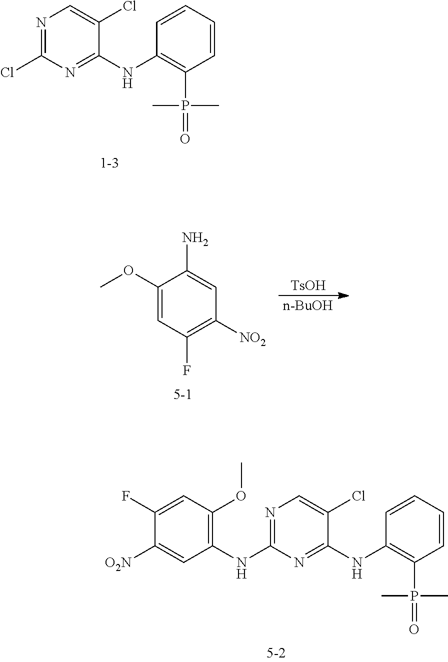

Following the same procedure as (2-((5-chloro-2-((4-fluoro-3-nitrophenyl)amino)pyrimidin-4-yl)amino)phenyl)dimethylphosphine oxide using (2-amino-5-fluoro-4-nitrophenoxy)methylium instead of 4-fluoro-3-nitroaniline to obtain (2-((5-chloro-2-((4-fluoro-2-methoxy-5-nitrophenyl)amino)pyrimidin-4-yl)amino)phenyl)dimethylphosphine oxide. MS: 466 [M+H]⁺.

### Step 2: Synthesis of 7-(4-((5-chloro-4-((2-(dimethylphosphoryl)phenyl)amino)pyrimidin-2-yl)amino)-5-methoxy-2-nitrophenyl)-7-azaspiro[3.5]nonan-2-one

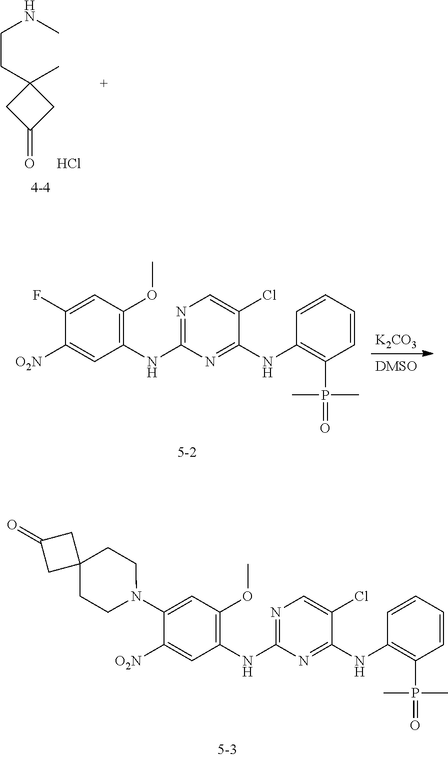

Following the same procedure as 7-(4-((5-chloro-4-((2-(dimethylphosphoryl)phenyl)amino)pyrimidin-2-yl)amino)-2-nitrophenyl)-7-azaspiro[3.5]nonan-2-one using (2-((5-chloro-2-((4-fluoro-2-methoxy-5-nitrophenyl)amino)pyrimidin-4-yl)amino)phenyl)dimethylphosphine oxide instead of (2-((2-((4-fluoro-3-nitrophenyl)amino)pyrimidin-4-yl)amino)phenyl)dimethylphosphine oxide to obtain 7-(4-((5-chloro-4-((2-(dimethylphosphoryl)phenyl)amino)pyrimidin-2-yl)amino)-5-methoxy-2-nitrophenyl)-7-azaspiro[3.5]nonan-2-one. MS: 551 [M+H]⁺.

### Step 3: Synthesis of (2-((5-chloro-2-((4-(2-(dimethylamino)spiro[3.5]nonan-7-yl)-2-methoxy-5-nitrophenyl)amino)pyrimidin-4-yl)amino)phenyl)dimethylphosphine oxide

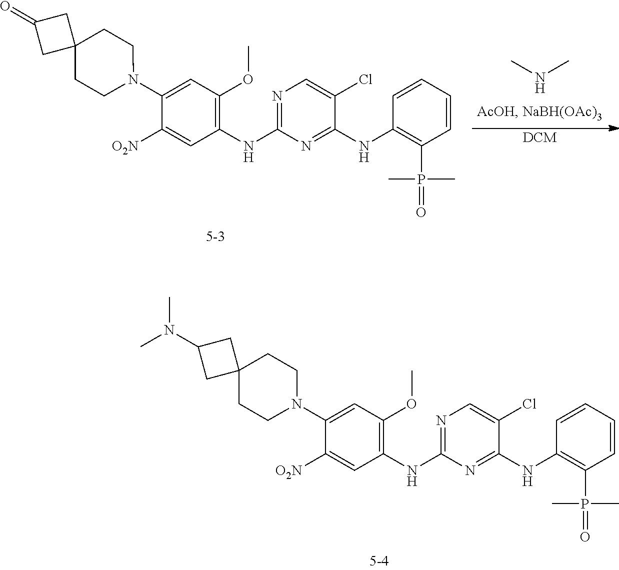

Following the same procedure as (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide using 7-(4-((5-chloro-4-((2-(dimethylphosphoryl)phenyl)amino)pyrimidin-2-yl)amino)-5-methoxy-2-nitrophenyl)-7-azaspiro[3.5]nonan-2-one instead of 3-(4-((5-chloro-4-((2-(dimethylphosphoryl)phenyl)amino)pyrimidin-2-yl)amino)-2-nitrophenyl)-3-azaspiro[5.5]undecan-9-one to obtain (2-((5-chloro-2-((4-(2-(dimethylamino)spiro[3.5]nonan-7-yl)-2-methoxy-5-nitrophenyl)amino)pyrimidin-4-yl)amino)phenyl)dimethylphosphine oxide. MS: 614 [M+H]⁺.

### Step 4: Synthesis of (2-((2-((5-amino-4-(2-(dimethylamino)-7-azaspiro[3.5]nonan-7-yl)-2-methoxyphenyl)amino)-5-chloropyrimidin-4-yl)amino)phenyl)dimethylphosphine oxide

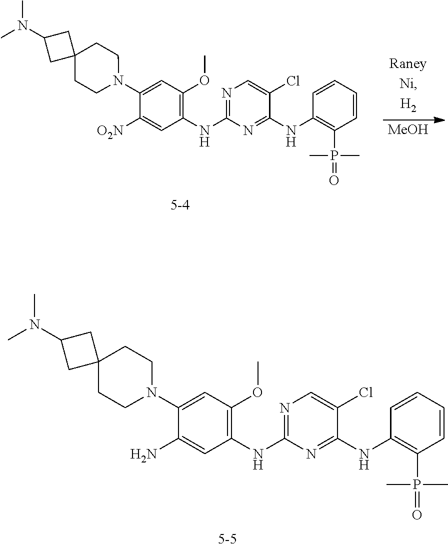

Following the same procedure as (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide using (2-((5-chloro-2-((4-(2-(dimethylamino)spiro[3.5]nonan-7-yl)-2-methoxy-5-nitrophenyl)amino)pyrimidin-4-yl)amino)phenyl)dimethylphosphine oxide instead of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide to obtain (2-((2-((5-amino-4-(2-(dimethylamino)-7-azaspiro[3.5]nonan-7-yl)-2-methoxyphenyl)amino)-5-chloropyrimidin-4-yl)amino)phenyl)dimethylphosphine oxide. MS: 584 [M+H]⁺.

### Step 5: Synthesis of N-(5-((5-chloro-4-((2-(dimethylphosphoryl)phenyl)amino)pyrimidin-2-yl)amino)-2-(2-(dimethylamino)-7-azaspiro[3.5]nonan-7-yl)-4-methoxyphenyl)acrylamide (Compound 5)

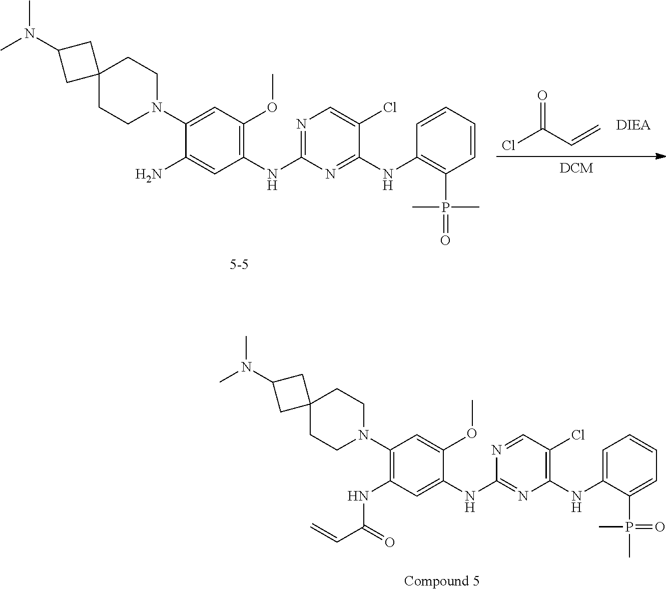

Following the same procedure as N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide using (2-((2-((5-amino-4-(2-(dimethylamino)-7-azaspiro[3.5]nonan-7-yl)-2-methoxyphenyl)amino)-5-chloropyrimidin-4-yl)amino)phenyl)dimethylphosphine oxide instead of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide to obtain N-(5-((5-chloro-4-((2-(dimethylphosphoryl)phenyl)amino)pyrimidin-2-yl)amino)-2-(2-(dimethylamino)-7-azaspiro[3.5]nonan-7-yl)-4-methoxyphenyl)acrylamide. (Compound 5). MS: 604 [M+H]⁺.

¹H NMR (500 MHz, DMSO-d₆) δ 11.21 (s, 1H), 8.97 (s, 1H), 8.45 (s, 1H), 8.27 (s, 1H), 8.10 (d, J=6.1 Hz, 2H), 7.51 (dd, J=14.3, 7.6 Hz, 1H), 7.30 (t, J=8.2 Hz, 1H), 7.05 (t, J=7.5 Hz, 1H), 6.82 (s, 1H), 6.64 (dd, J=17.0, 10.2 Hz, 1H), 6.17 (dd, J=17.0, 2.0 Hz, 1H), 5.70 (dd, J=10.1, 2.0 Hz, 1H), 3.77 (s, 3H), 2.80-2.74 (m, 2H), 2.74-2.68 (m, 2H), 2.59 (m, 1H), 2.03 (m, 8H), 1.76 (m, 8H), 1.69 (d, J=5.3 Hz, 2H), 1.57 (d, J=19.1 Hz, 2H).

### Example 6 Synthesis of Compound 6

### N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-N-methylacrylamide

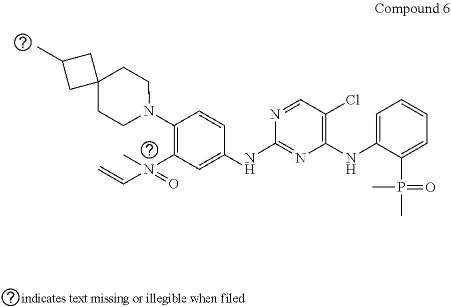 [SMILES: C=C[N+](C)([O-])c1cc(Nc2ncc(Cl)c(Nc3ccccc3P(C)(C)=O)n2)ccc1N1CCC2(CC1)CC(C)C2]

### Step 1: Synthesis of (2-((5-chloro-2-((4-(2-(dimethylamino)-7-azaspiro[3.5]nonan-7-yl)-3-(methylamino)phenyl)amino)pyrimidin-4-yl)amino)phenyl)dimethylphosphine oxide

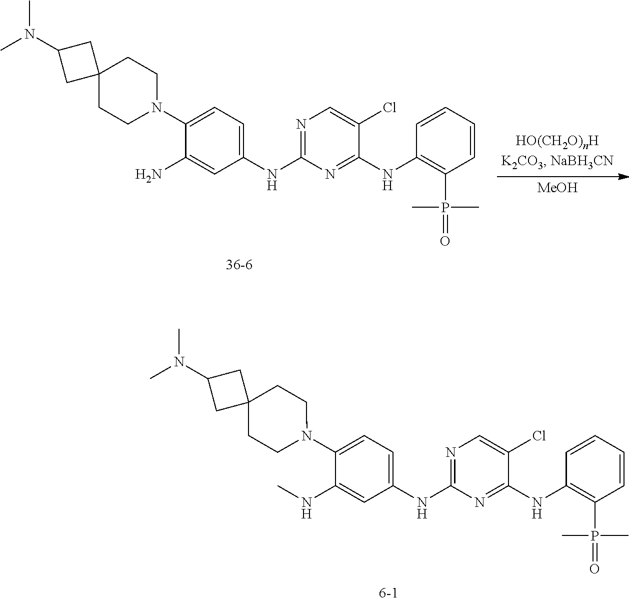

To a solution of (2-((2-((3-amino-4-(2-(dimethylamino)-7-azaspiro[3.5]nonan-7-yl)phenyl)amino)-5-chloropyrimidin-4-yl)amino)phenyl)dimethylphosphine oxide (200 mg) in MeOH (4 mL) was added paraformaldehyde (50 mg), K₂CO₃ (100 mg) and sodium cyanoborohydride (50 mg). The mixture is stirred at room temperature for 1 h. The reaction mixture was diluted with DCM (50 mL). The resulting solution was washed with H₂O and NaCl saturated aqueous solution. The mixture was dried over anhydrous magnesium sulfate and concentrated under vacuum. The residue was purified by column chromatography over silica gel with DCM/MeOH (8:1). This obtained 80 mg (2-((5-chloro-2-((4-(2-(dimethylamino)-7-azaspiro[3.5]nonan-7-yl)-3-(methyl amino)phenyl)amino)pyrimidin-4-yl)amino)phenyl)dimethylphosphine oxide. MS: 568 [M+H]⁺.

### Step 2: Synthesis of N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-N-methylacrylamid

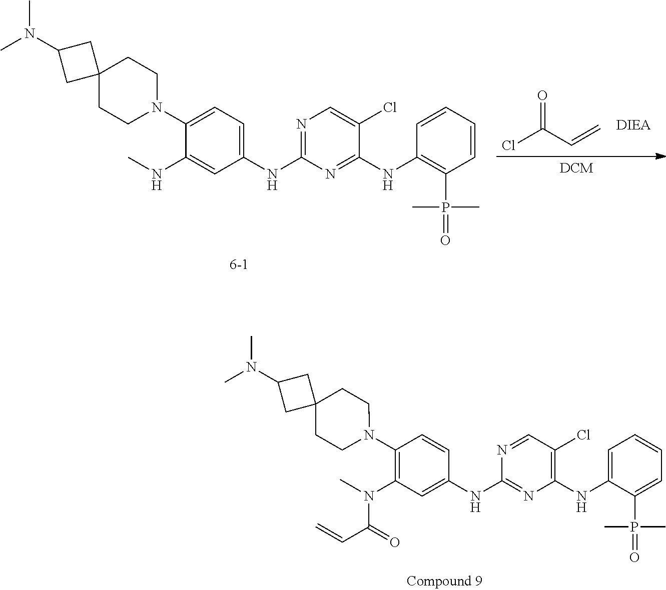

Following the same procedure as N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide using (2-((5-chloro-2-((4-(2-(dimethylamino)-7-azaspiro[3.5]nonan-7-yl)-3-(methylamino)phenyl)amino)pyrimidin-4-yl)amino)phenyl)dimethylphosphine oxide instead of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide to obtain N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-N-methylacrylamid. MS: 622 [M+H]⁺.

### Example 7 Synthesis of Compound 7

### N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)methacrylamide

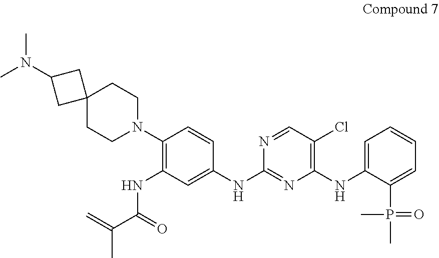 [SMILES: C=C(C)C(=O)Nc1cc(Nc2ncc(Cl)c(Nc3ccccc3P(C)(C)=O)n2)ccc1N1CCC2(CC1)CC(N(C)C)C2]

### Synthesis of N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)methacrylamide

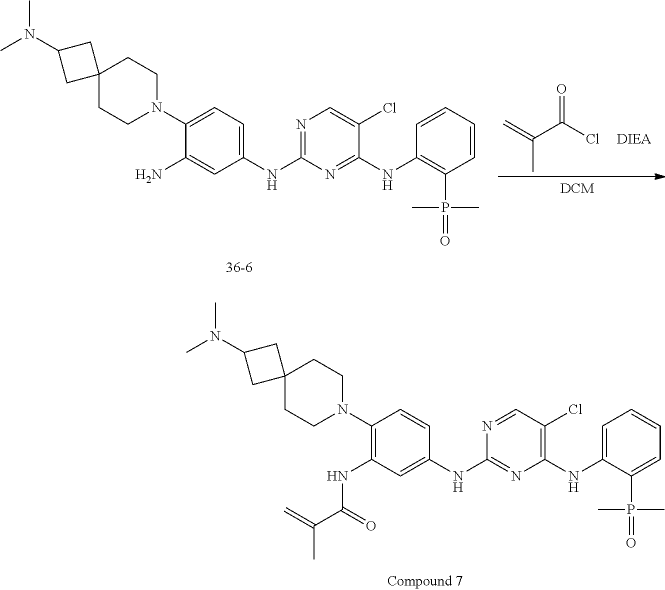

Following the same procedure as N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-N-methylacrylamid using methacryloyl chloride instead of acryloyl chloride to obtain N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)methacrylamide. MS: 622 [M+H]⁺.

¹H NMR (500 MHz, DMSO-d₆) δ 11.25 (s, 1H), 9.41 (s, 1H), 9.16 (s, 1H), 8.71 (s, 1H), 8.38 (d, J=2.5 Hz, 1H), 8.17 (s, 1H), 7.57-7.47 (m, 1H), 7.50-7.41 (m, 2H), 7.18-7.10 (m, 2H), 5.86 (s, 1H), 5.58-5.53 (m, 1H), 3.57 (s, 1H), 2.74-2.64 (m, 2H), 2.67-2.57 (m, 2H), 2.57 (s, 6H), 2.18-2.08 (m, 4H), 2.03-1.93 (m, 3H), 1.79-1.69 (m, 2H), 1.71-1.61 (m, 2H).

### Example 8 Synthesis of Compound 8

### (E)-N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-4-(dimethylamino)but-2-enamide

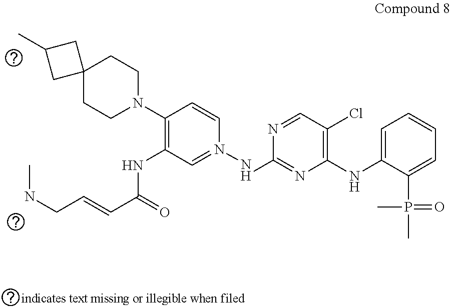

### Synthesis of (E)-N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-4-(dimethylamino)but-2-enamide

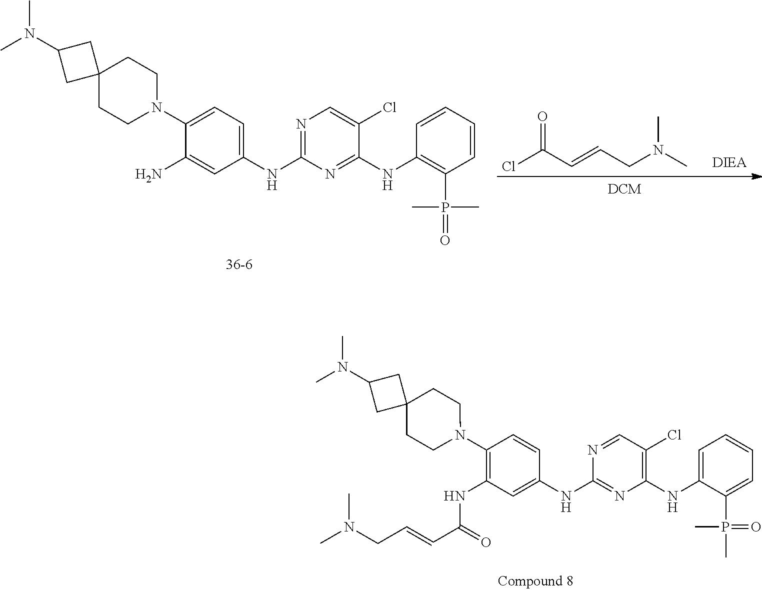

Following the same procedure as N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-N-methylacrylamid (Compound 6) using (E)-4-(dimethylamino)but-2-enoyl chloride instead of acryloyl chloride to obtain (E)-N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-4-(dimethylamino)but-2-enamide. MS: 665 [M+H]⁺.

### Example 9 Synthesis of Compound 9

### N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-2-fluoroacrylamide

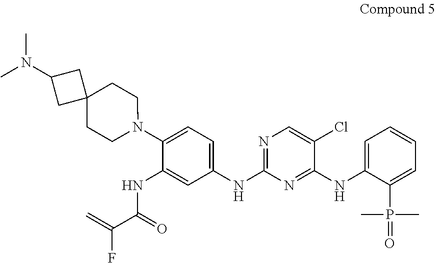

### Synthesis of N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-2-fluoroacrylamide

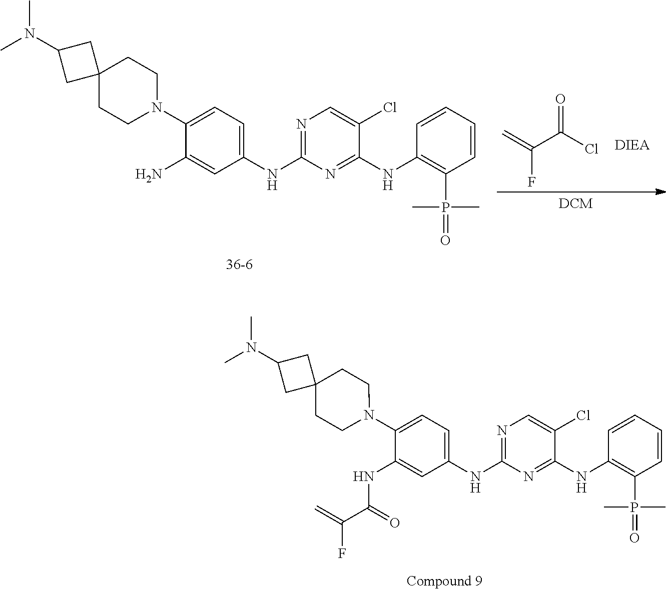

Following the same procedure as N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-N-methylacrylamid using 2-fluoroacryloyl chloride instead of acryloyl chloride to obtain N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-2-fluoroacrylamide. MS: 626 [M+H]⁺.

### Example 10 Synthesis of Compound 10

### N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)phenyl)acrylamide

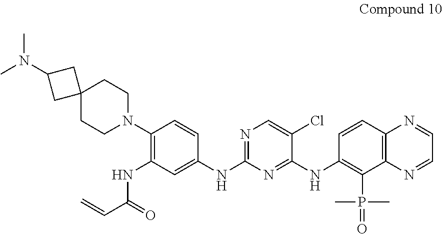 [SMILES: C=CC(=O)Nc1cc(Nc2ncc(Cl)c(Nc3ccc4nccnc4c3P(C)(C)=O)n2)ccc1N1CCC2(CC1)CC(N(C)C)C2]

### Step 1: Synthesis of (6-((2,5-dichloropyrimidin-4-yl)amino)quinoxalin-5-yl)dimethylphosphine oxide

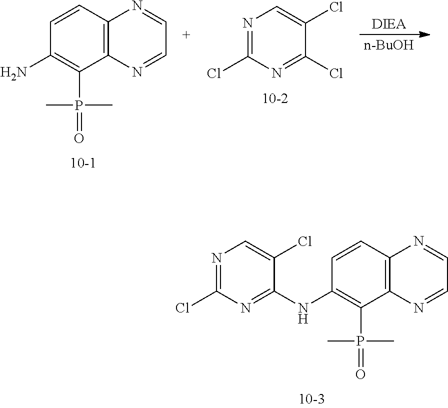

To a mixture of (6-aminoquinoxalin-5-yl)dimethylphosphine oxide (1 g) in n-BuOH (20 mL), 2,4,5-trichloropyrimidine (0.99 g) and DIEA (1.17 g) was added under stirring. The mixture was heated 120° C. for about 48 h. The mixture solution was poured into water and extracted with ethyl acetate (50 mL*3). The combined organic layer was washed with saturated sodium chloride aqueous solution (50 mL*2), dried over Na₂SO₄ and concentrated under reduced pressure. The crude product was recrystallized by hexane/ethyl acetate (10:1, 10 mL). After filtration, the solid was dried to obtain (6-((2,5-dichloropyrimidin-4-yl)amino)quinoxalin-5-yl)dimethylphosphine oxide (760 mg). MS: 368 [M+H]⁺.

### Step 2: Synthesis of (6-((5-chloro-2-((4-fluoro-3-nitrophenyl)amino)pyrimidin-4-yl)amino)quinoxalin-5-yl)dimethylphosphine oxide

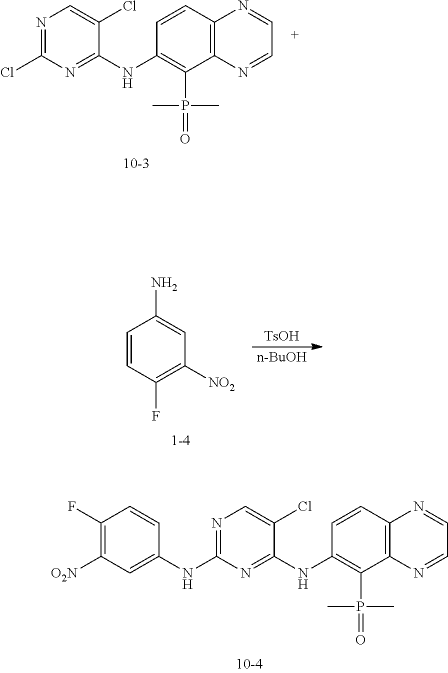

Following the same procedure as (2-((5-chloro-2-((4-fluoro-3-nitrophenyl)amino)pyrimidin-4-yl)amino)phenyl)dimethylphosphine oxide using (6-((2,5-dichloropyrimidin-4-yl)amino)quinoxalin-5-yl)dimethylphosphine oxide instead of (2-((2,5-dichloropyrimidin-4-yl)amino)phenyl)dimethylphosphine oxide to obtain (6-((5-chloro-2-((4-fluoro-3-nitrophenyl)amino)pyrimidin-4-yl)amino)quinoxalin-5-yl)dimethylphosphine oxide. MS: 488 [M+H]⁺.

### Step 3: Synthesis of 7-(4-((5-chloro-4-((5-(dimethylphosphoryl)quinoxalin-6-yl)amino)pyrimidin-2-yl)amino)-2-nitrophenyl)-7-azaspiro[3.5]nonan-2-one

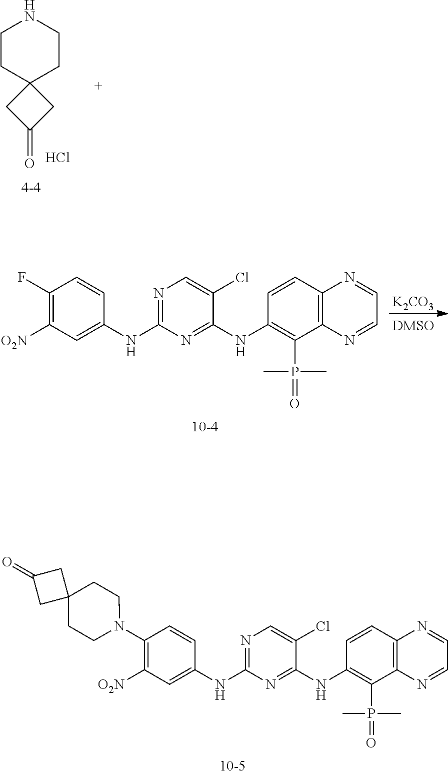

Following the same procedure as 7-(4-((5-chloro-4-((2-(dimethylphosphoryl)phenyl)amino)pyrimidin-2-yl)amino)-2-nitrophenyl)-7-azaspiro[3.5]nonan-2-one using (6-((5-chloro-2-((4-fluoro-3-nitrophenyl)amino)pyrimidin-4-yl)amino)quinoxalin-5-yl)dimethylphosphine oxide instead of (2-((2-((4-fluoro-3-nitrophenyl)amino)pyrimidin-4-yl)amino)phenyl)dimethylphosphine oxide to obtain 7-(4-((5-chloro-4-((5-(dimethylphosphoryl)quinoxalin-6-yl)amino)pyrimidin-2-yl)amino)-2-nitrophenyl)-7-azaspiro[3.5]nonan-2-one. MS: 607 [M+H]⁺

### Step 4: Synthesis of (6-((5-chloro-2-((4-(2-(dimethylamino)-7-azaspiro[3.5]nonan-7-yl)-3-nitrophenyl)amino)pyrimidin-4-yl)amino)quinoxalin-5-yl)dimethylphosphine oxide

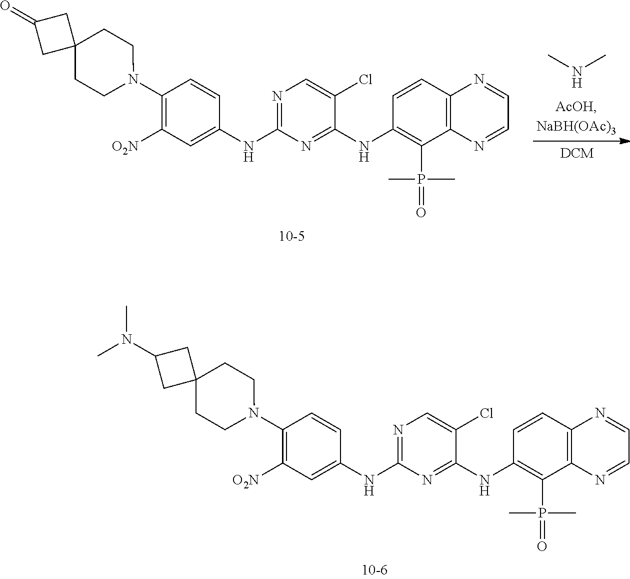

Following the same procedure as (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide using 7-(4-((5-chloro-4-((5-(dimethylphosphoryl)quinoxalin-6-yl)amino)pyrimidin-2-yl)amino)-2-nitrophenyl)-7-azaspiro[3.5]nonan-2-one instead of 3-(4-((5-chloro-4-((2-(dimethylphosphoryl)phenyl)amino)pyrimidin-2-yl)amino)-2-nitrophenyl)-3-azaspiro[5.5]undecan-9-one to obtain (6-((5-chloro-2-((4-(2-(dimethylamino)-7-azaspiro[3.5]nonan-7-yl)-3-nitrophenyl)amino)pyrimidin-4-yl)amino)quinoxalin-5-yl)dimethylphosphine oxide. MS: 636 [M+H]⁺.

### Step 5: Synthesis of (6-((2-((3-amino-4-(2-(dimethylamino)-7-azaspiro[3.5]nonan-7-yl)phenyl)amino)-5-chloropyrimidin-4-yl)amino)quinoxalin-5-yl)dimethylphosphine oxide

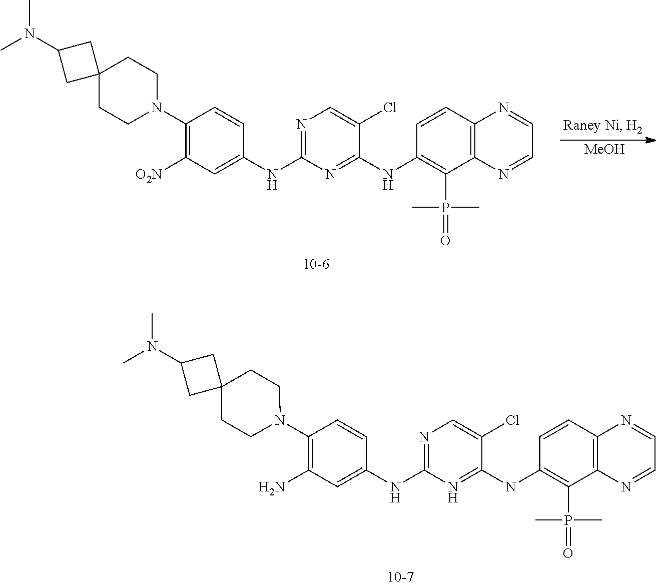 [SMILES: "MeOH" ; CN(C)C1CC2(CCN(c3ccc(Nc4ncc(Cl)c(Nc5ccc6nccnc6c5P(C)(C)=O)n4)cc3[N+](=O)[O-])CC2)C1 ; CN(C)C1CC2(CCN(c3ccc(Nc4ncc(Cl)c(Nc5ccc6nccnc6c5P(C)(C)=O)n4)cc3N)CC2)C1]

Following the same procedure as (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide using (6-((5-chloro-2-((4-(2-(dimethylamino)-7-azaspiro[3.5]nonan-7-yl)-3-nitrophenyl)amino)pyrimidin-4-yl)amino)quinoxalin-5-yl)dimethylphosphine oxide instead of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide to obtain (6-((2-((3-amino-4-(2-(dimethylamino)-7-azaspiro[3.5]nonan-7-yl)phenyl)amino)-5-chloropyrimidin-4-yl)amino)quinoxalin-5-yl)dimethylphosphine oxide. MS: 606 [M+H]⁺.

### Step 6: Synthesis of N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)phenyl)acrylamide

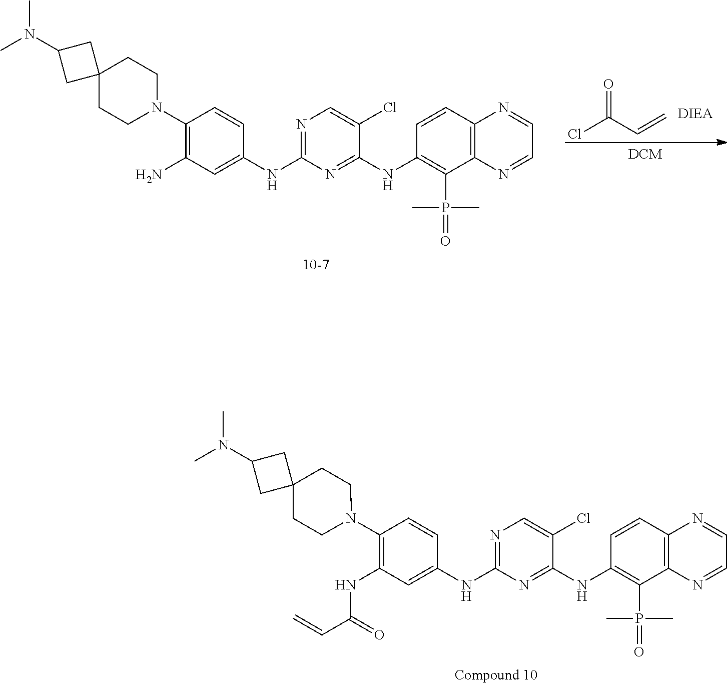

Following the same procedure as N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide using (6-((2-((3-amino-4-(2-(dimethylamino)-7-azaspiro[3.5]nonan-7-yl)phenyl)amino)-5-chloropyrimidin-4-yl)amino)quinoxalin-5-yl)dimethylphosphine oxide instead of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide to obtain N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)phenyl)acrylamide. MS: 660 [M+H]⁺.

¹H NMR (500 MHz, DMSO-d6) δ 12.94 (s, 1H), 9.49 (s, 1H), 9.22 (s, 1H), 9.03 (s, 1H), 8.87-8.86 (m, 2H), 8.28 (d, J=12.3 Hz, 2H), 8.04 (d, J=9.5 Hz, 1H), 7.41 (d, J=8.6 Hz, 1H), 7.10 (d, J=8.7 Hz, 1H), 6.61-6.60 (m, 1H), 6.10-6.09 (m, 1H), 5.66 (d, J=10.4 Hz, 1H), 3.60-6.59 (m, 1H), 2.96-2.95 (m, 2H), 2.74-2.68 (m, 2H), 2.66-2.64 (m, 2H), 2.60 (s, 6H), 2.17-2.07 (m, 2H), 2.15-2.05 (m, 2H), 1.91-1.90 (m, 2H).

### Example 11 Synthesis of Compound 11

### N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)-4-methoxyphenyl)acrylamide

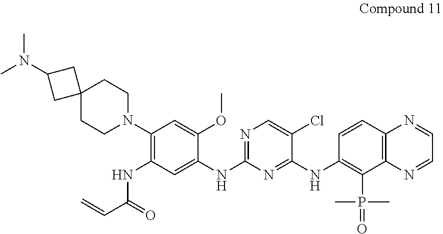 [SMILES: C=CC(=O)Nc1cc(Nc2ncc(Cl)c(Nc3ccc4nccnc4c3P(C)(C)=O)n2)c(OC)cc1N1CCC2(CC1)CC(N(C)C)C2]

### Step 1: Synthesis of (6-((5-chloro-2-((4-fluoro-2-methoxy-5-nitrophenyl)amino)pyrimidin-4-yl)amino)quinoxalin-5-yl)dimethylphosphine oxide

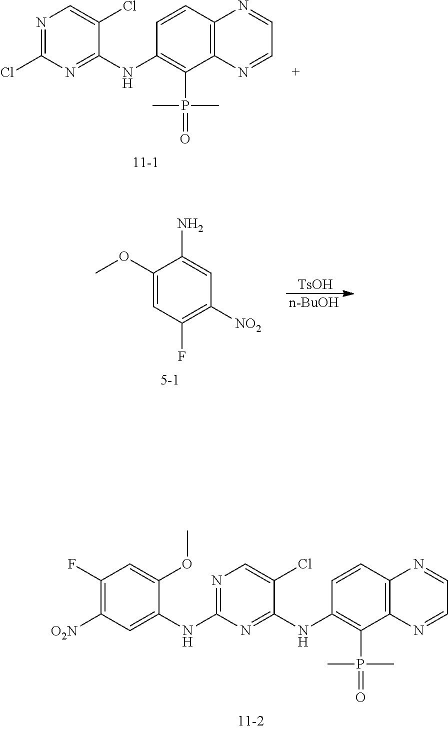

Following the same procedure as (6-((5-chloro-2-((4-fluoro-3-nitrophenyl)amino)pyrimidin-4-yl)amino)quinoxalin-5-yl)dimethylphosphine oxide using 4-fluoro-2-methoxy-5-nitroaniline oxide instead of 4-fluoro-3-nitroaniline to obtain (6-((5-chloro-2-((4-fluoro-2-methoxy-5-nitrophenyl)amino)pyrimidin-4-yl)amino)quinoxalin-5-yl)dimethylphosphine oxide. MS: 518 [M+H]⁺.

### Step 2: Synthesis of 7-(4-((5-chloro-4-((5-(dimethylphosphoryl)quinoxalin-6-yl)amino)pyrimidin-2-yl)amino)-5-methoxy-2-nitrophenyl)-7-azaspiro[3.5]nonan-2-one

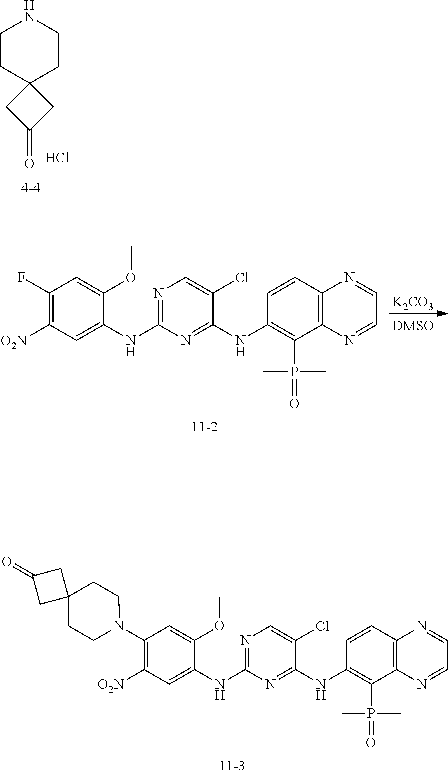

Following the same procedure as 7-(4-((5-chloro-4-((2-(dimethylphosphoryl)phenyl)amino)pyrimidin-2-yl)amino)-2-nitrophenyl)-7-azaspiro[3.5]nonan-2-one using (6-((5-chloro-2-((4-fluoro-2-methoxy-5-nitrophenyl)amino)pyrimidin-4-yl)amino)quinoxalin-5-yl)dimethylphosphine oxide instead of (2-((2-((4-fluoro-3-nitrophenyl)amino)pyrimidin-4-yl)amino)phenyl)dimethylphosphine oxide to obtain 7-(4-((5-chloro-4-((5-(dimethylphosphoryl)quinoxalin-6-yl)amino)pyrimidin-2-yl)amino)-5-methoxy-2-nitrophenyl)-7-azaspiro[3.5]nonan-2-one. MS: 637 [M+H]⁺.

### Step 3: Synthesis of (6-((5-chloro-2-((4-(2-(dimethylamino)-7-azaspiro[3.5]nonan-7-yl)-2-methoxy-5-nitrophenyl)amino)pyrimidin-4-yl)amino)quinoxalin-5-yl)dimethylphosphine oxide

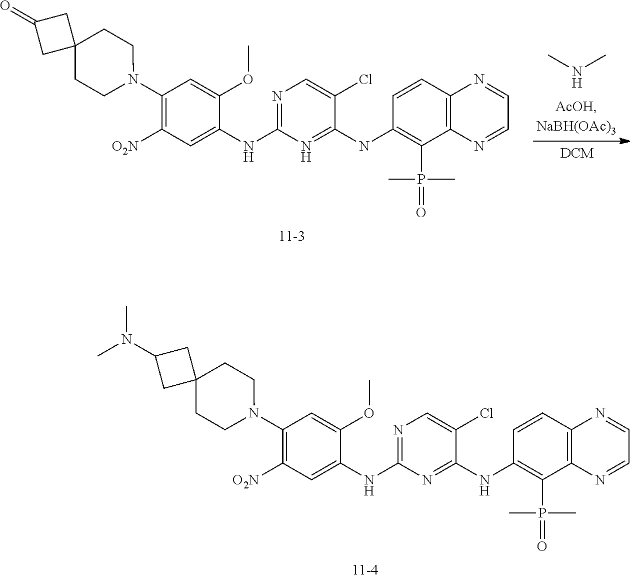

Following the same procedure as (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide using 7-(4-((5-chloro-4-((5-(dimethylphosphoryl)quinoxalin-6-yl)amino)pyrimidin-2-yl)amino)-5-methoxy-2-nitrophenyl)-7-azaspiro[3.5]nonan-2-one instead of 3-(4-((5-chloro-4-((2-(dimethylphosphoryl)phenyl)amino)pyrimidin-2-yl)amino)-2-nitrophenyl)-3-azaspiro[5.5]undecan-9-one to obtain (6-((5-chloro-2-((4-(2-(dimethylamino)-7-azaspiro[3.5]nonan-7-yl)-2-methoxy-5-nitrophenyl)amino)pyrimidin-4-yl)amino)quinoxalin-5-yl)dimethylphosphine oxide. MS: 666 [M+H]⁺.

### Step 4: Synthesis of (6-((2-((5-amino-4-(2-(dimethylamino)-7-azaspiro[3.5]nonan-7-yl)-2-methoxyphenyl)amino)-5-chloropyrimidin-4-yl)amino)quinoxalin-5-yl)dimethylphosphine oxide

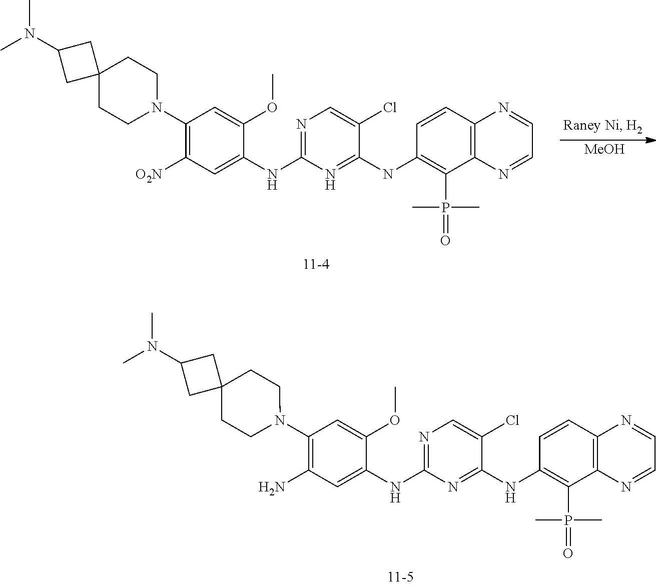 [SMILES: "MeOH" ; COc1cc(N2CCC3(CC2)CC(N(C)C)C3)c([N+](=O)[O-])cc1Nc1ncc(Cl)c(Nc2ccc3nccnc3c2P(C)(C)=O)n1 ; COc1cc(N2CCC3(CC2)CC(N(C)C)C3)c(N)cc1Nc1ncc(Cl)c(Nc2ccc3nccnc3c2P(C)(C)=O)n1]

Following the same procedure as (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide using (6-((5-chloro-2-((4-(2-(dimethylamino)-7-azaspiro[3.5]nonan-7-yl)-2-methoxy-5-nitrophenyl)amino)pyrimidin-4-yl)amino)quinoxalin-5-yl)dimethylphosphine oxide instead of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide to obtain (6-((2-((5-amino-4-(2-(dimethylamino)-7-azaspiro[3.5]nonan-7-yl)-2-methoxyphenyl)amino)-5-chloropyrimidin-4-yl)amino)quinoxalin-5-yl)dimethylphosphine oxide. MS: 636 [M+H]⁺.

### Step 5: Synthesis of N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)-4-methoxyphenyl)acrylamide

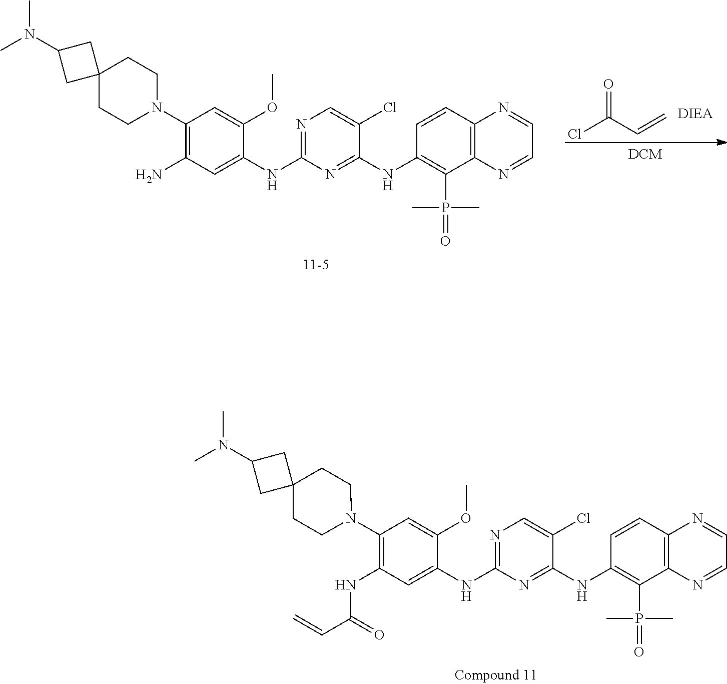

Following the same procedure as N-(5-((5-chloro-4-((2-(dimethylphosphoryl)phenyl)amino)pyrimidin-2-yl)amino)-2-(9-(dimethylamino)-3-azaspiro[5.5]undecan-3-yl)phenyl)acrylamide using (6-((2-((5-amino-4-(2-(dimethylamino)-7-azaspiro[3.5]nonan-7-yl)-2-methoxyphenyl)amino)-5-chloropyrimidin-4-yl)amino)quinoxalin-5-yl)dimethylphosphine oxide instead of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide to obtain N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)-4-methoxyphenyl)acrylamide. MS: 690 [M+H]⁺.

### Example 12 Synthesis of Compound 12

### N-(5-((5-bromo-4-((5-(dimethylphosphoryl)quinoxalin-6-yl)amino)pyrimidin-2-yl)amino)-2-(4-(4-methylpiperazin-1-yl)piperidin-1-yl)phenyl)acrylamide

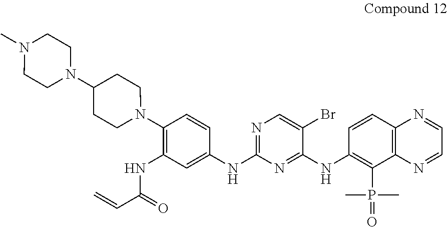 [SMILES: C=CC(=O)Nc1cc(Nc2ncc(Br)c(Nc3ccc4nccnc4c3P(C)(C)=O)n2)ccc1N1CCC(N2CCN(C)CC2)CC1]

### Step 1: Synthesis of (6-((5-bromo-2-((4-fluoro-3-nitrophenyl)amino)pyrimidin-4-yl)amino)quinoxalin-5-yl)dimethylphosphine oxide

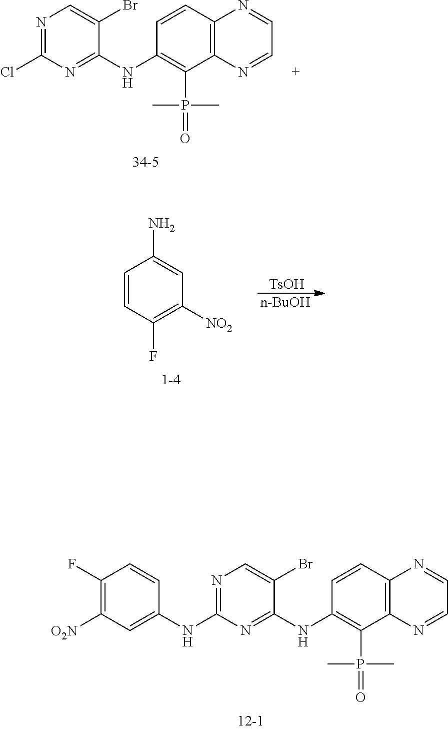

Following the same procedure as (2-((5-chloro-2-((4-fluoro-3-nitrophenyl)amino)pyrimidin-4-yl)amino)phenyl)dimethylphosphine oxide using (6-((5-bromo-2-chloropyrimidin-4-yl)amino)quinoxalin-5-yl)dimethylphosphine oxide instead of (2-((2,5-dichloropyrimidin-4-yl)amino)phenyl)dimethylphosphine oxide to obtain (6-((5-bromo-2-((4-fluoro-3-nitrophenyl)amino)pyrimidin-4-yl)amino)quinoxalin-5-yl)dimethylphosphine oxide. MS: 532 [M+H]⁺.

### Step 2: Synthesis of (6-((5-bromo-2-((4-(4-(4-methylpiperazin-1-yl)piperidin-1-yl)-3-nitrophenyl)amino)pyrimidin-4-yl)amino)quinoxalin-5-yl)dimethylphosphine oxide

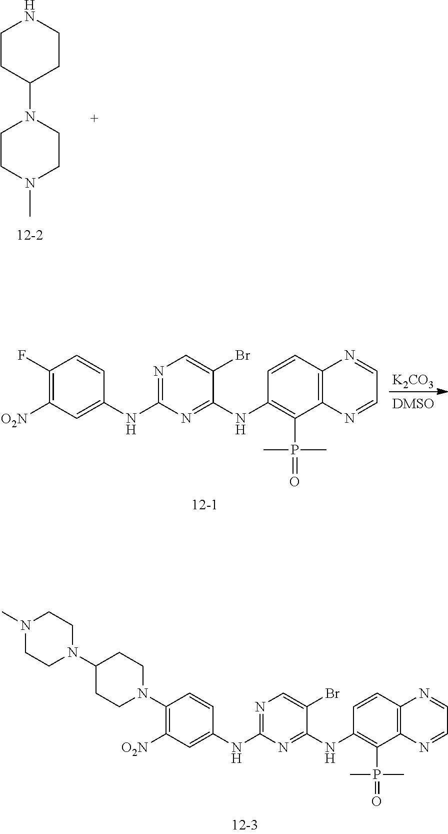

Following the same procedure as 7-(4-((5-chloro-4-((2-(dimethylphosphoryl)phenyl)amino)pyrimidin-2-yl)amino)-2-nitrophenyl)-7-azaspiro[3.5]nonan-2-one using (6-((5-bromo-2-((4-fluoro-3-nitrophenyl)amino)pyrimidin-4-yl)amino)quinoxalin-5-yl)dimethylphosphine oxide instead of (2-((2-((4-fluoro-3-nitrophenyl)amino)pyrimidin-4-yl)amino)phenyl)dimethylphosphine oxide, using 1-methyl-4-(piperidin-4-yl)piperazine instead of 7-azaspiro[3.5]nonan-2-one hydrogen chloride salt to obtain (6-((5-bromo-2-((4-(4-(4-methyl piperazin-1-yl)piperidin-1-yl)-3-nitrophenyl)amino)pyrimidin-4-yl)amino)quinoxalin-5-yl)dimethylphosphine oxide. MS: 695 [M+H]⁺.

### Step 3: Synthesis of (6-((2-((3-amino-4-(4-(4-methylpiperazin-1-yl)piperidin-1-yl)phenyl)amino)-5-bromopyrimidin-4-yl)amino)quinoxalin-5-yl)dimethylphosphine oxide

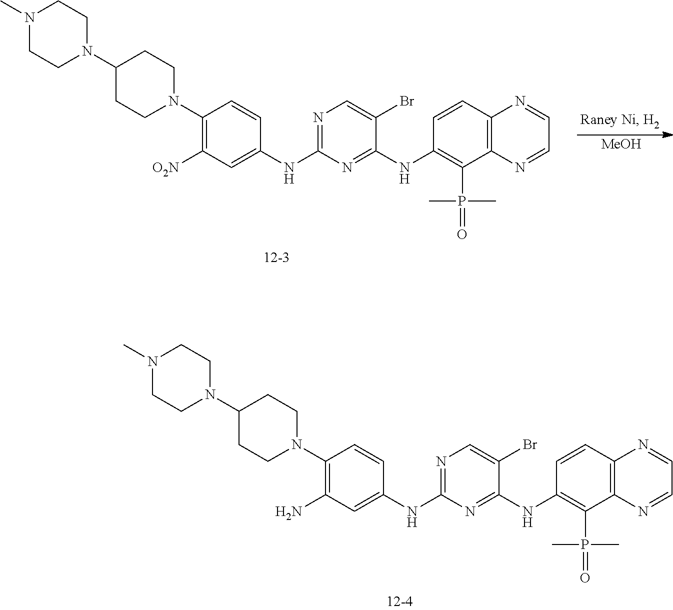 [SMILES: "MeOH" ; CN1CCN(C2CCN(c3ccc(Nc4ncc(Br)c(Nc5ccc6nccnc6c5P(C)(C)=O)n4)cc3[N+](=O)[O-])CC2)CC1 ; CN1CCN(C2CCN(c3ccc(Nc4ncc(Br)c(Nc5ccc6nccnc6c5P(C)(C)=O)n4)cc3N)CC2)CC1]

Following the same procedure as (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide using (6-((5-bromo-2-((4-(4-(4-methylpiperazin-1-yl)piperidin-1-yl)-3-nitrophenyl)amino)pyrimidin-4-yl)amino)quinoxalin-5-yl)dimethylphosphine oxide instead of (2-((5-chloro-2-((4-(9-(dimethylamino)-3-azaspiro[5.5]undecan-3-yl)-3-nitrophenyl)amino)pyrimidin-4-yl)amino)phenyl)dimethylphosphine oxide to obtain (6-((2-((3-amino-4-(4-(4-methylpiperazin-1-yl)piperidin-1-yl)phenyl)amino)-5-bromopyrimidin-4-yl)amino)quinoxalin-5-yl)dimethylphosphine oxide. MS: 665 [M+H]⁺.

### Step 4: Synthesis of N-(5-((5-bromo-4-((5-(dimethylphosphoryl)quinoxalin-6-yl)amino)pyrimidin-2-yl)amino)-2-(4-(4-methylpiperazin-1-yl)piperidin-1-yl)phenyl)acrylamide

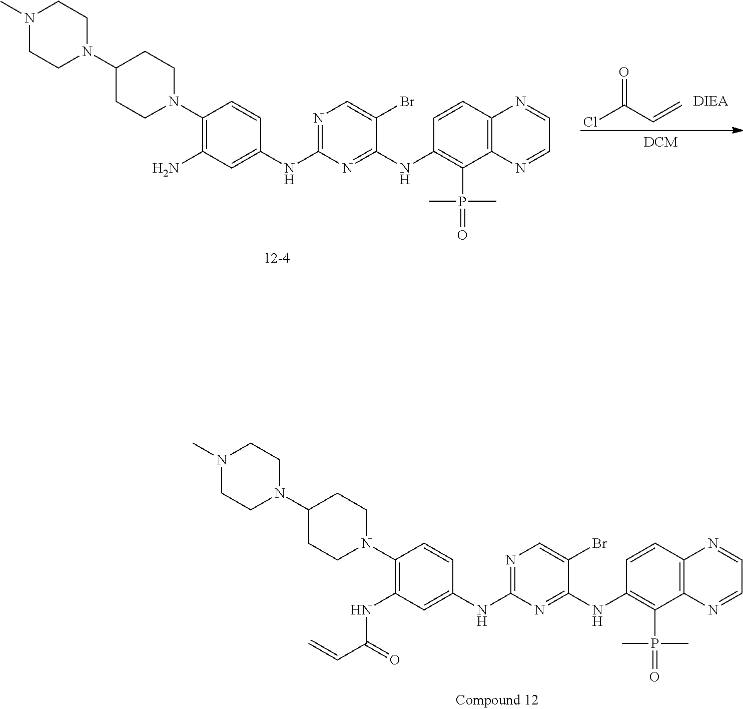

Following the same procedure as N-(5-((5-chloro-4-((2-(dimethylphosphoryl)phenyl)amino)pyrimidin-2-yl)amino)-2-(9-(di methyl amino)-3-azaspiro[5.5]undecan-3-yl)phenyl)acrylamide using (6-((2-((3-amino-4-(4-(4-methylpiperazin-1-yl)piperidin-1-yl)phenyl)amino)-5-bromopyrimidin-4-yl)amino)quinoxalin-5-yl)dimethylphosphine oxide instead of (2-((2-((3-amino-4-(9-(dimethylamino)-3-azaspiro[5.5]undecan-3-yl)phenyl)amino)-5-chloropyrimidin-4-yl)amino)phenyl)dimethylphosphine oxide to obtain N-(5-((5-bromo-4-((5-(dimethylphosphoryl)quinoxalin-6-yl)amino)pyrimidin-2-yl)amino)-2-(4-(4-methylpiperazin-1-yl)piperidin-1-yl)phenyl)acrylamide. MS: 719 [M+H]⁺.

¹H NMR (500 MHz, DMSO-d₆) δ 12.70 (s, 1H), 9.48 (s, 1H), 9.00 (s, 2H), 8.87-8.77 (m, 2H), 8.34 (s, 1H), 8.28 (s, 1H), 8.02 (d, J=9.5 Hz, 1H), 7.37 (s, 1H), 7.08 (d, J=8.7 Hz, 1H), 6.59 (dd, J=16.8, 10.3 Hz, 1H), 6.07 (d, J=17.0 Hz, 1H), 5.64 (d, J=10.3 Hz, 1H), 3.61 (s, 1H), 2.97-2.87 (m, 3H), 2.64-2.58 (m, 4H), 2.05-1.95 (m, 7H), 1.82-1.72 (m, 4H), 1.26-1.16 (m, 1H).

### Example 13 Synthesis of Compound 13

### N-(5-((5-bromo-4-((5-(dimethylphosphoryl)quinoxalin-6-yl)amino)pyrimidin-2-yl)amino)-4-methoxy-2-(4-(4-methylpiperazin-1-yl)piperidin-1-yl)phenyl)acrylamide

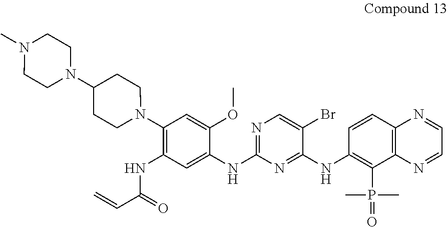 [SMILES: C=CC(=O)Nc1cc(Nc2ncc(Br)c(Nc3ccc4nccnc4c3P(C)(C)=O)n2)c(OC)cc1N1CCC(N2CCN(C)CC2)CC1]

### Step 1: Synthesis of (6-((5-bromo-2-((4-fluoro-2-methoxy-5-nitrophenyl)amino)pyrimidin-4-yl)amino)quinoxalin-5-yl)dimethylphosphine oxide

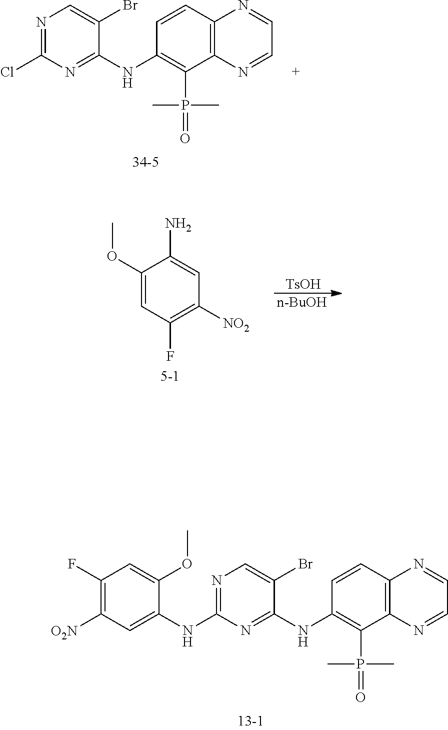

Following the same procedure as (6-((5-bromo-2-((4-fluoro-3-nitrophenyl)amino)pyrimidin-4-yl)amino)quinoxalin-5-yl)dimethylphosphine oxide using 4-fluoro-2-methoxy-5-nitroaniline oxide instead of 4-fluoro-3-nitroaniline to obtain (6-((5-bromo-2-((4-fluoro-2-methoxy-5-nitrophenyl)amino)pyrimidin-4-yl)amino)quinoxalin-5-yl)dimethylphosphine oxide. MS: 562 [M+H]⁺.

### Step 2: Synthesis of (6-((5-bromo-2-((2-methoxy-4-(4-(4-methylpiperazin-1-yl)piperidin-1-yl)-5-nitrophenyl)amino)pyrimidin-4-yl)amino)quinoxalin-5-yl)dimethylphosphine oxide

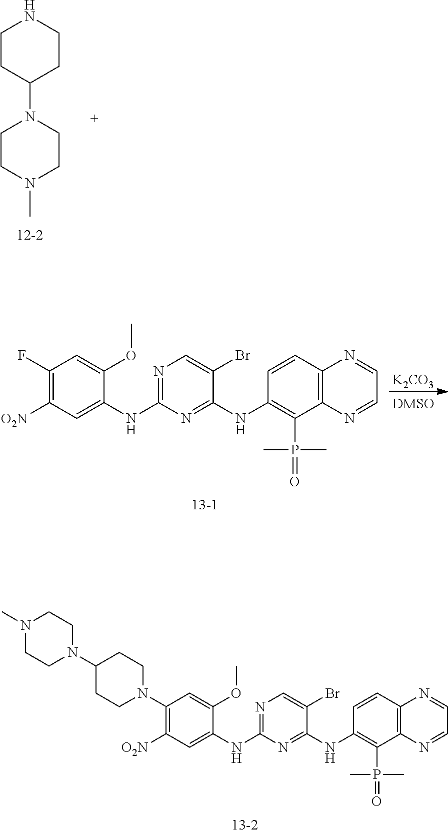

Following the same procedure as (6-((5-bromo-2-((4-(4-(4-methylpiperazin-1-yl)piperidin-1-yl)-3-nitrophenyl)amino)pyrimidin-4-yl)amino)quinoxalin-5-yl)dimethylphosphine oxide using (6-((5-bromo-2-((4-fluoro-2-methoxy-5-nitrophenyl)amino)pyrimidin-4-yl)amino)quinoxalin-5-yl)dimethylphosphine oxide instead of (6-((5-bromo-2-((4-fluoro-3-nitrophenyl)amino)pyrimidin-4-yl)amino)quinoxalin-5-yl)dimethylphosphine oxide to obtain (6-((5-bromo-2-((2-methoxy-4-(4-(4-methylpiperazin-1-yl)piperidin-1-yl)-5-nitrophenyl)amino)pyrimidin-4-yl)amino)quinoxalin-5-yl)dimethylphosphine oxide. MS: 725 [M+H]⁺.

### Step 3: Synthesis of (6-((2-((5-amino-2-methoxy-4-(4-(4-methylpiperazin-1-yl)piperidin-1-yl)phenyl)amino)-5-bromopyrimidin-4-yl)amino)quinoxalin-5-yl)dimethylphosphine oxide

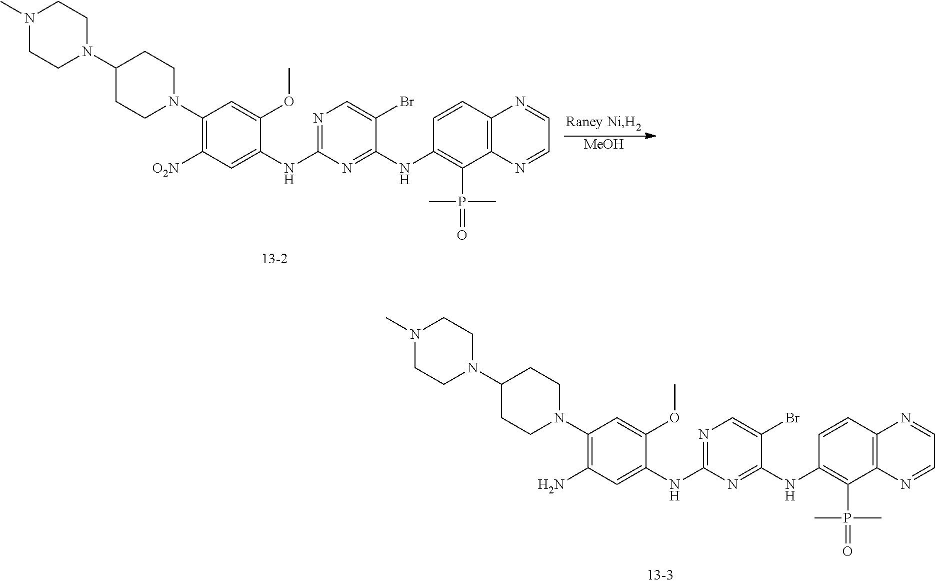 [SMILES: "MeOH" ; COc1cc(N2CCC(N3CCN(C)CC3)CC2)c([N+](=O)[O-])cc1Nc1ncc(Br)c(Nc2ccc3nccnc3c2P(C)(C)=O)n1 ; COc1cc(N2CCC(N3CCN(C)CC3)CC2)c(N)cc1Nc1ncc(Br)c(Nc2ccc3nccnc3c2P(C)(C)=O)n1]

Following the same procedure as (6-((2-((3-amino-4-(4-(4-methylpiperazin-1-yl)piperidin-1-yl)phenyl)amino)-5-bromopyrimidin-4-yl)amino)quinoxalin-5-yl)dimethylphosphine oxide using (6-((5-bromo-2-((2-methoxy-4-(4-(4-methylpiperazin-1-yl)piperidin-1-yl)-5-nitrophenyl)amino)pyrimidin-4-yl)amino)quinoxalin-5-yl)dimethylphosphine oxide instead of (6-((5-bromo-2-((4-(4-(4-methylpiperazin-1-yl)piperidin-1-yl)-3-nitrophenyl)amino)pyrimidin-4-yl)amino)quinoxalin-5-yl)dimethylphosphine oxide to obtain (6-((2-((5-amino-2-methoxy-4-(4-(4-methylpiperazin-1-yl)piperidin-1-yl)phenyl)amino)-5-bromopyrimidin-4-yl)amino)quinoxalin-5-yl)dimethylphosphine oxide. MS: 695 [M+H]⁺.

### Step 4: Synthesis of N-(5-((5-bromo-4-((5-(dimethylphosphoryl)quinoxalin-6-yl)amino)pyrimidin-2-yl)amino)-4-methoxy-2-(4-(4-methylpiperazin-1-yl)piperidin-1-yl)phenyl)acrylamide

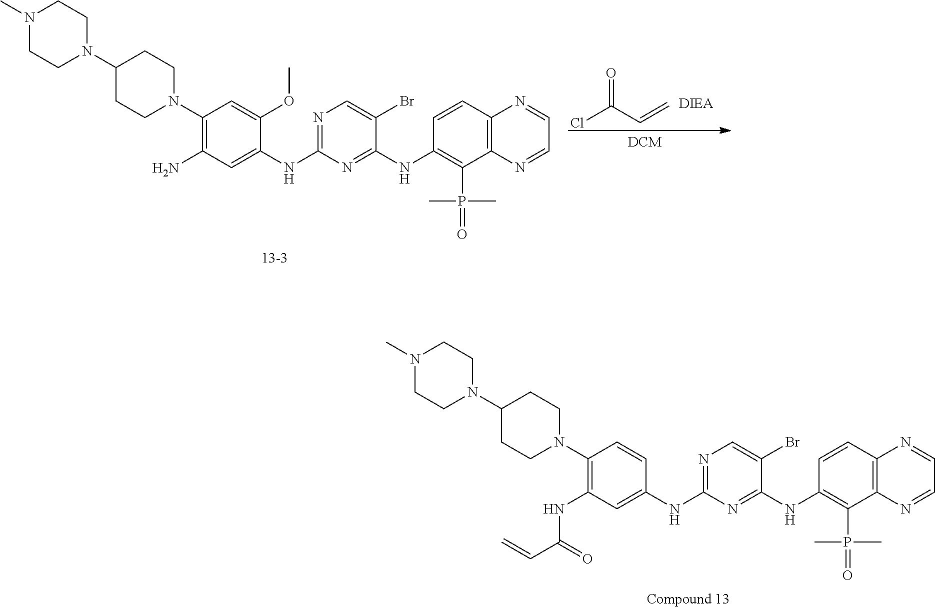

Following the same procedure as N-(5-((5-bromo-4-((5-(dimethylphosphoryl)quinoxalin-6-yl)amino)pyrimidin-2-yl)amino)-2-(4-(4-methylpiperazin-1-yl)piperidin-1-yl)phenyl)acrylamide using (6-((2-((5-amino-2-methoxy-4-(4-(4-methylpiperazin-1-yl)piperidin-1-yl)phenyl)amino)-5-bromopyrimidin-4-yl)amino)quinoxalin-5-yl)dimethylphosphine oxide instead of (6-((2-((3-amino-4-(4-(4-methylpiperazin-1-yl)piperidin-1-yl)phenyl)amino)-5-bromopyrimidin-4-yl)amino)quinoxalin-5-yl)dimethylphosphine oxide to obtain N-(5-((5-bromo-4-((5-(dimethylphosphoryl)quinoxalin-6-yl)amino)pyrimidin-2-yl)amino)-4-methoxy-2-(4-(4-methylpiperazin-1-yl)piperidin-1-yl)phenyl)acrylamide. MS: 719 [M+H]⁺.

¹H NMR (500 MHz, DMSO-d₆) δ 12.68 (s, 1H), 8.95 (s, 1H), 8.84-8.74 (m, 3H), 8.46 (s, 1H), 8.28 (s, 1H), 8.14 (s, 1H), 7.86 (d, J=9.4 Hz, 1H), 6.88 (s, 1H), 6.59 (dd, J=17.0, 10.2 Hz, 1H), 6.11-6.01 (m, 1H), 5.61 (d, J=10.4 Hz, 1H), 3.79 (s, 3H), 3.06-2.96 (m, 3H), 2.71-2.61 (m, 4H), 2.02-1.92 (m, 6H), 1.85-1.75 (m, 4H), 1.25-1.15 (m, 3H).

### Example 14 Synthesis of Compound 14

### N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt

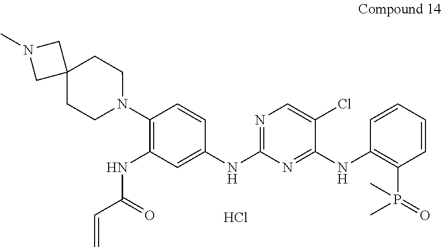 [SMILES: C=CC(=O)Nc1cc(Nc2ncc(Cl)c(Nc3ccccc3P(C)(C)=O)n2)ccc1N1CCC2(CC1)CN(C)C2 ; Cl]

### Step 1: Synthesis of tert-butyl 7-(4-((5-chloro-4-((2-(dimethylphosphoryl)phenyl)amino)pyrimidin-2-yl)amino)-2-nitrophenyl)-2,7-diazaspiro[3.5]nonane-2-carboxylate

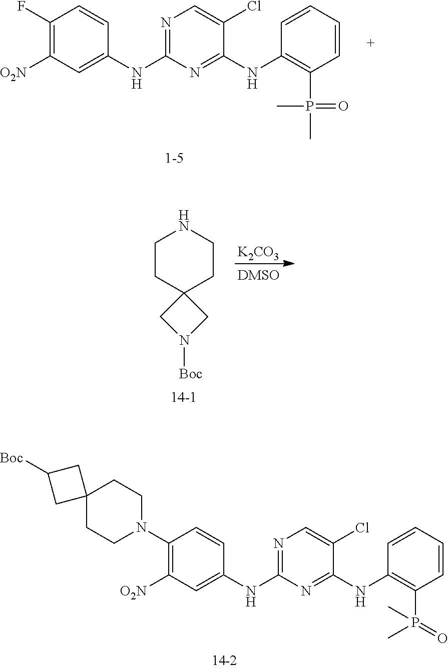

To a mixture of (2-((5-chloro-2-((4-fluoro-3-nitrophenyl)amino)pyrimidin-4-yl)amino)phenyl)dimethylphosphine oxide (500 mg) in DMSO (5 mL), tert-butyl 2,7-diazaspiro[3.5]nonane-2-carboxylate (312 mg) and potassium carbonate (317 mg) was added under stirring. The mixture was heated 90° C. for about 8 h. The mixture solution was poured into water and extracted with ethyl acetate (50 mL*3). The combined organic layer was washed with saturated sodium chloride aqueous solution (50 mL*2), dried over Na₂SO₄ and concentrated under reduced pressure to afford the tert-butyl 7-(4-((5-chloro-4-((2-(dimethylphosphoryl)phenyl)amino)pyrimidin-2-yl)amino)-2-nitrophenyl)-2,7-diazaspiro[3.5]nonane-2-carboxylate (600 mg) as red solid. MS: 642 [M+H]⁺.

### Step 2: Synthesis of (2-((5-chloro-2-((3-nitro-4-(2,7-diazaspiro[3.5]nonan-7-yl)phenyl)amino)pyrimidin-4-yl)amino)phenyl)dimethylphosphine oxide trifluoroacetic acid salt

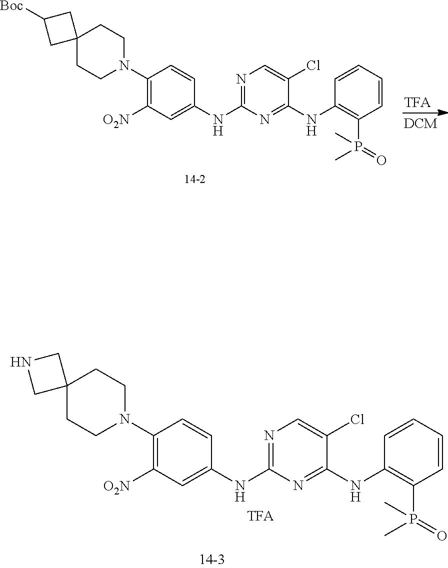 [SMILES: CC(C)(C)OC(=O)C1CC2(CCN(c3ccc(Nc4ncc(Cl)c(Nc5ccccc5P(C)(C)=O)n4)cc3[N+](=O)[O-])CC2)C1 ; CP(C)(=O)c1ccccc1Nc1nc(Nc2ccc(N3CCC4(CC3)CNC4)c([N+](=O)[O-])c2)ncc1Cl]

To a solution of tert-butyl 7-(4-((5-chloro-4-((2-(dimethylphosphoryl)phenyl)amino)pyrimidin-2-yl)amino)-2-nitrophenyl)-2,7-diazaspiro[3.5]nonane-2-carboxylate (600 mg) in DCM (5 mL) was added TFA (5 mL). The mixture was stirred 5 h at room temperature. The mixture was concentrated under reduced pressure to afford the (2-((5-chloro-2-((3-nitro-4-(2,7-diazaspiro[3.5]nonan-7-yl)phenyl)amino)pyrimidin-4-yl)amino)phenyl)dimethylphosphine oxide trifluoroacetic acid salt (600 mg) as red semi-solid. MS: 542 [M+H]⁺.

### Step 3: Synthesis of (2-((5-chloro-2-((4-(2-methyl-2,7-diazaspiro[3.5]nonan-7-yl)-3-nitrophenyl)amino)pyrimidin-4-yl)amino)phenyl)dimethylphosphine oxide

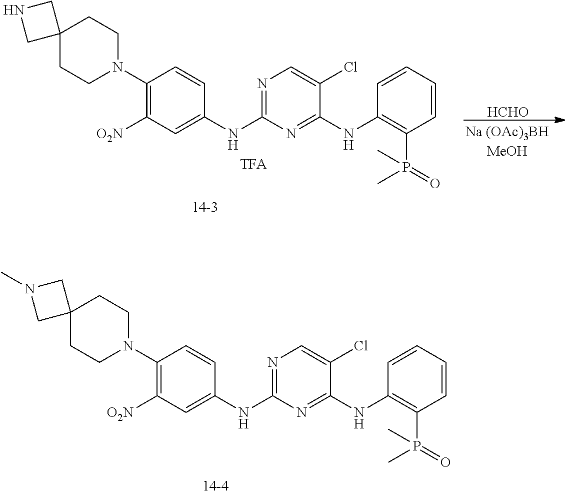

To a solution of (2-((5-chloro-2-((3-nitro-4-(2,7-diazaspiro[3.5]nonan-7-yl)phenyl)amino)pyrimidin-4-yl)amino)phenyl)dimethylphosphine oxide trifluoroacetic acid salt (600 mg) in methanol (10 mL) was added HCHO (1 mL). The mixture was stirred 30 min at room temperature. Then the reaction mixture was added Na(OAc)₃BH (970 mg), and stirred another 2 h. The mixture solution was poured into water and extracted with DCM (15 mL*3). The combined organic layer was washed with saturated sodium chloride aqueous solution (50 mL*2), dried over Na₂SO₄ and concentrated under reduced pressure to afford the (2-((5-chloro-2-((4-(2-methyl-2,7-diazaspiro[3.5]nonan-7-yl)-3-nitrophenyl)amino)pyrimidin-4-yl)amino)phenyl)dimethylphosphine oxide (500 mg) as red solid. MS: 556 [M+H]⁺.

### Step 4: Synthesis of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide

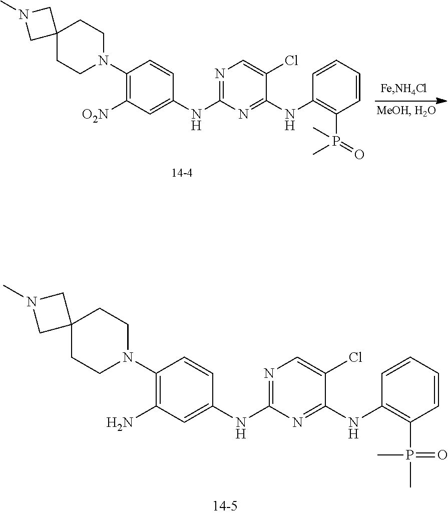 [SMILES: CN1CC2(CCN(c3ccc(Nc4ncc(Cl)c(Nc5ccccc5P(C)(C)=O)n4)cc3[N+](=O)[O-])CC2)C1 ; CN1CC2(CCN(c3ccc(Nc4ncc(Cl)c(Nc5ccccc5P(C)(C)=O)n4)cc3N)CC2)C1]

To a solution of (2-((5-chloro-2-((4-(2-methyl-2,7-diazaspiro[3.5]nonan-7-yl)-3-nitrophenyl)amino)pyrimidin-4-yl)amino)phenyl)dimethylphosphine oxide (500 mg) in methanol/H₂O (20 mL, 5:1) was added Fe (250 mg) and ammonium chloride (96 mg). The mixture was heated at 80° C. for 5 h. The mixture was then filtered through diatomaceous earth and washed with methanol; the filtrate was then concentrated under reduced pressure and purified by silica gel column chromatography using DCM/methanol (95:5) as the eluent, and to obtain (2-((2-((3-amino-4-(2-methyl-2,7-diazaspiro[3.5]nonan-7-yl)phenyl)amino)-5-chloropyrimidin-4-yl)amino)phenyl)dimethylphosphine oxide (400 mg) as off-white solid. MS: 526 [M+H]⁺.

### Step 5: Synthesis of N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt

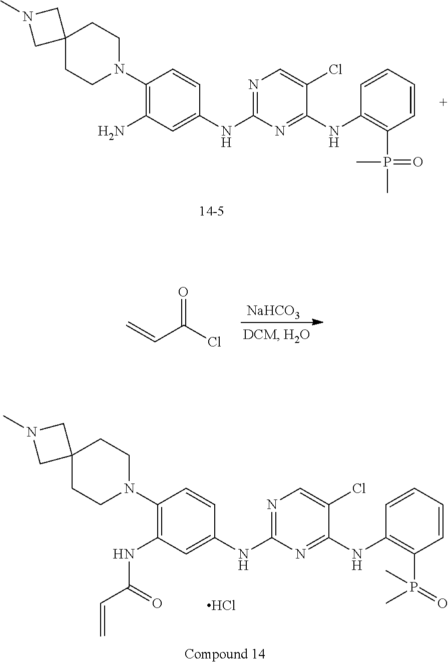

To a solution of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide (200 mg) in DCM/H₂O (10 mL, 5:1) was added NaHCO₃ (100 mg). The mixture was added acryloyl chloride (34 mg) dropwise at 0° C., and stirred another 2 h. The mixture solution was poured into water and extracted with DCM (15 mL*3). The combined organic layer was washed with saturated sodium chloride aqueous solution (50 mL*2), dried over Na₂SO₄ and concentrated under reduced pressure, purified by C18 silica gel column chromatography using H₂O (0.5% HCO/methanol (20%-30%) as the eluent to afford N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt (40 mg) as yellow solid. MS: 580 [M+H]⁺.

¹H NMR (500 MHz, Methanol-d₄) δ 8.24 (m, 1H), 8.18 (m, 1H), 7.79 (s, 1H), 7.69-7.68 (m, 2H), 7.54-7.53 (m, 1H), 7.41-7.40 (m, 2H), 6.62 (dd, J=17.0, 8.0 Hz, 1H), 6.53 (dd, J=17.0, 8.0 Hz, 1H), 5.99-5.98 (m, 1H), 4.55-4.30 (m, 2H), 4.03-3.82 (m, 2H), 3.60-3.45 (m, 2H), 3.20-2.97 (m, 2H), 2.30 (s, 3H), 1.88-1.31 (m, 4H).

### Example 15 Synthesis of Compound 15

### N-(5-((5-chloro-4-((2-(dimethylphosphoryl)phenyl)amino)pyrimidin-2-yl)amino)-2-((2-(dimethylamino)ethyl)(methyl)amino)phenyl)acrylamide

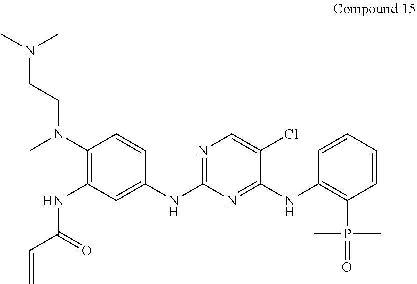

### Step 1: Synthesis of (2-((5-chloro-2-((4-((2-(dimethylamino)ethyl)(methyl)amino)-3-nitrophenyl)amino)pyrimidin-4-yl)amino)phenyl)dimethylphosphine oxide

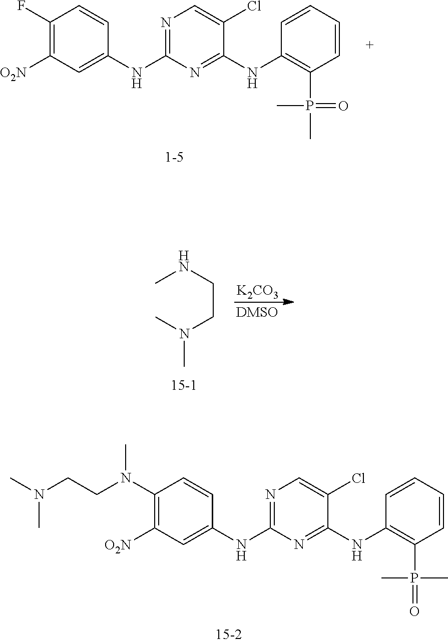

Following the same procedure as using N¹,N¹,N²-trimethylethane-1,2-diamine instead of tert-butyl 2,7-diazaspiro[3.5]nonane-2-carboxylate to obtain (2-((5-chloro-2-((4-((2-(dimethylamino)ethyl)(methyl)amino)-3-nitrophenyl)amino)pyrimidin-4-yl)amino)phenyl)dimethylphosphine oxide. MS: 518 [M+H]⁺.

### Step 2: Synthesis of (2-((2-((3-amino-4-((2-(dimethylamino)ethyl)(methyl)amino)phenyl)amino)-5-chloropyrimidin-4-yl)amino)phenyl)dimethylphosphine oxide

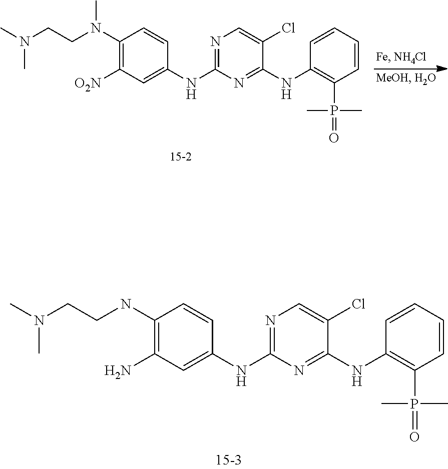

Following the same procedure as (2-((2-((3-amino-4-(2-methyl-2,7-diazaspiro[3.5]nonan-7-yl)phenyl)amino)-5-chloropyrimidin-4-yl)amino)phenyl)dimethylphosphine oxide using (2-((5-chloro-2-((4-((2-(dimethylamino)ethyl)(methyl)amino)-3-nitrophenyl)amino)pyrimidin-4-yl)amino)phenyl)dimethylphosphine oxide instead of (2-((5-chloro-2-((4-(2-methyl-2,7-diazaspiro[3.5]nonan-7-yl)-3-nitrophenyl)amino)pyrimidin-4-yl)amino)phenyl)dimethylphosphine oxide to obtain (2-((2-((3-amino-4-((2-(dimethylamino)ethyl)(methyl)amino)phenyl)amino)-5-chloropyrimidin-4-yl)amino)phenyl)dimethylphosphine oxide. MS: 488 [M+H]⁺.

### Step 3: Synthesis of N-(5-((5-chloro-4-((2-(dimethylphosphoryl)phenyl)amino)pyrimidin-2-yl)amino)-2-((2-(dimethylamino)ethyl)(methyl)amino)phenyl)acrylamide (Compound 15)

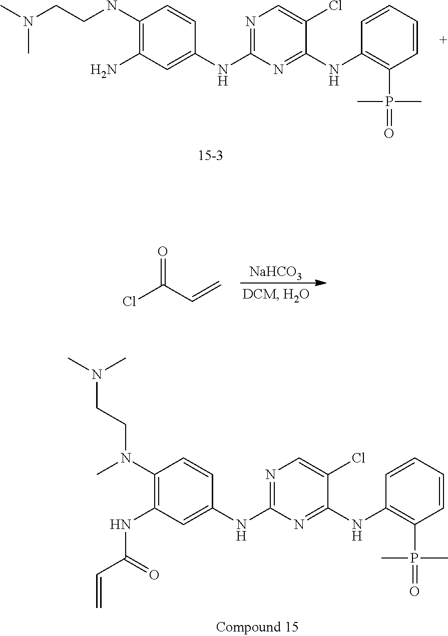

Following the same procedure as N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt using (2-((2-((3-amino-4-((2-(dimethylamino)ethyl)(methyl)amino)phenyl)amino)-5-chloropyrimidin-4-yl)amino)phenyl)dimethylphosphine oxide instead of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide to obtain N-(5-((5-chloro-4-((2-(dimethylphosphoryl)phenyl)amino)pyrimidin-2-yl)amino)-2-((2-(dimethylamino)ethyl)(methyl)amino)phenyl)acrylamide (Compound 15). MS: 542 [M+H]⁺.

¹H NMR (500 MHz, DMSO-d₆) δ 11.24 (s, 1H), 10.26 (s, 1H), 9.88 (s, 1H), 9.41 (s, 1H), 8.67 (s, 1H), 8.30 (s, 1H), 8.17 (s, 1H), 7.57-7.56 (m, 1H), 7.46-7.45 (m, 2H), 7.19 (d, J=8.7 Hz, 1H), 7.15-7.09 (m, 1H), 6.25 (dd, J=16.9, 2.0 Hz, 1H), 5.75 (dd, J=16.9, 2.0 Hz, 1H), 3.34 (s, 3H), 2.64-2.60 (m, 4H), 1.78 (s, 6H).

### Example 16 Synthesis of Compound 16

### N-(5-((5-chloro-4-((2-(dimethylphosphoryl)phenyl)amino)pyrimidin-2-yl)amino)-2-(4-(4-methylpiperazin-1-yl)piperidin-1-yl)phenyl)acrylamide

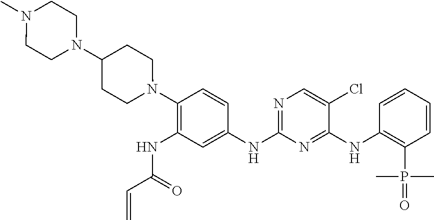

### Step 1: Synthesis of (2-((5-chloro-2-((4-(4-(4-methylpiperazin-1-yl)piperidin-1-yl)-3-nitrophenyl)amino)pyrimidin-4-yl)amino)phenyl)dimethylphosphine oxide

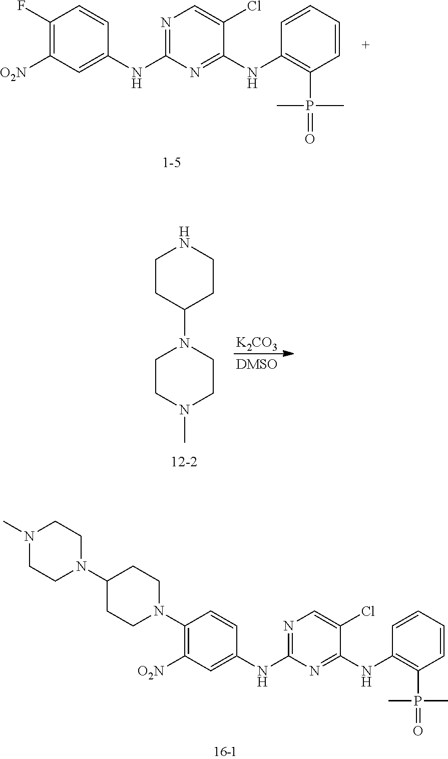

Following the same procedure as 7-(4-((5-chloro-4-((2-(dimethylphosphoryl)phenyl)amino)pyrimidin-2-yl)amino)-2-nitrophenyl)-2,7-diazaspiro[3.5]nonane-2-carboxylate using 1-methyl-4-(piperidin-4-yl)piperazine instead of tert-butyl 2,7-diazaspiro[3.5]nonane-2-carboxylate to obtain (2-((5-chloro-2-((4-(4-(4-methylpiperazin-1-yl)piperidin-1-yl)-3-nitrophenyl)amino)pyrimidin-4-yl)amino)phenyl)dimethylphosphine oxide. MS: 599 [M+H]⁺.

### Step 2: Synthesis of (2-((2-((3-amino-4-(4-(4-methylpiperazin-1-yl)piperidin-1-yl)phenyl)amino)-5-chloropyrimidin-4-yl)amino)phenyl)dimethylphosphine oxide

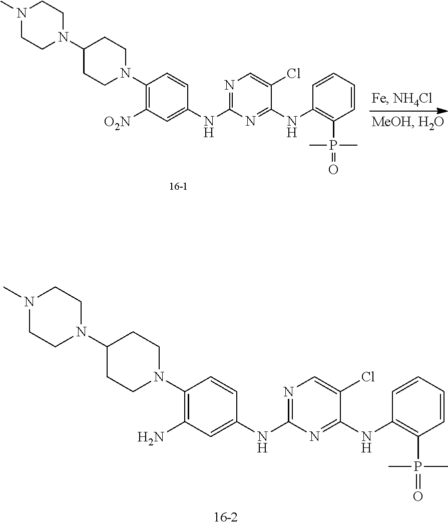

Following the same procedure as (2-((2-((3-amino-4-(2-methyl-2,7-diazaspiro[3.5]nonan-7-yl)phenyl)amino)-5-chloropyrimidin-4-yl)amino)phenyl)dimethylphosphine oxide using (2-((5-chloro-2-((4-(4-(4-methylpiperazin-1-yl)piperidin-1-yl)-3-nitrophenyl)amino)pyrimidin-4-yl)amino)phenyl)dimethylphosphine oxide instead of (2-((5-chloro-2-((4-(2-methyl-2,7-diazaspiro[3.5]nonan-7-yl)-3-nitrophenyl)amino)pyrimidin-4-yl)amino)phenyl)dimethylphosphine oxide to obtain (2-((2-((3-amino-4-(4-(4-methylpiperazin-1-yl)piperidin-1-yl)phenyl)amino)-5-chloropyrimidin-4-yl)amino)phenyl)dimethylphosphine oxide. MS: 569 [M+H]⁺.

### Step 3: Synthesis of N-(5-((5-chloro-4-((2-(dimethylphosphoryl)phenyl)amino)pyrimidin-2-yl)amino)-2-(4-(4-methylpiperazin-1-yl)piperidin-1-yl)phenyl)acrylamide

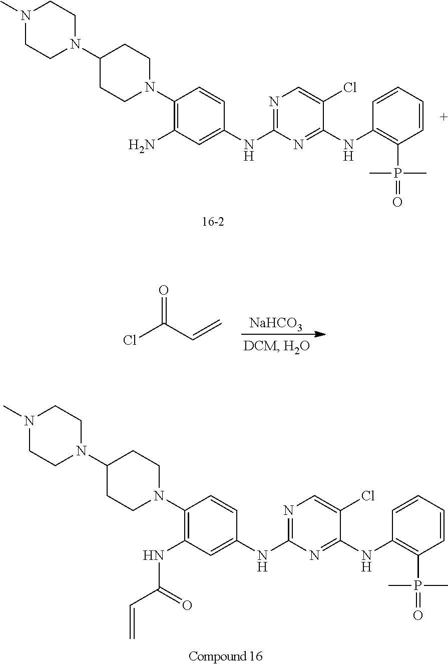

Following the same procedure as N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt using (2-((2-((3-amino-4-(4-(4-methyl piperazin-1-yl)piperidin-1-yl)phenyl)amino)-5-chloropyrimidin-4-yl)amino)phenyl)dimethylphosphine oxide instead of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide to obtain N-(5-((5-chloro-4-((2-(dimethylphosphoryl)phenyl)amino)pyrimidin-2-yl)amino)-2-(4-(4-methylpiperazin-1-yl)piperidin-1-yl)phenyl)acrylamide. MS: 623 [M+H]+.

¹H NMR (500 MHz, DMSO-d₆) δ 11.23 (s, 1H), 9.38 (s, 1H), 9.06 (s, 1H), 8.68 (d, J=7.5 Hz, 1H), 8.17 (d, J=11.8 Hz, 2H), 7.57-5.56 (m, 1H), 7.46-7.45 (m, 2H), 7.21-7.04 (m, 2H), 6.69-6.68 (m, 1H), 6.23-6.22 (m, 1H), 5.79-5.73 (m, 1H), 3.34 (s, 3H), 2.98-2.48 (m, 12H), 1.85-1.15 (m, 5H).

### Example 17 Synthesis of Compound 17

### (S)—N-(5-((5-chloro-4-((2-(dimethylphosphoryl)phenyl)amino)pyrimidin-2-yl)amino)-2-(hexahydropyrrolo[1,2-a]pyrazin-2(1H)-yl)phenyl)acrylamide

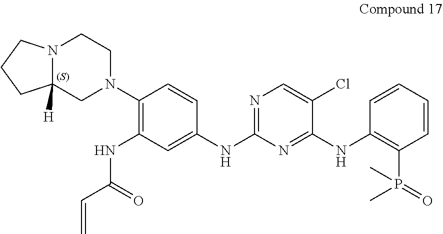

### Step 1: Synthesis of (S)-(2-((5-chloro-2-((4-(hexahydropyrrolo[1,2-a]pyrazin-2(1H)-yl)-3-nitrophenyl)amino)pyrimidin-4-yl)amino)phenyl)dimethylphosphine oxide

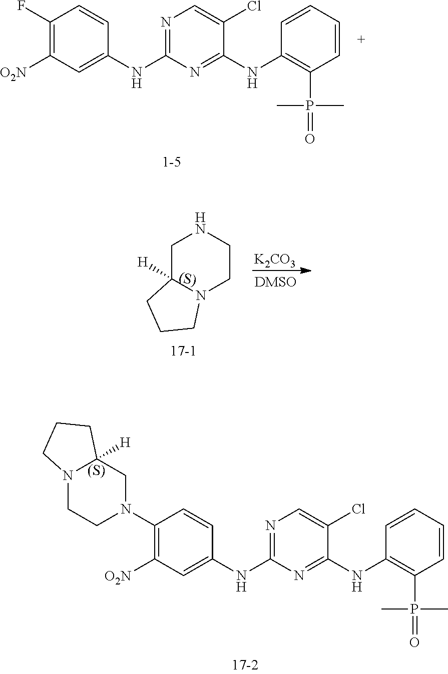

Following the same procedure as tert-butyl 7-(4-((5-chloro-4-((2-(dimethylphosphoryl)phenyl)amino)pyrimidin-2-yl)amino)-2-nitrophenyl)-2,7-diazaspiro[3.5]nonane-2-carboxylate using (S)-octahydropyrrolo[1,2-a]pyrazine instead of tert-butyl 2,7-diazaspiro[3.5]nonane-2-carboxylate to obtain (S)-(2-((5-chloro-2-((4-(hexahydropyrrolo[1,2-a]pyrazin-2(1H)-yl)-3-nitrophenyl)amino)pyrimidin-4-yl)amino)phenyl)dimethylphosphine oxide. MS: 542 [M+H]⁺.

### Step 2: Synthesis of (S)-(2-((2-((3-amino-4-(hexahydropyrrolo[1,2-a]pyrazin-2(1H)-yl)phenyl)amino)-5-chloropyrimidin-4-yl)amino)phenyl)dimethylphosphine oxide

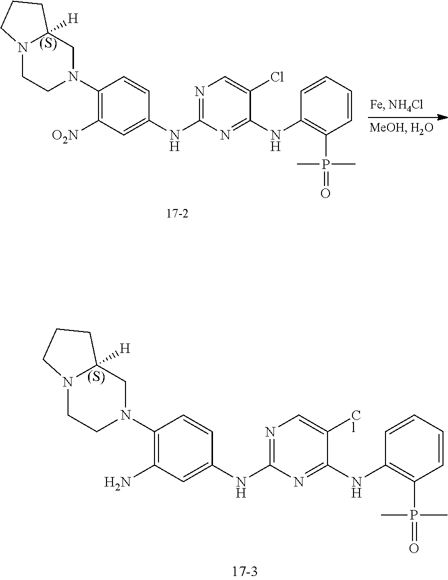

Following the same procedure as (2-((2-((3-amino-4-(2-methyl-2,7-diazaspiro[3.5]nonan-7-yl)phenyl)amino)-5-chloropyrimidin-4-yl)amino)phenyl)dimethylphosphine oxide using (S)-(2-((5-chloro-2-((4-(hexahydropyrrolo[1,2-a]pyrazin-2 (1H)-yl)-3-nitrophenyl)amino)pyrimidin-4-yl)amino)phenyl)dimethylphosphine oxide instead of (2-((5-chloro-2-((4-(2-methyl-2,7-diazaspiro[3.5]nonan-7-yl)-3-nitrophenyl)amino)pyrimidin-4-yl)amino)phenyl)dimethylphosphine oxide to obtain (S)-(2-((2-((3-amino-4-(hexahydropyrrolo[1,2-a]pyrazin-2(1H)-yl)phenyl)amino)-5-chloropyrimidin-4-yl)amino)phenyl)dimethylphosphine oxide. MS: 512 [M+H]⁺.

### Step 3: Synthesis of (S)—N-(5-((5-chloro-4-((2-(dimethylphosphoryl)phenyl)amino)pyrimidin-2-yl)amino)-2-(hexahydropyrrolo[1,2-a]pyrazin-2(1H)-yl)phenyl)acrylamide

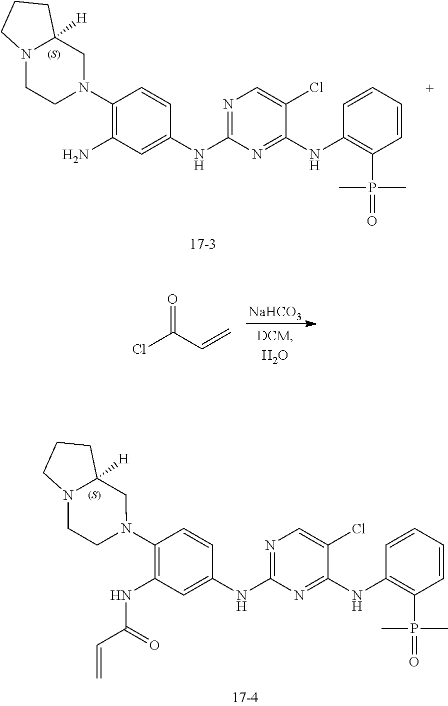

Following the same procedure as N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt using (S)-(2-((2-((3-amino-4-(hexahydropyrrolo[1,2-a]pyrazin-2 (1H)-yl)phenyl)amino)-5-chloropyrimidin-4-yl)amino)phenyl)dimethylphosphine oxide instead of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide to obtain (S)—N-(5-((5-chloro-4-((2-(dimethylphosphoryl)phenyl)amino)pyrimidin-2-yl)amino)-2-(hexahydropyrrolo[1,2-a]pyrazin-2(1H)-yl)phenyl)acrylamide. MS: 566 [M+H]⁺.

### Example 18 Synthesis of Compound 18

### N-(5-((5-chloro-4-((2-(dimethylphosphoryl)phenyl)amino)pyrimidin-2-yl)amino)-2-(methyl(2-(pyrrolidin-1-yl)ethyl)amino)phenyl)acrylamide

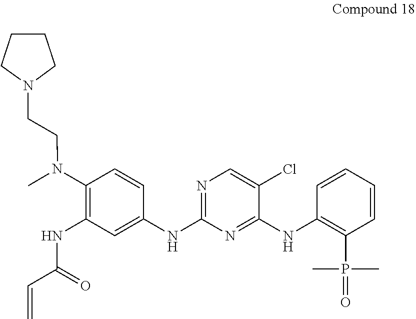 [SMILES: C=CC(=O)Nc1cc(Nc2ncc(Cl)c(Nc3ccccc3P(C)(C)=O)n2)ccc1N(C)CCN1CCCC1]

### Step 1: Synthesis of (2-((5-chloro-2-((4-(methyl(2-(pyrrolidin-1-yl)ethyl)amino)-3-nitrophenyl)amino)pyrimidin-4-yl)amino)phenyl)dimethylphosphine oxide

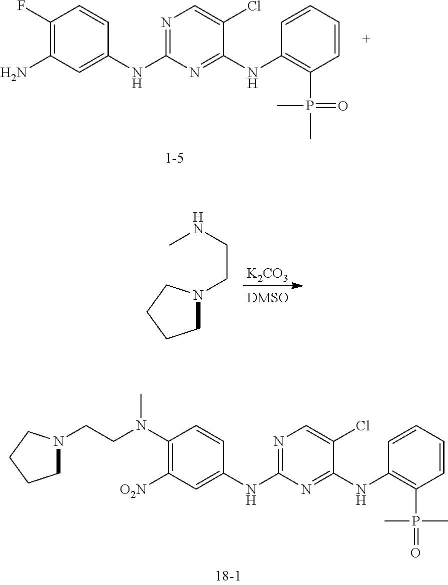

Following the same procedure as tert-butyl 7-(4-((5-chloro-4-((2-(dimethylphosphoryl)phenyl)amino)pyrimidin-2-yl)amino)-2-nitrophenyl)-2,7-diazaspiro[3.5]nonane-2-carboxylate using N-methyl-2-(pyrrolidin-1-yl)ethanamine instead of tert-butyl 2,7-diazaspiro[3.5]nonane-2-carboxylate to obtain (2-((5-chloro-2-((4-(methyl(2-(pyrrolidin-1-yl)ethyl)amino)-3-nitrophenyl)amino)pyrimidin-4-yl)amino)phenyl)dimethylphosphine oxide. MS: 544 [M+H]⁺.

### Step 2: Synthesis of (2-((2-((3-amino-4-(methyl(2-(pyrrolidin-1-yl)ethyl)amino)phenyl)amino)-5-chloropyrimidin-4-yl)amino)phenyl)dimethylphosphine oxide

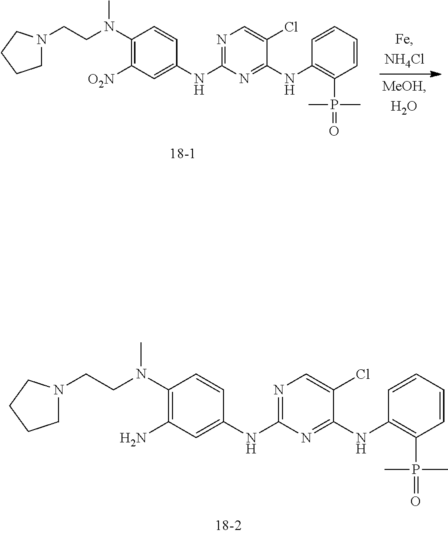 [SMILES: CN(CCN1CCCC1)c1ccc(Nc2ncc(Cl)c(Nc3ccccc3P(C)(C)=O)n2)cc1[N+](=O)[O-] ; CN(CCN1CCCC1)c1ccc(Nc2ncc(Cl)c(Nc3ccccc3P(C)(C)=O)n2)cc1N]

Following the same procedure as (2-((2-((3-amino-4-(2-methyl-2,7-diazaspiro[3.5]nonan-7-yl)phenyl)amino)-5-chloropyrimidin-4-yl)amino)phenyl)dimethylphosphine oxide using (2-((5-chloro-2-((4-(methyl(2-(pyrrolidin-1-yl)ethyl)amino)-3-nitrophenyl)amino)pyrimidin-4-yl)amino)phenyl)dimethylphosphine oxide instead of (2-((5-chloro-2-((4-(2-methyl-2,7-diazaspiro[3.5]nonan-7-yl)-3-nitrophenyl)amino)pyrimidin-4-yl)amino)phenyl)dimethylphosphine oxide to obtain (2-((2-((3-amino-4-(methyl(2-(pyrrolidin-1-yl)ethyl)amino)phenyl)amino)-5-chloropyrimidin-4-yl)amino)phenyl)dimethylphosphine oxide. MS: 514 [M+H]⁺.

### Step 3: Synthesis of N-(5-((5-chloro-4-((2-(dimethylphosphoryl)phenyl)amino)pyrimidin-2-yl)amino)-2-(methyl(2-(pyrrolidin-1-yl)ethyl)amino)phenyl)acrylamide (Compound 18)

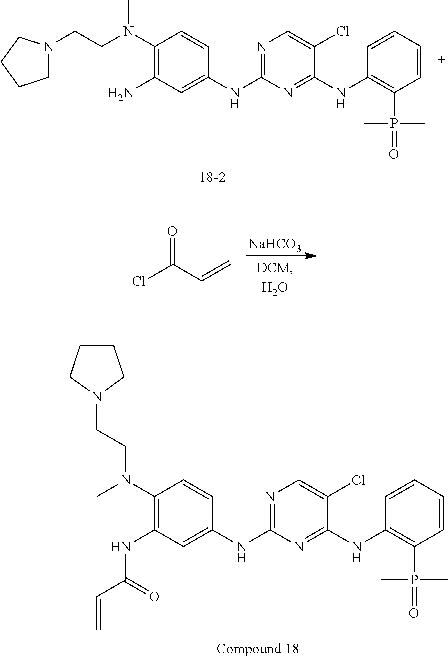

Following the same procedure as N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt using (2-((2-((3-amino-4-(methyl(2-(pyrrolidin-1-yl)ethyl)amino)phenyl)amino)-5-chloropyrimidin-4-yl)amino)phenyl)dimethylphosphine oxide instead of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide to obtain N-(5-((5-chloro-4-((2-(dimethylphosphoryl)phenyl)amino)pyrimidin-2-yl)amino)-2-(methyl(2-(pyrrolidin-1-yl)ethyl)amino)phenyl)acrylamide (Compound 18). MS: 568 [M+H]⁺.

### Example 19 Synthesis of Compound 19

### N-(2-((2-(dimethylamino)ethyl)(methyl)amino)-5-((4-((2-(dimethylphosphoryl)phenyl)amino)-5-methylpyrimidin-2-yl)amino)phenyl)acrylamide hydrochloric acid salt

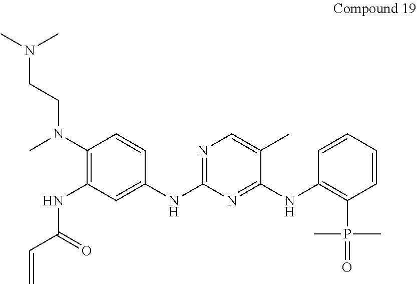

### Step 1: Synthesis of (2-((2-chloro-5-methylpyrimidin-4-yl)amino)phenyl)dimethylphosphine oxide

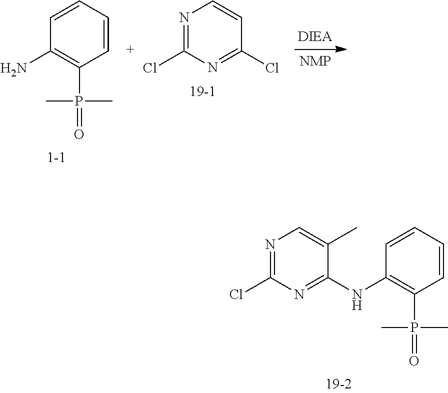 [SMILES: CP(C)(=O)c1ccccc1N ; Clc1ccnc(Cl)n1 ; Cc1cnc(Cl)nc1Nc1ccccc1P(C)(C)=O]

To a mixture of (2-aminophenyl)dimethylphosphine oxide (5.00 g) in NMP (50 mL), 2,4-dichloro-5-methylpyrimidine (5.78 g) and potassium carbonate (11.46 g) was added under stirring. The mixture was heated 130° C. for about 12 h. The mixture solution was poured into water and extracted with DCM (50 mL*3). The combined organic layer was washed with saturated sodium chloride aqueous solution (50 mL*2), dried over Na₂SO₄ a and concentrated to give crude product, which was purified by silica gel column chromatography using DCM/MeOH (5%-6%) as the eluent, and to obtain (2-((2-chloro-5-methylpyrimidin-4-yl)amino)phenyl)dimethylphosphine oxide (5.00 g) as brown solid. MS: 296 [M+H]⁺.

### Step 2: Synthesis of (2-((2-((4-fluoro-3-nitrophenyl)amino)-5-methylpyrimidin-4-yl)amino)phenyl)dimethylphosphine oxide

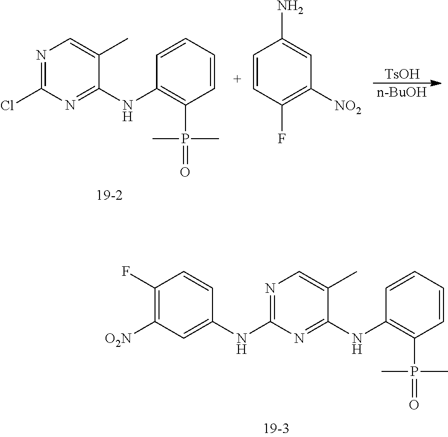

Following the same procedure as (2-((5-chloro-2-((4-fluoro-3-nitrophenyl)amino)pyrimidin-4-yl)amino)phenyl)dimethylphosphine oxide using (2-((2-chloro-5-methylpyrimidin-4-yl)amino)phenyl)dimethylphosphine oxide instead of (2-((2,5-dichloropyrimidin-4-yl)amino)phenyl)dimethylphosphine oxide to obtain (2-((2-((4-fluoro-3-nitrophenyl)amino)-5-methylpyrimidin-4-yl)amino)phenyl)dimethylphosphine oxide. MS: 415 [M+H]⁺.

### Step 3: Synthesis of (2-((2-((4-((2-(dimethylamino)ethyl)(methyl)amino)-3-nitrophenyl)amino)-5-methylpyrimidin-4-yl)amino)phenyl)dimethylphosphine oxide

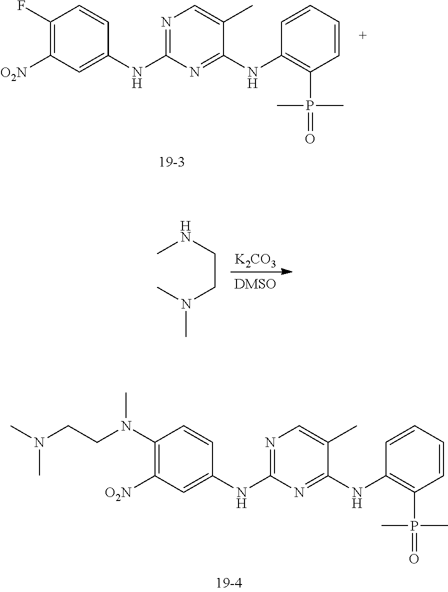

Following the same procedure as (2-((5-chloro-2-((4-((2-(dimethylamino)ethyl)(methyl)amino)-3-nitrophenyl)amino)pyrimidin-4-yl)amino)phenyl)dimethylphosphine oxide using (2-((2-((4-fluoro-3-nitrophenyl)amino)-5-methylpyrimidin-4-yl)amino)phenyl)dimethylphosphine oxide instead of (2-((5-chloro-2-((4-fluoro-3-nitrophenyl)amino)pyrimidin-4-yl)amino)phenyl)dimethylphosphine oxide to obtain (2-((2-((4-((2-(dimethylamino)ethyl)(methyl)amino)-3-nitrophenyl)amino)-5-methylpyrimidin-4-yl)amino)phenyl)dimethylphosphine oxide. MS: 544 [M+H]⁺.

### Step 4: Synthesis of (2-((2-((3-amino-4-((2-(dimethylamino)ethyl)(methyl)amino)phenyl)amino)-5-methylpyrimidin-4-yl)amino)phenyl)dimethylphosphine oxide

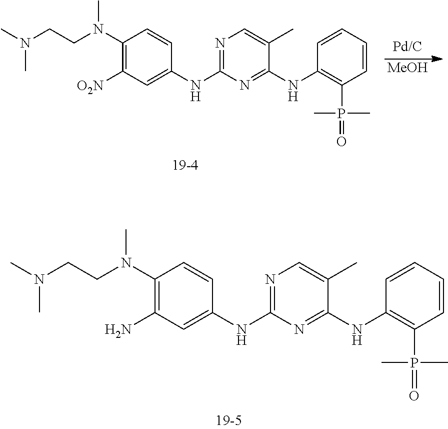

To a solution of (2-((2-((4-((2-(dimethylamino)ethyl)(methyl)amino)-3-nitrophenyl)amino)-5-methylpyrimidin-4-yl)amino)phenyl)dimethylphosphine oxide (500 mg) in methanol (20 mL) was added 10% palladium on carbon (100 mg) and the mixture hydrogenated (hydrogen balloon) at room temperature for 5 h. The mixture was then filtered through diatomaceous earth and washed with methanol; the filtrate was then concentrated under reduced pressure to afford the (2-((2-((3-amino-4-((2-(dimethylamino)ethyl)(methyl)amino)phenyl)amino)-5-methylpyrimidin-4-yl)amino)phenyl)dimethylphosphine oxide (200 mg) as white solid. MS: 468 [M+H]⁺.

### Step 5: Synthesis of N-(2-((2-(dimethylamino)ethyl)(methyl)amino)-5-((4-((2-(dimethylphosphoryl)phenyl)amino)-5-methylpyrimidin-2-yl)amino)phenyl)acrylamide hydrochloric acid salt

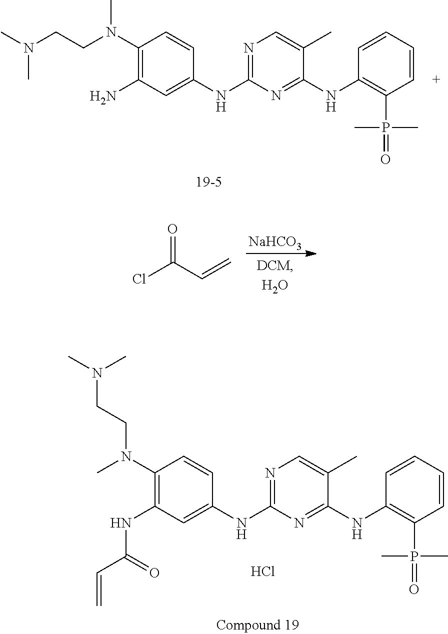

Following the same procedure as N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt using (2-((2-((3-amino-4-((2-(dimethylamino)ethyl)(methyl)amino)phenyl)amino)-5-methylpyrimidin-4-yl)amino)phenyl)dimethylphosphine oxide instead of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide to obtain N-(2-((2-(dimethylamino)ethyl)(methyl)amino)-5-((4-((2-(dimethylphosphoryl)phenyl)amino)-5-methylpyrimidin-2-yl)amino)phenyl)acrylamide hydrochloric acid salt. MS: 522 [M+H]⁺.

### Example 20 Synthesis of Compound 20

### N-(5-((4-((2-(dimethylphosphoryl)phenyl)amino)-5-methylpyrimidin-2-yl)amino)-2-(4-(4-methylpiperazin-1-yl)piperidin-1-yl)phenyl)acrylamide

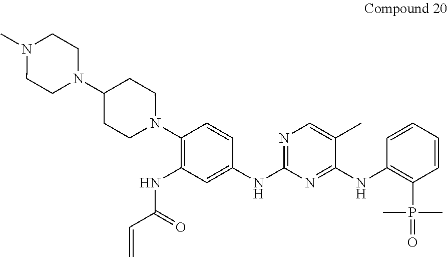

### Step 1: Synthesis of dimethyl(2-((5-methyl-2-((4-(4-(4-methylpiperazin-1-yl)piperidin-1-yl)-3-nitrophenyl)amino)pyrimidin-4-yl)amino)phenyl)phosphine oxide

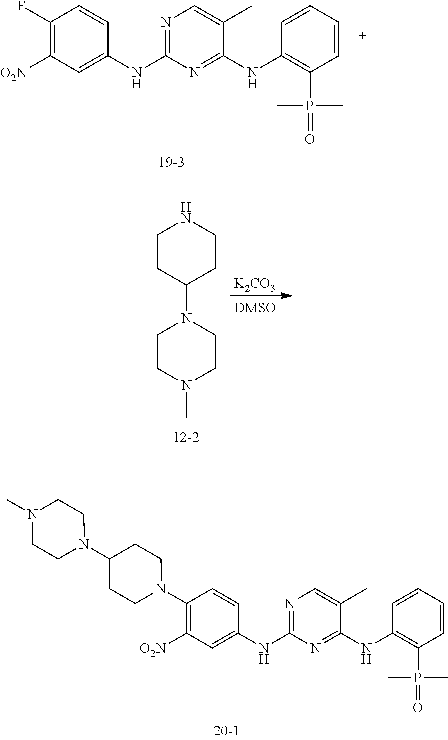

Following the same procedure as (2-((2-((4-((2-(dimethylamino)ethyl)(methyl)amino)-3-nitrophenyl)amino)-5-methylpyrimidin-4-yl)amino)phenyl)dimethylphosphine oxide using 1-methyl-4-(piperidin-4-yl)piperazine instead of N¹,N¹,N²-trimethylethane-1,2-diamine to obtain dimethyl(2-((5-methyl-2-((4-(4-(4-methylpiperazin-1-yl)piperidin-1-yl)-3-nitrophenyl)amino)pyrimidin-4-yl)amino)phenyl)phosphine oxide. MS: 579 [M+H]⁺.

### Step 2: Synthesis of (2-((2-((3-amino-4-(4-(4-methylpiperazin-1-yl)piperidin-1-yl)phenyl)amino)-5-methylpyrimidin-4-yl)amino)phenyl)dimethylphosphine oxide

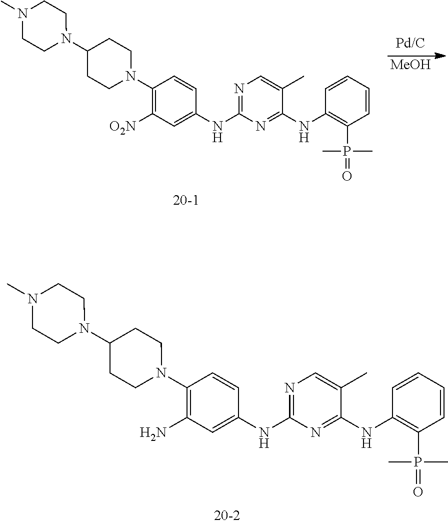

Following the same procedure as (2-((2-((3-amino-4-((2-(dimethylamino)ethyl)(methyl)amino)phenyl)amino)-5-methylpyrimidin-4-yl)amino)phenyl)dimethylphosphine oxide using dimethyl(2-((5-methyl-2-((4-(4-(4-methylpiperazin-1-yl)piperidin-1-yl)-3-nitrophenyl)amino)pyrimidin-4-yl)amino)phenyl)phosphine oxide instead of (2-((2-((4-((2-(dimethylamino)ethyl)(methyl)amino)-3-nitrophenyl)amino)-5-methylpyrimidin-4-yl)amino)phenyl)dimethylphosphine oxide to obtain (2-((2-((3-amino-4-(4-(4-methylpiperazin-1-yl)piperidin-1-yl)phenyl)amino)-5-methylpyrimidin-4-yl)amino)phenyl)dimethylphosphine oxide. MS: 549 [M+H]⁺.

### Step 3: Synthesis of N-(5-((4-((2-(dimethylphosphoryl)phenyl)amino)-5-methylpyrimidin-2-yl)amino)-2-(4-(4-methylpiperazin-1-yl)piperidin-1-yl)phenyl)acrylamide

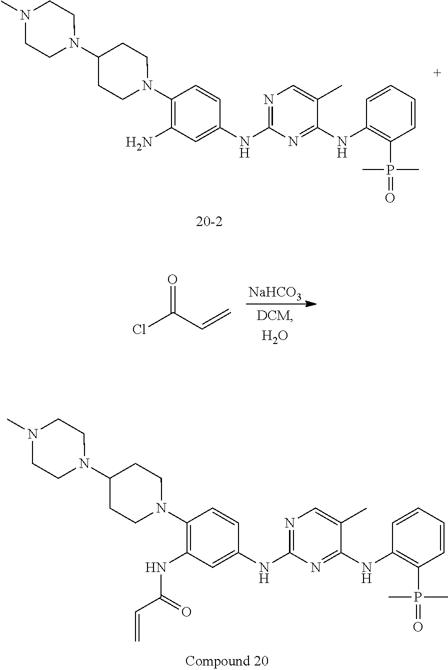

Following the same procedure as N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt using (2-((2-((3-amino-4-(4-(4-methylpiperazin-1-yl)piperidin-1-yl)phenyl)amino)-5-methylpyrimidin-4-yl)amino)phenyl)dimethylphosphine oxide instead of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide to obtain N-(5-((4-((2-(dimethylphosphoryl)phenyl)amino)-5-methylpyrimidin-2-yl)amino)-2-(4-(4-methylpiperazin-1-yl)piperidin-1-yl)phenyl)acrylamide. MS: 603[M+H]⁺.

### Example 21 Synthesis of Compound 21

### N-(2-((2-(dimethylamino)ethyl)(methyl)amino)-5-((4-((2-(dimethylphosphoryl)phenyl)amino)-5-methylpyrimidin-2-yl)amino)-4-methoxyphenyl)acrylamide

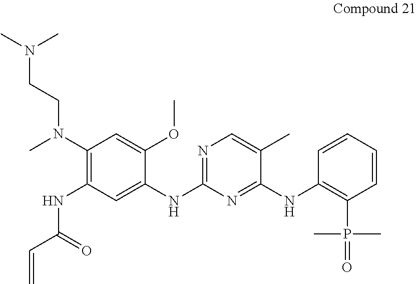

### Step 1: Synthesis of (2-((2-((4-fluoro-2-methoxy-5-nitrophenyl)amino)-5-methylpyrimidin-4-yl)amino)phenyl)dimethylphosphine oxide

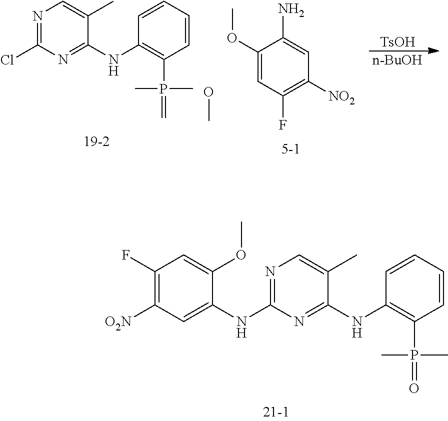

Following the same procedure as (2-((2-((4-fluoro-3-nitrophenyl)amino)-5-methylpyrimidin-4-yl)amino)phenyl)dimethylphosphine oxide using 4-fluoro-2-methoxy-5-nitroaniline instead of 4-fluoro-3-nitroaniline to obtain (2-((2-((4-fluoro-2-methoxy-5-nitrophenyl)amino)-5-methylpyrimidin-4-yl)amino)phenyl)dimethylphosphine oxide. MS: 445 [M+H]⁺.

### Step 2: Synthesis of (2-((2-((4-((2-(dimethylamino)ethyl)(methyl)amino)-2-methoxy-5-nitrophenyl)amino)-5-methylpyrimidin-4-yl)amino)phenyl)dimethylphosphine oxide

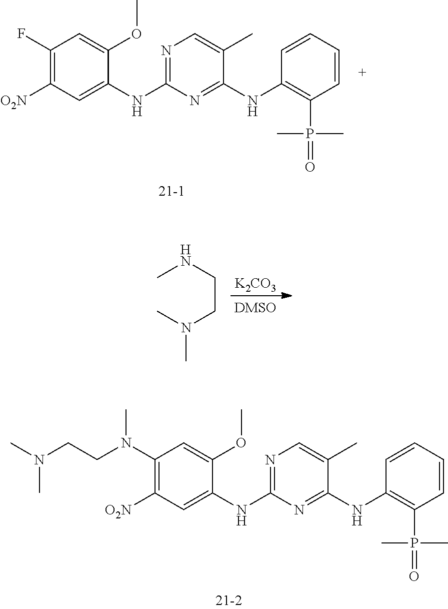

Following the same procedure as (2-((2-((4-((2-(dimethylamino)ethyl)(methyl)amino)-3-nitrophenyl)amino)-5-methylpyrimidin-4-yl)amino)phenyl)dimethylphosphine oxide using (2-((2-((4-fluoro-2-methoxy-5-nitrophenyl)amino)-5-methylpyrimidin-4-yl)amino)phenyl)dimethylphosphine oxide instead of (2-((5-chloro-2-((4-fluoro-3-nitrophenyl)amino)pyrimidin-4-yl)amino)phenyl)dimethylphosphine oxide to obtain (2-((2-((4-((2-(dimethylamino)ethyl)(methyl)amino)-2-methoxy-5-nitrophenyl)amino)-5-methylpyrimidin-4-yl)amino)phenyl)dimethylphosphine oxide. MS: 528 [M+H]⁺.

### Step 3: Synthesis of (2-((2-((5-amino-4-((2-(dimethylamino)ethyl)(methyl)amino)-2-methoxyphenyl)amino)-5-methylpyrimidin-4-yl)amino)phenyl)dimethylphosphine oxide

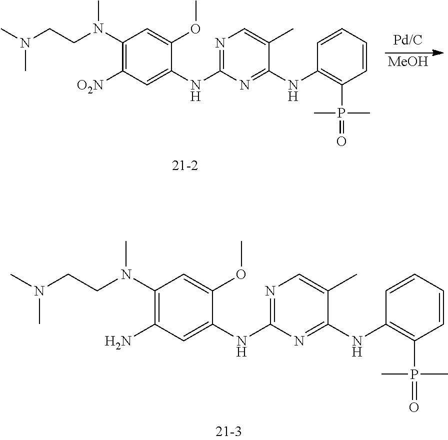

Following the same procedure as (2-((2-((3-amino-4-((2-(dimethylamino)ethyl)(methyl)amino)phenyl)amino)-5-methylpyrimidin-4-yl)amino)phenyl)dimethylphosphine oxide using (2-((2-((4-((2-(dimethylamino)ethyl)(methyl)amino)-2-methoxy-5-nitrophenyl)amino)-5-methylpyrimidin-4-yl)amino)phenyl)dimethylphosphine oxide instead of (2-((2-((4-((2-(dimethylamino)ethyl)(methyl)amino)-3-nitrophenyl)amino)-5-methylpyrimidin-4-yl)amino)phenyl)dimethylphosphine oxide to obtain (2-((2-((5-amino-4-((2-(dimethylamino)ethyl)(methyl)amino)-2-methoxyphenyl)amino)-5-methylpyrimidin-4-yl)amino)phenyl)dimethylphosphine oxide. MS: 498 [M+H]⁺.

### Step 4: Synthesis of N-(2-((2-(dimethylamino)ethyl)(methyl)amino)-5-((4-((2-(dimethylphosphoryl)phenyl)amino)-5-methylpyrimidin-2-yl)amino)-4-methoxyphenyl)acrylamide (Compound 21)

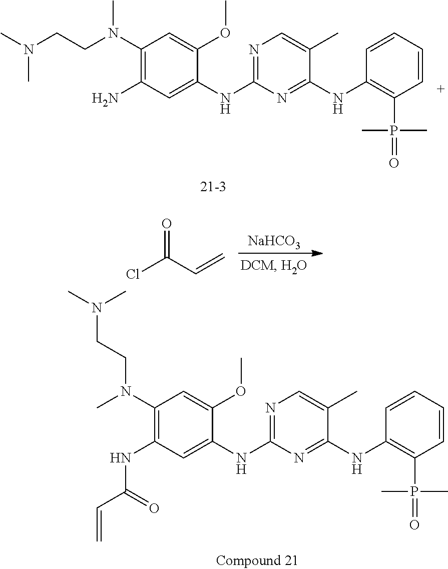

Following the same procedure as N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt using (2-((2-((5-amino-4-((2-(dimethylamino)ethyl)(methyl)amino)-2-methoxyphenyl)amino)-5-methylpyrimidin-4-yl)amino)phenyl)dimethylphosphine oxide instead of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide to obtain N-(2-((2-(dimethylamino)ethyl)(methyl)amino)-5-((4-((2-(dimethylphosphoryl)phenyl)amino)-5-methylpyrimidin-2-yl)amino)-4-methoxyphenyl)acrylamide (Compound 21). MS: 552 [M+H]⁺.

### Example 22 Synthesis of Compound 22

### N-(5-((4-((2-(dimethylphosphoryl)phenyl)amino)-5-methylpyrimidin-2-yl)amino)-4-methoxy-2-(4-(4-methylpiperazin-1-yl)piperidin-1-yl)phenyl)acrylamide

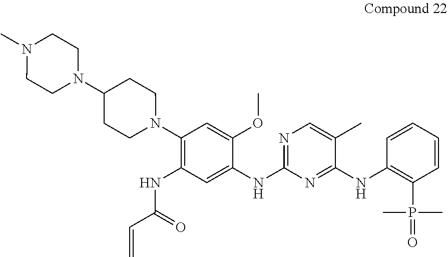

### Step 1: Synthesis of (2-((2-((2-methoxy-4-(4-(4-methylpiperazin-1-yl)piperidin-1-yl)-5-nitrophenyl)amino)-5-methylpyrimidin-4-yl)amino)phenyl)dimethylphosphine oxide

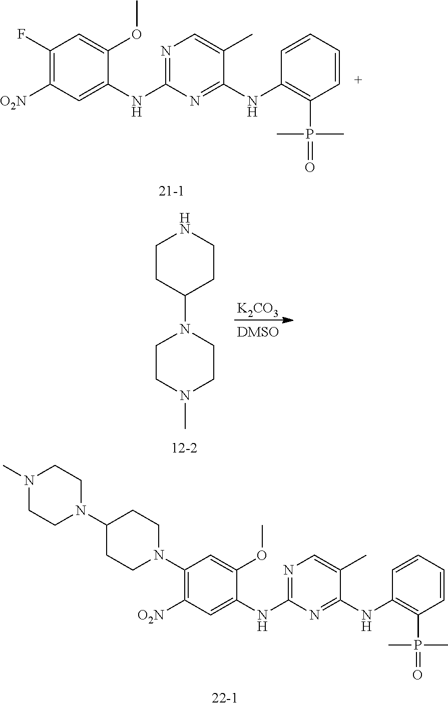

Following the same procedure as (2-((2-((4-((2-(dimethylamino)ethyl)(methyl)amino)-2-methoxy-5-nitrophenyl)amino)-5-methylpyrimidin-4-yl)amino)phenyl)dimethylphosphine oxide using 1-methyl-4-(piperidin-4-yl)piperazine instead of N¹,N¹,N²-trimethylethane-1,2-diamine to obtain (2-((2-((2-methoxy-4-(4-(4-methylpiperazin-1-yl)piperidin-1-yl)-5-nitrophenyl)amino)-5-methylpyrimidin-4-yl)amino)phenyl)dimethylphosphine oxide. MS: 609 [M+H]+.

### Step 2: Synthesis of (2-((2-((5-amino-2-methoxy-4-(4-(4-methylpiperazin-1-yl)piperidin-1-yl)phenyl)amino)-5-methylpyrimidin-4-yl)amino)phenyl)dimethylphosphine oxide

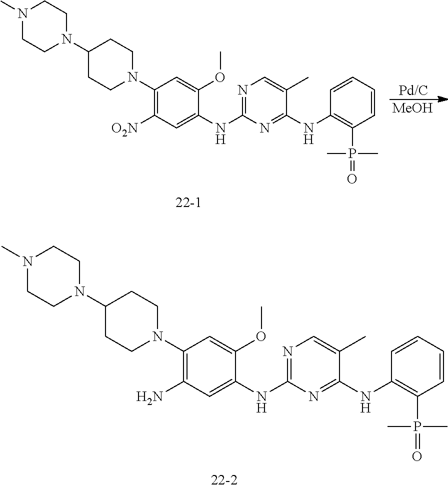

Following the same procedure as (2-((2-((3-amino-4-((2-(dimethylamino)ethyl)(methyl)amino)phenyl)amino)-5-methylpyrimidin-4-yl)amino)phenyl)dimethylphosphine oxide using (2-((2-((2-methoxy-4-(4-(4-methylpiperazin-1-yl)piperidin-1-yl)-5-nitrophenyl)amino)-5-methylpyrimidin-4-yl)amino)phenyl)dimethylphosphine oxide instead of (2-((2-((4-((2-(dimethylamino)ethyl)(methyl)amino)-3-nitrophenyl)amino)-5-methylpyrimidin-4-yl)amino)phenyl)dimethylphosphine oxide to obtain (2-((2-((5-amino-2-methoxy-4-(4-(4-methylpiperazin-1-yl)piperidin-1-yl)phenyl)amino)-5-methylpyrimidin-4-yl)amino)phenyl)dimethylphosphine oxide. MS: 579 [M+H]⁺.

### Step 3: Synthesis of N-(5-((4-((2-(dimethylphosphoryl)phenyl)amino)-5-methylpyrimidin-2-yl)amino)-4-methoxy-2-(4-(4-methylpiperazin-1-yl)piperidin-1-yl)phenyl)acrylamide

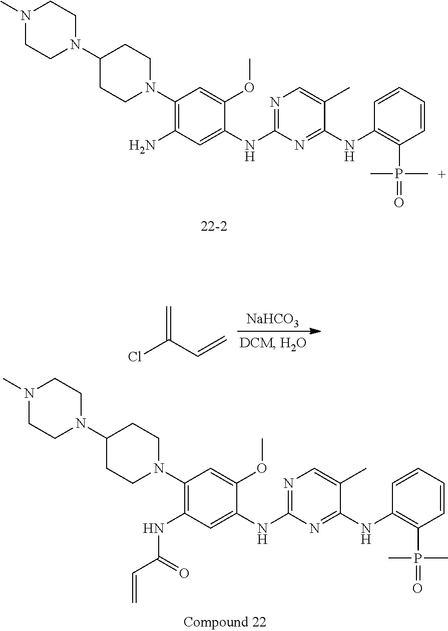

Following the same procedure as N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt using (2-((2-((5-amino-2-methoxy-4-(4-(4-methylpiperazin-1-yl)piperidin-1-yl)phenyl)amino)-5-methylpyrimidin-4-yl)amino)phenyl)dimethylphosphine oxide instead of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide to obtain N-(5-((4-((2-(dimethylphosphoryl)phenyl)amino)-5-methylpyrimidin-2-yl)amino)-4-methoxy-2-(4-(4-methylpiperazin-1-yl)piperidin-1-yl)phenyl)acrylamide. MS: 633 [M+H]⁺.

### Example 23 Synthesis of Compound 23

### N-(5-((5-chloro-4-((1-(dimethylphosphoryl)naphthalen-2-yl)amino)pyrimidin-2-yl)amino)-2-(2-(dimethylamino)-7-azaspiro[3.5]nonan-7-yl)phenyl)acrylamide hydrochloric acid salt

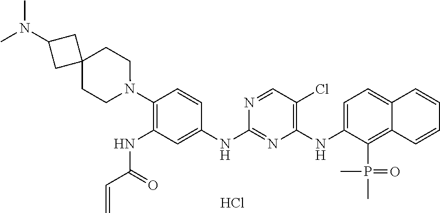 [SMILES: C=CC(=O)Nc1cc(Nc2ncc(Cl)c(Nc3ccc4ccccc4c3P(C)(C)=O)n2)ccc1N1CCC2(CC1)CC(N(C)C)C2 ; Cl]

### Step 1: Synthesis of 1-iodonaphthalen-2-amine

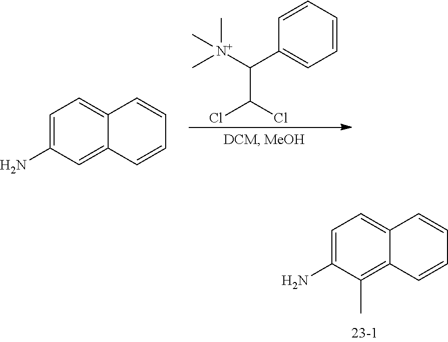

At the N₂ atmosphere benzyltrimethylammonium dichloroiodate was added to a mixture of naphthalen-2-amine (4.00 g) in DCM (120 mL) and MeOH (40 mL). The mixture was stirred 2 h at room temperature. The mixture solution was poured into sodium bicarbonate solution and DCM (50 mL*3). The combined organic layer was washed with saturated sodium chloride aqueous solution (50 mL*2), dried over Na₂SO₄ and concentrated under reduced pressure, purified by silica gel column chromatography using DCM/methanol (95:5) as the eluent to obtain 1-iodonaphthalen-2-amine (7.02 g) as brown oil. MS: 269 [M+H]⁺.

### Step 2: Synthesis of (2-aminonaphthalen-1-yl)dimethylphosphine oxide

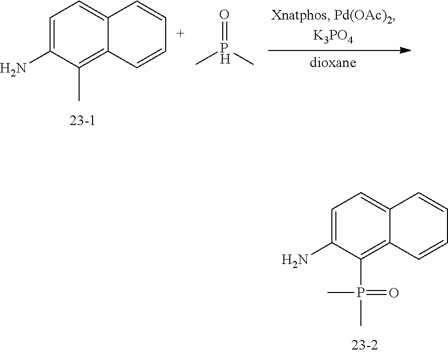

At the N₂ atmosphere, to a mixture of 1-iodonaphthalen-2-amine (2.00 g) in dioxane (20 mL), dimethylphosphine oxide (580 mg), Xnatphos (860 mg), Pd(OAc)₂ (167 mg) and K₃PO₄ (3.16 g) was added under stirring. The mixture was heated 100° C. for about 10 h. The mixture solution was poured into water and extracted with DCM (50 mL*3). The combined organic layer was washed with saturated sodium chloride aqueous solution (50 mL*2), dried over Na₂SO₄ and concentrated under reduced, purified by silica gel column chromatography using DCM/methanol (95:5) as the eluent to obtain (2-aminonaphthalen-1-yl)dimethylphosphine oxide (1.60 g) as brown solid. MS: 219 [M+H]⁺.

### Step 3: Synthesis of (2-((2,5-dichloropyrimidin-4-yl)amino)naphthalen-1-yl)dimethylphosphine oxide

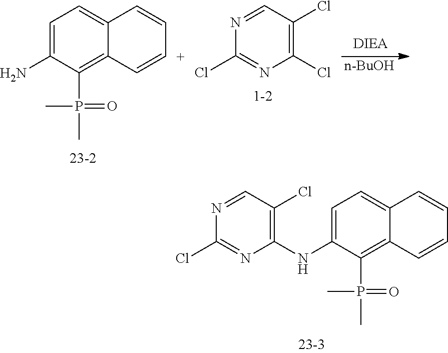

To a mixture of (2-aminonaphthalen-1-yl)dimethylphosphine oxide (1.0 g) in n-BuOH (20 mL), 2,4,5-trichloropyrimidine (1.67 g), DIEA (1.18 g), was added under stirring. The mixture was heated 120° C. for about 8 h. The mixture was then filtered through diatomaceous earth and washed with n-BuOH; the filter cake was then concentrated under reduced pressure to obtain (2-((2,5-dichloropyrimidin-4-yl)amino)naphthalen-1-yl)dimethylphosphine oxide (1.30 g) as white solid. MS: 366 [M+H]⁺.

### Step 4: Synthesis of 7-(4-amino-2-nitrophenyl)-7-azaspiro[3.5]nonan-2-one

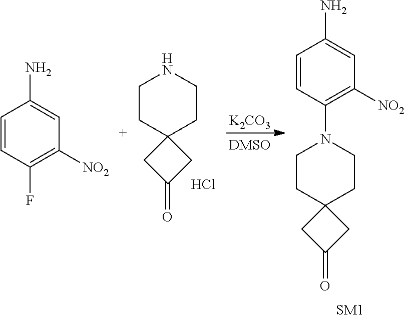

Following the same procedure as 7-(4-((5-chloro-4-((5-(dimethylphosphoryl)quinoxalin-6-yl)amino)pyrimidin-2-yl)amino)-2-nitrophenyl)-7-azaspiro[3.5]nonan-2-one using 4-fluoro-3-nitroaniline instead of (6-((5-chloro-2-((4-fluoro-3-nitrophenyl)amino)pyrimidin-4-yl)amino)quinoxalin-5-yl)dimethylphosphine oxide to obtain 7-(4-amino-2-nitrophenyl)-7-azaspiro[3.5]nonan-2-one. MS: 276 [M+H]+.

### Step 5: Synthesis of 7-(4-amino-2-nitrophenyl)-N,N-dimethyl-7-azaspiro[3.5]nonan-2-amine

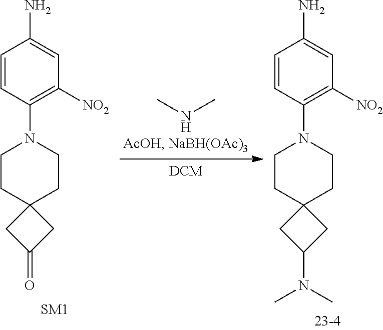

Following the same procedure as (6-((5-chloro-2-((4-(2-(dimethylamino)-7-azaspiro[3.5]nonan-7-yl)-3-nitrophenyl)amino)pyrimidin-4-yl)amino)quinoxalin-5-yl)dimethylphosphine oxide using 7-(4-amino-2-nitrophenyl)-7-azaspiro[3.5]nonan-2-one instead of 7-(4-((5-chloro-4-((5-(dimethylphosphoryl)quinoxalin-6-yl)amino)pyrimidin-2-yl)amino)-2-nitrophenyl)-7-azaspiro[3.5]nonan-2-one to obtain 7-(4-amino-2-nitrophenyl)-N,N-dimethyl-7-azaspiro[3.5]nonan-2-amine. MS: 305 [M+H]+.

### Step 6: Synthesis of (2-((5-chloro-2-((4-(2-(dimethylamino)-7-azaspiro[3.5]nonan-7-yl)-3-nitrophenyl)amino)pyrimidin-4-yl)amino)naphthalen-1-yl)dimethylphosphine oxide

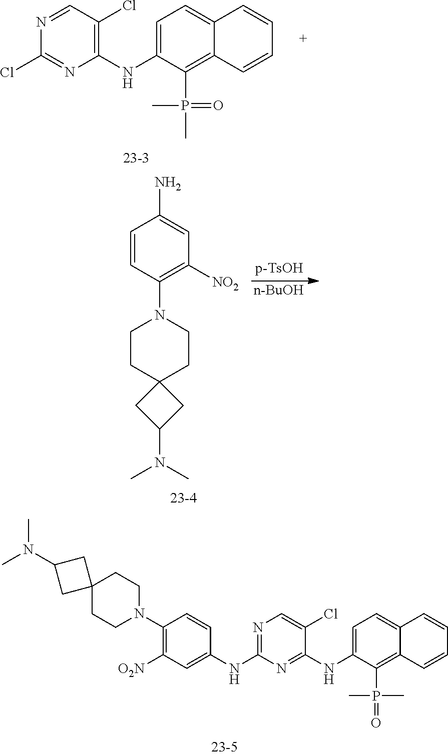 [SMILES: CP(C)(=O)c1c(Nc2nc(Cl)ncc2Cl)ccc2ccccc12 ; "n-BuOH" ; CN(C)C1CC2(CCN(c3ccc(N)cc3[N+](=O)[O-])CC2)C1 ; CN(C)C1CC2(CCN(c3ccc(Nc4ncc(Cl)c(Nc5ccc6ccccc6c5P(C)(C)=O)n4)cc3[N+](=O)[O-])CC2)C1]

Following the same procedure as (2-((5-chloro-2-((4-fluoro-3-nitrophenyl)amino)pyrimidin-4-yl)amino)phenyl)dimethylphosphine oxide using (2-((2,5-dichloropyrimidin-4-yl)amino)naphthalen-1-yl)dimethylphosphine oxide instead of (2-((2,5-dichloropyrimidin-4-yl)amino)phenyl)dimethylphosphine oxide and using 7-(4-amino-2-nitrophenyl)-N,N-dimethyl-7-azaspiro[3.5]nonan-2-amine instead of 4-fluoro-3-nitroaniline to obtain (2-((5-chloro-2-((4-(2-(dimethylamino)-7-azaspiro[3.5]nonan-7-yl)-3-nitrophenyl)amino)pyrimidin-4-yl)amino)naphthalen-1-yl)dimethylphosphine oxide. MS: 634 [M+H]⁺.

### Step 7: Synthesis of (2-((2-((3-amino-4-(2-(dimethylamino)-7-azaspiro[3.5]nonan-7-yl)phenyl)amino)-5-chloropyrimidin-4-yl)amino)naphthalen-1-yl)dimethylphosphine oxide

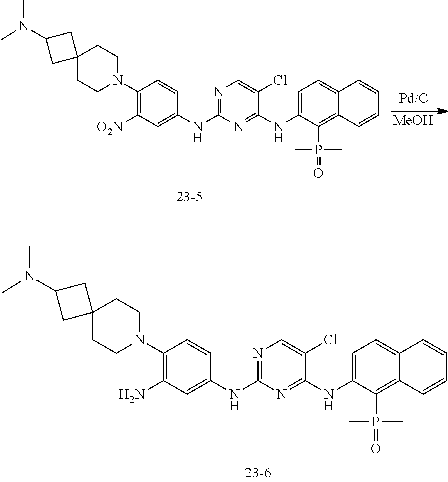 [SMILES: "MeOH" ; CN(C)C1CC2(CCN(c3ccc(Nc4ncc(Cl)c(Nc5ccc6ccccc6c5P(C)(C)=O)n4)cc3[N+](=O)[O-])CC2)C1 ; CN(C)C1CC2(CCN(c3ccc(Nc4ncc(Cl)c(Nc5ccc6ccccc6c5P(C)(C)=O)n4)cc3N)CC2)C1]

Following the same procedure as (2-((2-((3-amino-4-((2-(dimethylamino)ethyl)(methyl)amino)phenyl)amino)-5-methylpyrimidin-4-yl)amino)phenyl)dimethylphosphine oxide using (2-((5-chloro-2-((4-(2-(dimethylamino)-7-azaspiro[3.5]nonan-7-yl)-3-nitrophenyl)amino)pyrimidin-4-yl)amino)naphthalen-1-yl)dimethylphosphine oxide instead of (2-((2-((4-((2-(dimethylamino)ethyl)(methyl)amino)-3-nitrophenyl)amino)-5-methylpyrimidin-4-yl)amino)phenyl)dimethylphosphine oxide to obtain (2-((2-((3-amino-4-(2-(dimethylamino)-7-azaspiro[3.5]nonan-7-yl)phenyl)amino)-5-chloropyrimidin-4-yl)amino)naphthalen-1-yl)dimethylphosphine oxide. MS: 604 [M+H]⁺.

### Step 8: Synthesis of N-(5-((5-chloro-4-((1-(dimethylphosphoryl)naphthalen-2-yl)amino)pyrimidin-2-yl)amino)-2-(2-(dimethylamino)-7-azaspiro[3.5]nonan-7-yl)phenyl)acrylamide hydrochloric acid salt

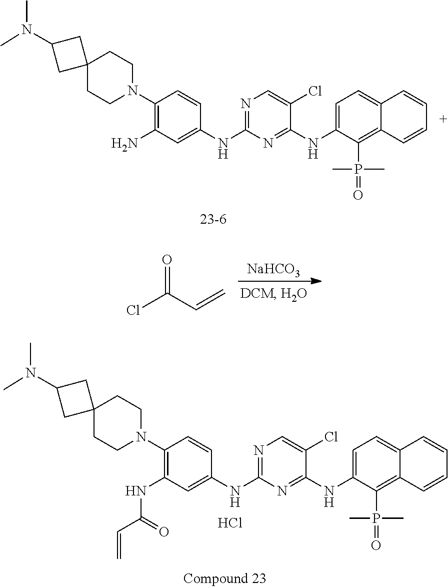

Following the same procedure as N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt using (2-((2-((3-amino-4-(2-(dimethylamino)-7-azaspiro[3.5]nonan-7-yl)phenyl)amino)-5-chloropyrimidin-4-yl)amino)naphthalen-1-yl)dimethylphosphine oxide instead of (2-((2-((3-amino-4-(2-methyl-2,7-diazaspiro[3.5]nonan-7-yl)phenyl)amino)-5-chloropyrimidin-4-yl)amino)phenyl)dimethylphosphine oxide to obtain N-(5-((5-chloro-4-((1-(dimethylphosphoryl)naphthalen-2-yl)amino)pyrimidin-2-yl)amino)-2-(2-(dimethylamino)-7-azaspiro[3.5]nonan-7-yl)phenyl)acrylamide hydrochloric acid salt. MS: 658 [M+H]⁺.

### Example 24 Synthesis of Compound 24

### N-(5-((5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acrylamide

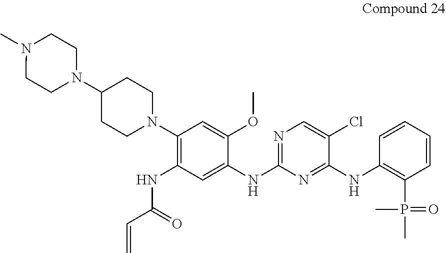 [SMILES: C=CC(=O)Nc1cc(Nc2ncc(Cl)c(Nc3ccccc3P(C)(C)=O)n2)c(OC)cc1N1CCC(N2CCN(C)CC2)CC1]

### Step 1: Synthesis of (2-((5-chloro-2-((2-methoxy-4-(4-(4-methylpiperazin-1-yl)piperidin-1-yl)-5-nitrophenyl)amino)pyrimidin-4-yl)amino)phenyl)dimethylphosphine oxide

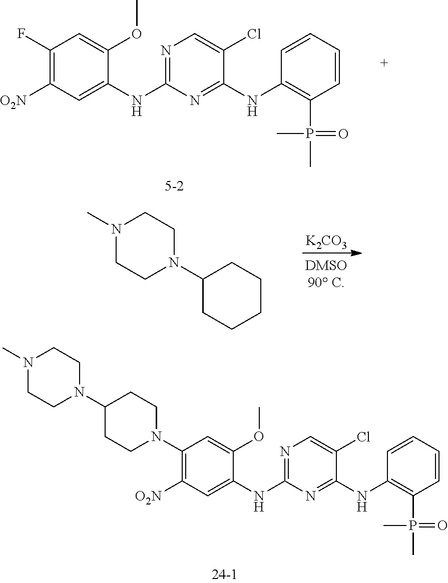

To a solution of 1-methyl-4-(4-piperidyl)piperazine (141.66 mg) and 5-chloro-N4-(2-dimethylphosphorylphenyl)-N2-(4-fluoro-2-methoxy-5-nitro-phenyl)pyrimidine-2,4-diamine (300 mg) dissolved in DMSO (10 mL) was added K₂CO₃ (267.03 mg). The reaction mixture was stirred at 90° C. overnight. The reaction mixture was cooled down to room temperature and diluted with DCM (20 mL). The resulting solution was washed with water and NaCl saturated aqueous solution. The mixture was dried over anhydrous sodium sulfate and concentrated under vacuum to obtained 5-chloro-N4-(2-dimethylphosphorylphenyl)-N2-[2-methoxy-4-[4-(4-methylpiperazin-1-yl)-1-piperidyl]-5-nitro-phenyl]pyrimidine-2,4-diamine (430 mg) as a yellow solid. MS: 629 [M+H]⁺.

### Step 2: Synthesis of (2-((2-((5-amino-2-methoxy-4-(4-(4-methylpiperazin-1-yl)piperidin-1-yl)phenyl)amino)-5-chloropyrimidin-4-yl)amino)phenyl)dimethylphosphine oxide

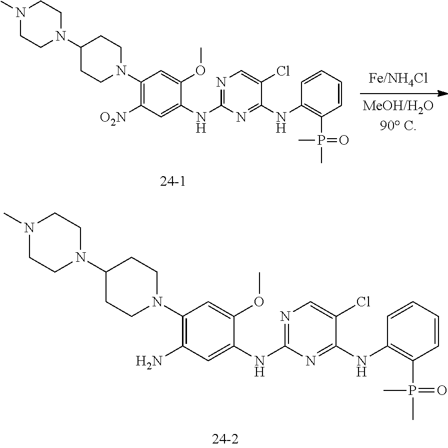 [SMILES: COc1cc(N2CCC(N3CCN(C)CC3)CC2)c([N+](=O)[O-])cc1Nc1ncc(Cl)c(Nc2ccccc2P(C)(C)=O)n1 ; COc1cc(N2CCC(N3CCN(C)CC3)CC2)c(N)cc1Nc1ncc(Cl)c(Nc2ccccc2P(C)(C)=O)n1]

To a solution of 5-chloro-N4-(2-dimethylphosphorylphenyl)-N2-[2-methoxy-4-[4-(4-methylpiperazin-1-yl)-1-piperidyl]-5-nitro-phenyl]pyrimidine-2,4-diamine (430 mg) dissolved in MeOH (20 mL) and H₂O (2 mL), was added Fe (190.88 mg, 3.42 mmol) and NH₄Cl (182.81 mg, 3.42 mmol). The reaction mixture was stirred at 90° C. for 5 hrs. The resulting solution was filtered and collect the filtrate. The filtrate was concentrated under vacuum. The crude product was purified by silica gel column chromatography using DCM/methanol (0-10%, 20 mins) as the eluent to obtained (2-((2-((5-amino-2-methoxy-4-(4-(4-methylpiperazin-1-yl)piperidin-1-yl)phenyl)amino)-5-chloropyrimidin-4-yl)amino)phenyl)dimethylphosphine oxide (260 mg). MS: 599 [M+H]⁺.

### Step 3: Synthesis of N-(5-((5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acrylamide

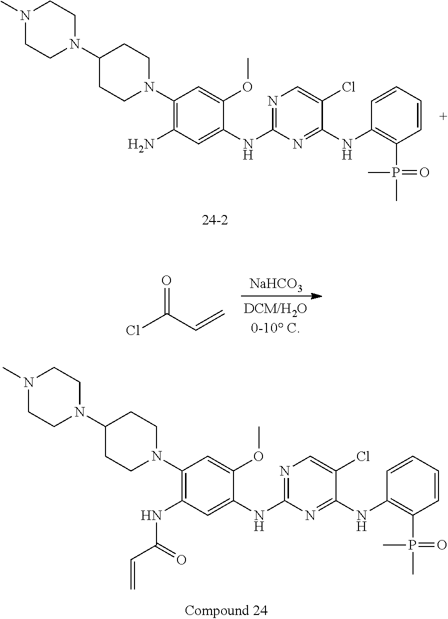

To a solution of (2-((2-((5-amino-2-methoxy-4-(4-(4-methylpiperazin-1-yl)piperidin-1-yl)phenyl)amino)-5-chloropyrimidin-4-yl)amino)phenyl)dimethylphosphine oxide (130 mg) in DCM (10 mL) and H₂O (5 mL), was dropwise added prop-2-enoyl chloride (22 mg) in DCM (1 ml) at 0-10° C. The resulting solution was stirred for 0.5 h at 0-10° C. The reaction was concentrated by vacuum. The residue was purified by column chromatography over silica gel with DCM/MeOH (8:1). This obtained N-(5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acrylamide (33.8 mg) MS: 653 [M+H]⁺.

### Example 25 Synthesis of Compound 25

### N-(5-((5-chloro-4-((2-(dimethylphosphoryl)phenyl)amino)pyrimidin-2-yl)amino)-2-((2-(dimethylamino)ethyl)(methyl)amino)-4-methoxyphenyl)acrylamide

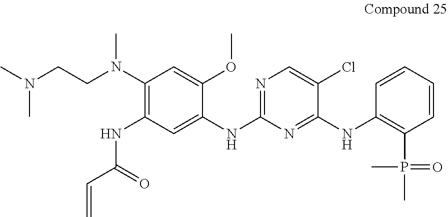 [SMILES: C=CC(=O)Nc1cc(Nc2ncc(Cl)c(Nc3ccccc3P(C)(C)=O)n2)c(OC)cc1N(C)CCN(C)C]

### Step 1: Synthesis of (2-((5-chloro-2-((4-((2-(dimethylamino)ethyl)amino)-2-methoxy-5-nitrophenyl)amino)pyrimidin-4-yl)amino)phenyl)dimethylphosphine oxide

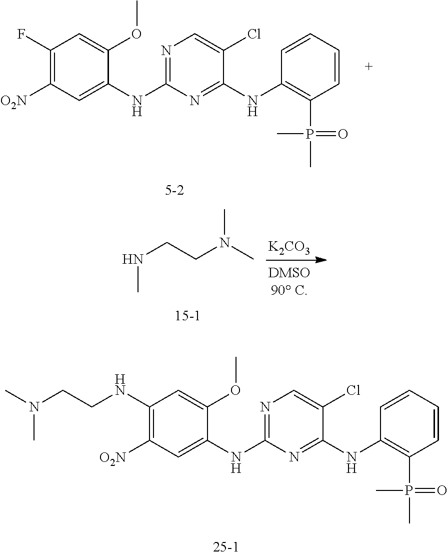

Following the same procedure as (2-((5-chloro-2-((2-methoxy-4-(4-(4-methylpiperazin-1-yl)piperidin-1-yl)-5-nitrophenyl)amino)pyrimidin-4-yl)amino)phenyl)dimethylphosphine oxide using N¹,N¹,N²-trimethylethane-1,2-diamine instead of 1-methyl-4-(4-piperidyl)piperazine to obtain (2-((5-chloro-2-((4-((2-(dimethylamino)ethyl)amino)-2-methoxy-5-nitrophenyl)amino)pyrimidin-4-yl)amino)phenyl)dimethylphosphine oxide. MS: 534 [M+H]⁺.

### Step 2: Synthesis of (2-((2-((5-amino-4-((2-(dimethylamino)ethyl)amino)-2-methoxyphenyl)amino)-5-chloropyrimidin-4-yl)amino)phenyl)dimethylphosphine oxide

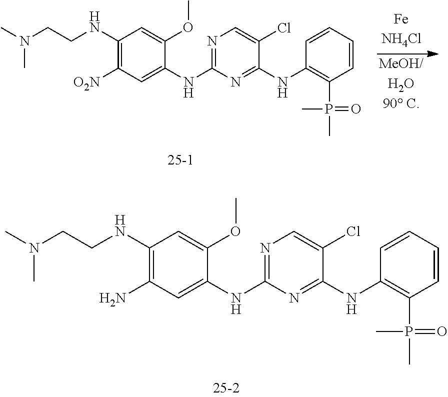 [SMILES: COc1cc(NCCN(C)C)c([N+](=O)[O-])cc1Nc1ncc(Cl)c(Nc2ccccc2P(C)(C)=O)n1 ; COc1cc(NCCN(C)C)c(N)cc1Nc1ncc(Cl)c(Nc2ccccc2P(C)(C)=O)n1]

Following the same procedure as Synthesis of (2-((2-((5-amino-2-methoxy-4-(4-(4-methylpiperazin-1-yl)piperidin-1-yl)phenyl)amino)-5-chloropyrimidin-4-yl)amino)phenyl)dimethylphosphine oxide using (2-((5-chloro-2-((4-((2-(dimethylamino)ethyl)amino)-2-methoxy-nitrophenyl)amino)pyrimidin-4-yl)amino)phenyl)dimethylphosphine oxide instead of 5-chloro-N4-(2-dimethylphosphorylphenyl)-N2-[2-methoxy-4-[4-(4-methylpiperazin-1-yl)-1-piperidyl]-5-nitro-phenyl]pyrimidine-2,4-diamine to obtain (2-((2-((5-amino-4-((2-(dimethylamino)ethyl)amino)-2-methoxyphenyl)amino)-5-chloropyrimidin-4-yl)amino)phenyl)dimethylphosphine oxide. MS: 504 [M+H]⁺.

### Step 3: Synthesis of N-(5-((5-chloro-4-((2-(dimethylphosphoryl)phenyl)amino)pyrimidin-2-yl)amino)-2-((2-(dimethylamino)ethyl)(methyl)amino)-4-methoxyphenyl)acrylamide

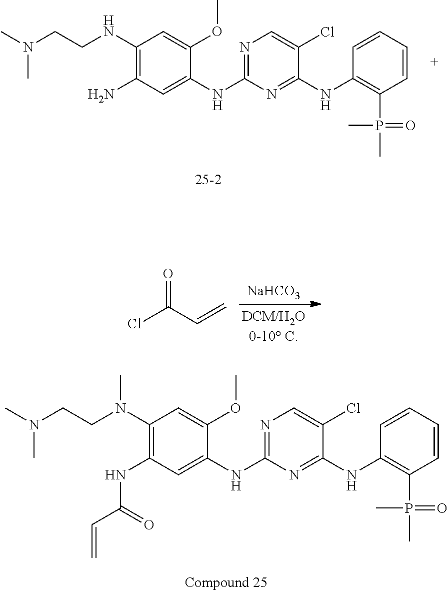

Following the same procedure as N-(5-((5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acryl amide using (2-((2-((5-amino-4-((2-(dimethylamino)ethyl)amino)-2-methoxyphenyl)amino)-5-chloropyrimidin-4-yl)amino)phenyl)dimethylphosphine oxide instead of N2-[5-amino-2-methoxy-4-[4-(4-methyl piperazin-1-yl)-1-piperidyl]phenyl]-5-chloro-N4-(2-dimethylphosphorylphenyl)pyrimidine-2,4-diamine to obtain N-(5-((5-chloro-4-((2-(dimethylphosphoryl)phenyl)amino)pyrimidin-2-yl)amino)-2-((2-(dimethylamino)ethyl)(methyl)amino)-4-methoxyphenyl)acrylamide. MS: 572 [M+H]⁺.

¹H NMR (500 MHz, DMSO-d6) δ 11.22 (s, 1H), 8.45 (m, 1H), 8.32 (s, 1H), 8.12 (s, 1H), 7.54 (m, 1H), 7.30 (s, 1H), 7.05 (m, 2H), 6.24 (m, 1H), 5.76 (m, 1H), 3.87 (m, 3H), 3.25 (s, 3H), 2.89 (m, 6H) 2.36 (m, 4H), 1.79 (s, 6H).

### Example 26 Synthesis of Compound 26

### N-(5-((5-chloro-4-((2-(dimethylphosphoryl)phenyl)amino)pyrimidin-2-yl)amino)-2-(7-(dimethylamino)-2-azaspiro[3.5]nonan-2-yl)-4-methoxyphenyl)acrylamide

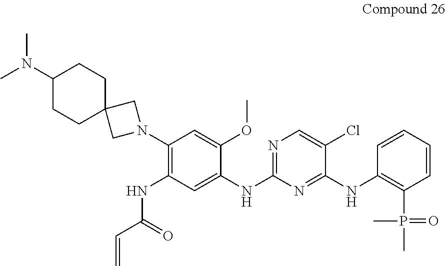 [SMILES: C=CC(=O)Nc1cc(Nc2ncc(Cl)c(Nc3ccccc3P(C)(C)=O)n2)c(OC)cc1N1CC2(CCC(N(C)C)CC2)C1]

### Step 1: Synthesis of 2-(4-((5-chloro-4-((2-(dimethylphosphoryl)phenyl)amino)pyrimidin-2-yl)amino)-5-methoxy-2-nitrophenyl)-2-azaspiro[3.5]nonan-7-one

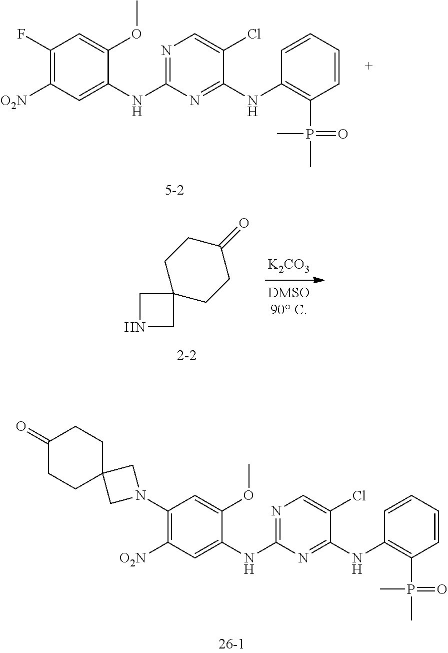

Following the same procedure as (2-((5-chloro-2-((2-methoxy-4-(4-(4-methylpiperazin-1-yl)piperidin-1-yl)-5-nitrophenyl)amino)pyrimidin-4-yl)amino)phenyl)dimethylphosphine oxide using 2-azaspiro[3.5]nonan-7-one trifluoroacetate instead of 1-methyl-4-(4-piperidyl)piperazine to obtain 2-(4-((5-chloro-4-((2-(dimethylphosphoryl)phenyl)amino)pyrimidin-2-yl)amino)-5-methoxy-2-nitrophenyl)-2-azaspiro[3.5]nonan-7-one. MS: 585 [M+H]⁺.

### Step 2: Synthesis of (2-((5-chloro-2-((4-(7-(dimethylamino)-2-azaspiro[3.5]nonan-2-yl)-2-methoxy-5-nitrophenyl)amino)pyrimidin-4-yl)amino)phenyl)dimethylphosphine oxide

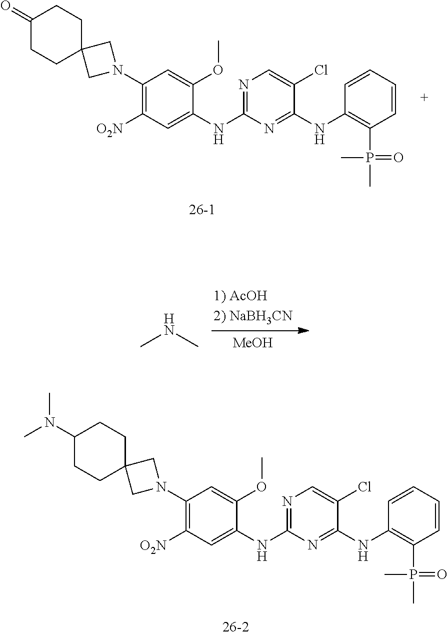

To a solution of 2-(4-((5-chloro-4-((2-(dimethylphosphoryl)phenyl)amino)pyrimidin-2-yl)amino)-5-methoxy-2-nitrophenyl)-2-azaspiro[3.5]nonan-7-one (700 mg) in MeOH (20 mL), was added N-methylmethanamine (2 M, 2.99 mL) and AcOH (143 mg). The mixture was stirred at 60° C. for 1 h. To the resulting solution, was added Na(CN)BH₃ (226 mg). The reaction mixture was stirred for 1 hr at r.t. The resulting mixture was concentrated under vacuum. The crude product was purificated by gel column using DCM/MeOH (0-10%, 20 mins) to obtain (2-((5-chloro-2-((4-(7-(dimethylamino)-2-azaspiro[3.5]nonan-2-yl)-2-methoxy-5-nitrophenyl)amino)pyrimidin-4-yl)amino)phenyl)dimethylphosphine oxide (255 mg) as a red solid. MS: 614 [M+H]⁺.

### Step 4: Synthesis of (2-((2-((5-amino-4-(7-(dimethylamino)-2-azaspiro[3.5]nonan-2-yl)-2-methoxyphenyl)amino)-5-chloropyrimidin-4-yl)amino)phenyl)dimethylphosphine oxide

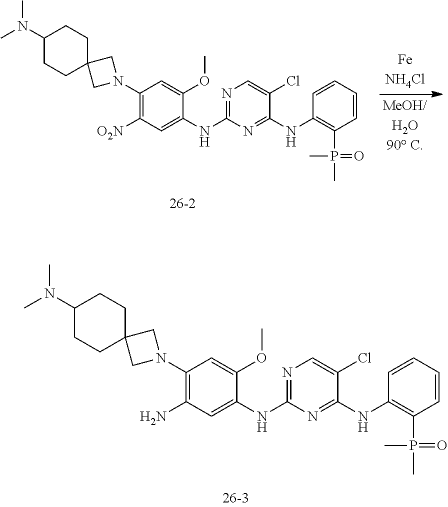 [SMILES: COc1cc(N2CC3(CCC(N(C)C)CC3)C2)c([N+](=O)[O-])cc1Nc1ncc(Cl)c(Nc2ccccc2P(C)(C)=O)n1 ; COc1cc(N2CC3(CCC(N(C)C)CC3)C2)c(N)cc1Nc1ncc(Cl)c(Nc2ccccc2P(C)(C)=O)n1]

Following the same procedure as Synthesis of (2-((2-((5-amino-2-methoxy-4-(4-(4-methylpiperazin-1-yl)piperidin-1-yl)phenyl)amino)-5-chloropyrimidin-4-yl)amino)phenyl)dimethylphosphine oxide using (2-((5-chloro-2-((4-(7-(dimethylamino)-2-azaspiro[3.5]nonan-2-yl)-2-methoxy-5-nitrophenyl)amino)pyrimidin-4-yl)amino)phenyl)dimethylphosphine oxide instead of 5-chloro-N4-(2-dimethylphosphorylphenyl)-N2-[2-methoxy-4-[4-(4-methylpiperazin-1-yl)-1-piperidyl]-5-nitro-phenyl]pyrimidine-2,4-diamine to obtain (2-((2-((5-amino-4-(7-(dimethylamino)-2-azaspiro[3.5]nonan-2-yl)-2-methoxyphenyl)amino)-5-chloropyrimidin-4-yl)amino)phenyl)dimethylphosphine oxide. MS: 584 [M+H]⁺.

### Step 5: Synthesis of N-(5-((5-chloro-4-((2-(dimethylphosphoryl)phenyl)amino)pyrimidin-2-yl)amino)-2-(7-(dimethylamino)-2-azaspiro[3.5]nonan-2-yl)-4-methoxyphenyl)acrylamide

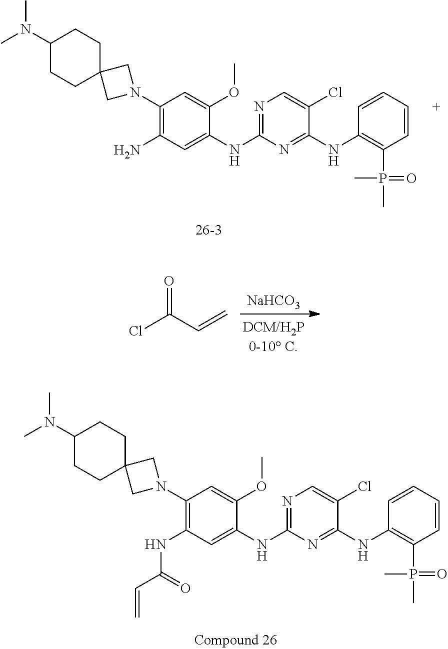

Following the same procedure as N-(5-((5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acrylamide using (2-((2-((5-amino-4-(7-(dimethylamino)-2-azaspiro[3.5]nonan-2-yl)-2-methoxyphenyl)amino)-5-chloropyrimidin-4-yl)amino)phenyl)dimethylphosphine oxide instead of N2-[5-amino-2-methoxy-4-[4-(4-methyl piperazin-1-yl)-1-piperidyl]phenyl]-5-chloro-N4-(2-dimethylphosphorylphenyl)pyrimidine-2,4-diamine to obtain N-(5-((5-chloro-4-((2-(dimethylphosphoryl)phenyl)amino)pyrimidin-2-yl)amino)-2-(7-(dimethylamino)-2-azaspiro[3.5]nonan-2-yl)-4-methoxyphenyl)acrylamide MS: 638[M+H]⁺.

### Example 27 Synthesis of Compound 27

### N-(2-((2-(dimethylamino)ethyl)(methyl)amino)-5-((4-((2-(dimethylphosphoryl)phenyl)amino)-5-fluoropyrimidin-2-yl)amino)-4-methoxyphenyl)acrylamide

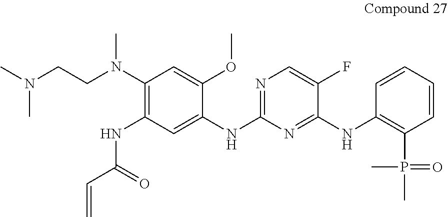 [SMILES: C=CC(=O)Nc1cc(Nc2ncc(F)c(Nc3ccccc3P(C)(C)=O)n2)c(OC)cc1N(C)CCN(C)C]

### Step 1: Synthesis of (2-((5-fluoro-2-((4-fluoro-2-methoxy-5-nitrophenyl)amino)pyrimidin-4-yl)amino)phenyl)dimethylphosphine oxide

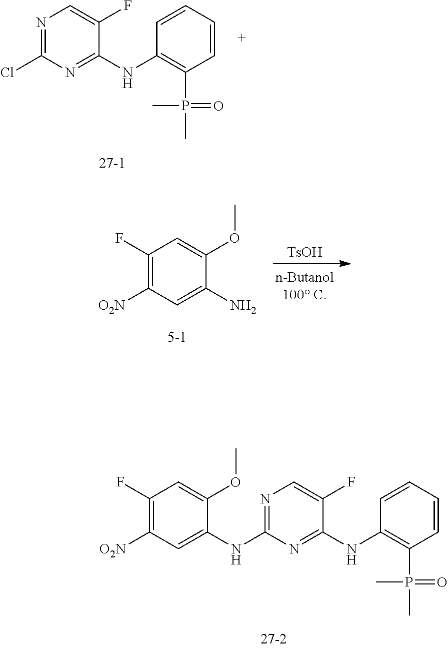

Following the same procedure as (2-((5-chloro-2-((4-fluoro-2-methoxy-5-nitrophenyl)amino)pyrimidin-4-yl)amino)phenyl)dimethylphosphine oxide using (2-((2-chloro-5-fluoropyrimidin-4-yl)amino)phenyl)dimethylphosphine oxide instead of 2,5-dichloro-N-(2-dimethylphosphorylphenyl)pyrimidin-4-amine to obtained (2-((5-fluoro-2-((4-fluoro-2-methoxy-5-nitrophenyl)amino)pyrimidin-4-yl)amino)phenyl)dimethylphosphine oxide. MS: 450 [M+H]⁺.

### Step 2: Synthesis of (2-((2-((4-((2-(dimethylamino)ethyl)(methyl)amino)-2-methoxy-5-nitrophenyl)amino)-5-fluoropyrimidin-4-yl)amino)phenyl)dimethylphosphine oxide

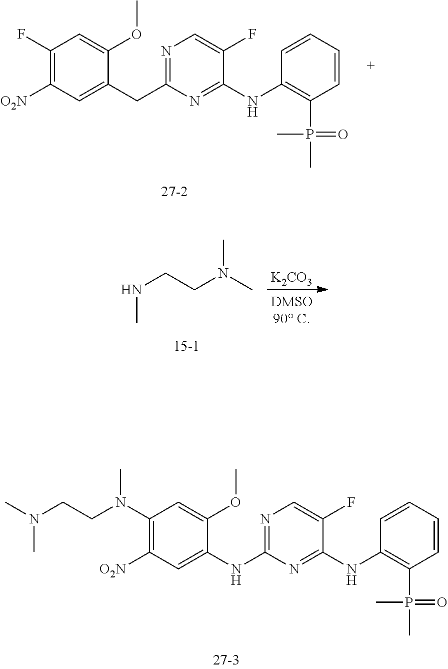

Following the same procedure as (2-((5-chloro-2-((2-methoxy-4-(4-(4-methylpiperazin-1-yl)piperidin-1-yl)-5-nitrophenyl)amino)pyrimidin-4-yl)amino)phenyl)dimethylphosphine oxide using (2-((5-fluoro-2-((4-fluoro-2-methoxy-5-nitrophenyl)amino)pyrimidin-4-yl)amino)phenyl)dimethylphosphine oxide instead of 5-chloro-N4-(2-dimethylphosphorylphenyl)-N2-(4-fluoro-2-methoxy-5-nitro-phenyl)pyrimidine-2,4-diamine to obtain (2-((2-((4-((2-(dimethylamino)ethyl)(methyl)amino)-2-methoxy-5-nitrophenyl)amino)-5-fluoropyrimidin-4-yl)amino)phenyl)dimethylphosphine oxide. MS: 532 [M+H]⁺.

### Step 3: Synthesis of (2-((2-((5-amino-4-((2-(dimethylamino)ethyl)(methyl)amino)-2-methoxyphenyl)amino)-5-fluoropyrimidin-4-yl)amino)phenyl)dimethylphosphine oxide

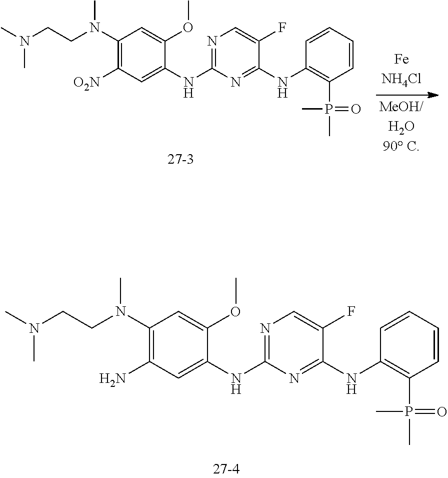 [SMILES: COc1cc(N(C)CCN(C)C)c([N+](=O)[O-])cc1Nc1ncc(F)c(Nc2ccccc2P(C)(C)=O)n1 ; COc1cc(N(C)CCN(C)C)c(N)cc1Nc1ncc(F)c(Nc2ccccc2P(C)(C)=O)n1]

Following the same procedure as Synthesis of (2-((2-((5-amino-2-methoxy-4-(4-(4-methylpiperazin-1-yl)piperidin-1-yl)phenyl)amino)-5-chloropyrimidin-4-yl)amino)phenyl)dimethylphosphine oxide using N2-(4-((2-(dimethylamino)ethyl)(methyl)amino)-2-methoxy-5-nitrophenyl)-N4-(2-(dimethylphosphanyl)phenyl)-5-fluoropyrimidine-2,4-diamine instead of 5-chloro-N4-(2-dimethylphosphorylphenyl)-N2-[2-methoxy-4-[4-(4-methylpiperazin-1-yl)-1-piperidyl]-5-nitro-phenyl]pyrimidine-2,4-diamine to obtain (2-((2-((5-amino-4-((2-(dimethylamino)ethyl)(methyl)amino)-2-methoxyphenyl)amino)-5-fluoropyrimidin-4-yl)amino)phenyl)dimethylphosphine oxide. MS: 502 [M+H]⁺.

### Step 4: Synthesis of N-(2-((2-(dimethylamino)ethyl)(methyl)amino)-5-((4-((2-(dimethylphosphoryl)phenyl)amino)-5-fluoropyrimidin-2-yl)amino)-4-methoxyphenyl)acrylamide

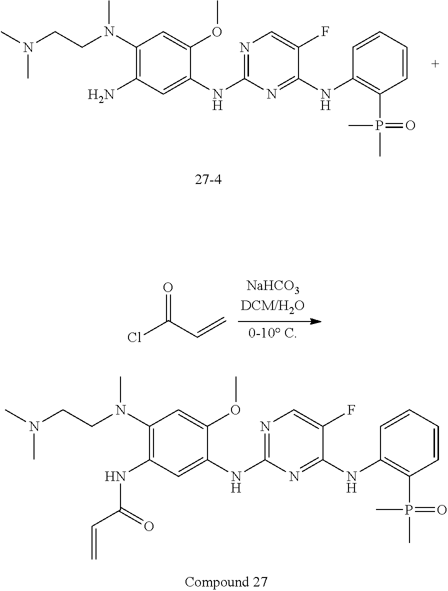

Following the same procedure as N-(5-((5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acrylamide using (2-((2-((5-amino-4-((2-(dimethylamino)ethyl)(methyl)amino)-2-methoxyphenyl)amino)-5-fluoropyrimidin-4-yl)amino)phenyl)dimethylphosphine oxide instead of N2-[5-amino-2-methoxy-4-[4-(4-methylpiperazin-1-yl)-1-piperidyl]phenyl]-5-chloro-N4-(2-dimethylphosphorylphenyl)pyrimidine-2,4-diamine to obtain N-(2-((2-(dimethylamino)ethyl)(methyl)amino)-5-((4-((2-(dimethylphosphoryl)phenyl)amino)-5-fluoropyrimidin-2-yl)amino)-4-methoxyphenyl)acrylamide. MS: 556 [M+H]⁺.

### Example 28 Synthesis of Compound 28

### N-(5-((4-((2-(dimethylphosphoryl)phenyl)amino)-5-fluoropyrimidin-2-yl)amino)-4-methoxy-2-(4-(4-methylpiperazin-1-yl)piperidin-1-yl)phenyl)acrylamide

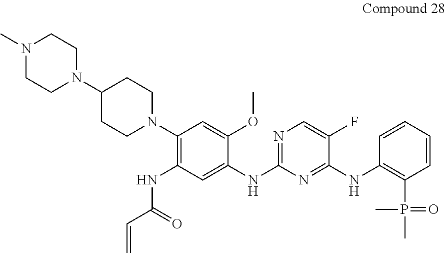 [SMILES: C=CC(=O)Nc1cc(Nc2ncc(F)c(Nc3ccccc3P(C)(C)=O)n2)c(OC)cc1N1CCC(N2CCN(C)CC2)CC1]

### Step 1: Synthesis of (2-((5-fluoro-2-((2-methoxy-4-(4-(4-methylpiperazin-1-yl)piperidin-1-yl)-5-nitrophenyl)amino)pyrimidin-4-yl)amino)phenyl)dimethylphosphine oxide

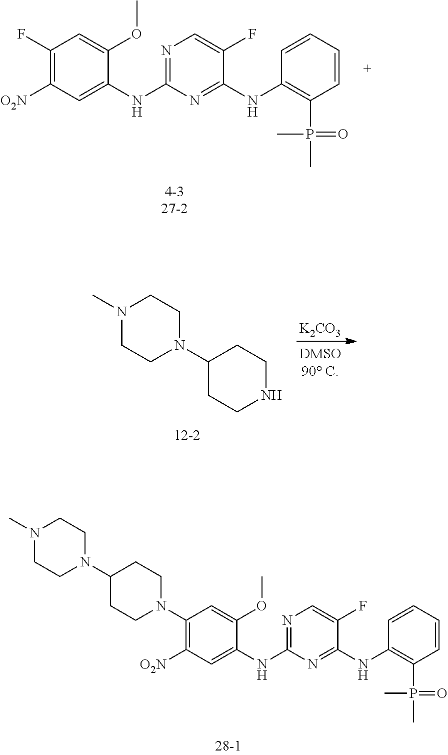

Following the same procedure as (2-((5-chloro-2-((2-methoxy-4-(4-(4-methylpiperazin-1-yl)piperidin-1-yl)-5-nitrophenyl)amino)pyrimidin-4-yl)amino)phenyl)dimethylphosphine oxide using (2-((5-fluoro-2-((4-fluoro-2-methoxy-5-nitrophenyl)amino)pyrimidin-4-yl)amino)phenyl)dimethylphosphine oxide instead of 5-chloro-N4-(2-dimethylphosphorylphenyl)-N2-(4-fluoro-2-methoxy-5-nitro-phenyl)pyrimidine-2,4-diamine to obtain (2-((5-fluoro-2-((2-methoxy-4-(4-(4-methylpiperazin-1-yl)piperidin-1-yl)-5-nitrophenyl)amino)pyrimidin-4-yl)amino)phenyl)dimethylphosphine oxide. MS: 613 [M+H]⁺.

### Step 2: Synthesis of (2-((2-((5-amino-2-methoxy-4-(4-(4-methylpiperazin-1-yl)piperidin-1-yl)phenyl)amino)-5-fluoropyrimidin-4-yl)amino)phenyl)dimethylphosphine oxide

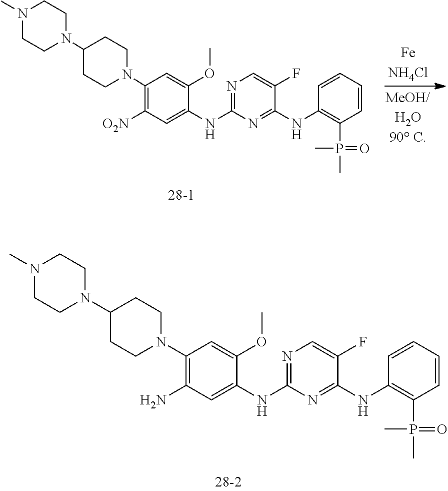 [SMILES: COc1cc(N2CCC(N3CCN(C)CC3)CC2)c([N+](=O)[O-])cc1Nc1ncc(F)c(Nc2ccccc2P(C)(C)=O)n1 ; COc1cc(N2CCC(N3CCN(C)CC3)CC2)c(N)cc1Nc1ncc(F)c(Nc2ccccc2P(C)(C)=O)n1]

Following the same procedure as Synthesis of (2-((2-((5-amino-2-methoxy-4-(4-(4-methylpiperazin-1-yl)piperidin-1-yl)phenyl)amino)-5-chloropyrimidin-4-yl)amino)phenyl)dimethylphosphine oxide using (2-((5-fluoro-2-((2-methoxy-4-(4-(4-methylpiperazin-1-yl)piperidin-1-yl)-5-nitrophenyl)amino)pyrimidin-4-yl)amino)phenyl)dimethylphosphine oxide instead of 5-chloro-N4-(2-dimethylphosphorylphenyl)-N2-[2-methoxy-4-[4-(4-methylpiperazin-1-yl)-1-piperidyl]-5-nitro-phenyl]pyrimidine-2,4-diamine to obtain (2-((2-((5-amino-2-methoxy-4-(4-(4-methyl piperazin-1-yl)piperidin-1-yl)phenyl)amino)-5-fluoropyrimidin-4-yl)amino)phenyl)dimethylphosphine oxide. MS: 583 [M+H]⁺.

### Step 3: Synthesis of N-(5-((4-((2-(dimethylphosphoryl)phenyl)amino)-5-fluoropyrimidin-2-yl)amino)-4-methoxy-2-(4-(4-methylpiperazin-1-yl)piperidin-1-yl)phenyl)acrylamide

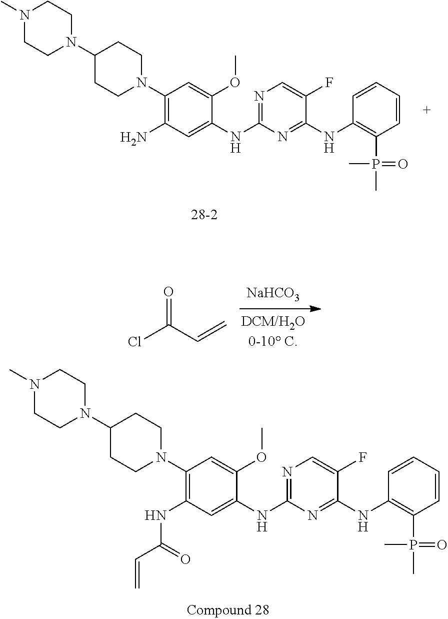

Following the same procedure as N-(5-((5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acrylamide using (2-((2-((5-amino-2-methoxy-4-(4-(4-methylpiperazin-1-yl)piperidin-1-yl)phenyl)amino)-5-fluoropyrimidin-4-yl)amino)phenyl)dimethylphosphine oxide instead of N2-[5-amino-2-methoxy-4-[4-(4-methyl piperazin-1-yl)-1-piperidyl]phenyl]-5-chloro-N4-(2-dimethylphosphorylphenyl)pyrimidine-2,4-diamine to obtain N-(5-((4-((2-(dimethylphosphoryl)phenyl)amino)-5-fluoropyrimidin-2-yl)amino)-4-methoxy-2-(4-(4-methylpiperazin-1-yl)piperidin-1-yl)phenyl)acrylamide. MS: 637 [M+H]⁺.

¹H NMR (500 MHz, DMSO-d6) δ 11.55 (s, 1H), 9.03 (s, 1H), 8.61 (m, 1H), 8.22 (s, 1H), 8.07 (m, 2H), 7.56 (m, 1H), 7.38 (m, 1H), 7.06 (m, 1H), 6.82 (s, 1H), 6.70 (m, 1H), 6.19 (m, 1H), 5.72 (m, 1H), 3.79 (s, 3H), 3.17 (s, 2H), 2.70 (m, 8H) 2.43 (m, 6H), 1.85 (m, 9H).

### Example 29 Synthesis of Compound 29

### N-(5-((4-((2-(dimethylphosphoryl)phenyl)amino)-5-fluoropyrimidin-2-yl)amino)-2-(4-(4-methylpiperazin-1-yl)piperidin-1-yl)phenyl)acrylamide

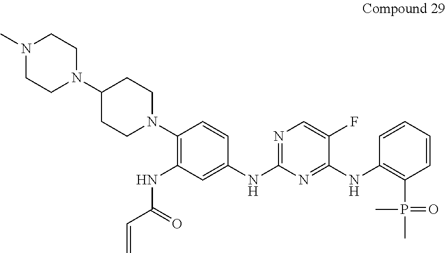 [SMILES: C=CC(=O)Nc1cc(Nc2ncc(F)c(Nc3ccccc3P(C)(C)=O)n2)ccc1N1CCC(N2CCN(C)CC2)CC1]

### Step 1: Synthesis of (2-((5-fluoro-2-((4-fluoro-3-nitrophenyl)amino)pyrimidin-4-yl)amino)phenyl)dimethylphosphine oxide

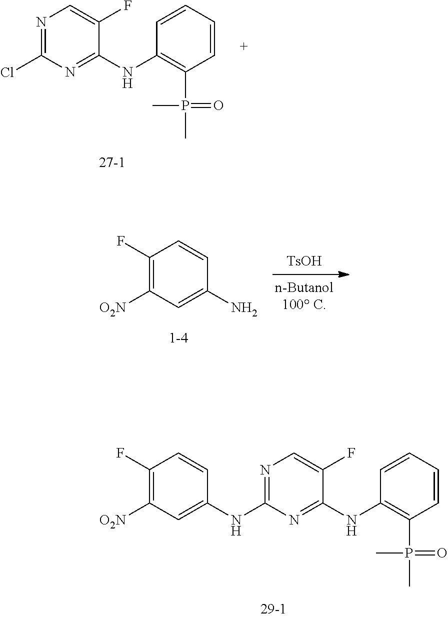 [SMILES: CP(C)(=O)c1ccccc1Nc1nc(Cl)ncc1F ; Nc1ccc(F)c([N+](=O)[O-])c1 ; "TsOH" ; CP(C)(=O)c1ccccc1Nc1nc(Nc2ccc(F)c([N+](=O)[O-])c2)ncc1F]

Following the same procedure as (2-((5-chloro-2-((4-fluoro-2-methoxy-5-nitrophenyl)amino)pyrimidin-4-yl)amino)phenyl)dimethylphosphine oxide using (2-((2-chloro-5-fluoropyrimidin-4-yl)amino)phenyl)dimethylphosphine oxide instead of 2,5-dichloro-N-(2-dimethylphosphorylphenyl)pyrimidin-4-amine and using 4-fluoro-3-nitroaniline instead of 4-fluoro-2-methoxy-5-nitro-aniline to obtain (2-((5-fluoro-2-((4-fluoro-3-nitrophenyl)amino)pyrimidin-4-yl)amino)phenyl)dimethylphosphine oxide. MS: 420 [M+H]⁺.

### Step 2: Synthesis of (2-((5-fluoro-2-((4-(4-(4-methylpiperazin-1-yl)piperidin-1-yl)-3-nitrophenyl)amino)pyrimidin-4-yl)amino)phenyl)dimethylphosphine oxide

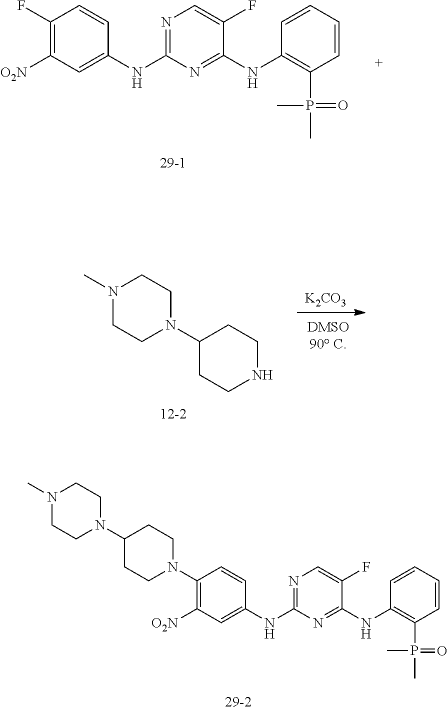

Following the same procedure as (2-((5-chloro-2-((2-methoxy-4-(4-(4-methylpiperazin-1-yl)piperidin-1-yl)-5-nitrophenyl)amino)pyrimidin-4-yl)amino)phenyl)dimethylphosphine oxide using (2-((5-fluoro-2-((4-fluoro-3-nitrophenyl)amino)pyrimidin-4-yl)amino)phenyl)dimethylphosphine oxide instead of 5-chloro-N4-(2-dimethylphosphorylphenyl)-N2-(4-fluoro-2-methoxy-5-nitro-phenyl)pyrimidine-2,4-diamine to obtain (2-((5-fluoro-2-((4-(4-(4-methylpiperazin-1-yl)piperidin-1-yl)-3-nitrophenyl)amino)pyrimidin-4-yl)amino)phenyl)dimethylphosphine oxide. MS: 583 [M+H]⁺.

### Step 3: Synthesis of (2-((2-((3-amino-4-(4-(4-methylpiperazin-1-yl)piperidin-1-yl)phenyl)amino)-5-fluoropyrimidin-4-yl)amino)phenyl)dimethylphosphine oxide

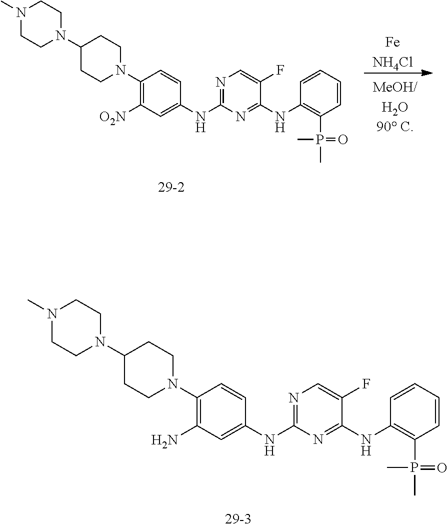 [SMILES: CN1CCN(C2CCN(c3ccc(Nc4ncc(F)c(Nc5ccccc5P(C)(C)=O)n4)cc3[N+](=O)[O-])CC2)CC1 ; "FeNH4Cl" ; "MeOH/H2O90° C." ; CN1CCN(C2CCN(c3ccc(Nc4ncc(F)c(Nc5ccccc5P(C)(C)=O)n4)cc3N)CC2)CC1]

Following the same procedure as Synthesis of (2-((2-((5-amino-2-methoxy-4-(4-(4-methylpiperazin-1-yl)piperidin-1-yl)phenyl)amino)-5-chloropyrimidin-4-yl)amino)phenyl)dimethylphosphine oxide using (2-((5-fluoro-2-((4-(4-(4-methylpiperazin-1-yl)piperidin-1-yl)-3-nitrophenyl)amino)pyrimidin-4-yl)amino)phenyl)dimethylphosphine oxide instead of 5-chloro-N4-(2-dimethylphosphorylphenyl)-N2-[2-methoxy-4-[4-(4-methylpiperazin-1-yl)-1-piperidyl]-5-nitro-phenyl]pyrimidine-2,4-diamine to obtain (2-((2-((3-amino-4-(4-(4-methylpiperazin-1-yl)piperidin-1-yl)phenyl)amino)-5-fluoropyrimidin-4-yl)amino)phenyl)dimethylphosphine oxide. MS: 553 [M+H]⁺.

### Step 4: Synthesis of N-(5-((4-((2-(dimethylphosphoryl)phenyl)amino)-5-fluoropyrimidin-2-yl)amino)-2-(4-(4-methylpiperazin-1-yl)piperidin-1-yl)phenyl)acrylamide

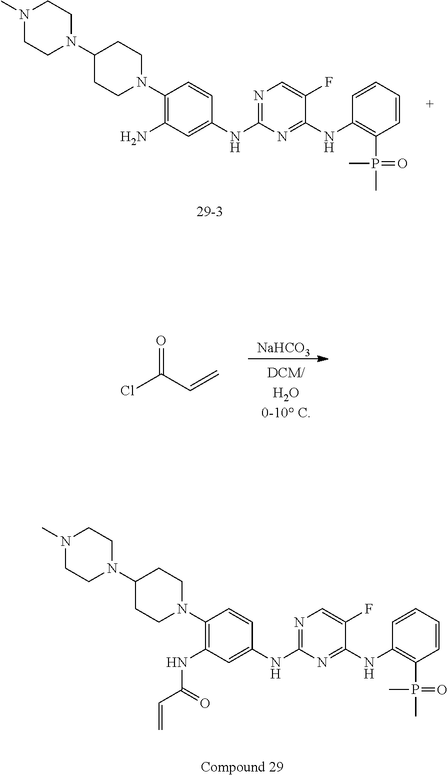

Following the same procedure as N-(5-((5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acrylamide using (2-((2-((3-amino-4-(4-(4-methylpiperazin-1-yl)piperidin-1-yl)phenyl)amino)-5-fluoropyrimidin-4-yl)amino)phenyl)dimethylphosphine oxide instead of N2-[5-amino-2-methoxy-4-[4-(4-methylpiperazin-1-yl)-1-piperidyl]phenyl]-5-chloro-N4-(2-dimethylphosphorylphenyl)pyrimidine-2,4-diamine to obtain N-(5-((4-((2-(dimethylphosphoryl)phenyl)amino)-5-fluoropyrimidin-2-yl)amino)-2-(4-(4-methylpiperazin-1-yl)piperidin-1-yl)phenyl)acrylamide. MS: 607 [M+H]⁺.

### Example 30 Synthesis of Compound 30

### N-(5-((5-chloro-4-((5-(dimethylphosphoryl)quinolin-6-yl)amino)pyrimidin-2-yl)amino)-2-(2-(dimethylamino)-7-azaspiro[3.5]nonan-7-yl)phenyl)acrylamide

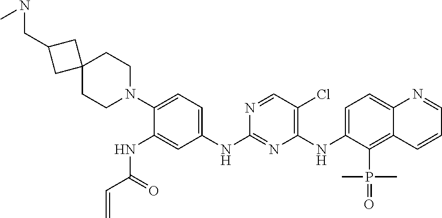 [SMILES: C=CC(=O)Nc1cc(Nc2ncc(Cl)c(Nc3ccc4ncccc4c3P(C)(C)=O)n2)ccc1N1CCC2(CC1)CC(CNC)C2]

### Step 1: Synthesis of 5-iodoquinolin-6-amine

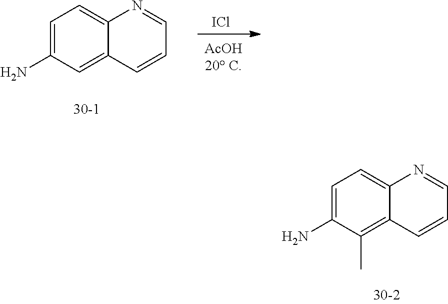

To a solution of quinolin-6-amine (2.50 g) in acetic acid (30 mL), was added a solution of ICl (4.08 g) in 10 ml of acetic acid at 10-15° C. The reaction solution was stirred at 20° C. for 1 hour. The reaction mixture was concentrated under vacuum. The residue was washed with ethyl acetate (50 ml), and then was filtered by a suction funnel. The solid was collected to obtain 5-iodoquinolin-6-amine (5.20 g). MS: 271 [M+H]⁺.

### Step 2: Synthesis of (6-aminoquinolin-5-yl)dimethylphosphine oxide

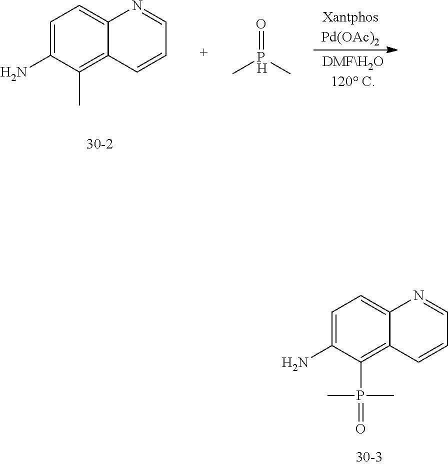

To a solution of 5-iodoquinolin-6-amine (4.70 g) in DMF/H2O (100 mL/20 ml), was added methylphosphonoylmethane (1.80 g), Xantphos (1.77 g), Pd(OAc)₂ (344 mg) and K₃PO₄ (9.76 g) at r.t. The reaction solution was stirred at 120° C. overnight. The reaction mixture was concentrated under vacuum. The residue was purificated by silica gel column (MeOH was changed from 0 to 10%, 20 mins) to obtained (6-aminoquinolin-5-yl)dimethylphosphine oxide (2.50 g). MS: 221 [M+H]⁺.

### Step 3: (6-((2,5-dichloropyrimidin-4-yl)amino)quinolin-5-yl)dimethylphosphine oxide

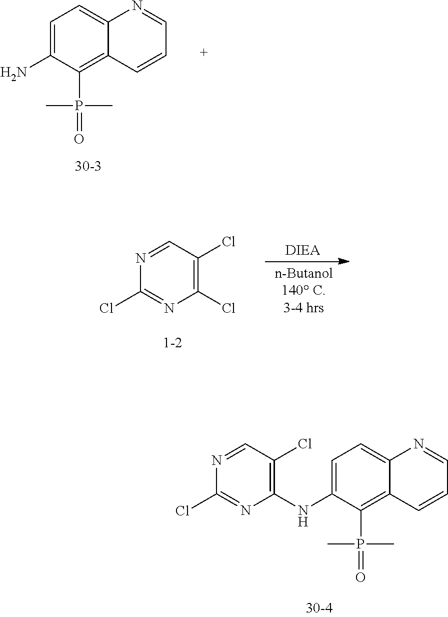 [SMILES: CP(C)(=O)c1c(N)ccc2ncccc12 ; Clc1ncc(Cl)c(Cl)n1 ; "DIEA" ; "n-Butanol140° C.3-4 hrs" ; CP(C)(=O)c1c(Nc2nc(Cl)ncc2Cl)ccc2ncccc12]

To a solution of 2,4,5-trichloropyrimidine (1.67 g, 9.08 mmol) in n-Butanol (15 ml), was added (6-aminoquinolin-5-yl)dimethylphosphine oxide (1.00 g) and DIEA (1.76 g, 13.62 mmol, 2.37 mL) at r.t. The reaction solution was stirred at 120° C. for 4 hours. The reaction mixture was cooled to room temperature. The reaction mixture was filtered by a suction funnel and the solid was collected to obtained (6-((2,5-dichloropyrimidin-4-yl)amino)quinolin-5-yl)dimethylphosphine oxide (0.90 g, 2.45 mmol, 53.98% yield). MS: 367 [M+H]⁺.

### Step 4: Synthesis of (6-((5-chloro-2-((4-fluoro-3-nitrophenyl)amino)pyrimidin-4-yl)amino)quinolin-5-yl)dimethylphosphine oxide

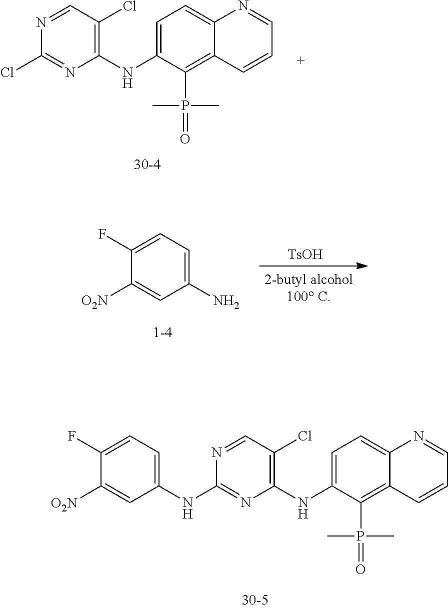 [SMILES: CP(C)(=O)c1c(Nc2nc(Cl)ncc2Cl)ccc2ncccc12 ; Nc1ccc(F)c([N+](=O)[O-])c1 ; "TsOH" ; "2-butyl alcohol100° C." ; CP(C)(=O)c1c(Nc2nc(Nc3ccc(F)c([N+](=O)[O-])c3)ncc2Cl)ccc2ncccc12]

To a solution of (6-((2,5-dichloropyrimidin-4-yl)amino)quinolin-5-yl)dimethylphosphine oxide (1.60 g) in 2-butyl alcohol (15 mL), was added 4-fluoro-3-nitro-aniline (680 mg) and TsOH (1.13 g). The reaction solution was stirred at 100° C. for overnight. The reaction mixture was cooled down to room temperature and was filtered by by a suction funnel. The solid was collected and dried to obtained (6-((5-chloro-2-((4-fluoro-3-nitrophenyl)amino)pyrimidin-4-yl)amino)quinolin-5-yl)dimethylphosphine oxide. MS: 487 [M+H]⁺.

### Step 5: Synthesis of 7-(4-((5-chloro-4-((5-(dimethylphosphoryl)quinolin-6-yl)amino)pyrimidin-2-yl)amino)-2-nitrophenyl)-7-azaspiro[3.5]nonan-2-one

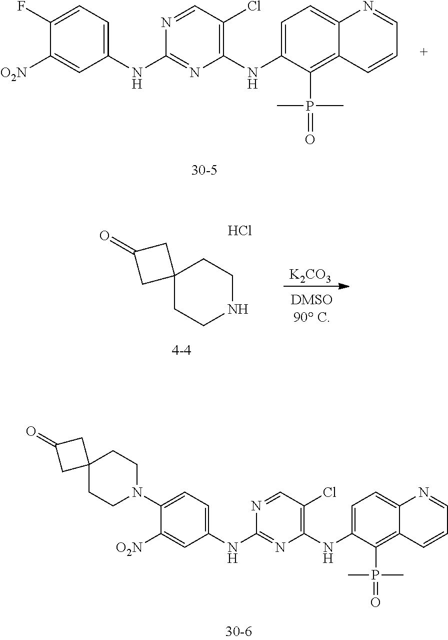

To a solution of (6-((5-chloro-2-((4-fluoro-3-nitrophenyl)amino)pyrimidin-4-yl)amino)quinolin-5-yl)dimethylphosphine oxide (1.60 g) in DMSO (20 mL), was added 7-azaspiro[3.5]nonan-2-one hydrochloride (693 mg) and anhydrous potassium carbonate (1.36 g). The reaction solution was stirred at 90° C. for overnight. The reaction mixture was cooled to room temperature and diluted with water (50 mL). The resulting mixture was extracted with dichloromethane for two times. The organic layers was dried over anhydrous sodium sulfate and concentrated under vacuum to obtained 7-(4-((5-chloro-4-((5-(dimethylphosphoryl)quinolin-6-yl)amino)pyrimidin-2-yl)amino)-2-nitrophenyl)-7-azaspiro[3.5]nonan-2-one (1.00 g) as a yellow solid. MS: 606 [M+H]⁺.

### Step 6: Synthesis of (6-((5-chloro-2-((4-(2-(dimethylamino)-7-azaspiro[3.5]nonan-7-yl)-3-nitrophenyl)amino)pyrimidin-4-yl)amino)quinolin-5-yl)dimethylphosphine oxide

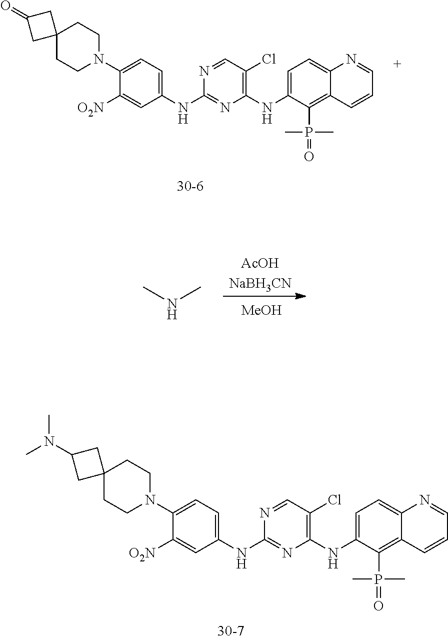

To a solution of 7-(4-((5-chloro-4-((5-(dimethylphosphoryl)quinolin-6-yl)amino)pyrimidin-2-yl)amino)-2-nitrophenyl)-7-azaspiro[3.5]nonan-2-one (1.00 g) in MeOH (20 mL), was added dimethylamine solution (372 mg) and AcOH (99 mg). The reaction mixture was stirred at 60° C. for 0.5 h. Then Na(CN)BH₃ (311 mg) was added to the resulting solution. The resulting mixture was stirred at r.t for 2 h. The resulting mixture was concentrated under vacuum. The crude product was purificated by flash silica gel column (MeOH from 0-10%, 20 mins) to obtained (6-((5-chloro-2-((4-(2-(dimethylamino)-7-azaspiro[3.5]nonan-7-yl)-3-nitrophenyl)amino)pyrimidin-4-yl)amino)quinolin-5-yl)dimethylphosphine oxide (0.95 g). MS: 635 [M+H]⁺.

### Step 7: Synthesis of (6-((2-((3-amino-4-(2-(dimethylamino)-7-azaspiro[3.5]nonan-7-yl)phenyl)amino)-5-chloropyrimidin-4-yl)amino)quinolin-5-yl)dimethylphosphine oxide

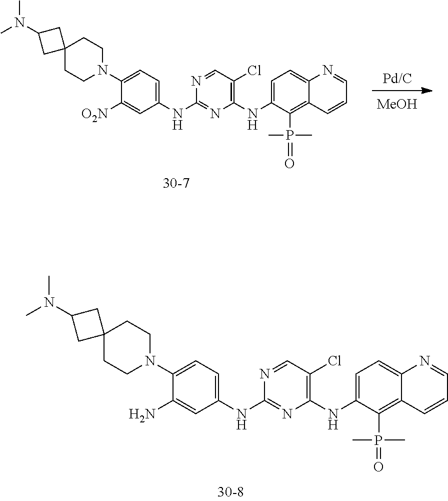 [SMILES: "MeOH" ; CN(C)C1CC2(CCN(c3ccc(Nc4ncc(Cl)c(Nc5ccc6ncccc6c5P(C)(C)=O)n4)cc3[N+](=O)[O-])CC2)C1 ; "Pd/C" ; CN(C)C1CC2(CCN(c3ccc(Nc4ncc(Cl)c(Nc5ccc6ncccc6c5P(C)(C)=O)n4)cc3N)CC2)C1]

To a solution of (6-((5-chloro-2-((4-(2-(dimethylamino)-7-azaspiro[3.5]nonan-7-yl)-3-nitrophenyl)amino)pyrimidin-4-yl)amino)quinolin-5-yl)dimethylphosphine oxide (1.00 g) in MeOH (20 ml), was added Pd/C (200 mg). The reaction solution was stirred under hydrogen pressure for 2 hours. The reaction mixture is filtered by a a suction funnel. The filtrate was collected and concentrated to obtained (6-((2-((3-amino-4-(2-(dimethylamino)-7-azaspiro[3.5]nonan-7-yl)phenyl)amino)-5-chloropyrimidin-4-yl)amino)quinolin-5-yl)dimethylphosphine oxide (380 mg) as a yellow solid. MS: 605 [M+H]⁺.

### Step 8: Synthesis of N-(5-((5-chloro-4-((5-(dimethylphosphoryl)quinolin-6-yl)amino)pyrimidin-2-yl)amino)-2-(2-(dimethylamino)-7-azaspiro[3.5]nonan-7-yl)phenyl)acrylamide

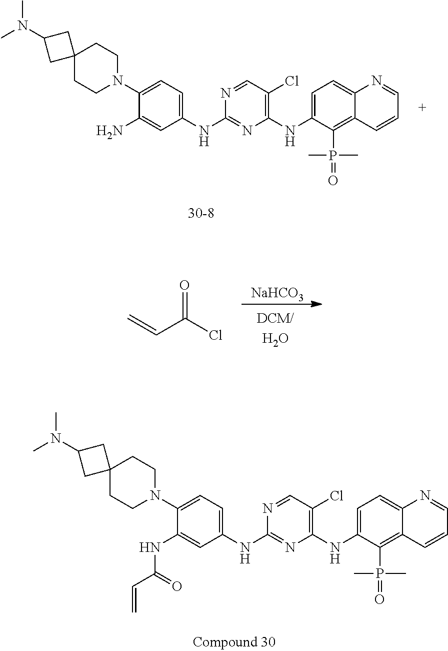

To a solution of (6-((2-((3-amino-4-(2-(dimethylamino)-7-azaspiro[3.5]nonan-7-yl)phenyl)amino)-5-chloropyrimidin-4-yl)amino)quinolin-5-yl)dimethylphosphine oxide (120 mg) NaHCO₃ (50 mg) in DCM (5 mL) and H₂O (5 mL), was dropwise added prop-2-enoyl chloride (22 mg) in DCM (0.5 ml) at 0-10° C. The resulting solution was stirred for 0.5 h at 0-10° C. The reaction was concentrated by vacuum. The residue was purified by column chromatography over silica gel with DCM/MeOH (8:1) to obtain Compound 30 N-(5-((5-chloro-4-((5-(dimethylphosphoryl)quinolin-6-yl)amino)pyrimidin-2-yl)amino)-2-(2-(dimethylamino)-7-azaspiro[3.5]nonan-7-yl)phenyl)acrylamide (27.6 mg) MS: 659 [M+H]⁺.

¹H NMR (500 MHz, DMSO-d6) δ 12.45 (s, 1H), 11.03 (s, 1H), 9.383 (d, J=7.5 Hz, 1H), 9.01 (s, 1H), 8.85 (m, 2H), 8.57 (d, J=8.5 Hz, 1H), 8.21 (m, 2H), 8.05 (d, J=9.5 Hz, 1H), 7.56 (m, 1H), 7.37 (d, J=7.0 Hz, 1H), 6.97 (d, J=8.5 Hz, 1H), 6.64 (m, 1H), 6.17 (m, 1H), 5.76 (m, 1H), 2.67 (m, 2H), 2.62 (m, 2H), 2.48 (s, 6H), 2.14 (s, 2H) 2.06-1.96 (m, 8H), 1.75 (m, 4H).

### Example 31 Synthesis of Compound 31

### N-(5-((5-chloro-4-((2-(dimethylphosphoryl)phenyl)amino)pyrimidin-2-yl)amino)-2-(2-(dimethylamino)-7-azaspiro[3.5]nonan-7-yl)-4-methylphenyl)acrylamide

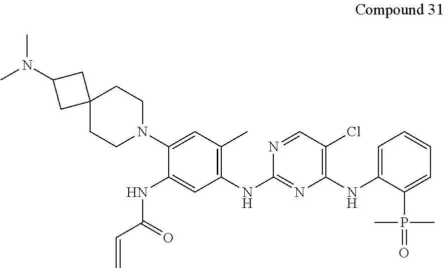

### Step 1: Synthesis of 4-fluoro-2-methyl-5-nitroaniline

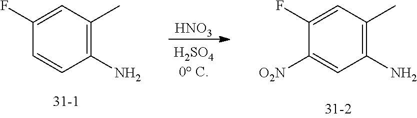

To a solution of 4-fluoro-2-methylaniline (5.25 g) in H₂SO₄ (45 mL), was dropwise added a solution of fuming nitric acid (3.17 g) in H₂SO₄ (5 mL) at 0-10° C. The resulting solution was stirred for 2 h at 0-10° C. Pour the reaction solution into ice water, then adjust pH to 9-10 with a aqueous solution of 8 N NaOH and a yellow solid was separated out. The solid was filtered by a suction funnel. The filter cake was dried to obtained 4-fluoro-2-methyl-5-nitroaniline (6.30 g). MS: 171 [M+14]⁺.

### Step 2: Synthesis of (2-((5-chloro-2-((4-fluoro-2-methyl-5-nitrophenyl)amino)pyrimidin-4-yl)amino)phenyl)dimethylphosphine oxide

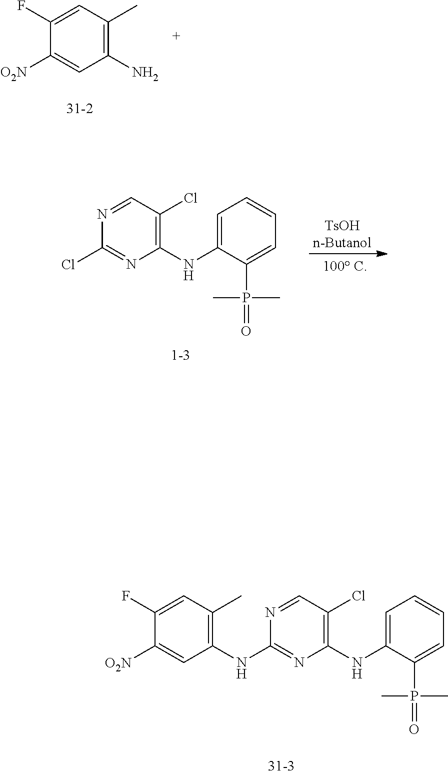 [SMILES: Cc1cc(F)c([N+](=O)[O-])cc1N ; CP(C)(=O)c1ccccc1Nc1nc(Cl)ncc1Cl ; "TsOHn-Butanol" ; "100° C." ; Cc1cc(F)c([N+](=O)[O-])cc1Nc1ncc(Cl)c(Nc2ccccc2P(C)(C)=O)n1]

Following the same procedure as (6-((5-chloro-2-((4-fluoro-3-nitrophenyl)amino)pyrimidin-4-yl)amino)quinolin-5-yl)dimethylphosphine oxide using 4-fluoro-2-methyl-5-nitro aniline instead of 4-fluoro-3-nitro-aniline and using (2-((2,5-dichloropyrimidin-4-yl)amino)phenyl)dimethylphosphine oxide instead of (6-((2,5-dichloropyrimidin-4-yl)amino)quinolin-5-yl)dimethylphosphine oxide (1.60 g) to obtain (2-((5-chloro-2-((4-fluoro-2-methyl-5-nitrophenyl)amino)pyrimidin-4-yl)amino)phenyl)dimethylphosphine oxide. MS: 450 [M+H]⁺.

### Step 3: Synthesis of 7-(4-((5-chloro-4-((2-(dimethylphosphoryl)phenyl)amino)pyrimidin-2-yl)amino)-5-methyl-2-nitrophenyl)-7-azaspiro[3.5]nonan-2-one

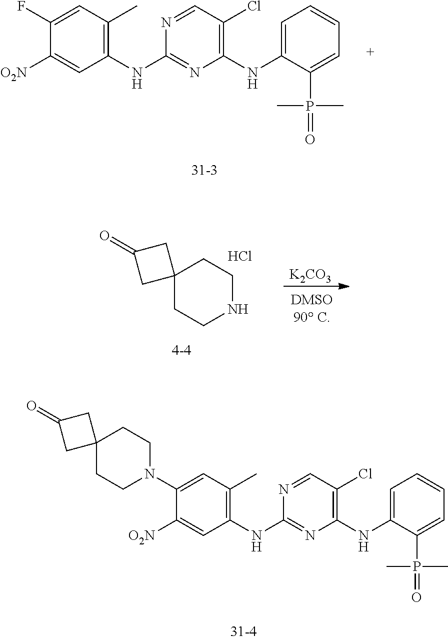

Following the same procedure as 7-(4-((5-chloro-4-((5-(dimethylphosphoryl)quinolin-6-yl)amino)pyrimidin-2-yl)amino)-2-nitrophenyl)-7-azaspiro[3.5]nonan-2-one using (2-((5-chloro-2-((4-fluoro-2-methyl-5-nitrophenyl)amino)pyrimidin-4-yl)amino)phenyl)dimethylphosphine oxide instead of (6-((5-chloro-2-((4-fluoro-3-nitrophenyl)amino)pyrimidin-4-yl)amino)quinolin-5-yl)dimethylphosphine oxide to obtain 7-(4-((5-chloro-4-((2-(dimethylphosphoryl)phenyl)amino)pyrimidin-2-yl)amino)-5-methyl-2-nitrophenyl)-7-azaspiro[3.5]nonan-2-one. MS: 569 [M+H]⁺.

### Step 4: Synthesis of (2-((5-chloro-2-((4-(2-(dimethylamino)-7-azaspiro[3.5]nonan-7-yl)-2-methyl-5-nitrophenyl)amino)pyrimidin-4-yl)amino)phenyl)dimethylphosphine oxide

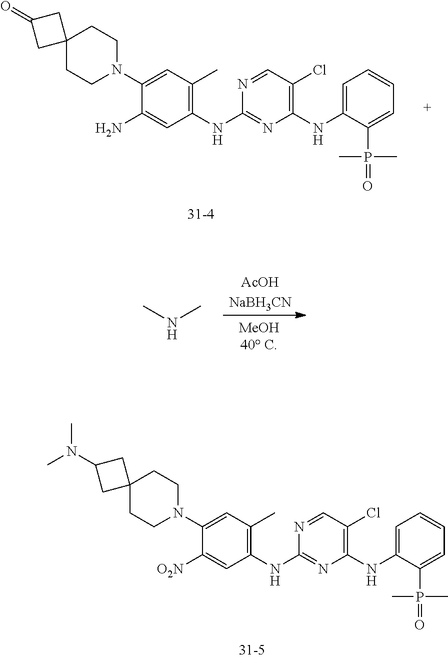

Following the same procedure as (6-((5-chloro-2-((4-(2-(dimethylamino)-7-azaspiro[3.5]nonan-7-yl)-3-nitrophenyl)amino)pyrimidin-4-yl)amino)quinolin-5-yl)dimethylphosphine oxide using 7-(4-((5-chloro-4-((2-(dimethylphosphoryl)phenyl)amino)pyrimidin-2-yl)amino)-5-methyl-2-nitrophenyl)-7-azaspiro[3.5]nonan-2-one instead of 7-(4-((5-chloro-4-((5-(dimethylphosphoryl)quinolin-6-yl)amino)pyrimidin-2-yl)amino)-2-nitrophenyl)-7-azaspiro[3.5]nonan-2-one to obtain (2-((5-chloro-2-((4-(2-(dimethylamino)-7-azaspiro[3.5]nonan-7-yl)-2-methyl-5-nitrophenyl)amino)pyrimidin-4-yl)amino)phenyl)dimethylphosphine oxide. MS: 598 [M+H]⁺

### Step 5: Synthesis of (2-((2-((5-amino-4-(2-(dimethylamino)-7-azaspiro[3.5]nonan-7-yl)-2-methylphenyl)amino)-5-chloropyrimidin-4-yl)amino)phenyl)dimethylphosphine oxide

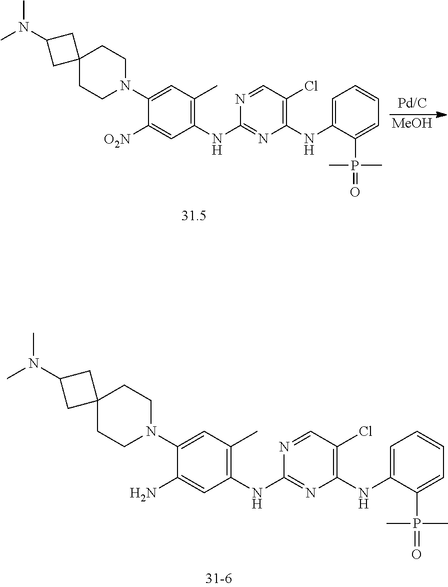

Following the same procedure as Synthesis of (6-((2-((3-amino-4-(2-(dimethylamino)-7-azaspiro[3.5]nonan-7-yl)phenyl)amino)-5-chloropyrimidin-4-yl)amino)quinolin-5-yl)dimethylphosphine oxide using (2-((5-chloro-2-((4-(2-(dimethylamino)-7-azaspiro[3.5]nonan-7-yl)-2-methyl-5-nitrophenyl)amino)pyrimidin-4-yl)amino)phenyl)dimethylphosphine oxide instead of (6-((5-chloro-2-((4-(2-(dimethylamino)-7-azaspiro[3.5]nonan-7-yl)-3-nitrophenyl)amino)pyrimidin-4-yl)amino)quinolin-5-yl)dimethylphosphine oxide to obtain (2-((2-((5-amino-4-(2-(dimethylamino)-7-azaspiro[3.5]nonan-7-yl)-2-methylphenyl)amino)-5-chloropyrimidin-4-yl)amino)phenyl)dimethylphosphine oxide. MS: 568 [M+H]⁺.

### Step 6: Synthesis of N-(5-((5-chloro-4-((2-(dimethylphosphoryl)phenyl)amino)pyrimidin-2-yl)amino)-2-(2-(dimethylamino)-7-azaspiro[3.5]nonan-7-yl)-4-methylphenyl)acrylamide

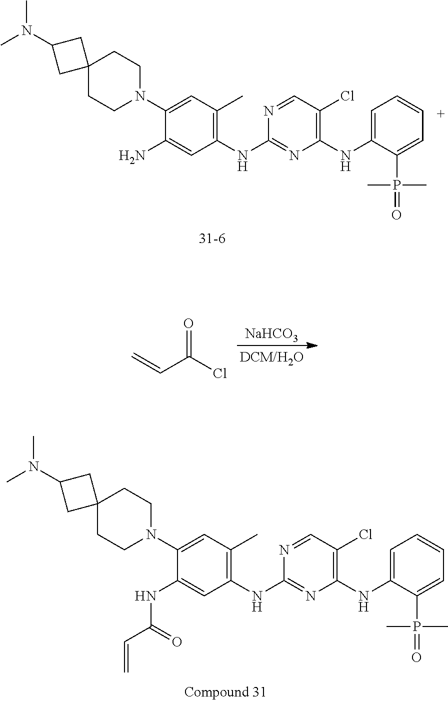

Following the same procedure as N-(5-((5-chloro-4-((5-(dimethylphosphoryl)quinolin-6-yl)amino)pyrimidin-2-yl)amino)-2-(2-(dimethylamino)-7-azaspiro[3.5]nonan-7-yl)phenyl)acrylamide using (2-((2-((5-amino-2-methyl-4-(2-(methyl amino)-7-azaspiro[3.5]nonan-7-yl)phenyl)amino)-5-chloropyrimidin-4-yl)amino)phenyl)dimethylphosphine oxide instead of (6-((2-((3-amino-4-(2-(dimethylamino)-7-azaspiro[3.5]nonan-7-yl)phenyl)amino)-5-chloropyrimidin-4-yl)amino)quinolin-5-yl)dimethylphosphine oxide to obtain N-(5-((5-chloro-4-((2-(dimethylphosphoryl)phenyl)amino)pyrimidin-2-yl)amino)-2-(2-(dimethylamino)-7-azaspiro[3.5]nonan-7-yl)-4-methylphenyl)acrylamide. MS: 622 [M+H]⁺.

¹H NMR (500 MHz, DMSO-d₆) δ 11.25 (s, 1H), 10.49 (s, 1H), 9.01 (s, 1H), 8.76 (s, 1H), 8.44 (s, 1H), 8.08 (s, 2H), 7.52 (m, 1H), 7.17 (s, 1H), 7.04 (m, 2H), 6.71 (m, 1H), 6.20 (m, 1H), 5.73 (d, J=11.5 Hz, 1H) 2.75 (m, 2H), 2.70 (m, 2H), 2.55 (s, 6H) 2.17 (m, 2H), 2.13 (s, 3H), 2.00 (m, 2H), 1.77 (m, 10H).

### Example 32 Synthesis of Compound 32

### N-(2-((2-(dimethylamino)ethyl)(methyl)amino)-5-(4-(2-(dimethylphosphoryl)phenylamino)-5-fluoropyrimidin-2-ylamino)phenyl)acrylamide

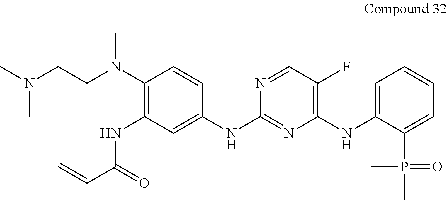 [SMILES: C=CC(=O)Nc1cc(Nc2ncc(F)c(Nc3ccccc3P(C)(C)=O)n2)ccc1N(C)CCN(C)C]

### Step 1: Synthesis of 2-(4-((2-(dimethylamino)ethyl)(methyl)amino)-3-nitrophenyl)-N4-(2-(dimethylphosphoryl)phenyl)-5-fluoropyrimidine-2,4-diamine

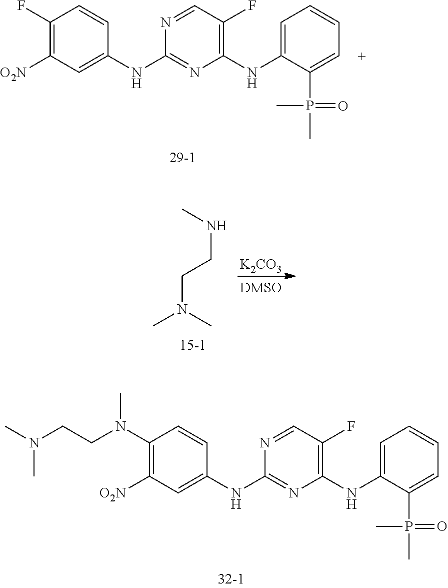

To a solution of N⁴-(2-(dimethylphosphoryl)phenyl)-5-fluoro-N²-(4-fluoro-3-nitrophenyl)pyrimidine-2,4-diamine (500 mg) in DMSO (5 mL) was added K₂CO₃ (330 mg), this was followed by addition of N¹,N¹,N²-trimethylethane-1,2-diamine (183 mg), The reaction mixture was stirred at 90° C. overnight, The reaction mixture was cooled down to room temperature and diluted with DCM (50 mL). The resulting solution was washed with water and NaCl saturated aqueous solution respectively, the organic phase was concentrated under vacuum, The crude product was re-crystallized from Et₂O to obtain 2-(4-((2-(dimethylamino)ethyl)(methyl)amino)-3-nitrophenyl)-N4-(2-(dimethylphosphoryl)phenyl)-5-fluoropyrimidine-2,4-diamine (520 mg) as yellow solid MS: 502 [M+14]⁺

### Step 2: Synthesis of N1-(2-(dimethylamino)ethyl)-N4-(4-(2-(dimethylphosphoryl)phenylamino)-5-fluoropyrimidin-2-yl)-N1-methylbenzene-1,2,4-triamine

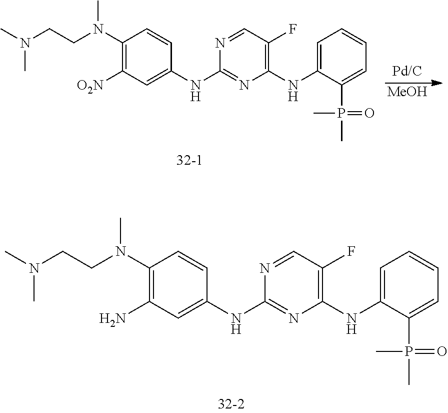 [SMILES: "MeOH" ; CN(C)CCN(C)c1ccc(Nc2ncc(F)c(Nc3ccccc3P(C)(C)=O)n2)cc1[N+](=O)[O-] ; CN(C)CCN(C)c1ccc(Nc2ncc(F)c(Nc3ccccc3P(C)(C)=O)n2)cc1N]

To a solution of 2-(4-((2-(dimethylamino)ethyl)(methyl)amino)-3-nitrophenyl)-N4-(2-(dimethylphosphoryl)phenyl)-5-fluoropyrimidine-2,4-diamine (520 mg) in MeOH (10 mL), was added Pd/C (100 mg), the mixture was stirred under H₂ atmosphere at 25° C. for 2 hrs, The solution was filtered through diatomite to remove the Pd/C, The solution was evaporated to give N1-(2-(dimethylamino)ethyl)-N4-(4-(2-(dimethylphosphoryl)phenylamino)-5-fluoropyrimidin-2-yl)-N1-methylbenzene-1,2,4-triamine (400 mg) as grey solid. MS: 472[M+H]⁺

### Step 3: Synthesis of N-(2-((2-(dimethylamino)ethyl)(methyl)amino)-5-(4-(2-(dimethylphosphoryl)phenylamino)-5-fluoropyrimidin-2-ylamino)phenyl)acrylamide

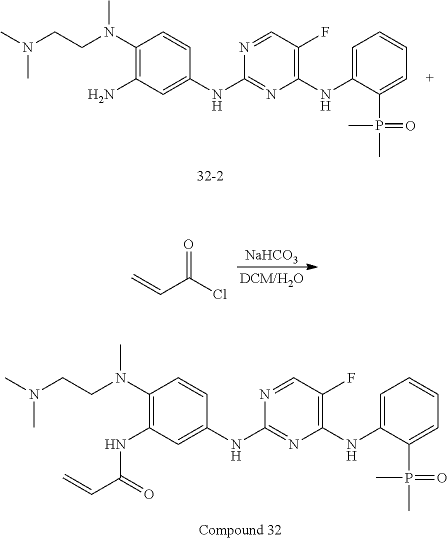

To a solution of N1-(2-(dimethylamino)ethyl)-N4-(4-(2-(dimethylphosphoryl)phenylamino)-5-fluoropyrimidin-2-yl)-N1-methylbenzene-1,2,4-triamine (400 mg) in DCM/H₂O (5 mL:5 mL), was added NaHCO₃ (160 mg), this was followed by addition of acryloyl chloride (80 mg) below 0° C., the mixture was stirred at 25° C. for 30 minutes, The resulting solution was extracted with 2*20 mL of DCM and the organic layers combined. The mixture was dried over anhydrous magnesium sulfate and concentrated under vacuum. The residue was purified by column chromatography over silica gel with DCM/MeOH (8:1) to obtained N-(2-((2-(dimethylamino)ethyl)(methyl)amino)-5-(4-(2-(dimethylphosphoryl)phenylamino)-5-fluoropyrimidin-2-ylamino)phenyl)acrylamide (186 mg) as off-white solid. MS: 526[M+H]⁺.

### Example 33 Synthesis of Compound 33

### N-(5-(5-chloro-4-(2-(dimethylphosphoryl)phenylamino)pyrimidin-2-ylamino)-2-(9-(dimethylamino)-3-azaspiro[5.5]undecan-3-yl)-4-methoxyphenyl)acrylamide

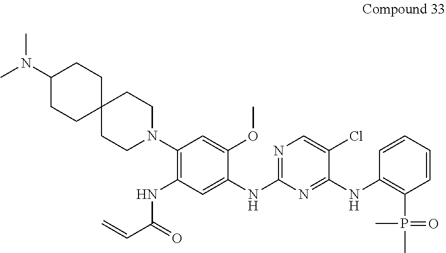 [SMILES: C=CC(=O)Nc1cc(Nc2ncc(Cl)c(Nc3ccccc3P(C)(C)=O)n2)c(OC)cc1N1CCC2(CCC(N(C)C)CC2)CC1]

### Step 1: Synthesis of 3-(4-(5-chloro-4-(2-(dimethylphosphoryl)phenylamino)pyrimidin-2-ylamino)-5-methoxy-2-nitrophenyl)-3-azaspiro[5.5]undecan-9-one

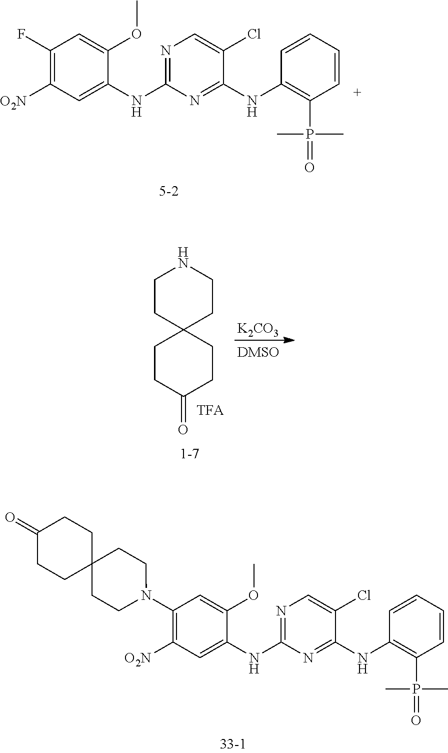

Following the same procedure as 2-(4-((2-(dimethylamino)ethyl)(methyl)amino)-3-nitrophenyl)-N4-(2-(dimethylphosphoryl)phenyl)-5-fluoropyrimidine-2,4-diamine, using 5-chloro-N4-(2-(dimethylphosphoryl)phenyl)-N2-(4-fluoro-2-methoxy-5-nitrophenyl)pyrimidine-2,4-diamine instead of N4-(2-(dimethylphosphoryl)phenyl)-5-fluoro-N2-(4-fluoro-3-nitrophenyl)pyrimidine-2,4-diamine, using 3-azaspiro[5.5]undecan-9-one trifluoroacetate instead of N¹,N¹,N²-trimethyl ethane-1,2-di amine to obtain 3-(4-(5-chloro 4-(2-(dimethylphosphoryl)phenylamino)pyrimidin-2-ylamino)-5-methoxy-2-nitrophenyl)-3-azaspiro[5.5]undecan-9-one. MS: 613[M+H]⁺

### Step 2: Synthesis of 5-chloro-N2-(4-(9-(dimethylamino)-3-azaspiro[5.5]undecan-3-yl)-2-methoxy-5-nitrophenyl)-N4-(2-(dimethylphosphoryl)phenyl)pyrimidine-2,4-diamine

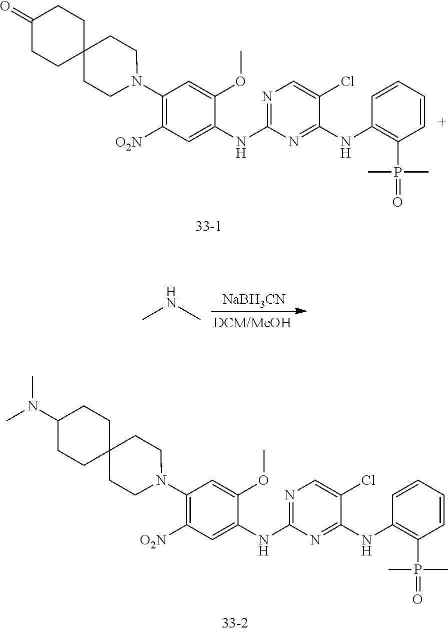

To a solution of 3-(4-(5-chloro-4-(2-(dimethylphosphoryl)phenylamino)pyrimidin-2-ylamino)-5-methoxy-2-nitrophenyl)-3-azaspiro[5.5]undecan-9-one (200 mg) in DCM/MeOH (5 mL:5 mL), this was followed by addition of HOAc (50 mg), then added dimethylamine (2 mL 2N in THF), the mixture was stirred at 65° C. for 1 hr, NaBH₃CN (200 mg) was added and the mixture was further stirred at room temperature for 16 hrs, After completion of the reaction (monitored by TLC), the reaction mixture was diluted with DCM (30 mL). The resulting solution was washed with 10% NaHCO₃ aqueous solution and NaCl saturated aqueous solution. The mixture was dried over anhydrous magnesium sulfate and concentrated under vacuum, The crude product was re-crystallized from Et₂O to obtained 100 mg of 5-chloro-N2-(4-(9-(dimethylamino)-3-azaspiro[5.5]undecan-3-yl)-2-methoxy-5-nitrophenyl)-N4-(2-(dimethylphosphoryl)phenyl)pyrimidine-2,4-diamine. MS: 642 [M+H]⁺

### Step 3: synthesis of N2-(5-amino-4-(9-(dimethylamino)-3-azaspiro[5.5]undecan-3-yl)-2-methoxyphenyl)-5-chloro-N4-(2-(dimethylphosphoryl)phenyl)pyrimidine-2,4-diamine

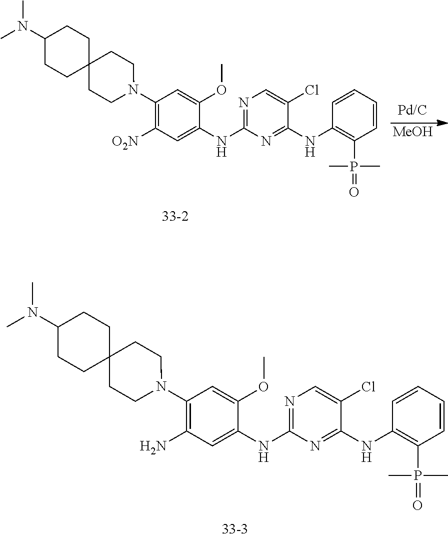

Following the same procedure as N1-(2-(dimethylamino)ethyl)-N4-(4-(2-(dimethylphosphoryl)phenylamino)-5-fluoropyrimidin-2-yl)-N1-methylbenzene-1,2,4-triamine, using 5-chloro-N2-(4-(9-(dimethylamino)-3-azaspiro[5.5]undecan-3-yl)-2-methoxy-5-nitrophenyl)-N4-(2-(dimethylphosphoryl)phenyl)pyrimidine-2,4-diamine instead of 2-(4-((2-(dimethylamino)ethyl)(methyl)amino)-3-nitrophenyl)-N4-(2-(dimethylphosphoryl)phenyl)-5-fluoropyrimidine-2,4-diamine to obtain N2-(5-amino-4-(9-(dimethylamino)-3-azaspiro[5.5]undecan-3-yl)-2-methoxyphenyl)-5-chloro-N4-(2-(dimethylphosphoryl)phenyl)pyrimidine-2,4-diamine. MS: 612 [M+H]⁺

### Step 4: synthesis of N-(5-(5-chloro-4-(2-(dimethylphosphoryl)phenylamino)pyrimidin-2-ylamino)-2-(9-(dimethylamino)-3-azaspiro[5.5]undecan-3-yl)-4-methoxyphenyl)acrylamide

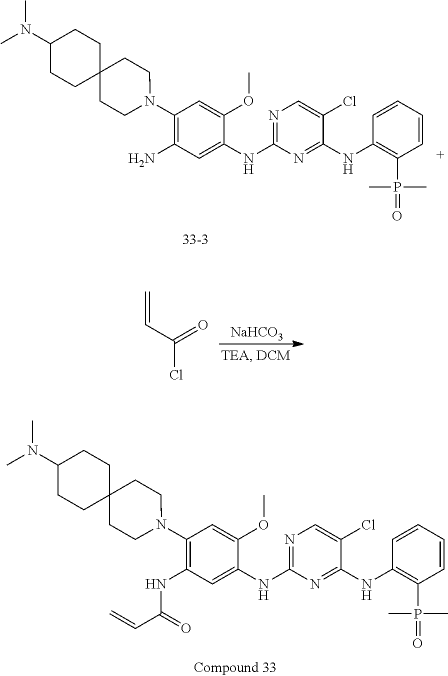

Following the same procedure as N-(2-((2-(dimethylamino)ethyl)(methyl)amino)-5-(4-(2-(dimethylphosphoryl)phenylamino)-5-fluoropyrimidin-2-ylamino)phenyl)acrylamide, using N2-(5-amino-4-(9-(dimethylamino)-3-azaspiro[5.5]undecan-3-yl)-2-methoxyphenyl)-5-chloro-N4-(2-(dimethylphosphoryl)phenyl)pyrimidine-2,4-diamine instead of N1-(2-(dimethylamino)ethyl)-N4-(4-(2-(dimethylphosphoryl)phenylamino)-5-fluoropyrimidin-2-yl)-N1-methyl benzene-1,2,4-triamine to obtain N-(5-(5-chloro-4-(2-(dimethylphosphoryl)phenylamino)pyrimidin-2-ylamino)-2-(9-(dimethylamino)-3-azaspiro[5.5]undecan-3-yl)-4-methoxy phenyl)acrylamide. MS: 666 [M+H]⁺

### Example 34 Synthesis of Compound 34

### N-(5-(5-bromo-4-(5-(dimethylphosphoryl)quinoxalin-6-ylamino)pyrimidin-2-ylamino)-2-((2-(dimethylamino)ethyl)(methyl)amino)phenyl)acrylamide

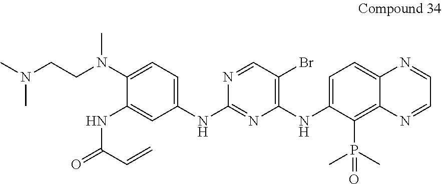 [SMILES: C=CC(=O)Nc1cc(Nc2ncc(Br)c(Nc3ccc4nccnc4c3P(C)(C)=O)n2)ccc1N(C)CCN(C)C]

### Step 1: synthesis of 5-iodoquinoxalin-6-amine

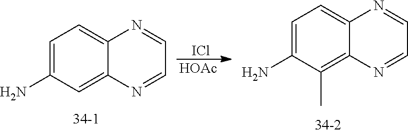

To a solution of quinoxalin-6-amine (7.5 g) in HOAc (150 mL), was added ICl (10 g), The reaction mixture was stirred at 25° C. 2 hrs, the mixture was poured into hexane, then filtered, got 5-iodoquinoxalin-6-amine (12 g). MS: 272[M+H]⁺

### Step 2: synthesis of 5-(dimethylphosphoryl)quinoxalin-6-amine

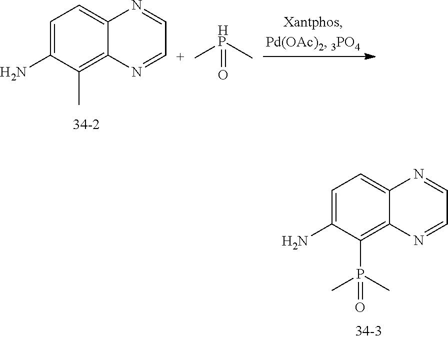

To a solution of 5-iodoquinoxalin-6-amine (12 g) in dioxane (120 mL), was added Dimethylphosphine oxide (5.2 g), it was followed by addition of Xantphos (2.6 g), Pd(OAc)₂ (990 mg), K₃PO₄ (2.4 g), The reaction mixture was stirred at 100° C. overnight, the organic phase was concentrated under vacuum, the mixture diluted with EA (500 mL). The resulting solution was washed with water and NaCl saturated aqueous solution respectively, the organic phase was concentrated under vacuum, The residue was purified by column chromatography over silica gel with DCM/MeOH (20:1) to obtained 5-(dimethylphosphoryl)quinoxalin-6-amine (6 g) MS: 222[M+H]⁺

### Step 3: Synthesis of N-(5-bromo-2-chloropyrimidin-4-yl)-5-(dimethylphosphoryl)quinoxalin-6-amine

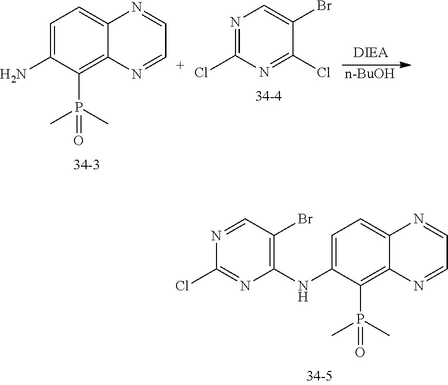

To a solution of 5-(dimethylphosphoryl)quinoxalin-6-amine (6 g) in n-BuOH (60 mL), was added DIEA (7 g), this was followed by 5-bromo-2,4-dichloro-pyrimidine (12 g), The reaction mixture was stirred at 140° C. for 48 hrs, cooled to 25° C., filtered, got N-(5-bromo-2-chloropyrimidin-4-yl)-5-(dimethylphosphoryl)quinoxalin-6-amine (1.7 g), MS: 414[M+H]⁺

### Step 4: Synthesis of 5-bromo-N4-(5-(dimethylphosphoryl)quinoxalin-6-yl)-N2-(4-fluoro-3-nitrophenyl)pyrimidine-2,4-diamine

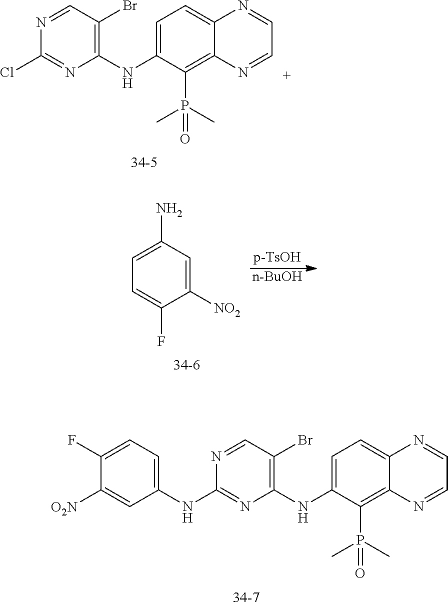

To a solution of N-(5-bromo-2-chloropyrimidin-4-yl)-5-(dimethylphosphoryl)quinoxalin-6-amine (600 mg) in n-BuOH (20 mL), was added 4-fluoro-3-nitroaniline (272 mg), this was followed by addition of p-TsOH (375 mg), The reaction mixture was stirred at 120° C. for 2 hrs, the mixture diluted with DCM (100 mL). The resulting solution was washed with water and NaCl saturated aqueous solution respectively, the organic phase was concentrated under vacuum, The residue was purified by column chromatography over silica gel with DCM/MeOH (20:1) to obtained 5-bromo-N4-(5-(dimethylphosphoryl)quinoxalin-6-yl)-N2-(4-fluoro-3-nitrophenyl)pyrimidine-2,4-diamine (400 mg). MS: 532[M+H]⁺

### Step 5: Synthesis of 5-bromo-N2-(4-((2-(dimethylamino)ethyl)(methyl)amino)-3-nitrophenyl)-N4-(5-(dimethylphosphoryl)quinoxalin-6-yl)pyrimidine-2,4-diamine

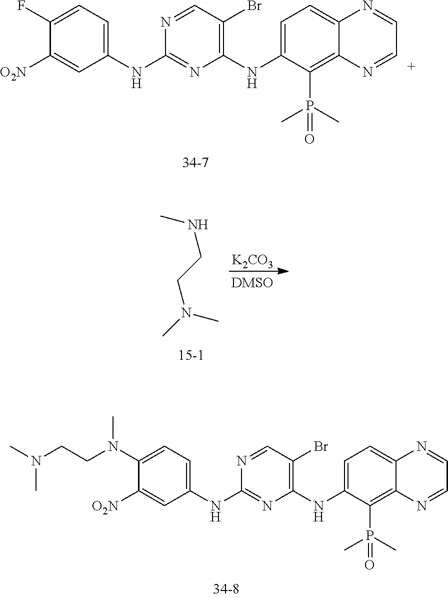

To a solution of 5-bromo-N4-(5-(dimethylphosphoryl)quinoxalin-6-yl)-N2-(4-fluoro-3-nitrophenyl)pyrimidine-2,4-diamine (400 mg) in DMSO (10 mL), was added K₂CO₃ (210 mg), this was followed by addition of N¹,N¹,N²-trimethylethane-1,2-diamine (153 mg), The reaction mixture was stirred at 100° C. overnight, The reaction mixture was cooled down to room temperature and diluted with DCM (50 mL). The resulting solution was washed with water and NaCl saturated aqueous solution respectively, the organic phase was concentrated under vacuum, The crude product was re-crystallized from Et₂O to obtained 5-bromo-N2-(4-((2-(dimethylamino)ethyl)(methyl)amino)-3-nitrophenyl)-N4-(5-(dimethylphosphoryl)quinoxalin-6-yl)pyrimidine-2,4-diamine (200 mg) as yellow solid. MS: 614[M+H]⁺

### Step 6: Synthesis of N4-(5-bromo-4-(5-(dimethylphosphoryl)quinoxalin-6-ylamino)pyrimidin-2-yl)-N1-(2-(dimethylamino)ethyl)-N1-methylbenzene-1,2,4-triamine

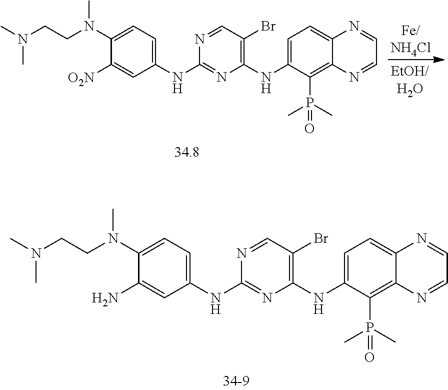 [SMILES: CN(C)CCN(C)c1ccc(Nc2ncc(Br)c(Nc3ccc4nccnc4c3P(C)(C)=O)n2)cc1[N+](=O)[O-] ; CN(C)CCN(C)c1ccc(Nc2ncc(Br)c(Nc3ccc4nccnc4c3P(C)(C)=O)n2)cc1N]

To a solution of 5-bromo-N2-(4-((2-(dimethylamino)ethyl)(methyl)amino)-3-nitrophenyl)-N4-(5-(dimethylphosphoryl)quinoxalin-6-yl)pyrimidine-2,4-diamine (200 mg) in EtOH/H₂O (10 mL/10 mL), was added Fe powder (180 mg), this was followed by addition of NH₄Cl (180 mg), the mixture was stirred at 80° C. for 2 hrs, The solution was filtered, the organic phase was concentrated under vacuum, The residue was purified by column chromatography over silica gel with DCM/MeOH (10:1) to obtained N4-(5-bromo-4-(5-(dimethylphosphoryl)quinoxalin-6-yl amino)pyrimidin-2-yl)-N1-(2-(dimethylamino)ethyl)-N1-methylbenzene-1,2,4-triamine (150 mg) MS: 586[M+14]⁺

### Step 7: synthesis of N-(5-(5-bromo-4-(5-(dimethylphosphoryl)quinoxalin-6-ylamino)pyrimidin-2-ylamino)-2-((2-(dimethylamino)ethyl)(methyl)amino)phenyl)acrylamide

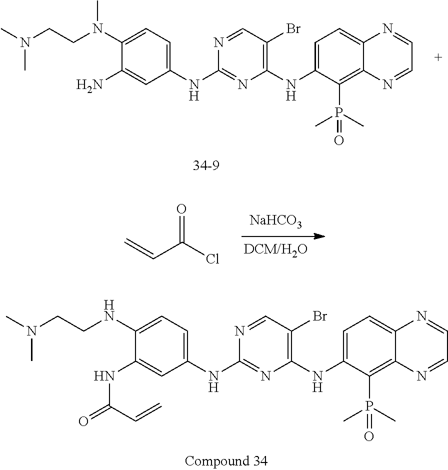

To a solution of N4-(5-bromo-4-(5-(dimethylphosphoryl)quinoxalin-6-ylamino)pyrimidin-2-yl)-N1-(2-(dimethylamino)ethyl)-N1-methylbenzene-1,2,4-triamine (150 mg) in DCM/H₂O (5 mL:5 mL), was added NaHCO₃ (160 mg), this was followed by addition of acryloyl chloride (23 mg) below 5° C., the mixture was stirred at 25° C. for 30 minutes, The resulting solution was extracted with 2*20 mL DCM and the organic layers combined. The mixture was dried over anhydrous magnesium sulfate and concentrated under vacuum. The residue was purified by column chromatography over silica gel with DCM/MeOH (8:1) to obtained N-(5-(5-bromo-4-(5-(dimethylphosphoryl)quinoxalin-6-ylamino)pyrimidin-2-ylamino)-2-((2-(dimethylamino)ethyl)(methyl)amino)phenyl)acrylamide (33.2 mg) as off-white solid. MS: 624 [M+H]⁺

### Example 35 Synthesis of Compound 35

### N-(5-(5-bromo-4-(5-(dimethylphosphoryl)quinoxalin-6-ylamino)pyrimidin-2-ylamino)-2-(2-(dimethylamino)-7-azaspiro[3.5]nonan-7-yl)phenyl)acrylamide

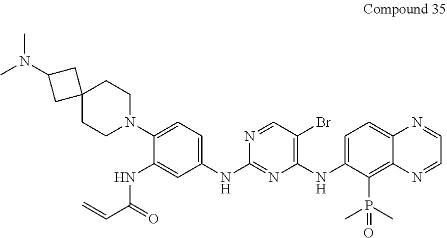 [SMILES: C=CC(=O)Nc1cc(Nc2ncc(Br)c(Nc3ccc4nccnc4c3P(C)(C)=O)n2)ccc1N1CCC2(CC1)CC(N(C)C)C2]

### Step 1: synthesis of 7-(4-(5-bromo-4-(5-(dimethylphosphoryl)quinoxalin-6-ylamino)pyrimidin-2-ylamino)-2-nitrophenyl)-7-azaspiro[3.5]nonan-2-one

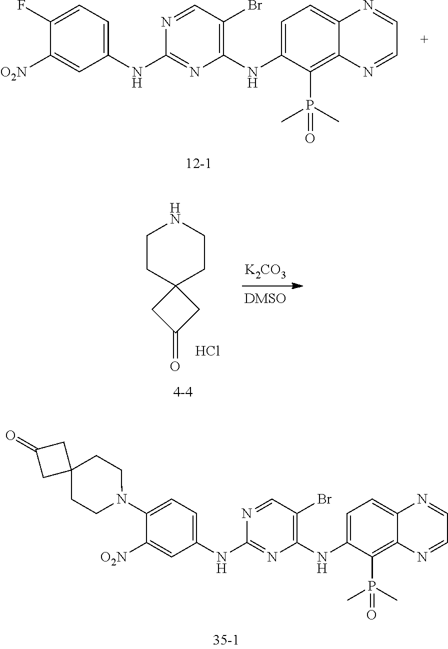

Following the same procedure as 5-bromo-N2-(4-((2-(dimethylamino)ethyl)(methyl)amino)-3-nitrophenyl)-N4-(5-(dimethylphosphoryl)quinoxalin-6-yl)pyrimidine-2,4-diamine, using (6-((5-bromo-2-((4-fluoro-3-nitrophenyl)amino)pyrimidin-4-yl)amino)quinoxalin-5-yl)dimethylphosphine oxide instead of 5-bromo-N4-(5-(dimethylphosphoryl)quinoxalin-6-yl)-N2-(4-fluoro-3-nitrophenyl)pyrimidine-2,4-diamine, using 7-azaspiro[3.5]nonan-2-one hydrogen chloride salt instead of N¹,N¹,N²-trimethylethane-1,2-diamine to obtain 7-(4-(5-bromo-4-(5-(dimethylphosphoryl)quinoxalin-6-ylamino)pyrimidin-2-ylamino)-2-nitrophenyl)-7-azaspiro[3.5]nonan-2-one. MS 651 [M+H]⁺

### Step 2: synthesis of 5-bromo-N2-(4-(2-(dimethylamino)-7-azaspiro[3.5]nonan-7-yl)-3-nitrophenyl)-N4-(5-(dimethylphosphoryl)quinoxalin-6-yl)pyrimidine-2,4-diamine

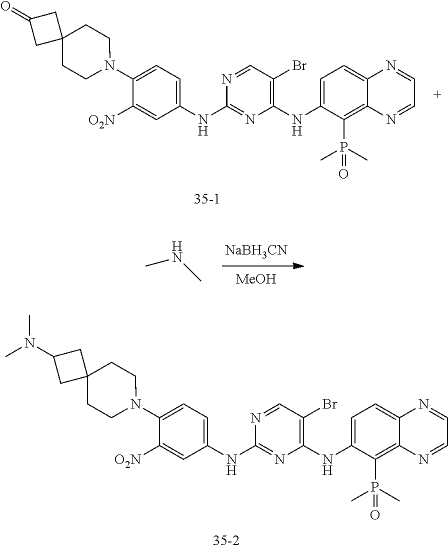

To a solution of 7-(4-(5-bromo-4-(5-(dimethylphosphoryl)quinoxalin-6-ylamino)pyrimidin-2-ylamino)-2-nitrophenyl)-7-azaspiro[3.5]nonan-2-one (200 mg) in DCM/MeOH (5 mL:5 mL), this was followed by addition of HOAc (50 mg), then added dimethylamine (2 mL 2N in THF), the mixture was stirred at 65° C. for 1 hr, NaBH₃CN (200 mg) was added and the mixture was further stirred at room temperature for 16 hrs, After completion of the reaction (monitored by TLC), the reaction mixture was diluted with DCM (30 mL). The resulting solution was washed with 10% NaHCO₃ aqueous solution and NaCl saturated aqueous solution. The mixture was dried over anhydrous magnesium sulfate and concentrated under vacuum, The crude product was re-crystallized from Et₂O to obtained 100 mg 5-bromo-N2-(4-(2-(dimethylamino)-7-azaspiro[3.5]nonan-7-yl)-3-nitrophenyl)-N4-(5-(dimethylphosphoryl)quinoxalin-6-yl)pyrimidine-2,4-diamine. MS: 680 [M+H]⁺

### Step 3: Synthesis of (6-((2-((3-amino-4-(2-(dimethylamino)-7-azaspiro[3.5]nonan-7-yl)phenyl)amino)-5-bromopyrimidin-4-yl)amino)quinoxalin-5-yl)dimethylphosphine oxide

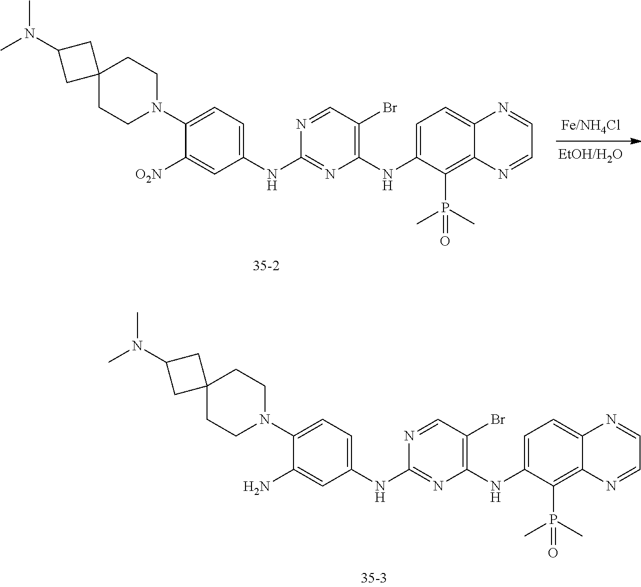

Following the same procedure as N4-(5-bromo-4-(5-(dimethylphosphoryl)quinoxalin-6-ylamino)pyrimidin-2-yl)-N1-(2-(dimethylamino)ethyl)-N1-methylbenzene-1,2,4-triamine, using 5-bromo-N2-(4-(2-(dimethylamino)-7-azaspiro[3.5]nonan-7-yl)-3-nitrophenyl)-N4-(5-(dimethylphosphoryl)quinoxalin-6-yl)pyrimidine-2,4-diamine instead of 5-bromo-N2-(4-((2-(dimethylamino)ethyl)(methyl)amino)-3-nitrophenyl)-N4-(5-(dimethylphosphoryl)quinoxalin-6-yl)pyrimidine-2,4-diamine to obtain (6-((2-((3-amino-4-(2-(dimethylamino)-7-azaspiro[3.5]nonan-7-yl)phenyl)amino)-5-bromopyrimidin-4-yl)amino)quinoxalin-5-yl)dimethylphosphine oxide. MS: 650 [M+H]⁺

### Step 4: synthesis of N-(5-(5-bromo-4-(5-(dimethylphosphoryl)quinoxalin-6-ylamino)pyrimidin-2-ylamino)-2-(2-(dimethylamino)-7-azaspiro[3.5]nonan-7-yl)phenyl)acrylamide

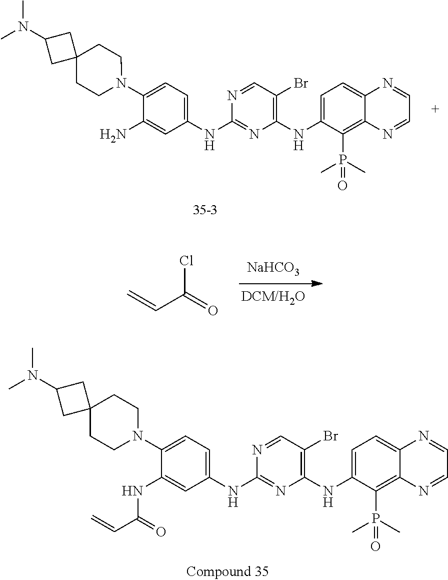

Following the same procedure as N-(5-(5-bromo-4-(5-(dimethylphosphoryl)quinoxalin-6-ylamino)pyrimidin-2-ylamino)-2-((2-(dimethylamino)ethyl)(methyl)amino)phenyl)acrylamide, using (6-((2-((3-amino-4-(2-(dimethylamino)-7-azaspiro[3.5]nonan-7-yl)phenyl)amino)-5-bromopyrimidin-4-yl)amino)quinoxalin-5-yl)dimethylphosphine oxide instead of N4-(5-bromo-4-(5-(dimethylphosphoryl)quinoxalin-6-ylamino)pyrimidin-2-yl)-N1-(2-(dimethylamino)ethyl)-N1-methylbenzene-1,2,4-triamine to obtain N-(5-(5-bromo-4-(5-(dimethylphosphoryl)quinoxalin-6-ylamino)pyrimidin-2-ylamino)-2-(2-(dimethylamino)-7-azaspiro[3.5]nonan-7-yl)phenyl)acrylamide. MS: 704 [M+H]⁺

### Example 36 Synthesis of Compound 36

### N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)acrylamide

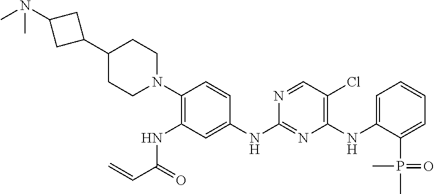 [SMILES: C=CC(=O)Nc1cc(Nc2ncc(Cl)c(Nc3ccccc3P(C)(C)=O)n2)ccc1N1CCC(C2CC(N(C)C)C2)CC1]

### Step 1: Synthesis of tert-butyl (7-(4-amino-2-nitrophenyl)-7-azaspiro[3.5]nonan-2-yl)carbamate

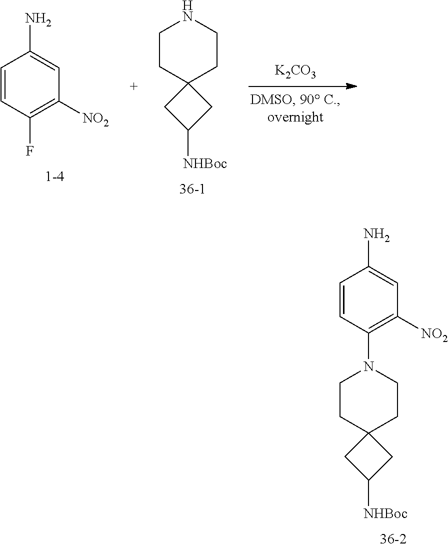

To a solution of 4-fluoro-3-nitroaniline (2.0 g) in DMSO (20 mL) was added K₂CO₃ (3.5 g) at room temperature, then tert-butyl (7-azaspiro[3.5]nonan-2-yl)carbamate (3.3 g) was added into the mixture in portions. The mixture was heated to 90° C. overnight. TLC showed the reaction was completed. The solution was poured into water and extracted with ethyl acetate (30 mL*3). The combined organic layer was washed with saturated sodium chloride aqueous solution (50 mL*2), dried over Na₂SO₄ and concentrated under reduced pressure. The crude product was purified by column chromatography (hexane/ethyl acetate from 0 to 20%). After concentration, the solid was dried to obtain tert-butyl (7-(4-amino-2-nitrophenyl)-7-azaspiro[3.5]nonan-2-yl)carbamate (2.1 g) as yellow solid. MS: 376.46 [M+H]⁺.

### Step 2: Synthesis of tert-butyl (7-(4-((5-chloro-4-((2-(dimethylphosphoryl)phenyl)amino)pyrimidin-2-yl)amino)-2-nitrophenyl)-7-azaspiro[3.5]nonan-2-yl)carbamate

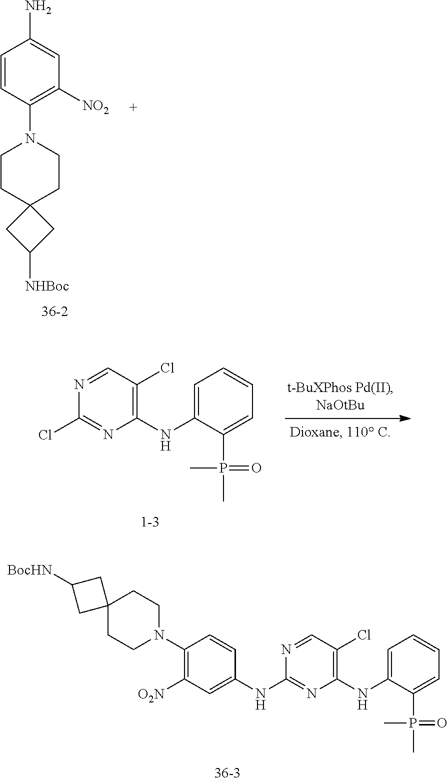 [SMILES: CC(C)(C)OC(=O)NC1CC2(CCN(c3ccc(N)cc3[N+](=O)[O-])CC2)C1 ; CP(C)(=O)c1ccccc1Nc1nc(Cl)ncc1Cl ; "t-BuXPhos Pd(II),NaOtBu" ; CC(C)(C)OC(=O)NC1CC2(CCN(c3ccc(Nc4ncc(Cl)c(Nc5ccccc5P(C)(C)=O)n4)cc3[N+](=O)[O-])CC2)C1]

To a mixture of tert-butyl (7-(4-amino-2-nitrophenyl)-7-azaspiro[3.5]nonan-2-yl)carbamate (1.5 g) and (2-((2,5-dichloropyrimidin-4-yl)amino)phenyl)dimethylphosphine oxide (1.26 g) in Dioxane (20 mL) was added Methanesulfonato(2-di-t-butylphosphino-2′,4′,6′-tri-i-propyl-1,1′-biphenyl)(2′-amino-1,1′-biphenyl-2-yl)palladium(II) (633 mg) and NaOtBu (765 mg) at room temperature under nitrogen atmosphere. Then the mixture was heated to 110° C. overnight. LCMS showed the reaction was completed. The solution was diluted with EtOAc (20 mL) and then filtrated under reduced pressure. The filtrate was concentrated by vacuum. The crude product was purified by column chromatography (hexane/ethyl acetate from 1:0 to 0:1). After concentration, the solid was dried to obtain tert-butyl (7-(4-((5-chloro-4-((2-(dimethylphosphoryl)phenyl)amino)pyrimidin-2-yl)amino)-2-nitrophenyl)-7-azaspiro[3.5]nonan-2-yl)carbamate (1.3 g) as brown solid. MS: 656.12 [M+H]⁺.

### Step 3: Synthesis of (2-((2-((4-(2-amino-7-azaspiro[3.5]nonan-7-yl)-3-nitrophenyl)amino)-5-chloropyrimidin-4-yl)amino)phenyl)dimethylphosphine oxide

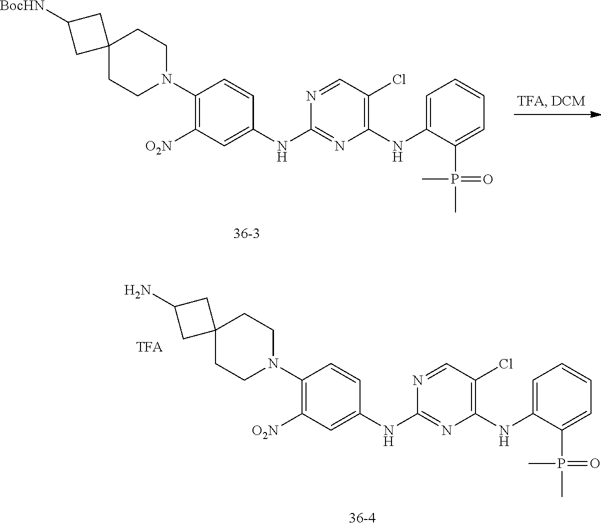 [SMILES: CC(C)(C)OC(=O)NC1CC2(CCN(c3ccc(Nc4ncc(Cl)c(Nc5ccccc5P(C)(C)=O)n4)cc3[N+](=O)[O-])CC2)C1 ; CP(C)(=O)c1ccccc1Nc1nc(Nc2ccc(N3CCC4(CC3)CC(N)C4)c([N+](=O)[O-])c2)ncc1Cl]

To a solution of tert-butyl (7-(4-((5-chloro-4-((2-(dimethylphosphoryl)phenyl)amino)pyrimidin-2-yl)amino)-2-nitrophenyl)-7-azaspiro[3.5]nonan-2-yl)carbamate (1.3 g) in DCM (30 mL) was added 2,2,2-trifluoroacetic acid (10 mL) at room temperature under nitrogen atmosphere. Then the mixture was stirred at room temperature for 2 hours. LCMS showed the reaction was completed. The solution was concentrated by vacuum. The crude product was without further purification and applied to the next step directly. After concentration, the solid was dried to obtain (2-((2-((4-(2-amino-7-azaspiro[3.5]nonan-7-yl)-3-nitrophenyl)amino)-5-chloropyrimidin-4-yl)amino)phenyl)dimethylphosphine oxide (1.2 g) as colorless oil. MS: 556.00 [M+H]⁺.

### Step 4: Synthesis of (2-((5-chloro-2-((4-(2-(dimethylamino)-7-azaspiro[3.5]nonan-7-yl)-3-nitrophenyl)amino)pyrimidin-4-yl)amino)phenyl)dimethylphosphine oxide

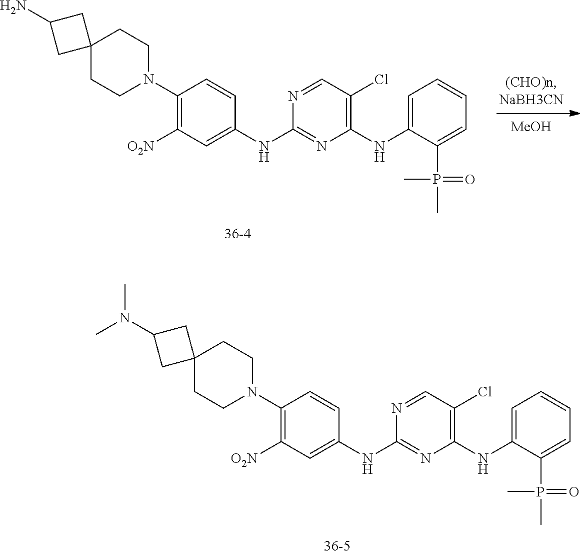 [SMILES: "MeOH" ; CP(C)(=O)c1ccccc1Nc1nc(Nc2ccc(N3CCC4(CC3)CC(N)C4)c([N+](=O)[O-])c2)ncc1Cl ; CN(C)C1CC2(CCN(c3ccc(Nc4ncc(Cl)c(Nc5ccccc5P(C)(C)=O)n4)cc3[N+](=O)[O-])CC2)C1]

To a solution of (2-((2-((4-(2-amino-7-azaspiro[3.5]nonan-7-yl)-3-nitrophenyl)amino)-5-chloropyrimidin-4-yl)amino)phenyl)dimethylphosphine oxide (600 mg) in MeOH (30 mL) was added K₂CO₃ that adjusted pH to 8, then Paraformaldehyde was added. After stirred for 10 min, NaBH₃CN was added in portions. Then the mixture was stirred at room temperature for 1 hour. LCMS showed the reaction was completed. The solution was poured into water and extracted with DCM (30 mL*3). The combined organic layer was washed with saturated sodium chloride aqueous solution (50 mL*2), dried over Na₂SO₄ and concentrated under reduced pressure. The crude product was purified by column chromatography (DCM/MeOH=10:1). After concentration, the solid was dried to obtain (2-((5-chloro-2-((4-(2-(dimethylamino)-7-azaspiro[3.5]nonan-7-yl)-3-nitrophenyl)amino)pyrimidin-4-yl)amino)phenyl)dimethylphosphine oxide (500 mg) as yellow solid. MS: 584.06 [M+H]⁺.

### Step 5: Synthesis of (2-((2-((3-amino-4-(2-(dimethylamino)-7-azaspiro[3.5]nonan-7-yl)phenyl)amino)-5-chloropyrimidin-4-yl)amino)phenyl)dimethylphosphine oxide

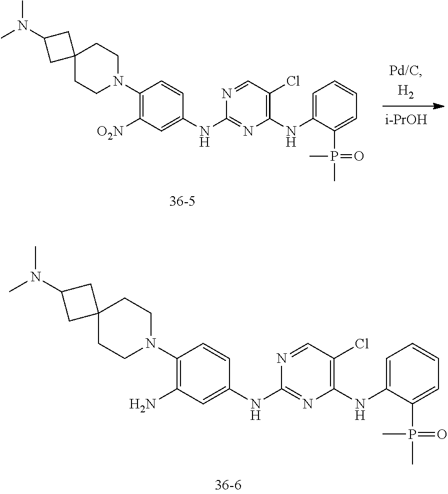 [SMILES: "i-PrOH" ; CN(C)C1CC2(CCN(c3ccc(Nc4ncc(Cl)c(Nc5ccccc5P(C)(C)=O)n4)cc3[N+](=O)[O-])CC2)C1 ; CN(C)C1CC2(CCN(c3ccc(Nc4ncc(Cl)c(Nc5ccccc5P(C)(C)=O)n4)cc3N)CC2)C1]

To a solution of (2-((5-chloro-2-((4-(2-(dimethylamino)-7-azaspiro[3.5]nonan-7-yl)-3-nitrophenyl)amino)pyrimidin-4-yl)amino)phenyl)dimethylphosphine oxide (500 mg) in i-PrOH (20 mL) was added Pd/C in one portion. Then the mixture was stirred under H₂ (balloon, 15 psi) at room temperature overnight. LCMS showed the reaction was completed. The solution was filtrated and the filtrate was concentrated by vacuum. The crude product was without further purification and applied to the next step directly. After concentration, the solid was dried to obtain (2-((2-((3-amino-4-(2-(dimethylamino)-7-azaspiro[3.5]nonan-7-yl)phenyl)amino)-5-chloropyrimidin-4-yl)amino)phenyl)dimethylphosphine oxide (240 mg) as gray solid. MS: 554.08 [M+H]⁺.

### Step 6: Synthesis of N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)acrylamide

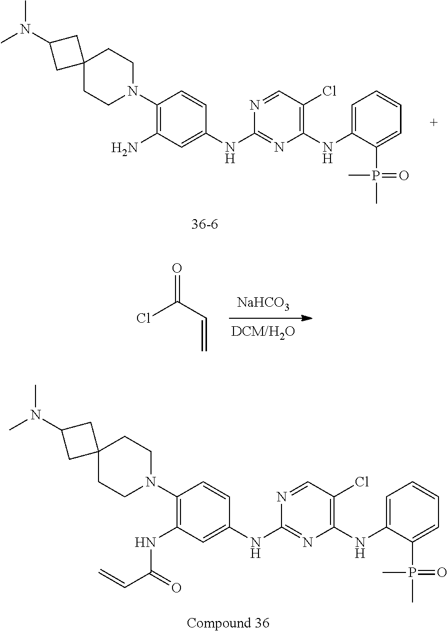

To a solution of (2-((2-((3-amino-4-(2-(dimethylamino)-7-azaspiro[3.5]nonan-7-yl)phenyl)amino)-5-chloropyrimidin-4-yl)amino)phenyl)dimethylphosphine oxide (300 mg) in DCM (5 mL) and H₂O (5 mL) was added NaHCO₃ (68.23 mg) in one portion at 0° C. Then acryloyl chloride (54 mg) was added in dropwise. The mixture was stirred at this temperature for 10 min. LCMS showed the reaction was completed. The reaction mixture was quenched by MeOH and then concentrated by vacuum. The crude product was purified by reversed phase chromatography (MeOH:H₂O=30%). After concentration, the solid was dried to obtain N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)acrylamide (170 mg) as off-white solid. MS: 608.54 [M+H]⁺.

### Example 37 Synthesis of Compound 37

### N-(2-((2-(dimethylamino)ethyl)(methyl)amino)-5-((4-((2-(dimethylphosphoryl)phenyl)amino)pyrimidin-2-yl)amino)phenyl)acrylamide

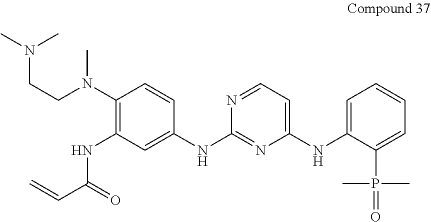

### Step 1: Synthesis of (2-((2-((4-fluoro-3-nitrophenyl)amino)pyrimidin-4-yl)amino)phenyl)dimethylphosphine oxide

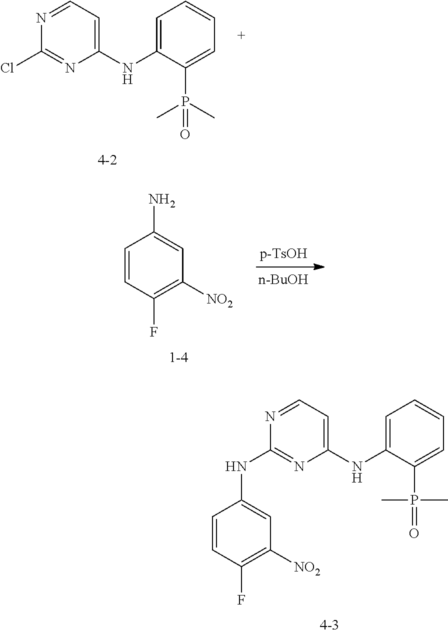

To a mixture of (2-((2-chloropyrimidin-4-yl)amino)phenyl)dimethylphosphine oxide (1.0 g) and 4-fluoro-3-nitroaniline (609 mg) in n-BuOH (20 mL) was added p-TsOH (917 mg) in one portion at room temperature. Then the mixture was heated to 110° C. and stirred at this temperature for 1 hour. LCMS showed the reaction was completed. The reaction mixture was concentrated by vacuum. The crude product was purified by column chromatography (MeOH:DCM=10%). After concentration, the solid was dried to obtain (2-((2-((4-fluoro-3-nitrophenyl)amino)pyrimidin-4-yl)amino)phenyl)dimethylphosphine oxide (1.0 g) as gray solid. MS: 401.34 [M+H]⁺.

### Step 2: Synthesis of (2-((2-((4-((2-(dimethylamino)ethyl)(methyl)amino)-3-nitrophenyl)amino)pyrimidin-4-yl)amino)phenyl)dimethylphosphine oxide

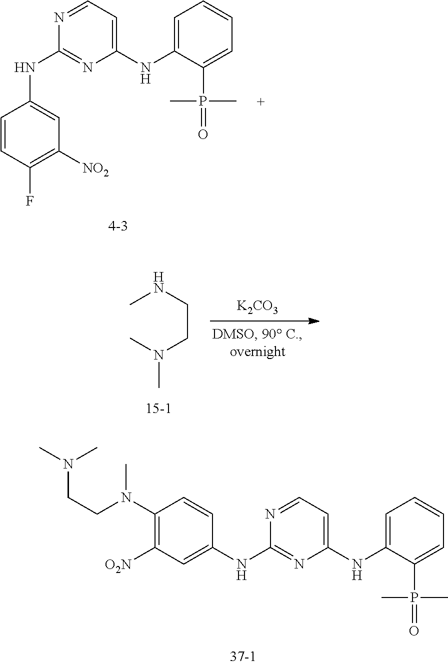

To a mixture of (2-((2-((4-fluoro-3-nitrophenyl)amino)pyrimidin-4-yl)amino)phenyl)dimethylphosphine oxide (300 mg) and N¹,N¹,N²-trimethylethane-1,2-diamine (114 mg) in DMSO (10 mL) was added K₂CO₃ (206 mg) in one portion at room temperature. Then the mixture was heated to 90° C. and stirred at this temperature for 16 h. LCMS showed the reaction was completed. The solution was poured into water and extracted with ethyl acetate (30 mL*3). The combined organic layer was washed with saturated sodium chloride aqueous solution (50 mL*2), dried over Na₂SO₄ and concentrated under reduced pressure. The crude product was purified by column chromatography (DCM:MeOH from 0 to 30%). After concentration, the solid was dried to obtain (2-((2-((4-((2-(dimethylamino)ethyl)(methyl)amino)-3-nitrophenyl)amino)pyrimidin-4-yl)amino)phenyl)dimethylphosphine oxide (200 mg) as red solid. MS: 483.51 [M+H]⁺.

### Step 3: Synthesis of (2-((2-((3-amino-4-((2-(dimethylamino)ethyl)(methyl)amino)phenyl)amino)pyrimidin-4-yl)amino)phenyl)dimethylphosphine oxide

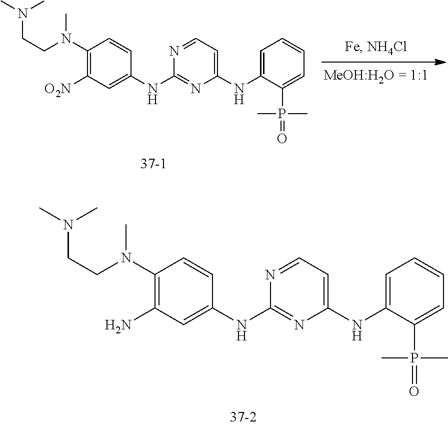

To a solution of (2-((2-((4-((2-(dimethylamino)ethyl)(methyl)amino)-3-nitrophenyl)amino)pyrimidin-4-yl)amino)phenyl)dimethylphosphine oxide (200 mg) in MeOH (5 mL) and H₂O (5 mL) was added Fe (115 mg) and NH₄Cl (44 mg) in one portion. Then the mixture was heated to 90° C. and stirred for 2 hours. LCMS showed the reaction was completed and the desired MS was detected. The solution was filtrated and the filtrate was concentrated by vacuum. The crude product was without further purification and applied to the next step directly. After concentration, the solid was dried to obtain (2-((2-((3-amino-4-((2-(dimethylamino)ethyl)(methyl)amino)phenyl)amino)pyrimidin-4-yl)amino)phenyl)dimethylphosphine oxide (100 mg) as red solid. MS: 453.53 [M+H]⁺.

### Step 4: Synthesis of N-(2-((2-(dimethylamino)ethyl)(methyl)amino)-5-((4-((2-(dimethylphosphoryl)phenyl)amino)pyrimidin-2-yl)amino)phenyl)acrylamide

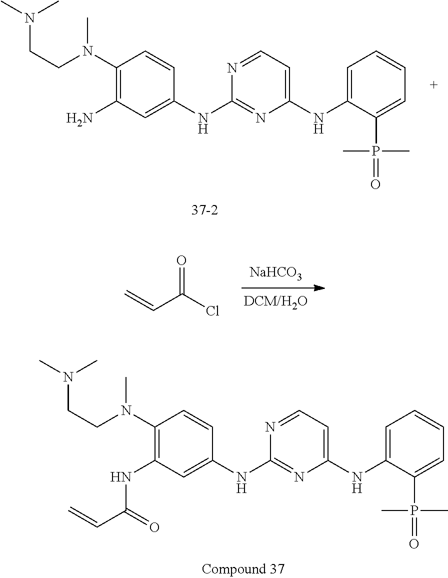

To a solution of (2-((2-((3-amino-4-((2-(dimethylamino)ethyl)(methyl)amino)phenyl)amino)pyrimidin-4-yl)amino)phenyl)dimethylphosphine oxide (100 mg) in DCM (5 mL) and H₂O (5 mL) was added NaHCO₃ (37 mg) in one portion at 0° C. Then acryloyl chloride (24 mg) was added in dropwise. The mixture was stirred at this temperature for 10 min. LCMS showed the reaction was completed. The reaction mixture was quenched by MeOH and then concentrated by vacuum. The crude product was purified by reversed phase chromatography (MeOH:H₂O=50%). After concentration, the obtained product was adjusted pH to 9 and then extracted by EtOAc (5 mL*3) and the combined organic phase were further purified by prep-TLC and obtain N-(2-((2-(dimethylamino)ethyl)(methyl)amino)-5-((4-((2-(dimethylphosphoryl)phenyl)amino)pyrimidin-2-yl)amino)phenyl)acrylamide (6 mg) as yellow solid. MS: 508.97 [M+H]⁺.

### Example 38 Synthesis of Compound 38

### N-(5-((4-((2-(dimethylphosphoryl)phenyl)amino)pyrimidin-2-yl)amino)-2-(4-(4-methylpiperazin-1-yl)piperidin-1-yl)phenyl)acrylamide

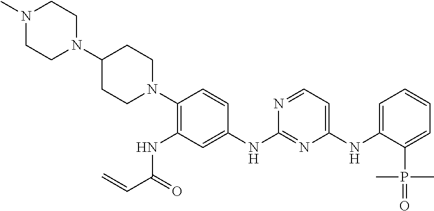

### Step 1: Synthesis of dimethyl(2-((2-((4-(4-(4-methylpiperazin-1-yl)piperidin-1-yl)-3-nitrophenyl)amino)pyrimidin-4-yl)amino)phenyl)phosphine oxide

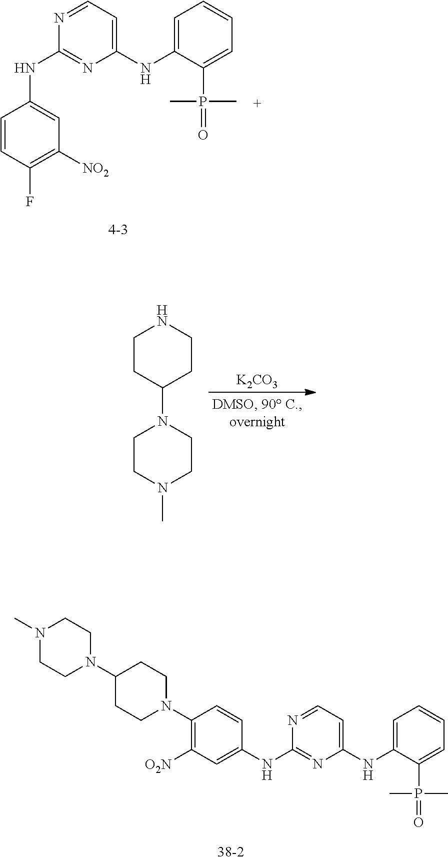

To a mixture of (2-((2-((4-fluoro-3-nitrophenyl)amino)pyrimidin-4-yl)amino)phenyl)dimethylphosphine oxide (500 mg) and 1-methyl-4-(piperidin-4-yl)piperazine (342 mg) in DMSO (10 mL) was added K₂CO₃ (344 mg) in one portion at room temperature. Then the mixture was heated to 90° C. and stirred at this temperature for 16 hours. LCMS showed the reaction was completed. The solution was poured into water and extracted with ethyl acetate (30 mL*3). The combined organic layer was washed with saturated sodium chloride aqueous solution (50 mL*2), dried over Na₂SO₄ and concentrated under reduced pressure. The crude product was purified by column chromatography (DCM:MeOH from 0 to 15%). After concentration, the solid was dried to obtain dimethyl(2-((2-((4-(4-(4-methylpiperazin-1-yl)piperidin-1-yl)-3-nitrophenyl)amino)pyrimidin-4-yl)amino)phenyl)phosphine oxide (260 mg) as red solid. MS: 564.63 [M+H]⁺.

### Step 2: Synthesis of (2-((2-((3-amino-4-(4-(4-methylpiperazin-1-yl)piperidin-1-yl)phenyl)amino)pyrimidin-4-yl)amino)phenyl)dimethylphosphine oxide

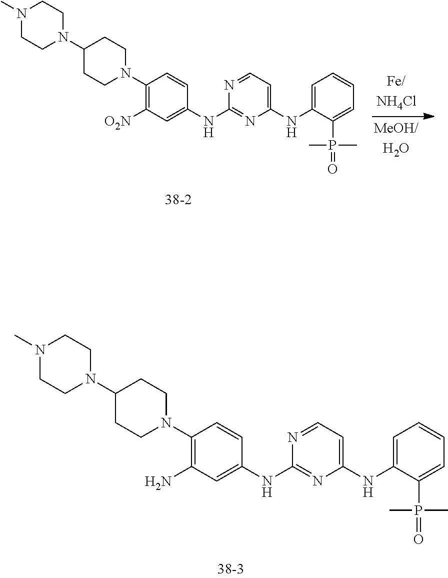

To a solution of dimethyl(2-((2-((4-(4-(4-methylpiperazin-1-yl)piperidin-1-yl)-3-nitrophenyl)amino)pyrimidin-4-yl)amino)phenyl)phosphine oxide (260 mg) in MeOH (5 mL) and H₂O (5 mL) was added Fe (128 mg) and NH₄Cl (50 mg) in one portion. Then the mixture was heated to 90° C. and stirred for 2 hours. LCMS showed the reaction was completed and the desired MS was detected. The solution was filtrated and the filtrate was concentrated by vacuum. The crude product was without further purification and applied to the next step directly. After concentration, the solid was dried to obtain (2-((2-((3-amino-4-(4-(4-methyl piperazin-1-yl)piperidin-1-yl)phenyl)amino)pyrimidin-4-yl)amino)phenyl)dimethylphosphine oxide (100 mg, crude) as red solid.

### Step 3: Synthesis of N-(5-((4-((2-(dimethylphosphoryl)phenyl)amino)pyrimidin-2-yl)amino)-2-(4-(4-methylpiperazin-1-yl)piperidin-1-yl)phenyl)acrylamide

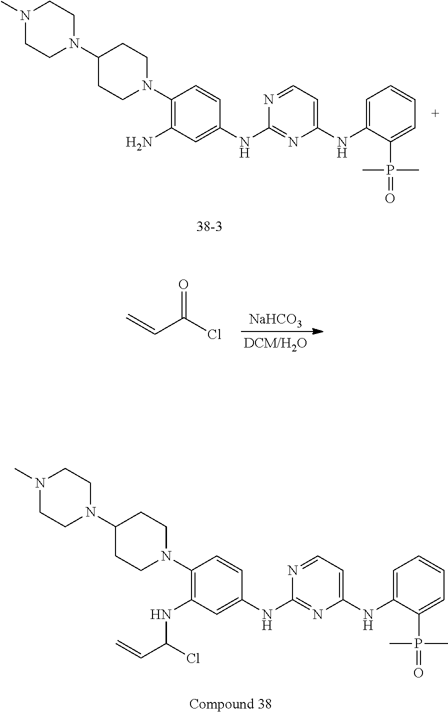

To a solution of (2-((2-((3-amino-4-(4-(4-methylpiperazin-1-yl)piperidin-1-yl)phenyl)amino)pyrimidin-4-yl)amino)phenyl)dimethylphosphine oxide (100 mg) in DCM (5 mL) and H₂O (5 mL) was added NaHCO₃ (24 mg) in one portion at 0° C. Then acryloyl chloride (21 mg) was added in dropwise. The mixture was stirred at this temperature for 10 min. LCMS showed the reaction was completed. The reaction mixture was quenched by MeOH and then concentrated by vacuum. The crude product was purified by reversed phase chromatography (MeOH:H₂O=30%). After concentration, the obtained product was adjusted pH to 9 and then extracted by EtOAc (5 mL*3) and the combined organic phase were adjusted to HCl salt obtain N-(5-((4-((2-(dimethylphosphoryl)phenyl)amino)pyrimidin-2-yl)amino)-2-(4-(4-methylpiperazin-1-yl)piperidin-1-yl)phenyl)acrylamide (8 mg) as yellow solid. MS: 588.68 [M+H]⁺.

### Example 39 Synthesis of Compound 39

### N-(2-((2-(dimethylamino)ethyl)(methyl)amino)-5-((4-((2-(dimethylphosphoryl)phenyl)amino)pyrimidin-2-yl)amino)-4-methoxyphenyl)acrylamide

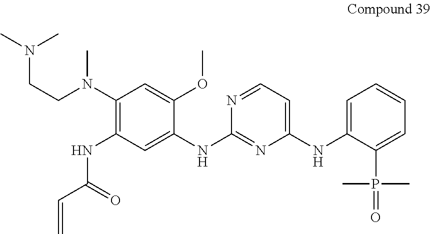

### Step 1: Synthesis of (2-((2-((4-fluoro-2-methoxy-5-nitrophenyl)amino)pyrimidin-4-yl)amino)phenyl)dimethylphosphine oxide

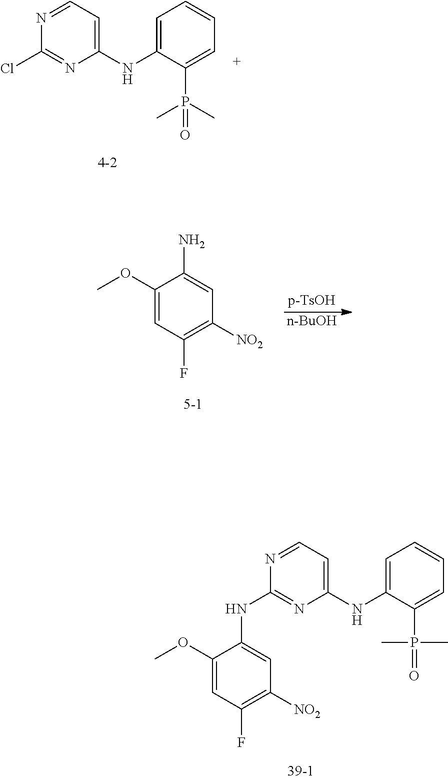

To a mixture of (2-((2-chloropyrimidin-4-yl)amino)phenyl)dimethylphosphine oxide (1.0 g) and 4-fluoro-3-nitroaniline (727 mg) in n-BuOH (20 mL) was added p-TsOH (917 mg) in one portion at room temperature. Then the mixture was heated to 110° C. and stirred at this temperature for 1 hour. LCMS showed the reaction was completed. The reaction mixture was concentrated by vacuum. The crude product was washed by H₂O and EtOAc and then filtrated. The filtrate cake was concentrated under reduced pressure, the solid was dried to obtain (2-((2-((4-fluoro-2-methoxy-5-nitrophenyl)amino)pyrimidin-4-yl)amino)phenyl)dimethylphosphine oxide (1.3 g, crude) as yellow solid.

### Step 2: Synthesis of (2-((2-((4-((2-(dimethylamino)ethyl)(methyl)amino)-2-methoxy-5-nitrophenyl)amino)pyrimidin-4-yl)amino)phenyl)dimethylphosphine oxide

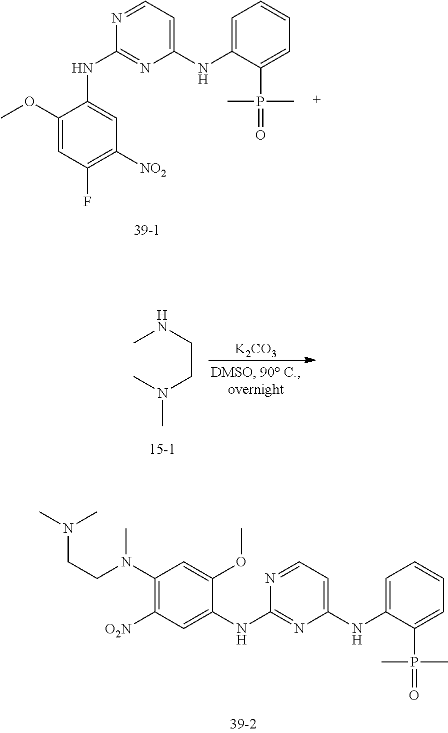

To a mixture of (2-((2-((4-fluoro-2-methoxy-5-nitrophenyl)amino)pyrimidin-4-yl)amino)phenyl)dimethylphosphine oxide (500 mg) and N1,N1,N2-trimethylethane-1,2-diamine (177 mg) in DMSO (10 mL) was added K₂CO₃ (320 mg) in one portion at room temperature. Then the mixture was heated to 90° C. and stirred at this temperature for 16 hours. LCMS showed the reaction was completed. The solution was poured into water and extracted with DCM:MeOH=10:1 (30 mL*3). The combined organic layer was washed with saturated sodium chloride aqueous solution (50 mL*2), dried over Na₂SO₄ and concentrated under reduced pressure. The crude product was without further purification and applied to the next step directly. After concentration, the solid was dried to obtain (2-((2-((4-((2-(dimethylamino)ethyl)(methyl)amino)-2-methoxy-5-nitrophenyl)amino)pyrimidin-4-yl)amino)phenyl)dimethylphosphine oxide (500 mg, crude) as red solid.

### Step 3: Synthesis of (2-((2-((5-amino-4-((2-(dimethylamino)ethyl)(methyl)amino)-2-methoxyphenyl)amino)pyrimidin-4-yl)amino)phenyl)dimethylphosphine oxide

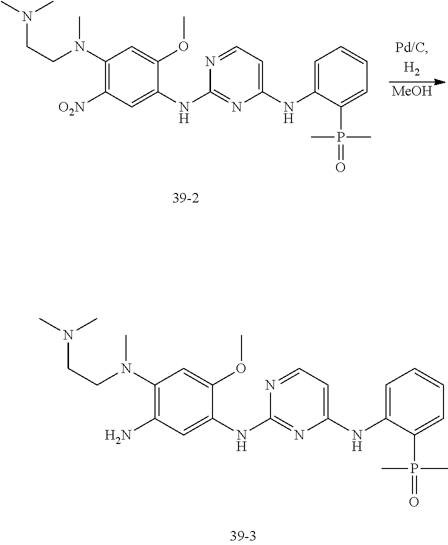

To a solution of (2-((2-((4-((2-(dimethylamino)ethyl)(methyl)amino)-2-methoxy-5-nitrophenyl)amino)pyrimidin-4-yl)amino)phenyl)dimethylphosphine oxide (500 mg) in MeOH (5 mL) was added Pd/C (200 mg) in one portion. Then the mixture was stirred under H₂ balloon for 2 hours. LCMS showed the reaction was completed and the desired MS was detected. The solution was filtrated and the filtrate was concentrated by vacuum. The crude product was purified by reversed phase chromatography (MeOH:H₂O from 0 to 30%). After concentration, the solid was dried to obtain (2-((2-((5-amino-4-((2-(dimethylamino)ethyl)(methyl)amino)-2-methoxyphenyl)amino)pyrimidin-4-yl)amino)phenyl)dimethylphosphine oxide (200 mg) as gray solid. MS: 483.56 [M+H]⁺.

### Step 4: Synthesis of N-(2-((2-(dimethylamino)ethyl)(methyl)amino)-5-((4-((2-(dimethylphosphoryl)phenyl)amino)pyrimidin-2-yl)amino)-4-methoxyphenyl)acrylamide

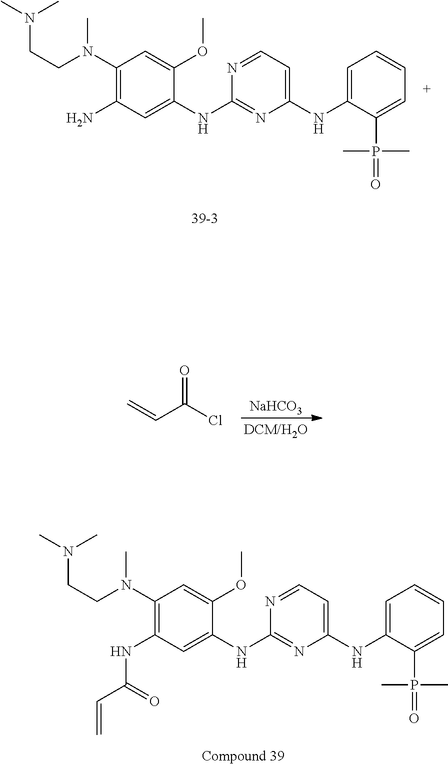

To a solution of (2-((2-((5-amino-4-((2-(dimethylamino)ethyl)(methyl)amino)-2-methoxyphenyl)amino)pyrimidin-4-yl)amino)phenyl)dimethylphosphine oxide (150 mg) in DCM (5 mL) and H₂O (5 mL) was added NaHCO₃ (39 mg) in one portion at 0° C. Then acryloyl chloride (34 mg) was added in dropwise. The mixture was stirred at this temperature for 10 min. LCMS showed the reaction was completed. The reaction mixture was quenched by MeOH and then concentrated by vacuum. The crude product was purified by reversed phase chromatography (MeOH:H₂O=30%). After concentration, the obtained product was adjusted pH to 9 and then extracted by EtOAc (5 mL*3) and the combined organic phase were adjusted to HCl salt obtain N-(2-((2-(dimethylamino)ethyl)(methyl)amino)-5-((4-((2-(dimethylphosphoryl)phenyl)amino)pyrimidin-2-yl)amino)-4-methoxyphenyl)acrylamide (20 mg) as white solid. MS: 539.00 [M+H]⁺.

### Example 40 Synthesis of Compound 40

### N-(5-((4-((2-(dimethylphosphoryl)phenyl)amino)pyrimidin-2-yl)amino)-4-methoxy-2-(4-(4-methylpiperazin-1-yl)piperidin-1-yl)phenyl)acrylamide

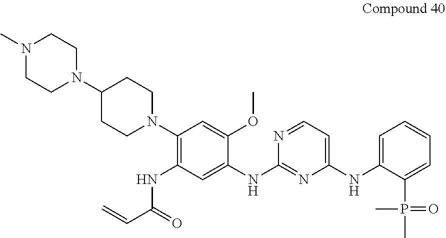 [SMILES: C=CC(=O)Nc1cc(Nc2nccc(Nc3ccccc3P(C)(C)=O)n2)c(OC)cc1N1CCC(N2CCN(C)CC2)CC1]

### Step 1: Synthesis of (2-((2-((2-methoxy-4-(4-(4-methylpiperazin-1-yl)piperidin-1-yl)-5-nitrophenyl)amino)pyrimidin-4-yl)amino)phenyl)dimethylphosphine oxide

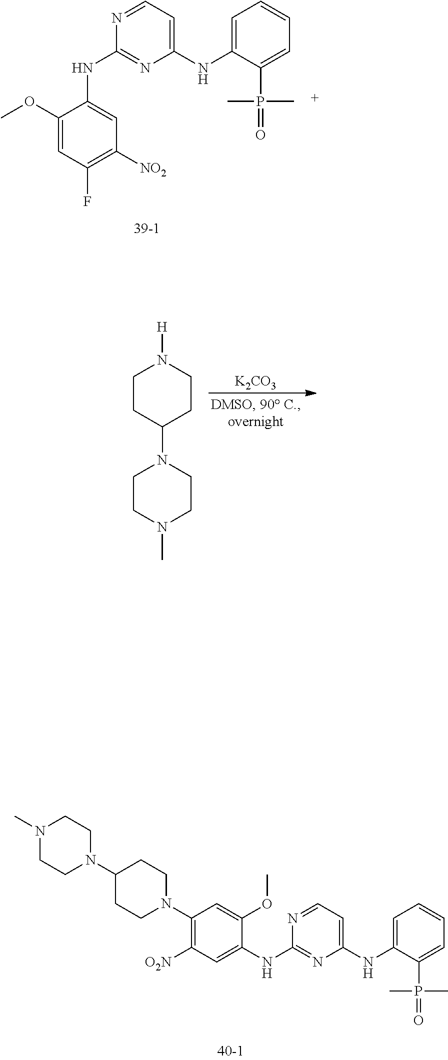

To a mixture of (2-((2-((4-fluoro-2-methoxy-5-nitrophenyl)amino)pyrimidin-4-yl)amino)phenyl)dimethylphosphine oxide (500 mg) and 1-methyl-4-(piperidin-4-yl)piperazine (318 mg) in DMSO (10 mL) was added K₂CO₃ (320 mg) in one portion at room temperature. Then the mixture was heated to 90° C. and stirred at this temperature for 16 hours. LCMS showed the reaction was completed. The solution was poured into water and extracted with DCM:MeOH=10:1 (30 mL*3). The combined organic layer was washed with saturated sodium chloride aqueous solution (50 mL*2), dried over Na₂SO₄ and concentrated under reduced pressure. The crude product was without further purification and applied to the next step directly. After concentration, the solid was dried to obtain (2-((2-((2-methoxy-4-(4-(4-methylpiperazin-1-yl)piperidin-1-yl)-5-nitrophenyl)amino)pyrimidin-4-yl)amino)phenyl)dimethylphosphine oxide (500 mg, crude) as red solid.

### Step 3: Synthesis of (2-((2-((5-amino-2-methoxy-4-(4-(4-methylpiperazin-1-yl)piperidin-1-yl)phenyl)amino)pyrimidin-4-yl)amino)phenyl)dimethylphosphine oxide

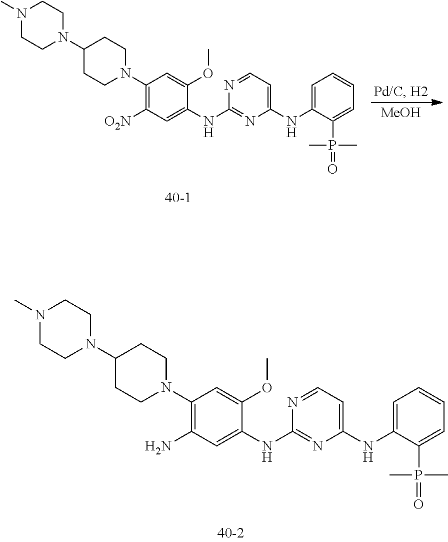

To a solution of (2-((2-((2-methoxy-4-(4-(4-methylpiperazin-1-yl)piperidin-1-yl)-5-nitrophenyl)amino)pyrimidin-4-yl)amino)phenyl)dimethylphosphine oxide (500 mg) in MeOH (5 mL) was added Pd/C (200 mg) in one portion. Then the mixture was stirred under H₂ balloon for 2 hours. LCMS showed the reaction was completed and the desired MS was detected. The solution was filtrated and the filtrate was concentrated by vacuum. The crude product was purified by reversed phase chromatography (MeOH:H₂O from 0 to 30%). After concentration, the solid was dried to obtain (2-((2-((5-amino-2-methoxy-4-(4-(4-methylpiperazin-1-yl)piperidin-1-yl)phenyl)amino)pyrimidin-4-yl)amino)phenyl)dimethylphosphine oxide (200 mg) as gray solid.

### Step 4: Synthesis of N-(5-((4-((2-(dimethylphosphoryl)phenyl)amino)pyrimidin-2-yl)amino)-4-methoxy-2-(4-(4-methylpiperazin-1-yl)piperidin-1-yl)phenyl)acrylamide

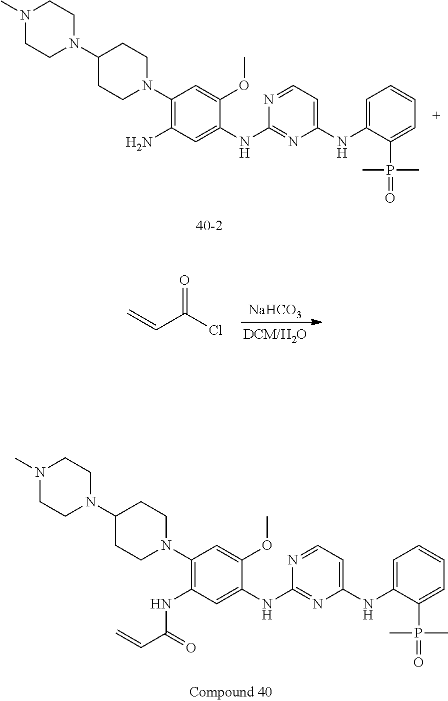

To a solution of (2-((2-((5-amino-2-methoxy-4-(4-(4-methylpiperazin-1-yl)piperidin-1-yl)phenyl)amino)pyrimidin-4-yl)amino)phenyl)dimethylphosphine oxide (150 mg) in DCM (5 mL) and H₂O (5 mL) was added NaHCO₃ (34 mg) in one portion at 0° C. Then acryloyl chloride (29 mg) was added in dropwise. The mixture was stirred at this temperature for 10 min. LCMS showed the reaction was completed. The reaction mixture was quenched by MeOH and then concentrated by vacuum. The crude product was purified by reversed phase chromatography (MeOH:H₂O=30%). After concentration, obtain N-(5-((4-((2-(dimethylphosphoryl)phenyl)amino)pyrimidin-2-yl)amino)-4-methoxy-2-(4-(4-methylpiperazin-1-yl)piperidin-1-yl)phenyl)acrylamide (26 mg) as pink solid. MS: 620.03 [M+H]⁺.

### Example 41 Synthesis of Compound 41

### N-(5-((5-chloro-4-((2-(dimethylphosphoryl)phenyl)amino)pyrimidin-2-yl)amino)-2-((1-methylpiperidin-4-yl)oxy)phenyl)acrylamide

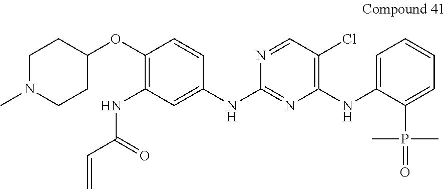

### Step 1: 4-((1-methylpiperidin-4-yl)oxy)-3-nitroaniline

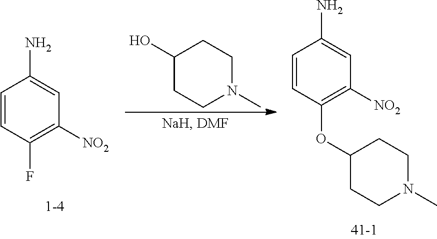

To a mixture of 1-methylpiperidin-4-ol (1.48 g) in DMF (20 mL) was added NaH (338 mg) in one portion at room temperature. Then 4-fluoro-3-nitroaniline (2.0 g) was added in portions. The mixture was stirred at this temperature for 16 hours. LCMS showed the reaction was completed. The reaction mixture was poured into water (50 mL) and extracted by EtOAc (30 mL*3). The combined organic phase was dried by Na₂SO₄, then filtered and concentrated in vacuum. The residue was purified by silica gel chromatography PE:EA from 0 to 80% to obtain 4-((1-methylpiperidin-4-yl)oxy)-3-nitroaniline (1.0 g, crude) as black solid. MS: 251.29 [M+H]⁺

### Step 2: Synthesis of (2-((5-chloro-2-((4-((1-methylpiperidin-4-yl)oxy)-3-nitrophenyl)amino)pyrimidin-4-yl)amino)phenyl)dimethylphosphine oxide

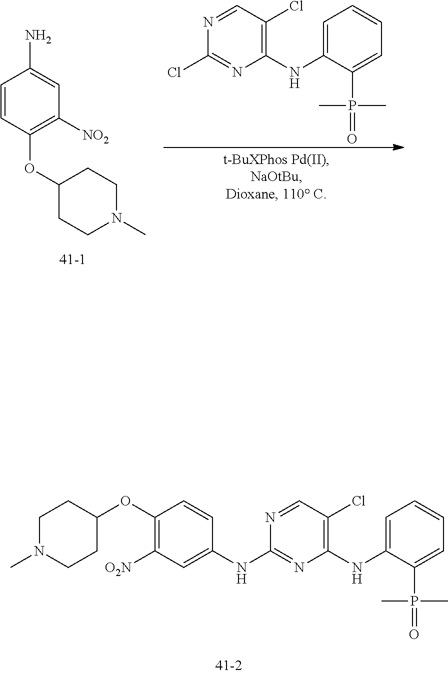

To a mixture of 4-((1-methylpiperidin-4-yl)oxy)-3-nitroaniline (300 mg) and (2-((2,5-dichloropyrimidin-4-yl)amino)phenyl)dimethylphosphine oxide (415 mg) in Dioxane (10 mL) was added Methanesulfonato(2-di-t-butylphosphino-2′,4′,6′-tri-i-propyl-1,1′-biphenyl)(2′-amino-1,1′-biphenyl-2-yl)palladium(II) (189.92 mg) and tert-butoxysodium (114.73 mg) in one portion at room temperature. Then the mixture was reacted under microwave 110° C. for 1.5 hours. LCMS showed the reaction was completed. The solution was concentrated under reduced pressure. The crude product was purified by reversed phase chromatography (MeOH:H₂O from 0 to 50%). After concentration, the solid was dried to obtain (2-((5-chloro-2-((4-((1-methylpiperidin-4-yl)oxy)-3-nitrophenyl)amino)pyrimidin-4-yl)amino)phenyl)dimethylphosphine oxide (300 mg) as yellow solid. MS: 530.95 [M+H]⁺

### Step 3: Synthesis of (2-((2-((3-amino-4-((1-methylpiperidin-4-yl)oxy)phenyl)amino)-5-chloropyrimidin-4-yl)amino)phenyl)dimethylphosphine oxide

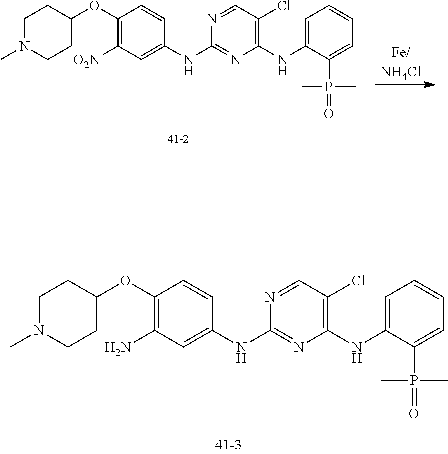

To a solution of (2-((5-chloro-2-((4-((l-methylpiperidin-4-yl)oxy)-3-nitrophenyl)amino)pyrimidin-4-yl)amino)phenyl)dimethylphosphine oxide (300 mg) in MeOH (5 mL) and H₂O (10 mL) was added Fe (157.77 mg) and NH₄Cl (60.45 mg) in one portion. Then the mixture was heated to 90° C. and stirred for 2 hours. LCMS showed the reaction was completed and the desired MS was detected. The solution was filtrated and the filtrate was concentrated by vacuum. The crude product was without further purification and applied to the next step directly. After concentration, the solid was dried to obtain (2-((2-((3-amino-4-((1-methylpiperidin-4-yl)oxy)phenyl)amino)-5-chloropyrimidin-4-yl)amino)phenyl)dimethylphosphine oxide (300 mg) as yellow solid.

### Step 4: Synthesis of N-(5-((5-chloro-4-((2-(dimethylphosphoryl)phenyl)amino)pyrimidin-2-yl)amino)-2-((1-methylpiperidin-4-yl)oxy)phenyl)acrylamide

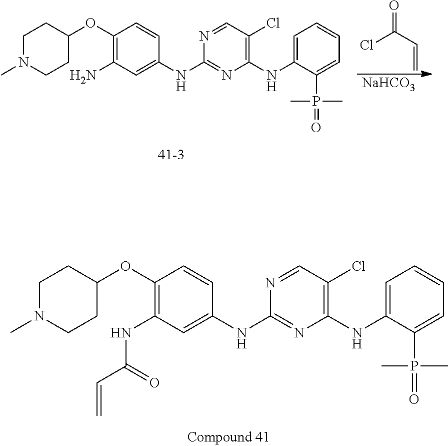

To a solution of (2-((2-((3-amino-4-((l-methylpiperidin-4-yl)oxy)phenyl)amino)-5-chloropyrimidin-4-yl)amino)phenyl)dimethylphosphine oxide (200 mg) in DCM (5 mL) and H₂O (5 mL) was added NaHCO₃ (50 mg) in one portion at 0° C. Then acryloyl chloride (90 mg) was added in dropwise. The mixture was stirred at this temperature for 10 min. LCMS showed the reaction was completed. The reaction mixture was quenched by MeOH and then concentrated by vacuum. The crude product was purified by reversed phase chromatography (MeOH:H₂O=50%). After concentration, obtain N-(5-((5-chloro-4-((2-(dimethylphosphoryl)phenyl)amino)pyrimidin-2-yl)amino)-2-((1-methylpiperidin-4-yl)oxy)phenyl)acrylamide (40 mg) as pink solid. MS: 555.40 [M+H]⁺.

### Comparative Compound A

### (2-((5-chloro-2-((4-(6-ethyl-2,6-diazaspiro[3.3]heptan-2-yl)-3-methylphenyl)amino)pyrimidin-4-yl)amino)phenyl)dimethylphosphine oxide

Prepare the following Comparative compound A as described for Example 36 in WO2018108064.

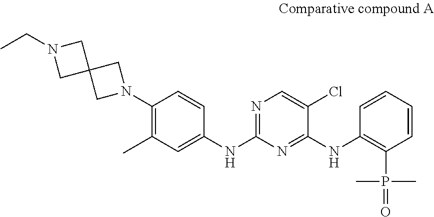 [SMILES: CCN1CC2(C1)CN(c1ccc(Nc3ncc(Cl)c(Nc4ccccc4P(C)(C)=O)n3)cc1C)C2]

### Comparative Compound B

### N-(2-((2-(dimethylamino)ethyl)(methyl)amino)-4-methoxy-54(4-(1-methyl-1H-indol-3-yl)pyrimidin-2-yl)amino)phenyl)acrylamide

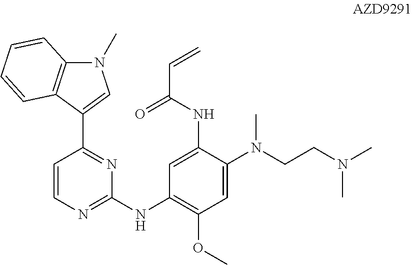 [SMILES: C=CC(=O)Nc1cc(Nc2nccc(-c3cn(C)c4ccccc34)n2)c(OC)cc1N(C)CCN(C)C]

### Comparative Compound C

### (6-((5-bromo-2-((2-methoxy-5-methyl-4-(4-(4-methylpiperazin-1-yl)piperidin-1-yl)phenyl)amino)pyrimidin-4-yl)amino)quinoxalin-5-yl)dimethylphosphine oxide

Prepare the following Comparative compound C as described for Example 34 in WO2019015655.

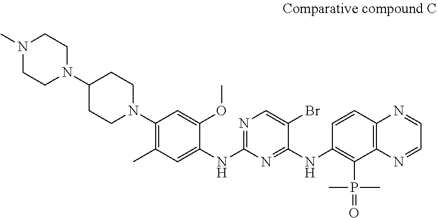 [SMILES: COc1cc(N2CCC(N3CCN(C)CC3)CC2)c(C)cc1Nc1ncc(Br)c(Nc2ccc3nccnc3c2P(C)(C)=O)n1]

### Comparative Compound D

### (6-((5-bromo-2-((5-ethyl-2-methoxy-4-(4-(4-methylpiperazin-1-yl)piperidin-1-yl)phenyl)amino)pyrimidin-4-yl)amino)quinoxalin-5-yl)dimethylphosphine oxide

Prepare the following Comparative compound D as described for Example 41 in WO2019015655.

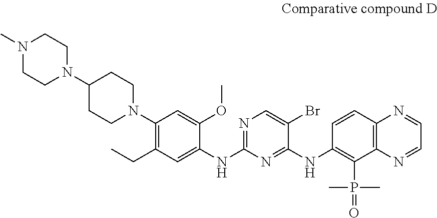 [SMILES: CCc1cc(Nc2ncc(Br)c(Nc3ccc4nccnc4c3P(C)(C)=O)n2)c(OC)cc1N1CCC(N2CCN(C)CC2)CC1]

### Pharmacological Testing

Test 1 Kinase Assay for EGFR Δ19del/T790M/C797S, EGFR T790M/L858R and EGFR L858R

Mobility shift assay was performed to determine that the compounds exhibit affinity for EGFR Δ19del/T790M/C797S, EGFR T790M/L858R and EGFR L858R. Enzyme reaction protocol are as followed:

1. Preparing 1* Kinase buffer as followed.

| 1*kinase buffer | Final |
|---|---|
| HEPES PH7.5 (mM) | 50 |
| Brij-35 | 0.0150% |
| DTT (mM) | 2 |
| Mgcl₂, Mncl₂ (mM) | 10 |

2. Preparing Compound Concentration Gradient: Compounds were tested at a concentration of 300 nM, diluted to 100-fold final concentration in 100% DMSO solution in 96-well plates, and compounds were diluted 3 times with Precision, 10 concentrations. Each concentration of the compound was then further diluted to a 5-fold final concentration of the intermediate dilution solution using 1* Kinase buffer.

3. 5 μL of each of the prepared intermediate dilution compounds was separately added to the compound wells of the 384-well plate, and each concentration was tested for duplicate wells; 5 μL of 5% DMSO was added to the negative control wells and the positive control wells, respectively.

4. 2.5-fold final concentration of the kinase solution was prepared using 1*Kinase buffer.

5. Add 10 μL of 2.5-fold final concentration of kinase solution to the compound well and positive control well; add 10 μL of 1*Kinase buffer to the negative control well.

6. Centrifuge at 1000 rpm for 30 seconds, shake the reaction plate and incubate for 10 minutes at room temperature.

7. A mixed solution of 2.5 times the final concentration of ATP and Kinase substrate (5-FAM-EEPLYWSFPAKKK-CONH2) was prepared using 1*Kinase buffer.

8. 10 μL of a 2.5-fold final concentration of a mixed solution of ATP and a substrate was added to initiate the reaction.

9. Centrifuge the 384-well plate at 1000 rpm for 30 seconds, mix by shaking, and incubate at room temperature for the corresponding time.

10. Add 30 μL of the stop solution to stop the kinase reaction, centrifuge at 1000 rpm for 30 seconds, and mix by shaking.

11. Read the conversion rate with Caliper EZ Reader.

Convert conversion values to inhibition values:

Percent inhibition=(max−conversion % sample)/(max−min)*100.

“max” stands for the mean value of the positive control well ratio; “min” stands for the mean value of the negative control well; conversion % sample: sample conversion reading.

Fit the data in log(inhibitor) vs. response Variable slope of GraphPad Prism 5 to obtain IC50 values.

Equation used is: Y=Bottom+(Top−Bottom)/(1+(IC50/X)*HillSlope)

The result is expressed with IC50, shown as Table 1, Compounds of the present disclosure, as exemplified in the Examples, showed IC50 values in the following ranges: “A” stands for “IC50≤10 nM”; “B” stands for “10 nM<IC₅₀≤100 nM”; “C” stands for “IC₅₀>100 nM”.

TABLE 1

| | EGFR Δ 19del/ | EGFR | EGFR |
|---|---|---|---|
| | T790M/C797S | L858R | T790M/L858R |
| | IC₅₀ | IC₅₀ | IC₅₀ |
| No. | (nm) | (nm) | (nm) |
| 1 | A | B | A |
| 2 | A | B | A |
| 3 | A | B | A |
| 4 | A | B | A |
| 5 | A | B | A |
| 6 | A | — | — |
| 7 | A | — | — |
| 8 | A | — | — |
| 9 | A | — | — |
| 10 | A | — | — |
| 11 | A | — | — |
| 12 | — | — | — |
| 13 | — | — | — |
| 14 | A | A | A |
| 15 | A | A | A |
| 16 | A | A | A |
| 17 | A | A | A |
| 18 | A | A | A |
| 19 | A | A | A |
| 20 | A | A | A |
| 21 | B | A | A |
| 22 | A | B | A |
| 23 | A | — | — |
| 24 | A | B | A |
| 25 | B | A | A |
| 26 | A | B | A |
| 27 | B | A | A |
| 28 | B | B | B |
| 29 | A | B | A |
| 30 | A | — | — |
| 31 | — | — | — |
| 32 | B | A | A |
| 33 | A | B | A |
| 34 | A | — | — |
| 35 | A | — | — |
| 36 | A | A | A |
| 37 | B | A | A |
| 38 | B | B | B |
| 39 | C | A | A |
| 40 | B | B | B |
| 41 | A | B | A |

Note:
“—” stands for “not tested”

Test 2 Ba/F3-Δ19del/T790M/C797S and Ba/F3-L858R/T790M/C797S Cells Proliferation Assay

1. Cell culture

Cell line: Ba/F3 cells with Δ19del/T790M/C797S or L858R/T790M/C797S mutation gene stably overexpressed named Ba/F3-Δ19del/T790M/C797S and Ba/F3-L858R/T790M/C797S.

A. Culture medium

RPMI 1640 and 10% FBS and 1% PS.

B. Cell recovery

a) The medium was preheated in a 37 water bath in advance.

b) Remove the cryogenic vials from the liquid nitrogen tank, quickly put it into a 37° C. water bath, and completely melt it in 1 min.

c) Transfer the cell suspension to a 15 mL centrifuge tube containing 8 mL medium, centrifuge 1000 rpm, 5 min,

d) Discard the supernatant, resuspend the cells in 1 mL medium, and transfer to a 75 cm² flask containing 15 mL medium, culture the cells in an incubator at 37° C., 5% CO₂.

C. Cell passage

a) The medium was preheated in a 37° C.; water bath in advance.

b) Collect cell to a 15 mL centrifuged tube, centrifuge at 1000 rpm for 5 min. Discard the supernatant, count, and make the cell density at 1×10⁴ cells/mL, then place it in a 37° C., 5% CO₂ incubator.

2. Compound preparation

a) The test compound (20 mM stock solution) was diluted to 200 uM with 100% DMSO as starting concentration then 3-fold serial diluted with “9+0” concentrations. in a 96-well dilution plate (Cat #P-05525, Labcyte);

b) The above compound solution was diluted 1:20 times with culture medium to prepare a 10 fold working solution;

3. Cell plating

a) Take cells in log phase growth, centrifuge at 1000 rpm for 5 min, then resuspend the cells with culture medium, then count cells;

b) Cells were seeded to 96-well cell culture plate with density at 2000 cells/well;

4. Compound treatment

a) Compounds prepared at step 2 were added to cell plate with 15 μL per well, the final concentrations were 1000,333, 111.1, 37,12.3, 4.1, 1.4, 0.5, 0.2 and 0 nM, and the final concentration of DMSO was 0.5%. The blank control well was a culture medium (0.5% DMSO);

b) The cells were incubated for an additional 72 h in the incubator.

5. Detection

a) Remove the 96-well cell culture plate and add 50 μl of CTG reagent (CellTiter Glo kit, promega, Cat #G7573).

b) Plate was shaked for 2 min and then let it cool for 10 min at room temperature.

c) The Luminescence signal value was read using a PerkinElmer reader.

Experimental Data Analysis

Data were analyzed using GraphPad Prism 6.0 software to obtain a fitted curve of compound activity.

Fit the Compound IC50 from non-linear regression equation:

Y=Bottom+(Top−Bottom)/(1+10\hat{ }((Log IC50−X)*Hill Slope));

X: The log of the concentration of the compound; Y: Luminescence value.

Test 3 H1975 and HCC827 Cells Proliferation Assay

1. Cell culture

Cell line: H1975(L858R/T790M) and HCC827(Δ19del)

A. Culture medium

RPMI 1640 and 10% FBS and 1% PS.

B. Cell recovery

a) The medium was preheated in a 37° C. water bath in advance.

b) Remove the cryogenic vials from the liquid nitrogen tank, quickly put it into a 37° C. water bath, and completely melt it in 1 min.

c) Transfer the cell suspension to a 15 mL centrifuge tube containing 8 mL medium, centrifuge 1000 rpm, 5 min.

d) Discard the supernatant, resuspend the cells in 1 mL medium, and transfer to a 75 cm² flask containing 15 mL medium, culture the cells in an incubator at 37° C., 5% CO₂.

C. Cell passage

a) The medium was preheated in a 37° C. water bath in advance.

b) Collect cell to a 15 mL centrifuged tube, centrifuge at 1000 rpm for 5 min. Discard the supernatant, count, and make the cell density at 1×10⁴ cells/mL, then place it in a 37° C., 5% CO₂ incubator.

2. Compound preparation

a) The test compound (20 mM stock solution) was diluted to 2 mM with 100% DMSO as starting concentration then 3-fold serial diluted with “9+0” concentrations. in a 96-well dilution plate (Cat #P-05525, Labcyte);

b) The above compound solution was diluted 1:20 times with culture medium to prepare a 10 fold working solution;

3. Cell plating

a) Take cells in log phase growth, centrifuge at 1000 rpm for 5 min, then resuspend the cells with culture medium, then count cells;

b) Cells were seeded to 96-well cell culture plate with density at 2000 cells/well (suitable for H1975 cell) and at 2500 cells/well (suitable for HCC827 cell);

4. Compound treatment

a) Compounds prepared at step 2 were added to cell plate with 15 μL per well, the final concentrations were 10000, 3333,1111.1, 370.4, 123.5, 41.2, 13.7, 4.6, 1.5 and 0 nM, and the final concentration of DMSO was 0.5%. The blank control well was a culture medium (0.5% DMSO);

b) The cells were incubated for an additional 72 h in the incubator.

5. Detection

a) Remove the 96-well cell culture plate and add 50 μl of CTG reagent (CellTiter Glo kit, promega, Cat #G7573).

b) Plate was shaked for 2 min and then let it cool for 10 min at room temperature.

c) The Luminescence signal value was read using a PerkinElmer reader.

Experimental Data Analysis

Data were analyzed using GraphPad Prism 6.0 software to obtain a fitted curve of compound activity.

Fit the Compound IC50 from non-linear regression equation:

Y=Bottom+(Top−Bottom)/(1+10\hat{ }((Log IC50−X)*HillSlope));

X: The log of the concentration of the compound; Y: Luminescence value.

The cells proliferation assay results are expressed with IC50, shown as Table 2. Compounds of the present disclosure, as exemplified in the Examples, showed IC50 values in the following ranges: “A” stands for “IC₅₀≤10 nM”; “B” stands for “10 nM<IC₅₀<50 nM”; “C” stands for “IC₅₀≥50 nM”.

TABLE 2

| | BaF3 | BaF3 Δ 19del/ | | |
|---|---|---|---|---|
| | L858R/T790M/C797S | T790M/C797S | H1975 | HCC827 |
| | IC₅₀ | IC₅₀ | IC₅₀ | IC₅₀ |
| No. | (nm) | (nm) | (nm) | (nm) |
| 1 | A | A | A | A |
| 2 | — | — | — | — |
| 3 | — | — | — | — |
| 4 | — | — | — | — |
| 5 | — | — | A | B |
| 6 | — | B | — | — |
| 7 | B | B | — | B |
| 8 | B | B | B | B |
| 9 | B | B | — | — |
| 10 | A | A | A | B |
| 11 | — | B | B | B |
| 12 | B | B | B | B |
| 13 | — | — | B | B |
| 14 | — | — | B | B |
| 15 | B | B | A | A |
| 16 | B | B | A | B |
| 17 | B | B | B | B |
| 18 | B | B | A | A |
| 19 | — | B | B | A |
| 20 | — | B | B | A |
| 21 | — | — | B | A |
| 22 | — | — | — | A |
| 23 | A | A | B | B |
| 24 | — | — | — | B |
| 25 | — | — | — | A |
| 26 | — | — | — | — |
| 27 | — | — | A | A |
| 28 | — | — | — | B |
| 29 | — | B | — | B |
| 30 | B | A | B | B |
| 31 | — | — | B | B |
| 32 | — | — | B | A |
| 33 | — | — | B | B |
| 34 | A | A | A | A |
| 35 | A | A | A | A |
| 36 | A | A | A | A |
| 37 | — | — | B | A |
| 38 | — | — | — | — |
| 39 | — | — | B | A |
| 40 | — | — | — | — |
| 41 | — | — | B | B |
| Comparative | A | A | 61.7 | 89.5 |
| compound A | | | | |
| Comparative | C | C | A | A |
| compound B | | | | |
| Comparative | 16.8 | 9.6 | 71.4 | — |
| compound C | | | | |
| Comparative | 12.9 | 5.7 | 83.58 | — |
| compound D | | | | |

Note:
“—” stands for “not tested”

As shown in the Table 2, we can see Comparative compound A disclosed in WO2018108064, Comparative compound C and D disclosed in WO2019015655 have higher inhibition of triple mutant (L858R/T790M/C797S or Δ19del/T790M/C797S), while at the same time having relatively low inhibition of double mutant (L858R/T790M) and single mutant (L858R). Additionally, We can see Comparative compound B (also known as AZD 9291) have higher inhibition of double mutant (L858R/T790M) and single mutant (L858R), while at the same time having relatively low inhibition of triple mutant (L858R/T790M/C797S or Δ19del/T790M/C797S). However, the exemplified compounds of this invention display high inhibition of triple mutant (L858R/T790M/C797S or 19del/T790M/C797S), double mutant (L858R/T790M) and single mutant (L858R).

1. A compound of Formula I, 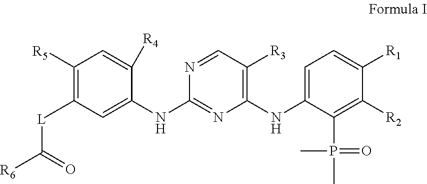
wherein,
R₁ and R₂ are each independently selected from is H, halogen, CN, —C₁₋₆ alkyl or —C₁₋₆ alkoxyl; or
R₁ and R₂ together with the atoms to which they are attached form a 5- to 6-membered heteroaryl ring optionally comprising for 2 hetero atoms independently selected from N, S, or O; or
R₁ and R₂ together with the atoms to which they are attached form an aryl ring;
R₃ is H, halogen, —C₁₋₆ alkyl;
R₄ is H, halogen, —C₁₋₆ alkyl or —C₁₋₆ alkoxyl;
R₅ is —OR₇, —O(CH₂)ₜ—NR₈R₉, —NR₈R₉, 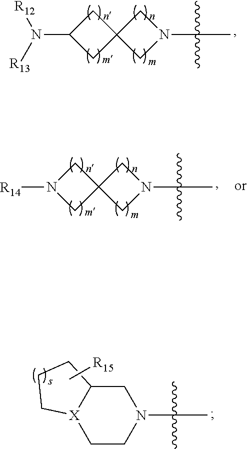
R₆ is H, —C₁₋₆ alkyl, C₂₋₆ alkenyl, C₂₋₆ alkynyl; wherein —C₁₋₆ alkyl, C₂₋₆ alkenyl, C₂₋₆ alkynyl optionally substituted with one or more substituents independently selected from halogen, —C₁₋₆ alkyl, —C₁₋₄ haloalkyl or —NR₁₆R₁₇;
R₇ is C₁₋₆ alkyl, C₃₋₁₀ heteocyclyl, or C₃₋₁₀ heteroaryl;
R₈ and R₉ are each independently selected from —C₁₋₆ alkyl, or —C₁₋₆ alkylene-NR₁₀R₁₁, wherein R₁₀ and R₁₁ are each independently selected from H or —C₁₋₆ alkyl; or R₁₀ and R₁₁ together with the atoms to which they are attached form a 5- to 6-membered heterocyclic ring; or
R₈ and R₉ together with the atoms to which they are attached form a 5- to 6-membered heterocyclic ring;
R₁₂, R₁₃, R₁₄ and R₁₅ are each independently selected from H or —C₁₋₆ alkyl;
R₁₂ and R₁₃ together with the atoms to which they are attached form a 4- to 6-membered ring;
L is a bond, NR₁₈ or (CH₂)ₜ;
R₁₆, R₁₇ and R₁₈ are each independently selected from H, or —C₁₋₆ alkyl.
X is CH or N;
m, n, m′, n′ are each independently selected from 1 or 2;
s and t are each independently selected from 1, 2 or 3,
or a stereoisomer, tautomer, deuterinated compound, pharmaceutically acceptable salt, prodrug, chelate, non-covalent complex, or solvate thereof.

2. The compound of claim 1, wherein R₁ and R₂ are each independently selected from is H, CN, and —CH₃.

3. The compound of claim 1, wherein R₁ and R₂ together with the atoms to which they are attached form

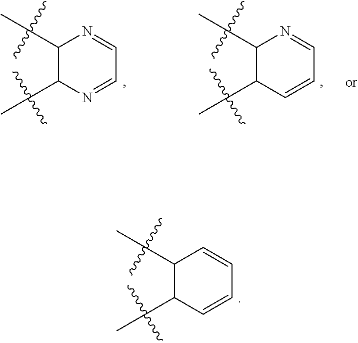 [SMILES: *C1N=CC=NC1* ; *C1C=CC=NC1* ; *C1C=CC=CC1*]

4. The compound of claim 1, wherein R₃ is selected from H, F, Cl, Br, and CH₃.

5. The compound of claim 1, wherein R₄ is selected from H, —CH₃, and —OCH₃.

6. The compound of claim 1, wherein R₅ is selected from

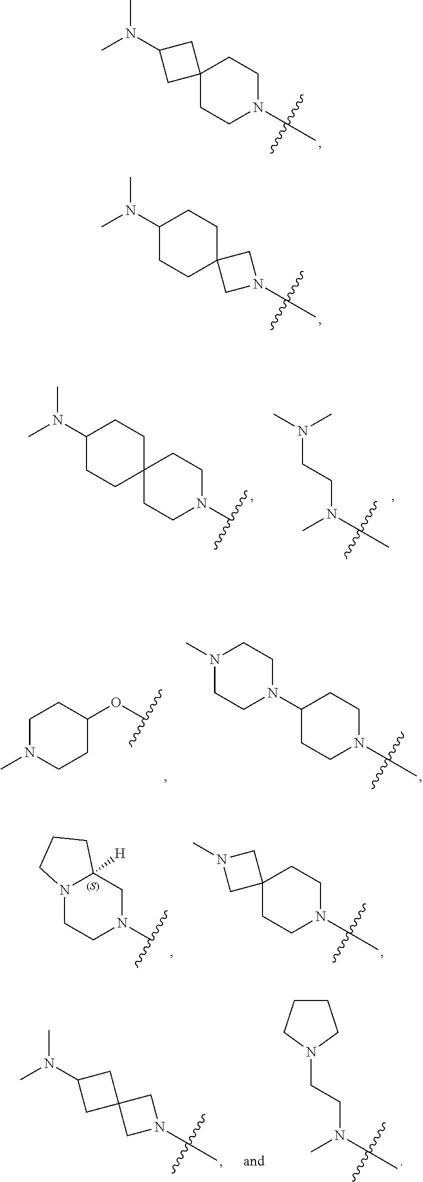

7. The compound of claim 1, wherein L is selected from —NH—, and —NCH₃—.

8. The compound of claim 1, wherein R₆ is selected from

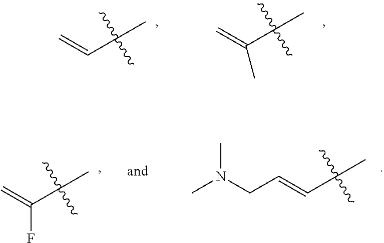

9. The compound of claim 1, wherein the compound is Formula II, or a stereoisomer, tautomer, deuterinated compound, pharmaceutically acceptable salt, prodrug, chelate, non-covalent complex, or solvate thereof, 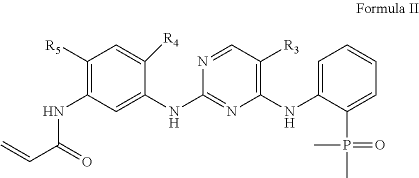
wherein,
R₃ is H, halogen, —C₁₋₆ alkyl;
R₄ is H, halogen, —C₁₋₆ alkyl or —C₁₋₆ alkoxyl;
R₅ is —OR₇, —O(CH₂)ₜ—NR₈R₉, —NR₈R₉, 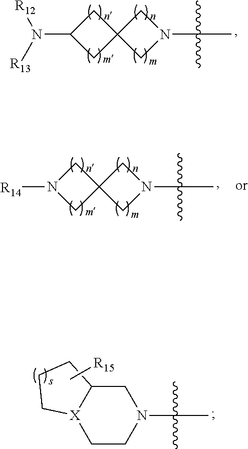
R₇ is C₁₋₆ alkyl, C₃₋₁₀ heteocyclyl, or C₃₋₁₀ heteroaryl;
R₈ and R₉ are each independently selected from —C₁₋₆ alkyl, or —C₁₋₆ alkylene-NR₁₀R₁₁, wherein
R₁₀ and R₁₁ are each independently selected from H or —C₁₋₆ alkyl; or R₁₀ and R₁₁ together with the atoms to which they are attached form a 5- to 6-membered heterocyclic ring; or
R₈ and R₉ together with the atoms to which they are attached form a 5- to 6-membered heterocyclic ring;
R₁₂, R₁₃, R₁₄ and R₁₅ are each independently selected from H or —C₁₋₆ alkyl;
R₁₂ and R₁₃ together with the atoms to which they are attached form a 4- to 6-membered ring;
X is CH or N;
m, n, m′, n′ are each independently selected from 1 or 2;
s and t are each independently selected from 1, 2 or 3.

10. The compound of claim 9, wherein R₃ is selected from H, F, Cl, and CH₃.

11. The compound of claim 9, wherein R₄ is selected from H, and —OCH₃.

12. The compound of claim 9, wherein R₅ is selected from —OR₇, —NR₈R₉,

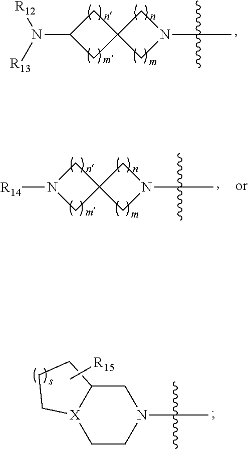

13. The compound of claim 9, wherein R₇ is C₃₋₁₀ heteocyclyl.

14. (canceled)

15. (canceled)

16. (canceled)

17. The compound of claim 1, wherein the compound is Formula II, or a stereoisomer, tautomer, deuterinated compound, pharmaceutically acceptable salt, prodrug, chelate, non-covalent complex, or solvate thereof, 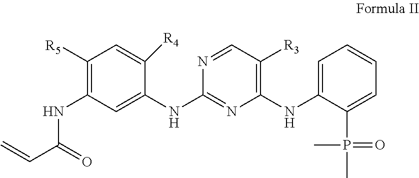
wherein,
R₃ is H, halogen, or C₁₋₆ alkyl;
R₄ is H, halogen, C₁₋₆ alkyl or C₁₋₆ alkoxyl;
R₅ is 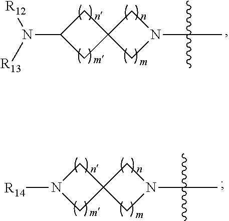
R₁₂, R₁₃, R₁₄ are each independently selected from H or C₁₋₆ alkyl;
R₁₂ and R₁₃ together with the atoms to which they are attached form a 4- to 6-membered ring;
m, n, m′, n′ are each independently selected from 1 or 2.

18. (canceled)

19. (canceled)

20. (canceled)

21. (canceled)

22. (canceled)

23. (canceled)

24. (canceled)

25. (canceled)

26. The compound of claim 1, wherein the compound is Formula IV, or a stereoisomer, tautomer, deuterinated compound, pharmaceutically acceptable salt, prodrug, chelate, non-covalent complex, or solvate thereof, 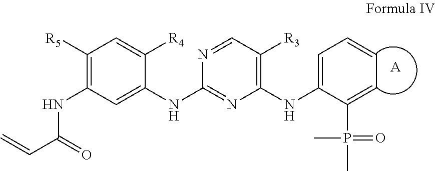
wherein,
Ring A is selected from aryl ring or 5- to 6-membered heteroaryl ring optionally comprising for 2 hetero atoms independently selected from N, S, or O;
R₃ is H, halogen, or —C₁₋₆ alkyl;
R₄ is H, halogen, —C₁₋₆ alkyl or —C₁₋₆ alkoxyl;
R₅ is —OR₇, —O(CH₂)ₜ—NR₈R₉, —NR₈R₉, 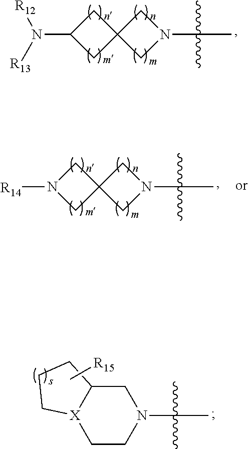
R₇ is C₁₋₆ alkyl, C₃₋₁₀ heteocyclyl, or C₃₋₁₀ heteroaryl;
R₈ and R₉ are each independently selected from —C₁₋₆ alkyl, or —C₁₋₆ alkyl-NR₁₀R₁₁, wherein R₁₀ and R₁₁ are each independently selected from H or —C₁₋₆ alkyl; or R₁₀ and R₁₁ together with the atoms to which they are attached form a 5- to 6-membered heterocyclic ring; or
R₈ and R₉ together with the atoms to which they are attached form a 5- to 6-membered heterocyclic ring;
R₁₂, R₁₃, R₁₄ and R₁₅ are each independently selected from H or —C₁₋₆ alkyl;
R₁₂ and R₁₃ together with the atoms to which they are attached form a 4- to 6-membered ring;
m, n, m′, n′ are each independently selected from 1 or 2;
s and t are each independently selected from 1, 2 or 3.

27. (canceled)

28. (canceled)

29. (canceled)

30. (canceled)

31. (canceled)

32. (canceled)

33. (canceled)

34. (canceled)

35. (canceled)

36. (canceled)

37. The compound of claim 1, wherein the compound is
1) N-(5-((5-chloro-4-((2-(dimethylphosphoryl)phenyl)amino)pyrimidin-2-yl)amino)-2-(9-(dim ethylamino)-3-azaspiro[5.5]undecan-3-yl)phenyl)acrylamide;
2) N-(5-((5-chloro-4-((2-(dimethylphosphoryl)phenyl)amino)pyrimidin-2-yl)amino)-2-(7-(dim ethylamino)-2-azaspiro[3.5]nonan-2-yl)phenyl)acrylamide;
3) N-(5-((5-chloro-4-((2-(dimethylphosphoryl)phenyl)amino)pyrimidin-2-yl)amino)-2-(6-(dim ethylamino)-2-azaspiro[3.3]heptan-2-yl)phenyl)acrylamide;
4) N-(2-(2-(dimethylamino)-7-azaspiro[3.5]nonan-7-yl)-5-((4-((2-(dimethylphosphoryl)phenyl)amino)pyrimidin-2-yl)amino)phenyl)acrylamide;
5) N-(5-((5-chloro-4-((2-(dimethylphosphoryl)phenyl)amino)pyrimidin-2-yl)amino)-2-(2-(dim ethylamino)-7-azaspiro[3.5]nonan-7-yl)-4-methoxyphenyl)acrylamide;
6) N-(5-((5-chloro-4-((2-(dimethylphosphoryl)phenyl)amino)pyrimidin-2-yl)amino)-2-(2-(dim ethylamino)-7-azaspiro[3.5]nonan-7-yl)phenyl)-N-methylacrylamid;
7) N-(5-((5-chloro-4-((2-(dimethylphosphoryl)phenyl)amino)pyrimidin-2-yl)amino)-2-(2-(dim ethylamino)-7-azaspiro[3.5]nonan-7-yl)phenyl)methacrylamide;
8) (E)-N-(5-((5-chloro-4-((2-(dimethylphosphoryl)phenyl)amino)pyrimidin-2-yl)amino)-2-(2-(dimethylamino)-7-azaspiro[3.5]nonan-7-yl)phenyl)-4-(dimethylamino)but-2-enamide;
9) N-(5-((5-chloro-4-((2-(dimethylphosphoryl)phenyl)amino)pyrimidin-2-yl)amino)-2-(2-(dim ethylamino)-7-azaspiro[3.5]nonan-7-yl)phenyl)-2-fluoroacrylamide;
10) N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)phenyl)acrylamide;
11) N-(5-((5-chloro-4-((5-(dimethylphosphoryl)quinoxalin-6-yl)amino)pyrimidin-2-yl)amino)-2-(2-(dimethylamino)-7-azaspiro[3.5]nonan-7-yl)-4-methoxyphenyl)acrylamide;
12) N-(5-((5-bromo-4-((5-(dimethylphosphoryl)quinoxalin-6-yl)amino)pyrimidin-2-yl)amino)-2-(4-(4-methylpiperazin-1-yl)piperidin-1-yl)phenyl)acrylamide;
13) N-(5-((5-bromo-4-((5-(dimethylphosphoryl)quinoxalin-6-yl)amino)pyrimidin-2-yl)amino)-4-methoxy-2-(4-(4-methylpiperazin-1-yl)piperidin-1-yl)phenyl)acrylamide;
14) N-(5-((5-chloro-4-((2-(dimethylphosphoryl)phenyl)amino)pyrimidin-2-yl)amino)-2-(2-methyl-2,7-diazaspiro[3.5]nonan-7-yl)phenyl)acrylamide hydrochloric acid salt;
15) N-(5-((5-chloro-4-((2-(dimethylphosphoryl)phenyl)amino)pyrimidin-2-yl)amino)-2-((2-(dimethylamino)ethyl)(methyl)amino)phenyl)acrylamide;
16) N-(5-((5-chloro-4-((2-(dimethylphosphoryl)phenyl)amino)pyrimidin-2-yl)amino)-2-(4-(4-methylpiperazin-1-yl)piperidin-1-yl)phenyl)acrylamide;
17) (S)—N-(5-((5-chloro-4-((2-(dimethylphosphoryl)phenyl)amino)pyrimidin-2-yl)amino)-2-(hexahydropyrrolo[1,2-a]pyrazin-2(1H)-yl)phenyl)acrylamide;
18) N-(5-((5-chloro-4-((2-(dimethylphosphoryl)phenyl)amino)pyrimidin-2-yl)amino)-2-(methyl(2-(pyrrolidin-1-yl)ethyl)amino)phenyl)acrylamide;
19) N-(2-((2-(dimethylamino)ethyl)(methyl)amino)-5-((4-((2-(dimethylphosphoryl)phenyl)amino)-5-methylpyrimidin-2-yl)amino)phenyl)acrylamide hydrochloric acid salt;
20) N-(5-((4-((2-(dimethylphosphoryl)phenyl)amino)-5-methylpyrimidin-2-yl)amino)-2-(4-(4-methylpiperazin-1-yl)piperidin-1-yl)phenyl)acrylamide;
21) N-(2-((2-(dimethylamino)ethyl)(methyl)amino)-5-((4-((2-(dimethylphosphoryl)phenyl)amino)-5-methylpyrimidin-2-yl)amino)-4-methoxyphenyl)acrylamide;
22) N-(5-((4-((2-(dimethylphosphoryl)phenyl)amino)-5-methylpyrimidin-2-yl)amino)-4-methoxy-2-(4-(4-methylpiperazin-1-yl)piperidin-1-yl)phenyl)acrylamide;
23) N-(5-((5-chloro-4-((1-(dimethylphosphoryl)naphthalen-2-yl)amino)pyrimidin-2-yl)amino)-2-(2-(dimethylamino)-7-azaspiro[3.5]nonan-7-yl)phenyl)acrylamide hydrochloric acid salt;
24) N-(5-((5-chloro-4-((2-(dimethylphosphoryl)phenyl)amino)pyrimidin-2-yl)amino)-4-methoxy-2-(4-(4-methylpiperazin-1-yl)piperidin-1-yl)phenyl)acrylamide;
25) N-(5-((5-chloro-4-((2-(dimethylphosphoryl)phenyl)amino)pyrimidin-2-yl)amino)-2-((2-(di methylamino)ethyl)(methyl)amino)-4-methoxyphenyl)acrylamide;
26) N-(5-((5-chloro-4-((2-(dimethylphosphoryl)phenyl)amino)pyrimidin-2-yl)amino)-2-(7-(dim ethylamino)-2-azaspiro[3.5]nonan-2-yl)-4-methoxyphenyl)acrylamide;
27) N-(2-((2-(dimethylamino)ethyl)(methyl)amino)-5-((4-((2-(dimethylphosphoryl)phenyl)amino)-5-fluoropyrimidin-2-yl)amino)-4-methoxyphenyl)acrylamide;
28) N-(5-((4-((2-(dimethylphosphoryl)phenyl)amino)-5-fluoropyrimidin-2-yl)amino)-4-methoxy-2-(4-(4-methylpiperazin-1-yl)piperidin-1-yl)phenyl)acrylamide;
29) N-(5-((4-((2-(dimethylphosphoryl)phenyl)amino)-5-fluoropyrimidin-2-yl)amino)-2-(4-(4-m ethylpiperazin-1-yl)piperidin-1-yl)phenyl)acrylamide;
30) N-(5-((5-chloro-4-((5-(dimethylphosphoryl)quinolin-6-yl)amino)pyrimidin-2-yl)amino)-2-(2-(dimethylamino)-7-azaspiro[3.5]nonan-7-yl)phenyl)acrylamide;
31) N-(5-((5-chloro-4-((2-(dimethylphosphoryl)phenyl)amino)pyrimidin-2-yl)amino)-2-(2-(dim ethylamino)-7-azaspiro[3.5]nonan-7-yl)-4-methylphenyl)acrylamide;
32) N-(2-((2-(dimethylamino)ethyl)(methyl)amino)-5-(4-(2-(dimethylphosphoryl)phenylamino)-5-fluoropyrimidin-2-ylamino)phenyl)acrylamide;
33) N-(5-(5-chloro-4-(2-(dimethylphosphoryl)phenylamino)pyrimidin-2-ylamino)-2-(9-(dimeth ylamino)-3-azaspiro[5.5]undecan-3-yl)-4-methoxyphenyl)acrylamide;
34) N-(5-(5-bromo-4-(5-(dimethylphosphoryl)quinoxalin-6-ylamino)pyrimidin-2-ylamino)-2-((2-(dimethylamino)ethyl)(methyl)amino)phenyl)acrylamide;
35) N-(5-(5-bromo-4-(5-(dimethylphosphoryl)quinoxalin-6-ylamino)pyrimidin-2-ylamino)-2-(2-(dimethylamino)-7-azaspiro[3.5]nonan-7-yl)phenyl)acrylamide;
36) N-(5-((5-chloro-4-((2-(dimethylphosphoryl)phenyl)amino)pyrimidin-2-yl)amino)-2-(2-(dim ethylamino)-7-azaspiro[3.5]nonan-7-yl)phenyl)acrylamide;
37) N-(2-((2-(dimethylamino)ethyl)(methyl)amino)-5-((4-((2-(dimethylphosphoryl)phenyl)amino)pyrimidin-2-yl)amino)phenyl)acrylamide;
38) N-(5-((4-((2-(dimethylphosphoryl)phenyl)amino)pyrimidin-2-yl)amino)-2-(4-(4-methylpiperazin-1-yl)piperidin-1-yl)phenyl)acrylamide;
39) N-(2-((2-(dimethylamino)ethyl)(methyl)amino)-5-((4-((2-(dimethylphosphoryl)phenyl)amino)pyrimidin-2-yl)amino)-4-methoxyphenyl)acrylamide;
40) N-(5-((4-((2-(dimethylphosphoryl)phenyl)amino)pyrimidin-2-yl)amino)-4-methoxy-2-(4-(4-methylpiperazin-1-yl)piperidin-1-yl)phenyl)acrylamide;
41) N-(5-((5-chloro-4-((2-(dimethylphosphoryl)phenyl)amino)pyrimidin-2-yl)amino)-2-((l-methylpiperidin-4-yl)oxy)phenyl)acrylamide.

38. A pharmaceutical composition comprising a compound of claim 1, or a pharmaceutically acceptable salt or a stereoisomer thereof, and at least one pharmaceutically acceptable carrier or excipient.

39. (canceled)

40. A method of treating an EGFR-driven cancer, said method comprising administering to a patient in need thereof a therapeutically effective amount of a compound of claim 1, or a pharmaceutically acceptable salt or a stereoisomer thereof.

41. The method of claim 40, wherein the EGFR-driven cancer is characterized by the presence of one or more mutations selected from: (i) C797S, (ii) both L858R and C797S, (iii) both C797S and T790M, (iv) L858R, T790M, and C797S, and (v) Δ19del, T790M and C797S.

42. The method of claim 40, wherein the EGFR-driven cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

43. (canceled)

44. (canceled)

45. (canceled)

46. (canceled)
